SEQUENCE LISTING (140 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, artificial
  mature human IL-15-AQ
NWVNVISDLKKIEDLIQSMHIDATLYTESDVHPSCKVTAMKCFLLELQVISLESGDASIHDTVENLIILANNSLSSNAQVTESGCKECEELEEKNIKEFLQSFVHIVQMFINTS

SEQ ID NO: 4 - protein, 267 aa, homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 5 - protein, 61 aa, homo sapiens
CPPPMSVEHADIWVKSYSLYSRERYICNSGFKRKAGTSSLTECVLNKATNVAHWTTPSLKC

SEQ ID NO: 6 - protein, 77 aa, homo sapiens
ITCPPPMSVEHADIWVKSYSLYSRERYICNSGFKRKAGTSSLTECVLNKATNVAHWTTPSLKCIRDPALVHQRPAPP

SEQ ID NO: 7 - protein, 20 aa, artificial
  linker
SGGSGGGGSGGGSGGGGSGG

SEQ ID NO: 8 - protein, 211 aa, artificial
  RLI2
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - protein, 211 aa, artificial
  RLI2-AQ
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNAQVTESGCKECEELEEKNIKEFLQSFVHIVQMFINTS

SEQ ID NO: 10 - protein, 211 aa, artificial
  RLI2AQ N162A (N65A)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 11 - protein, 211 aa, artificial
  RLI2AQ D158A/N162A (D158A/N162A)
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHATVEALIILANNSLSSNAQVTESGCKECEELEEKNIKEFLQSFVHIVQMFINTS

SEQ ID NO: 12 - protein, 446 aa, artificial
  pembrolizumab heavy chain (HC) - human IgG4 k isotype
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 5 aa, artificial
  pembrolizumab HC CDR1
NYYMY

SEQ ID NO: 14 - protein, 17 aa, artificial
  pembrolizumab HC CDR2
GINPSNGGTNFNEKFKN

SEQ ID NO: 15 - protein, 11 aa, artificial
  pembrolizumab HC CDR3
RDYRFDMGFDY

SEQ ID NO: 16 - protein, 218 aa, artificial
  pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 15 aa, artificial
  pembrolizumab LC CDR1
RASKGVSTSGYSYLH

SEQ ID NO: 18 - protein, 7 aa, artificial
  pembrolizumab LC CDR2
LASYLES

SEQ ID NO: 19 - protein, 9 aa, artificial
  pembrolizumab LC CDR3
QHSRDLPLT

SEQ ID NO: 20 - protein, 446 aa, artificial
  pembrolizumab HC knob
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 657 aa, artificial
  SOT201 HC knob
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 22 - protein, 447 aa, artificial
  pembrolizumab/SOT201 HC hole
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 23 - protein, 218 aa, artificial; identical to SEQ ID NO: 16
  SOT201 LC

SEQ ID NO: 24 - protein, 113 aa, artificial
  IgG1 CH1 with hinge
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 25 - protein, 217 aa, artificial
  IgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 217 aa, artificial
  IgG1 Fc LALA
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 217 aa, artificial
  IgG1 Fc LALA PG
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 217 aa, artificial
  IgG1 Fc KiH - knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 217 aa, artificial
  IgG1 Fc KiH - hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 217 aa, artificial
  IgG1 Fc DE (S239D/I332E)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 217 aa, artificial
  IgG1 Fc DLE (S239D/A330L/I332E)
APELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 217 aa, artificial
  IgG1 Fc DAE (S239D/I332E/G236A)
APELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 217 aa, artificial
  IgG1 Fc GASDALIE (G236A/S239D/A330L/I332E)
APELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 217 aa, artificial
  IgG1 Fc AAA (S298A/E333A/K334A)
APELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 217 aa, artificial
  IgG1 Fc YTE (M252Y/S254T/T256E)
APELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 217 aa, artificial
  IgG1 Fc TL (K392T/P396L)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYTTTPLVLDSDGSFFLYSKLTVDKSRWQQGNVFSCSVMHEALHNHYTQKSLSLSPGK

SEQ ID NO: 37 - protein, 217 aa, artificial
  IgG1 Fc IE (V264I/I332E)
APELLGGPSVFLFPPKPKDTLMISRTPEVTCVVIDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 107 aa, artificial [sequence omitted]

SEQ ID NO: 39 - protein, 110 aa, artificial
  IgG4 CH1 hinge S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCP

SEQ ID NO: 40 - protein, 217 aa, artificial
  IgG4 Fc
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 217 aa, artificial
  IgG4 Fc KiH - knob
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 217 aa, artificial
  IgG4 Fc KiH - hole
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 43 - protein, 217 aa, artificial
  IgG4 Fc LE (L235E)
APEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 217 aa, artificial
  IgG4 Fc YTE (M252Y/S254T/T256E)
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 318 aa, artificial
  LC (kappa) - RLI AQ
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANNSLSSNAQVTESGCKECEELEEKNIKEFLQSFVHIVQMFINTS

SEQ ID NO: 46 - protein, 119 aa, artificial
  hCl1a VH
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSS

SEQ ID NO: 47 - protein, 107 aa, artificial
  hCl1a VL
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIK

SEQ ID NO: 48 - protein, 119 aa, artificial
  hCl1b VH
QVQLVQSGAEVKKPGASVKVSCKASGYTFTDYAMHWVRQAPGQRLEWMGWINTYTGKPTYSQKFQGRVTITRDTSASTAYMELSSLRSEDTAVYYCARAVFYGYTMDAWGQGTLVTVSS

SEQ ID NO: 49 - protein, 107 aa, artificial; identical to SEQ ID NO: 47
  hCl1b VL

SEQ ID NO: 50 - protein, 119 aa, artificial; identical to SEQ ID NO: 48
  hCl1c VH

SEQ ID NO: 51 - protein, 107 aa, artificial
  hCl1c VL
DIQMTQSPSSLSASVGDRVTITCRTSEDIYSNLAWYQQKPGKAPKLLIFAIKRLQDGVPSRFSGSGSGTDFTLTISSLQPEDFATYYCLQGSKFPLTFGQGTKVEIK

SEQ ID NO: 52 - protein, 119 aa, artificial; identical to SEQ ID NO: 48
  hCl1d VH

SEQ ID NO: 53 - protein, 107 aa, artificial
  hCl1d VL
DIQMTQSPSSLSASVGDRVTITCRTSEDIYSNFAWYQQKPGKAPKLLIYSVNRLQDGVPSRFSGSGSGTDFTLTISSLQPEDFATYYCLQGSKFPLTFGQGTKVEIK

SEQ ID NO: 54 - protein, 119 aa, artificial
  hCl1e VH
QVQLVQSGAEVKKPGASVKVSCKASGYTFTDYAMYWVRQAPGQRLEWMGWINTYTGKPTYAQKFQGRVTITRDTSASTAYMELSSLRSEDTAVYYCARAVFYGYTMDAWGQGTLVTVSS

SEQ ID NO: 55 - protein, 107 aa, artificial; identical to SEQ ID NO: 47
  hCl1e VL

SEQ ID NO: 56 - protein, 119 aa, artificial
  hCl1f VH
QVQLVQSGAEVKKPGASVKVSCKASGYTFTDYAMHWVRQAPGQRLEWMGWINAYTGKPTYAQKFQGRVTITRDTSASTAYMELSSLRSEDTAVYYCARAVFYGYTMDAWGQGTLVTVSS

SEQ ID NO: 57 - protein, 107 aa, artificial
  hCl1f VL
DIQMTQSPSSLSASVGDRVTITCRASEDIYSNLAWYQQKPGKAPKLLIYSVKRLQDGVPSRFSGSGSGTDFTLTISSLQPEDFATYYCLQGSNFPLTFGQGTKVEIK

SEQ ID NO: 58 - protein, 119 aa, artificial; identical to SEQ ID NO: 56
  hCl1g VH

SEQ ID NO: 59 - protein, 107 aa, artificial; identical to SEQ ID NO: 53
  hCl1g VL

SEQ ID NO: 60 - protein, 119 aa, artificial
  hCl1h VH
QVQLVQSGAEVKKPGASVKVSCKASGYTFTDYAMYWVRQAPGQRLEWMGWINAYTGKPTYAQKFQGRVTITRDTSASTAYMELSSLRSEDTAVYYCARAVYYGYTMDAWGQGTLVTVSS

SEQ ID NO: 61 - protein, 107 aa, artificial; identical to SEQ ID NO: 57
  hCl1h VL

SEQ ID NO: 62 - protein, 119 aa, artificial; identical to SEQ ID NO: 60
  hCl1i VH

SEQ ID NO: 63 - protein, 107 aa, artificial; identical to SEQ ID NO: 47
  hCl1i VL

SEQ ID NO: 64 - protein, 119 aa, artificial; identical to SEQ ID NO: 60
  hCl1j VH

SEQ ID NO: 65 - protein, 107 aa, artificial; identical to SEQ ID NO: 51
  hCl1j VL

SEQ ID NO: 66 - protein, 118 aa, artificial
  hGBA-1 VH
EVQLVQSGAEVKKPGESLKISCKGSGYSFTSYWIGWVRQMPGKGLEWMGNIYPGASDTRYAPSFQGQVTISADKSISTAYLQWSSLKASDTAMYYCARLWRGNSFDYWGQGTLVTVSS

SEQ ID NO: 67 - protein, 113 aa, artificial
  hGBA-1 VL
DIVMTQSPDSLAVSLGERATINCKSSQSLLNSGNQKNYLAWYQQKPGQPPKLLIYWASTRESGVPDRFSGSGSGTDFTLTISSLQAEDVAVYYCQNDYSYPFTFGQGTKVEIK

SEQ ID NO: 68 - protein, 118 aa, artificial
  hGBA-2 VH
EVQLVQSGAEVKKPGESLKISCKGSGYSFTSYWIGWVRQMPGKGLEWMGNIYPGDADTRYAPSFQGQVTISADKSISTAYLQWSSLKASDTAMYYCARMWRGNSFDYWGQGTLVTVSS

SEQ ID NO: 69 - protein, 113 aa, artificial; identical to SEQ ID NO: 67
  hGBA-2 VL

SEQ ID NO: 70 - protein, 118 aa, artificial
  hGBA-3 VH
EVQLVQSGAEVKKPGESLKISCKGSGYSFTSYWIGWVRQMPGKGLEWMGIIYPGASDTNYAPSFQGQVTISADKSISTAYLQWSSLKASDTAMYYCARIWRGNSFDYWGQGTLVTVSS

SEQ ID NO: 71 - protein, 113 aa, artificial
  hGBA-3 VL
DIVMTQSPDSLAVSLGERATINCKSSQSLLNSGNQKNYLAWYQQKPGQPPKLLIYWASTRESGVPDRFSGSGSGTDFTLTISSLQAEDVAVYYCQNDYSYPLTFGQGTKVEIK

SEQ ID NO: 72 - protein, 118 aa, artificial
  hGBA-4 VH
EVQLVQSGAEVKKPGESLKISCKGSGYSFTSYWIGWVRQMPGKGLEWMGIIYPGDAYTRYSPSFQGQVTISADKSISTAYLQWSSLKASDTAMYYCTRLWRGNSFDAWGQGTLVTVSS

SEQ ID NO: 73 - protein, 113 aa, artificial
  hGBA-4 VL
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPLTFGQGTKVEIK

SEQ ID NO: 74 - protein, 118 aa, artificial
  hGBA-5 VH
EVQLVQSGAEVKKPGESLKISCKGSGYSFTSYWIGWVRQMPGKGLEWMGIIYPGAAYTRYAPSFQGQVTISADKSISTAYLQWSSLKASDTAMYYCARLWRGNSFDYWGQGTLVTVSS

SEQ ID NO: 75 - protein, 113 aa, artificial
  hGBA-5 VL
DIVMTQSPDSLAVSLGERATINCKSSQSLLNSGNQKNYLAWYQQKPGQPPKLLIYWASTRESGVPDRFSGSGSGTDFTLTISSLQAEDVAVYYCQQDYSYPFTFGQGTKVEIK

SEQ ID NO: 76 - protein, 118 aa, artificial
  hGBA-6 VH
EVQLVQSGAEVKKPGESLKISCKGSGYTFTSYWIGWVRQMPGKGLEWMGNIYPGASYTRYSPSFQGQVTISADKSISTAYLQWSSLKASDTAMYYCTRQWRGNSFDYWGQGTLVTVSS

SEQ ID NO: 77 - protein, 113 aa, artificial; identical to SEQ ID NO: 67
  hGBA-6 VL

SEQ ID NO: 78 - protein, 118 aa, artificial
  hGBA-7 VH
EVQLVQSGAEVKKPGESLKISCKGSGYSFTSYWIGWVRQMPGKGLEWMGNIYPGEAYTRYSPSFQGQVTISADKSISTAYLQWSSLKASDTAMYYCTRLWRGNSFDYWGQGTLVTVSS

SEQ ID NO: 79 - protein, 113 aa, artificial
  hGBA-7 VL
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQNDYSYPFTFGQGTKVEIK

SEQ ID NO: 80 - protein, 118 aa, artificial
  hGBA-8 VH
EVQLVQSGAEVKKPGESLKISCKGSGYSFTSYWIGWVRQMPGKGLEWMGNIYPSESYTNYAPSFQGQVTISADKSISTAYLQWSSLKASDTAMYYCTRLWRGNSFDYWGQGTLVTVSS

SEQ ID NO: 81 - protein, 113 aa, artificial; identical to SEQ ID NO: 67
  hGBA-8 VL

SEQ ID NO: 82 - protein, 118 aa, artificial
  hGBA-9 VH
EVQLVQSGAEVKKPGESLKISCKGSGYTFTSYWIGWVRQMPGKGLEWMGIIYPSAAYTRYAPSFQGQVTISADKSISTAYLQWSSLKASDTAMYYCTRMWRGNSFDYWGQGTLVTVSS

SEQ ID NO: 83 - protein, 113 aa, artificial
  hGBA-9 VL
DIVMTQSPDSLAVSLGERATINCKSSQSVLNSGNQKNYLAWYQQKPGQPPKLLIYWASTRESGVPDRFSGSGSGTDFTLTISSLQAEDVAVYYCQQDYSYPFTFGQGTKVEIK

SEQ ID NO: 84 - protein, 448 aa, artificial
  hCl1a HC AAA Knob
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 659 aa, artificial
  hCl1a HC AAA Knob RLI2 DANA
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHATVEALIILANNSLSSNAQVTESGCKECEELEEKNIKEFLQSFVHIVQMFINTS

SEQ ID NO: 86 - protein, 659 aa, artificial
  hCl1a HC AAA Knob RLI2 NA
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 87 - protein, 448 aa, artificial
  hCl1a HC AAA Hole
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 88 - protein, 214 aa, artificial
  hCl1a LC
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 118 aa, artificial
  Zolbetuximab (IMAB362) VH
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 90 - protein, 113 aa, artificial
  Zolbetuximab (IMAB362) VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 91 - protein, 119 aa, artificial
  Cetuximab VH
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 92 - protein, 107 aa, artificial
  Cetuximab VL
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 93 - protein, 448 aa, artificial
  Cetuximab HC AAA Knob
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 659 aa, artificial
  Cetuximab HC AAA Knob RLI2 DANA
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHATVEALIILANNSLSSNAQVTESGCKECEELEEKNIKEFLQSFVHIVQMFINTS

SEQ ID NO: 95 - protein, 448 aa, artificial
  Cetuximab HC AAA Hole
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 96 - protein, 450 aa, artificial
  Rituximab heavy chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 661 aa, artificial
  Rituximab HC AAA Knob RLI AQNA
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 98 - protein, 450 aa, artificial
  Rituximab HC AAA Hole
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 99 - protein, 213 aa, artificial
  Rituximab LC
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 40 aa, artificial
  L40 linker
GGGGSGGGGSGGGGSGGGGSGGGGSGGGGSGGGGSGGGGS

SEQ ID NO: 101 - protein, 446 aa, Artificial Sequence
  SOT201 HC hole S228P.L235E.T366S.L368A.Y407V/dK
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 102 - protein, 653 aa, Artificial Sequence
  mPD1.VH-h1.HC.D265A.E356K.N399K.dk-RLI.N162A.G175A.N176Q murine antiPD-1 (mIgG1 D265A HC1 - RLI-15AQA)
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 103 - protein, 442 aa, Artificial Sequence
  mPD1.VH-h1.HC.D265A.K409E.K439D.dk murine antiPD-1 (mIgG1 D265A HC2)
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYKNTQPIMNTDGSYFVYSELNVQKSNWEAGNTFTCSVLHEGLHNHHTEDSLSHSP

SEQ ID NO: 104 - protein, 218 aa, Artificial Sequence
  mPD1.VL-hk.LC murine antiPD-1 (mIgG1 Light Chain)
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 105 - protein, 133 aa, Homo sapiens [sequence omitted]

SEQ ID NO: 106 - protein, 133 aa, Artificial Sequence
  IL-2v
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 107 - protein, 114 aa, Artificial Sequence
  IL-15 M1
AWVNVISDLKKIEDLIQSMHIDATLYTESNVHPSCKVTAMKCFLLGLQRISLESGDASIHDTVENLIILANNSLSSNGNVTESGCKECEELEEKNIKEFLQSFVHIVQMFINTS

SEQ ID NO: 108 - protein, 114 aa, Artificial Sequence
  IL-15 M2
GWVNVISDLKKIEDLIQSMHIDATLYTESNVHPSCKVTAMKCFLLGLQRISLESGDASIHDTVQNLIILANNSLSSNGNVTESGCKECEELEEKNIKEFLQSFVHIVQMFINTS

SEQ ID NO: 109 - protein, 448 aa, Artificial Sequence
  hCl1a HC Knob
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 110 - protein, 448 aa, Artificial Sequence
  hCl1a HC Hole
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 111 - protein, 659 aa, Artificial Sequence
  hCl1a HC Knob RLI2 NA
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 112 - protein, 597 aa, Artificial Sequence
  PD1-IL2v HC1  HC with IL2v (Fc knob, LALAPG), IL2v.T3A.F42A.Y45A.L72G.C125A
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 113 - protein, 448 aa, Artificial Sequence
  PD1-IL2v HC2  HC (Fc hole LALAPG)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 114 - protein, 218 aa, Artificial Sequence
  PD1-IL2v LC
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 575 aa, Artificial Sequence
  mPD1-IL2v HC1 mPD-1.VH-h1.HC.D265A.E356K.N399K.dk-IL2v.T3A.F42A.Y45A.L72G.C125A murine antiPD-1 (mIgG1 D265A HC1 - IL-2v)
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 116 - protein, 442 aa, Artificial Sequence; identical to SEQ ID NO: 103
  mPD1-IL2v HC2 mPD-1.VH-h1.HC.D265A.K409E.K439D.dk murine antiPD-1 (mIgG1 D265A HC2)

SEQ ID NO: 117 - protein, 218 aa, Artificial Sequence; identical to SEQ ID NO: 104
  mPD1-IL2v LC mPD-1.VL-hk.LC murine antiPD-1 (mIgG1 Light Chain)

SEQ ID NO: 118 - protein, 575 aa, Artificial Sequence
  hPD-1-IL15 (M1) HC1
QVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGAWVNVISDLKKIEDLIQSMHIDATLYTESNVHPSCKVTAMKCFLLGLQRISLESGDASIHDTVENLIILANNSLSSNGNVTESGCKECEELEEKNIKEFLQSFVHIVQMFINTS

SEQ ID NO: 119 - protein, 447 aa, Artificial Sequence
  hPD-1-IL15 (M1) HC2 xhPD-1.VH-h1.HC.L234A.L235A.G237A.T366W.dk anti-human PD-1 (Fc LALA KiH knob)
QVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 220 aa, Artificial Sequence
  hPD-1-IL15 (M1) LC xhPD-1.VL-hk.LC anti-human PD-1 (Light Chain)
DIQMTQSPSSLSASVGDRVTITCKSSQSLWDSGNQKNFLTWYQQKPGKAPKLLIYWTSYRESGVPSRFSGSGSGTDFTLTISSLQPEDFATYYCQNDYFYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 575 aa, Artificial Sequence
  hPD-1-IL15 (M2) HC1
QVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSGGGGGWVNVISDLKKIEDLIQSMHIDATLYTESNVHPSCKVTAMKCFLLGLQRISLESGDASIHDTVQNLIILANNSLSSNGNVTESGCKECEELEEKNIKEFLQSFVHIVQMFINTS

SEQ ID NO: 122 - protein, 447 aa, Artificial Sequence; identical to SEQ ID NO: 119
  hPD-1-IL15 (M2) HC2 xhPD-1.VH-h1.HC.L234A.L235A.G237A.T366W.dk anti-human PD-1 (Fc LALA KiH knob)

SEQ ID NO: 123 - protein, 220 aa, Artificial Sequence; identical to SEQ ID NO: 120
  hPD-1-IL15 (M2) LC xhPD-1.VL-hk.LC anti-human PD-1 (Light Chain)

SEQ ID NO: 124 - protein, 688 aa, Artificial Sequence
  Kadmon HC1 2-8 S354C/T366W LALAPG improved linker
ITCPPPMSVEHADIWVKSYSLYSRERYICNSGFKRKAGTSSLTECVLNKATNVAHWTTPSLKCIRDPALVHQRPAPPSGGGGSGGGGSGGGSGGGGSNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTSGGGGSGGGGSGGGGSGGGGSGGGGSGGGGSEVQLLESGGGLVQPGGSLRLSCAASGFTFSSYWMSWVRQAPGKGLEWVSAISGSGGSTYYADSVKGRFTISRDNSKNTLYLQMNSLRAEDTAVYYCASSPLQWVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 447 aa, Artificial Sequence
  Kadmon HC2 SEQ ID 223 1-4 Y349C/T366S/L368A/Y407V LALAPG
EVQLLESGGGLVQPGGSLRLSCAASGFTFSSYWMSWVRQAPGKGLEWVSAISGSGGSTYYADSVKGRFTISRDNSKNTLYLQMNSLRAEDTAVYYCASSPLQWVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 126 - protein, 214 aa, Artificial Sequence
  Kadmon LC SEQ ID NO 219 - 38B2
DIQMTQSPSSLSASVGDRVTITCRASESISSWLAWYQQKPGKAPKLLIYDASSLESGVPSRFSGSGSGTDFTLTISSLQPEDFATYYCQQGDSFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 659 aa, Artificial Sequence
  mSOT202 HC knob
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 128 - protein, 448 aa, Artificial Sequence
  mSOT202 HC hole
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPG

SEQ ID NO: 129 - protein, 214 aa, Artificial Sequence
  mSOT202 LC
DIQMTQSPSSLSASVGDRVTITCRASEDIYSNLAWYQQKPGKAPKLLIFSVKRLQDGVPSRFSGSGSGTDFTLTISSLQPEDFATYYCLQGSNFPLTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 130 - protein, 659 aa, Artificial Sequence
  mSOT202 LALAPG HC knob
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLWCMVTDFMPEDIYVEWTNNGKTELNYKNTEPVLDSDGSYFMYSKLRVEKKNWVERNSYSCSVVHEGLHNHHTTKSFSRTPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 131 - protein, 448 aa, Artificial Sequence
  mSOT202 LALAPG HC hole
QVQLVQSGAEVKKPGASVKVSCKASGYTFTDYAMHWVRQAPGQRLEWMGWINTYTGKPTYAQKFQGRVTITRDTSASTAYMELSSLRSEDTAVYYCARAVFYGYTMDAWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLSCAVTDFMPEDIYVEWTNNGKTELNYKNTEPVLDSDGSYFMVSKLRVEKKNWVERNSYSCSVVHEGLHNHHTTKSFSRTPG

SEQ ID NO: 132 - protein, 665 aa, Artificial Sequence
  mSOT202 isotype HC knob
EVQLVESGGGLVKPGGSLKLSCAVSGFTFSDYAMSWIRQTPENRLEWVASINIGATYAYYPDSVKGRFTISRDNAKNTLFLQMSSLGSEDTAMYYCARPGSPYEYDKAYYSMAYWGPGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEKEMTKKQVTLTCMVTDFMPEDIYVEWTNNGKTELNYKNTEPVLKSDGSYFMYSKLRVEKKNWVERNSYSCSVVHEGLHNHHTTKSFSRTPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 133 - protein, 454 aa, Artificial Sequence
  mSOT202 isotype HC hole
EVQLVESGGGLVKPGGSLKLSCAVSGFTFSDYAMSWIRQTPENRLEWVASINIGATYAYYPDSVKGRFTISRDNAKNTLFLQMSSLGSEDTAMYYCARPGSPYEYDKAYYSMAYWGPGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSELRVEKKNWVERNSYSCSVVHEGLHNHHTTDSFSRTPG

SEQ ID NO: 134 - protein, 214 aa, Artificial Sequence
  mSOT202 isotype LC
DVQMTQSTSSLSASLGDRVTISCRASQDIKNYLNWYQQKPGGTVKLLIYYSSTLLSGVPSRFSGRGSGTDFSLTITNLEREDIATYFCQQSITLPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 135 - protein, 665 aa, Artificial Sequence
  mSOT202 isotype LALAPG HC knob
EVQLVESGGGLVKPGGSLKLSCAVSGFTFSDYAMSWIRQTPENRLEWVASINIGATYAYYPDSVKGRFTISRDNAKNTLFLQMSSLGSEDTAMYYCARPGSPYEYDKAYYSMAYWGPGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEKEMTKKQVTLTCMVTDFMPEDIYVEWTNNGKTELNYKNTEPVLKSDGSYFMYSKLRVEKKNWVERNSYSCSVVHEGLHNHHTTKSFSRTPGITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVEALIILANNSLSSNAQVTESGCKECEELEEKNIKEFLQSFVHIVQMFINTS

SEQ ID NO: 136 - protein, 454 aa, Artificial Sequence
  mSOT202 isotype LALAPG HC hole
EVQLVESGGGLVKPGGSLKLSCAVSGFTFSDYAMSWIRQTPENRLEWVASINIGATYAYYPDSVKGRFTISRDNAKNTLFLQMSSLGSEDTAMYYCARPGSPYEYDKAYYSMAYWGPGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSELRVEKKNWVERNSYSCSVVHEGLHNHHTTDSFSRTPG

SEQ ID NO: 137 - protein, 35 aa, Artificial Sequence
  RLI-15AQ peptide
ELQVISLESGDASIHDTVENLIILANNSLSSNAQV

SEQ ID NO: 138 - protein, 35 aa, Artificial Sequence
  RLI-15AQA peptide
ELQVISLESGDASIHDTVEALIILANNSLSSNAQV

SEQ ID NO: 139 - protein, 32 aa, Artificial Sequence
  RLI-15 NA peptide
VEALIILANNSLSSNGNVTESGCKECEELEEK

SEQ ID NO: 140 - protein, 32 aa, Artificial Sequence
  RLI-15AQA peptide
VEALIILANNSLSSNAQVTESGCKECEELEEK

(19) United States
(12) Patent Application Publication
(10) Pub. No.: US 2024/0294594 A1
(43) Pub. Date: Sep. 5, 2024

(54) INTERLEUKIN-15 BASED IMMUNOCYTOKINES

(71) Applicants: Cytune Pharma, Nantes (FR); Sotio Biotech a.s., Prague 9 (CZ)

(72) Inventors: Irena Adkins, Prezletice (CZ); Eva Nedvedová, Karlovy Vary (CZ); Guy Luc Michel De Martynoff, Mont-St-Guibert (BE); Ulrich Moebius, Gauting-Unterbrunn (DE); David Béchard, Saint-Etienne de Montluc (FR); Šárka Pechoucková, Praha (CZ); Zuzana Antošová, Rícany (CZ); Lenka Kyrych Sadilkova, Horomerice (CZ); Roger Renzo Beerli, Adlikon bei Regensdorf (CH); Lukas Bammert, Basel (CH); Lorenz Waldmeier, Nidau (CH); Iva Valentová, Ceske Budejovice (CZ); Simona Hošková, Prague 9-Vinor (CZ)

(21) Appl. No.: 18/573,397

(22) PCT Filed: Jun. 23, 2022
(86) PCT No.: PCT/EP2022/067236

§ 371 Date: Dec. 21, 2023

(30) Foreign Application Priority Data
Jun. 23, 2021 (EP) 21181271.4
Mar. 23, 2022 (EP) 22163748.1

(51) Int. Cl. C07K 14/54; A61K 39/00; C07K 14/715; C07K 16/28

(52) U.S. Cl. CPC C07K 14/5443; C07K 14/7155; C07K 16/28; C07K 16/2818; C07K 16/2887; A61K 2039/505; C07K 2317/24; C07K 2317/40; C07K 2317/732; C07K 2317/76; C07K 2317/92; C07K 2317/94; C07K 2319/30; C07K 2319/32

(57) ABSTRACT

The present invention inter alia relates to immunocytokines involving IL-15 superagonists (based on IL-15 and the sushi domain of IL-15Rα) and an antibody. The invention also provides nucleic acids, vectors, methods and medical uses.

### BACKGROUND OF THE INVENTION

Interleukin 15 (IL-15) is a naturally occurring cytokine that induces the generation of cytotoxic lymphocytes and memory phenotype CD8⁺ T cells, and stimulates proliferation and maintenance of natural killer (NK) cells but—in contrast to interleukin 2—does not mediate activation-induced cell death, does not consistently activate Tregs and causes less capillary leak syndrome (Waldmann, Dubois et al. 2020). Extensive preclinical and clinical studies demonstrating the effectiveness and limitation of IL-15 and of an increasing number of IL-15 analogs/superagonists especially in the treatment of cancer have been conducted, reviewed by Robinson and Schluns (Robinson and Schluns 2017).

IL-15, like interleukin 2 (IL-2), acts through a heterotrimeric receptor having α, ρ and γ subunits, whereas they share the common gamma-chain receptor (γ_{c} or γ)—also shared with IL-4, IL-7, IL-9 and IL-21—and the IL-2/IL-15Rβ (also known as IL-2Rβ, CD122). As a third subunit, the heterotrimeric receptors contain a specific subunit for IL-2 or IL-15, i.e., the IL-2Rα (CD25) or the IL-15Rα (CD215). Downstream, IL-2 and IL-15 heterotrimeric receptors share JAK1 (Janus kinase 1), JAK 3, and STAT3/5 (signal transducer and activator of transcription 3 and 5) molecules for intracellular signalling leading to similar functions, but both cytokines also have distinct roles as reviewed in Waldmann (2015, see e.g. table 1) and Conlon (2019).

Accordingly, the activation of different heterotrimeric receptors by binding of IL-2, IL-15 or derivatives thereof potentially leads to a specific modulation of the immune system and potential side effects. Recently, novel compounds comprising IL-15 or IL-15 variants were designed aiming at specifically targeting the activation of NK cells and CD8⁺ T cells. These are compounds targeting the mid-affinity IL-2/IL-15Rβγ, i.e., the receptor consisting of the IL-2/IL-15Rβ and the γ_{c} subunits, which is expressed on NK cells, CD8⁺ T cells, NKT cells and γδ T cells. This is critical for safe and potent immune stimulation mediated by IL-15 trans-presentation, whereas the designed compounds SOT101 (SO-C101, RLI-15), ALT-803 and hetIL-15 already contain (part of) the IL-15Rα subunit and therefore simulate trans-presentation of the α subunit by antigen presenting cells. SO-C101 binds to the mid-affinity IL-15Rβγ only, as it comprises the covalently attached sushi+domain of IL-15Rα. In turn, SO-C101 does bind neither to IL-15Rα nor to IL-2Rα. Similarly, ALT-803 and hetIL-15 carry an IL-15Rα sushi domain or the soluble IL-15Rα, respectively, and therefore bind to the mid-affinity IL-15Rβγ receptor. Accordingly, IL-15 and IL-15 analogues/superagonists are promising clinical stage development candidate for the treatment of cancer and infectious diseases.

As a method of targeted cytokine delivery, antibody-cytokine fusion molecules termed immunocytokines have been developed. Such proteins retain both antigen-binding properties and cytokine activity. By targeting the antibody portion to the tumour-associated antigens, neovascularization antigens, tumour microenvironment antigens or immune check points, the immunocytokines are sequestered in the tumour microenvironment, where the cytokine portion can signal through its cognate receptors expressed on immune cells and induce an anti-tumour response. For example, when the antibody is a check point inhibitor (CPI), the combination of the antibody and cytokine will boost the immune response against cancer by lifting the “brakes” on the immune system through the CPI and stimulating the immune cells through the cytokine. When the antibody is targeting tumour antigens, the antibody effector functions may be enhanced by the presence of cytokines activating the immune cells involved in the antibody effector functions.

However, there is no approved IL-15-based immunocytokine yet on the market. Recently IL-2-based immunocytokine targeted against CEA and FAP appear to have been discontinued.

Accordingly, there is a continued need to improve the design of immunocytokines.

### SUMMARY OF THE INVENTION

The inventors have now developed a mutation/protein modification toolbox allowing modulation of the IL-15 superagonists (based on IL-15 and the sushi domain of IL-15Rα) activity and of the antibody forming the immunocytokine. The inventors identified suitable single or double mutations reducing the binding of the IL-15 superagonists to the IL-2/IL-15Rβ and/or to the γ_{c} receptor, to minimize target mediated drug deposition due to too high affinity to immune effector cells, resulting in increasing the half-life of the immunocytokine. Different mutations allow to tune the level of reduced binding. Other mutations in IL-15 superagonists may improve the homogeneity of the IL-15 variant with respect to post-translational modifications. Modulating the IL-15 superagonists' activity may also include varying the presence of one or two cytokines fused to the antibody. Therefore, the toolbox also includes mutations allowing heterodimeric antibodies. Toolbox mutations to modulate the antibody effector functions may include Fc mutations enhancing or reducing antibody-dependent cell toxicity and/or mutations increasing in vivo half-life or stability of the antibody. The toolbox also includes enhancing antibody-dependent cell toxicity by producing afucosylated antibodies. The toolbox further includes different formats of antibodies adapted to specific needs, such as IgG1 or IgG4 antibodies. The inventors have now also designed exemplary immunocytokines aimed to combine CPI activity or tumour-antigen targeting antibodies and IL-15 superagonists' activity. An immunocytokine based on a heterodimerized pembrolizumab, with decreased ADCC activity fused to an RLI molecule with reduced binding to the IL-2/IL-15Rβ is a combination of a CPI with a cytokine. An immunocytokine based on a heterodimerized hC11a, an anti-CLDN18.2 antibody, optionally with enhanced ADCC activity, fused to an RLI molecule with reduced binding to the IL-2/IL-15Rβ is a combination of a tumour-antigen targeting antibody with a cytokine.

### FIGURES

FIG. 1: (A) LMW SDS-PAGE and Western-blot (anti-RLI-15) analysis of RLI2 (RLI2 wt), RLI2 with G78A substitution (RLI2 A) and RLI2 with G78A/N79Q substitutions (RLI2 AQ) under non-reducing conditions. For Coomassie staining 0.5 μg or 2 μg or protein were used (lanes 2, 4, 6, 8, 10 and 12) and for Western blotting 25 ng of protein were used (lanes 3, 7, 11).
(B) Capillary Electrophoresis, denaturing, analysis of RLI2 (RLI2 wt), RLI2 with G78A substitution (RLI2 A) and RLI2 with G78A/N79Q substitutions (RLI2 AQ) under reducing (R) and non-reducing (NR) conditions. Dotted box 1 represents band for glycosylation site #2 (main), box 2 represents band for glycosylation site #1 (minor), dotted box 3 represents new glycosylation site for RLI2 A. Non-named lanes are marker with 16, 21, 30, 48 and 68 kDa.

FIG. 2: Analysis of the 3 deglycosylated RLI variants expressed in CHO cells by SDS-PAGE (7.5-18%) stained by Coomassie blue (left pane), by silver nitrate (middle pane) and detected by an anti-IL15 western blot (right pane): lanes 1: molecular weight marker; lanes 2: RLI2_{N176Q}, lanes 3: RLI2_{N168S/N176Q/N209S}, lanes 4: RLI1_{N168S/N176Q/N209S}.

FIG. 3: Potency of RLI2 and RLI2_{AQ} from supernatants determined by activation of 32 Db cells or Kit225 cells. (A) 32 Db cells, 21 h, (B) Kit225 cells, 4 h.

FIG. 4: Relative potency of RLI2 purified or from supernatant compared to RLI2_{AQ} from supernatant determined by activation of Kit225 cells.

FIG. 5: Comparison of highly glycosylated RLI2 and low glycosylated RLI2
(A) CPI HIC elution profile in dependence of Concentration of Buffer B measured at 280 nm. Left box indicates pooled fraction 2B1 1-3 for highly glycosylated RLI2 (“RLI-15-HG”) and right box indicates pooled fractions 4B1 1-3 for low glycosylated RLI2 (“RLI-15-LG”). (B) SDS PAGE of fractions 2B1 1-3 of RLI-15-HG, RLI2 reference standards and molecular weight ladders of given kDa. (C) SDS PAGE of fraction 4B1 1-3 of RLI-15-LG, RLI2 reference standards and molecular weight ladders of given kDa.

FIG. 6: Capillary electrophoresis of selected PEM-RLI constructs.

FIG. 7: Therapeutic efficacy in vivo of PEM-RLI NA x1 in comparison to pembrolizumab in treating HuCell MC38-hPD-L1 tumour cell line implanted in female hPD-1 single KI HuGEMM mice.

FIG. 8: Mixed lymphocyte reaction (hPBMC donors): INFγ secretion in pg/ml for control (PBMC only), pembrolizumab and PEM LE-RLI2_{AQ} NA x1.

FIG. 9: Pharmacokinetics and pharmacodynamics of PEM-RLI construct with reduced IL-2Rβγ binding compared to PEM-RLI with normal binding in cynomolgus monkeys after the IV administration of 10 or 30 μg/kg PEM-RLI x1, and 30 or 90 μg/kg PEM LE/YTE-RLI NA x1 on day 1 and day 15, respectively.
(A) Concentration of construct in serum in dependence of time in hours after administration. 10 μg/kg PEM-RLI x1 (solid grey line), 30 μg/kg PEM-RLI x1 (dotted grey line), 30 μg/kg PEM LE/YTE-RLI NA x1 (dotted black line), or 90 μg/kg PEM LE/YTE-RLI NA x1 (solid black line); LLOQ for lower limit of quantitation. (B) Count of lymphocytes (fold change) in dependence of time in days. (C) % of Ki67⁺ NK cells. (D) % of Ki67⁺ CD8⁺ T cells: PEM-RLI x1 in grey, PEM LE/YTE-RLI NA x1 in black, with two animals per group (full and open circles for individual animals in group).

FIG. 10: Comparison of pharmacokinetics and pharmacodynamics of PEM-RLI NA x1 and PEM-RLI NA x2 in cynomolgus monkeys after a single IV administration of 30 μg/kg. (A) Concentration of construct in serum in dependence of time in hours with LLOQ for lower limit of quantitation; (B) Count of lymphocytes (fold change) in dependence of time in days. (C) % of Ki67+NK cells; (D) % of Ki67⁺ CD8⁺ T cells: PEM-RLI NA x1 in black with two individual animals with filled circles and empty circles, and PEM-RLI NA x2 in grey with two individual animals with filled circles and empty circles.

FIG. 11: Comparison of pharmacokinetics of PEM LE/YTE-RLI NA x1 and PEM LE-RLI NA x1 in cynomolgus monkeys after a single IV administration of 600 μg/kg. Concentration of construct in serum is depicted in dependence of time in hours with LLOQ for lower limit of quantitation: PEM LE/YTE-RLI NA x1 with black circles/dotted line and PEM LE-RLI NA x1 with grey circles and solid line.

FIG. 12: Comparison of pharmacokinetics of PEM LE-RLI NA x1 and PEM-RLI NQD x1 in cynomolgus monkeys after a single IV administration of 600 μg/kg. Concentration of construct in serum is depicted in dependence of time in hours with LLOQ for lower limit of quantitation: PEM LE-RLI NA x1 with grey circles/line and PEM-RLI NQD x1 with black circles/line.

FIG. 13: Comparison of ADCC activity of immunocytokines based on the hCl1a antibody with non-modified effector functions to immunocytokines with reduced ADCC activity and antibodies hCl1a and Zolbetuximab. ADCC target cells were A549 cells overexpressing CLDN18.2 (A549-CLDN18.2) or PA-TU-8988S cells (PATU) endogenously expressing CLDN18.2.

FIG. 14: Comparison of ADCC activity of immunocytokines based on the hCl1a antibody with non-modified effector functions to immunocytokines with enhanced ADCC activity and antibodies hCl1a and Zolbetuximab. ADCC target cells were A549 cells overexpressing CLDN18.2 (A549-CLDN18.2) or PA-TU-8988S cells (PATU) endogenously expressing CLDN18.2. (A): DLE mutation; (B): DE mutation; (C): AAA mutation; (D): TL mutation; (E): IE mutation; (F): afucosylated immunocytokines.

FIG. 15: PD activity of RTX-RLI2_{AQ} immunocytokines in Balb/c mice: percent of (left panels) or activated (Ki67⁺—right panels) CD8⁺ T cells or NK cells was determined by flow cytometry.

FIG. 16: Anti-metastatic activity of RTX-RLI2_{AQ} immunocytokines in Renca mouse metastatic model in vivo as determined by lung weight.

FIG. 17: Anti-tumor efficacy of RTX-RLI immunocytokines in A20-hCD20/Balb/c mice: Tumor growth is depicted for individual mice in dependence of time. A: buffer control injected s.c. day 1-4; B: RLI2 control with 1 mg/kg injected s.c. day 1-4; C: RTX-RLI2_{AQ} injected i.v. with 0.15 mg/kg at day 1 and day 8.

FIG. 18: ADCC activity of RTX-RLI immunocytokines based on rituximab compared to rituximab alone. % of dead tumor cells determined by DAPI positivity was determined in dependence of the concentration of the tested polypeptide: black circles: rituximab, grey circles: rituximab+NK92 cells; black triangles with dotted line: RTX-RLI2_{AQ} x2, black triangles with solid line: RTX-RLI2_{AQ} x2+NK92 cells; grey squares: RTX-RLI2_{AQ} x1, black squares: RTX-RLI2_{AQ} x1 with NK92 cells.

FIG. 19: (A) % of PD-1/PD-L1 blocking is shown in dependence of increasing concentrations in pM of Keytruda and SOT201
(B) % of Ki67⁺ NK cells and CD8⁺ T cells determined by flow cytometry after 7 days stimulation in vitro of human PBMC from healthy donors with increasing amounts of SOT201 or SOT201 wt having an IL-15 moiety without reduced binding to the IL-2/IL-15Rβγ.
(C) Cell proliferation (Ki67⁺) of CD8⁺ T cells or NK cells detected in spleen of healthy C57BL/6 mice (n=2/group) by flow cytometry 5 days after IV injection of compounds at equimolar amount to 5 mg/kg of the murine surrogate molecule mSOT201 (anti-murine PD-1 antibody RMP1-14 fused RLI-15_{AQA}) compared to the anti-murine PD-1 antibody alone or to the anti-human PD1 mouse IgG1-RLI-15_{AQA} (hPD1-mSOT201) as single activity controls.

FIG. 20: (A) Tumor volume in mm³ over a time course of 17 days of C57BL/6 mice bearing syngeneic MC38 tumor cells treated IV with a single injection of control (NaCl, mSOT201, hPD1-mSOT201 or mPD1 at equimolar amount to mSOT201 (5 mg/kg) on day 1 (randomization day of tumor volumes 80-100 mm³) (n=10 mice/group).
(B) corresponding % of surviving MC38 tumor bearing mice up to 100 days post treatment.

FIG. 21: (A) Relative expression levels of gene sets associated with indicated adaptive and innate immune cells and cancer associated fibroblasts (CAFs) across mSOT201 treated tumor samples (N=3) and control samples (n=4) of MC38 tumor bearing mice as determined by metagenes on RNA seq data. Box plots: minimum, median, maximum.
(B) Cell proliferation as determined by % Ki67⁺ cells by flow cytometry of indicated cells in spleen or lymph nodes in MC38 tumor bearing mice on day 7 after mSOT201 (5 mg/kg) IV treatment of established tumors (80-100 mm³) (n=2).

FIG. 22: (A) Tumor volume in mm³ over a time course of 21 days of C57BL/6 mice bearing MC38 tumors treated IV with a single injection of control (NaCl), mSOT201, the mPD1-IL-2βγ agonist (IL-2v fused to the anti-murinePD-1 antibody RMP1-14) with abolished CD25 binding or the combination of RLI-15_{AQA} with the anti-murine PD-1 antibody mPD1 (RMP1-14) (n=10 mice/group).
(B) Cell proliferation as determined by % Ki67⁺ cells of CD8⁺ T cells and NK cells detected by flow cytometry after IV administration in healthy C57/BL6 mice at day 5 and day 8.
(C) % Ki67⁺ cells of CD8⁺ T cells in spleen or lymph nodes at day 7 of C57BL/6 mice bearing MC38 tumors treated IV with mSOT201, mPD1-IL-2v or the combination of RLI-15_{AQA} and mPD-1. Randomization day 1, tumor volumes 100 mm³ (n=10/group).

FIG. 23: (A) % of Ki67⁺ and fold change of absolute cell counts of NK and CD8⁺ T cells in blood of cynomolgus monkeys after a single IV administration of 0.6 mg/kg of SOT201 at day 1 determined at indicated days by flow cytometry and haematology, each graph curve representing one animal.
(B) % of Ki67⁺ of NK and CD8⁺ T cells in blood of cynomolgus monkeys after administration on days 1 and 21 (indicated by arrows) IV administration of 0.3 mg/kg of SOT201 determined at indicated days by flow cytometry, each graph curve representing one animal.

FIG. 24: NK and CD8⁺ T cell proliferation upon treatment with mouse SOT201 surrogates in vivo.
(A) Proliferation of CD8⁺ T cells and NK cells in spleen of healthy C57BL/6 mice at day 5 and 8 after treatment with hPD1-mSOT201, mPD-1, mSOT201, mSOT201 wt and mPD1-IL2v. The expression of Ki67 in CD8⁺ T cells and NK cells was detected by flow cytometry. The molecules were administered i.v. on day 1 at doses equimolar to 5 mg/kg of mSOT201: hPD1-mSOT201 at 5.37 mg/kg, mPD-1 at 4.51 mg/kg, and at a dose equimolar to 0.25 mg/kg of mSOT201 wt: mPD1-IL2v at 0.26 mg/kg. Flow cytometry analysis was performed on day 5 and day 8. The data represent mean SEM for 2 individuals per group per day.
(B) Proliferation of CD8⁺ T cells and NK cells in spleen of healthy C57BL/6 mice at day 5 and 8 after treatment with hPD1-mSOT201, mPD-1, mSOT201, mSOT201 wt and mPD1-IL2v. The expression of Ki67 in CD8⁺ T cells and NK cells was detected by flow cytometry. The molecules were administered i.v. on day 1 at doses equimolar to 10 mg/kg of mSOT201: hPD1-mSOT201 at 10.74 mg/kg, mPD-1 at 9.02 mg/kg, and at dose equimolar to 0.1 mg/kg of mSOT201 wt: mPD1-IL2v at 0.1 mg/kg. Flow cytometry analysis was performed on day 5 and day 8. The data represent mean SEM for 2 individuals per group per day.

FIG. 25: mouse SOT201 surrogates in PD-1 sensitive and PD-1 resistant tumor models in vivo.
(A) Anti-PD-1 sensitive tumor models
MC38/C57BL/6 mouse model: single i.v. administration at Day 0 of 4.51 mg/kg mPD-1 (sub-optimal dose as compared to literature, selected as equimolar to mSOT201), 5 mg/kg mSOT201 or 5.37 mg/kg hPD1-mSOT201 (equimolar to mSOT201); D0=randomization day with tumor volume of ˜80-100 mm³, 10 mice/group;
CT26 BALB c mouse model: four i.p. administrations at Day 0, 3, 6 and 9 with 9.02 mg/kg mPD-1 (effective dose as compared to literature), 10 mg/kg mSOT201, 10.74 mg/kg hPD1-mSOT201 (equimolar to mSOT201); D0=randomization day with tumor volume of ˜100 mm³, 10 mice/group.
(B) Anti-PD-1 resistant tumor models
CT26 STK11 ko mouse model: four i.p. administrations at Day 0, 3, 6 and 9 with 9.02 mg/kg mPD-1 (effective dose as compared to literature), 10 mg/kg mSOT201, 10.74 mg/kg hPD1-mSOT201 (equimolar to mSOT201); D0=randomization day with tumor volume of ˜100 mm³, 10 mice/group. B16F10/C57BL/6 mouse model: four i.p. administrations at Day 0, 3, 6 and 9 with 9.02 mg/kg mPD-1 (effective dose as compared to literature), 10 mg/kg mSOT201, 10.74 mg/kg hPD1-mSOT201 (equimolar to mSOT201); Day 0=randomization day with tumor volume of ˜100 mm³, 10 mice/group Cut-off day for all mice present in the control groups, CR=complete response

FIG. 26: Comparison of mSOT201 vs. RLI-15_{AQA} mutein+anti-PD-1 in vivo.
MC38/C57BL/6 mouse model with following groups:
   G1 mock control
   G4: a single administration of 0.64 mg/kg RLI-15_{AQA}, s.c. at Day 0+a single administration of 4.51 mg/kg mPD-1, i.p. at Day 0.
   G2 a single administration of 5 mg/kg mSOT201, i.v. at Day 0
   G3 a single administration of 2 mg/kg mSOT201, i.v. at Day 0
   G6 a single administration of 4.51 mg/kg single mPD1, i.p. at Day 0 (suboptimal dose as compared to literature, selected as equimolar to mSOT201),
   G11 a single administration of 5 mg/kg hPD1-mSOT201, i.v. at Day 0+a single administration of 4.36 mg/kg mPD-1, i.p. at Day 0,
Day 0=randomization day with tumor volume of ˜80-100 mm³, 10 mice/group
Cut-off day for all mice present in the control groups, CR=complete response.

FIG. 27: MC38/C57BL/6 mouse model−D0=randomization day □80-100 mm3, 10 mice/group. CR=complete response
G1 mock control
G2 single administration of 5 mg/kg of mSOT201, i.v. at Day 0
G3 single administration of 2 mg/kg of mSOT201, i.v. at Day 0
G7 4 administrations of 1 mg/kg of RLI2_{AQ}, s.c. at Day 0, 1, 2 and 3
G5 single administration of 1 mg/kg RLI2_{AQ}, s.c. at Day 0+single administration of 5 mg/kg mPD1, i.p. at Day 0
G8 4 administrations of 1 mg/kg of RLI2_{AQ}, s.c. at Day 0, 1, 2 and 3+single administration of 5 mg/kg mPD1, i.p. at Day 0
G9 4 administrations of 1 mg/kg of RLI2_{AQ}, s.c. at Day 0, 1, 2, and 3+4 administrations of 5 mg/kg mPD1, i.p. at Day 0, 3, 6 and 9
G6 single administration of 5 mg/kg mPD1, i.p. at Day 0
G10 4 administrations of 5 mg/kg mPD1, i.p. at Day 0, 3, 6 and 9
Cut-off day for all mice present in the control groups

FIG. 28: Comparison of mSOT201 vs. RLI2_{AQ}+anti-PD-1 tumor growth in vivo. MC38/C57BL/6 mouse model
(A) average tumor volume in mm³ in dependence of time and shown for individual animals at day 16, with the horizontal line showing the mean tumor volume.
   G1 mock control
   G2 single administration of 2 mg/kg of mSOT201, i.v. at Day 0,
   G3 two administration of 2 mg/kg RLI2_{AQ}, s.c. at Day 0 and 1+4 administrations of 2 mg/kg mPD1, i.p. at Day 0, 3, 6 and 9.
1 experiment only, D0=randomization day at tumor volume of ˜80-100 mm³, 10 mice/group.
CR=complete response
The relative expansion of NK cells, CD8⁺ T cells and cells expressing αβTCR and γδTCR (T cells) was investigated in spleen, lymph nodes and tumor at day 7 after SOT201 (G2 from above) and RLI2_{AQ}+anti-PD-1 (G3 from above) treatment using flow cytometry. 3 tumor samples were pooled and 3 spleen and lymph node samples were analyzed separately.
(B) Frequency of parent (relative percentage compared to parent population) in % is shown for CD8⁺ T cells (top row) and NK cells (bottom row) from lymph nodes, spleen and tumor.
(C) Frequency of parent in % is shown for αβTCR+CD3⁺ T cells (top row) and βγTCR+CD3⁺ T cells (bottom row) from lymph nodes, spleen and tumor.

FIG. 29: (A) Immunogenicity in DC-T cell-based assay. T cell response to PEM-RLI-15 candidate molecules shown as % CFSE^{low} stained CD4⁺ T cells after loading of iDCs with candidate molecules, incubation with autologous CD4⁺ T cells pre-stained with CFSE and detection of CFSE staining with CFSE^{low} as a surrogate for cycling cells. Mean of 11 donors±SEM is shown. Significant differences compared to control DCs incubated with no protein and thus inducing non-specific T cell proliferation are shown. * p≤0.05, *** p≤0.001.
(B) FluoroSpot assay for IFN-γ and TNF-α of RLI-15 peptides spanning the introduced substitutions N65A and G175A/N176Q. Estimation of the effect of Mut2 or Mut3 peptides vs. respective wildtype peptides on the average dSFU in the test population of 40 donors with 95% confidence intervals (CI). SFU=Spot-forming Units, dSFU=SFU of restimulated well minus SFU of non-restimulated well.

FIG. 30: Comparison of the capacity to induce proliferation of hPBMCs of SOT202 molecules with modified effector functions. Proliferation of isolated hPBMC was assessed for SOT202-DANA, SOT202-afuc-DANA, SOT202-DLE-DANA, SOT202-DE-DANA and SOT202-LALAPG-DANA. Cells were stimulated in vitro for 7 days. Mean of 6 donors±SEM is shown. Proliferation of NK (top) and CD8⁺ T cells (bottom) was measured by counting Ki67⁺ cells by flow cytometry.

FIG. 31: Comparison of the capacity to induce proliferation of hPBMCs of SOT202 molecules and SOT201. Proliferation of isolated hPBMC was assessed for SOT202, SOT202-afuc, SOT201-DANA, SOT202-DANA and SOT202-afuc-DANA. Proliferation of NK (top) and CD8⁺ T cells (bottom) was measured by counting Ki67⁺ cells by flow cytometry.

FIG. 32: Comparison of the capacity to induce proliferation of hPBMCs of SOT202-DANA molecules with modified effector functions and SOT201-DANA. Proliferation of isolated hPBMC was assessed for SOT201-DANA, SOT202-DANA, SOT202-afuc-DANA, SOT202-LALAPG-DANA and hCl1a (also labelled SOT202-mab). Proliferation of NK (top) and CD8⁺ T cells (bottom) was measured by counting Ki67⁺ cells by flow cytometry.

FIG. 33: (A) Cell proliferation (Ki67⁺) of CD8⁺ T cells or NK cells detected in spleen of healthy C57BL/6 mice after stimulation with mSOT202. Cell proliferation was detected by Ki67 staining and measured by flow cytometry 5 days after IV injection of compounds of mSOT202 (hCl1a-mIgG2a-NA 1×) at 5, 10 or 20 mg/kg or of hCl1a-mIgG2a. (B) Percentage of NK cell and CD8⁺ T cell under the same experimental conditions as in (A).

FIG. 34: Cell proliferation of NK cells (A) or CD8⁺ T cells (B) detected in spleen of healthy C57BL/6 mice after stimulation with mSOT202, mSOT202-LALAPG and hCl1a-mIgG2a. Top: Cell proliferation was detected by Ki67 staining and measured by flow cytometry 5 and 10 days after IV injection of the compounds at 5 mg/kg. Bottom: Percentage of NK cell and CD8⁺ T cell.

### DEFINITIONS

“Antibody” also known as an immunoglobulin (Ig) is a large, Y-shaped protein composed in humans and most mammals of two heavy chains (HC) and two light chains (LC) connected by disulfide bonds. Light chains consist of one variable domain V_{L} and one constant domain C_{L}, while heavy chains contain one variable domain V_{H} and three constant domains C_{H}1, C_{H}2, C_{H}3. Structurally an antibody is also partitioned into two antigen-binding fragments (Fab), containing one V_{L}, V_{H}, C_{L}, and C_{H}1 domain each, as well as the Fc fragment or domain containing the two C_{H}2 and C_{H}3 of the two heavy chains.

An “antibody variant” or “antibody functional variant”, as used herein, relates to antibodies with modifications for e.g., modulating their effector functions, modulating the antibody stability and in vivo half-life and/or inducing heterodimerization of the antibody Fc domains. Such variants may be achieved by mutations and/or posttranslational modifications. Antibody variants also include antibody heavy chains with truncation of the N-terminal lysine. Other included variations are N- or C-terminal tags of the heavy and/or light chains for chemical or enzymatic coupling to other moieties such as dyes, radionuclides, toxins or other binding moieties. Further, antibody variants may comprise chemical modifications, modifications of their glycosylation or substitutions with artificial amino acids for chemical linkage to other moieties.

Antibody variant, as used herein, also relates to immunoglobulin gamma (IgG)-based bispecific antibodies that potentially recognize two or more different epitopes. Various formats of bispecific antibodies are known in the art, e.g. reviewed by Godar et al. (2018) and Spiess et al. (2015). Bispecific formats according to this invention include an Fc domain. With respect to the immunocytokines of the inventions, two RLI conjugates may, if not otherwise linked to a moiety, be either fused to the C-terminus of both light chains or to the C-terminus of both heavy chains; alternatively, one RLI conjugate may be fused to the C-terminus of one heavy chain for heterodimeric bispecific formats, or to the heavy chain or one light chain of heterodimeric bispecific formats with different light chains. Antibody functional variants are capable of binding to the same epitope or target as their corresponding non-modified antibody. The term “antibody” when generically used includes the antibody variants as defined herein.

A “conjugate”, as used herein, relates to either a non-covalent or a covalent complex of an interleukin 15 (IL-15) or a derivative thereof and the sushi domain of an interleukin 15-receptor alpha (IL-15Rα) or a derivative thereof. The non-covalent complex may be formed either by co-expression of the two polypeptides or by separate expression, (partial) purification and subsequent combination to form such complex due to the affinity of such polypeptides. Preferably, the conjugate is a fusion protein, where the two polypeptides are genetically fused and recombinantly expressed to result in a single polypeptide chain to form the intact complex.

An “immunocytokine”, as used herein, relates to polypeptide comprising an antibody or a functional variant thereof, genetically fused to a conjugate according to the invention.

When RLI is mentioned within a specific immunocytokine construct, it is RLI2.

The EU numbering scheme has been applied to the disclosed antibodies or partial antibody sequences.

“In vivo half-life” or T½ refers to the (terminal) plasma half-life or T½ is the half-life of elimination or half-life of the terminal phase, i.e. following administration the in vivo half-life is the time required for plasma/blood concentration to decrease by 50% after pseudo-equilibrium of distribution has been reached (Toutain and Bousquet-Melou 2004). The determination of the drug, here the immunocytokine agonist being a polypeptide, in the blood/plasma is typically done through a polypeptide-specific ELISA. The in vivo half-life of a particular drug can be determined in any mammal. For example, the in vivo half-life can be determined in humans, primates or mice. While the in vivo half-life determined in humans may considerably differ from the in vivo half-life in mice, i.e., the in vivo half-life in mice for a certain drug is commonly shorter than the in vivo half-life determined for the same drug in humans, such in vivo half-life determined in mice still gives an indication for a certain in vivo half-life in humans. Hence, from the in vivo half-life determined for a particular drug in mice, the in vivo half-life of the drug in humans can be extrapolated. This is particularly important since the direct determination of the in vivo half-life of a certain drug in humans is rarely possible due to prohibitions of experiments for merely scientific purposes involving humans. Alternatively, the half-life can be determined in primates (e.g., cynomolgus monkeys) which is more similar to the half-life in humans.

When it is stated “administered in combination” this typically does not mean that the two agents are co-formulated and co-administered, but rather one agent has a label that specifies its use in combination with the other. So, for example the immunocytokine is for use in treating or managing cancer, wherein the use comprises simultaneously, separately, or sequentially administering the immunocytokine and a further therapeutic agent, or vice e versa. But nothing in this application should exclude that the two combined agents are provided as a bundle or kit, or even are co-formulated and administered together where dosing schedules match. So, “administered in combination” includes (i) that the drugs are administered together in a joint infusion, in a joint injection or alike, (ii) that the drugs are administered separately but in parallel according to the given way of administration of each drug, and (iii) that the drugs are administered separately and sequentially.

Parallel administration in this context preferably means that both treatments are initiated together, e.g. the first administration of each drug within the treatment regimen are administered on the same day. Given potential different treatment schedules it is clear that during following days/weeks/months administrations may not always occur on the same day. In general, parallel administration aims for both drugs being present in the body at the same time at the beginning of each treatment cycle. Sequential administration in this context preferably means that both treatments are started sequentially, e.g., the first administration of the first drug occurs at least one day, preferably a few days or one week, earlier than the first administration of the second drug in order to allow a pharmacodynamic response of the body to the first drug before the second drug becomes active. Treatment schedules may then be overlapping or intermittent, or directly following each other.

The term “about”, when used together with a value, means the value plus/minus 10%, preferably 5% and especially 1% of its value.

Where the term “comprising” is used in the present description and claims, it does not exclude other elements. For the purposes of the present invention, the term “consisting of′ is considered to be a preferred embodiment of the term “comprising of′. If hereinafter a group is defined to comprise at least a certain number of embodiments, this is also to be understood to disclose a group, which preferably consists only of these embodiments.

Where an indefinite or definite article is used when referring to a singular noun, e.g., “a”, “an” or “the”, this includes a plural of that noun unless something else is specifically stated.

The term “at least one” such as in “at least one chemotherapeutic agent” may thus mean that one or more chemotherapeutic agents are meant. The term “combinations thereof” in the same context refers to a combination comprising more than one chemotherapeutic agents.

Technical terms are used by their common sense. If a specific meaning is conveyed to certain terms, definitions of terms will be given in the following in the context of which the terms are used.
“qxw”, from Latin quaque/each, every for every x week, e.g., q2w for every second week.
“s.c.” or “SC” for subcutaneously.
“i.v.” or “IV” for intravenously.
“ip” or “IP” for intraperitoneally.
cₘₐₓ for maximal concentration
AUC for area under the curve.

TABLE 1
list of molecules

| Molecule | Molecule characteristics |
|---|---|
| RLI x1 | RLI2 conjugated to the Cₜ of the “knob” antibody heavy chain |
| RLI x2 | RLI2 conjugated to the Cₜ of both antibody heavy chains |
| IL-15_{AQ} | IL-15 mutations: G78A, N79Q |
| IL-15_{AQA} | IL-15 mutations: N65A, G78A, N79Q |
| RLI2_{AQ} | RLI-15 mutations: G175A, N176Q |
| RLI2_{AQA} | RLI-15 mutations: N162A, G175A, N176Q |
| SOT201 | RLI-15 mutations: N162A (N65A), G175A (G78A), N176Q |
| | (N79Q) Pembrolizumab (“PEM”) with Fc modification: |
| | L235E, S228P, |
| (PEM L-RLI NA x1) | T366W (C_{H}3A chain “Knob”), |
| | T366S, L368A, Y407V (C_{H}3B chain “hole”) |
| PEM L-RLI DANA x1 | RLI-15 mutations: D158A (D61A), N162A, G175A, N176Q |
| (SOT201-DANA) | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LY-RLI DANA | RLI-15 mutations: D158A, N162A , G175A, N176Q |
| x1 | Pembrolizumab (“PEM”) with Fc modification: |
| | L235E, M252Y/S254T/T256E, S228P, |
| | T366W (C_{H}3A chain “Knob”), |
| | T366S, L368A, Y407V (C_{H}3B chain “hole”) |
| PEM LY-RLI | RLI-15 mutations: D158A, N162A (N65A), |
| DANAQD x1 | Q198D (Q101D), G175A, N176Q |
| | Pembrolizumab (“PEM”) with Fc modification: |
| | L235E, M252Y/S254T/T256E, S228P, |
| | T366W (C_{H}3A chain “Knob”), |
| | T366S, L368A, Y407V (C_{H}3B chain “hole”) |
| PEM LE-RLI x1 | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LE-RLI x2 | Pembrolizumab (“PEM”) with Fc modification: |
| | L235E, S228P, |
| | Pembrolizumab with RLI2 conjugated to the |
| | Cₜ of both antibody heavy chains |
| PEM LE-RLI-AD x1 | RLI-15 mutations: K10A, Q101D |
| | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LE-RLI-AD x2 | RLI-15 mutations: K10A, Q101D |
| | Pembrolizumab (“PEM”) with Fc modification: |
| | L235E, S228P |
| PEM LE-RLI-DA x1 | RLI-15 mutations: D61A |
| | Pembrolizumab (“PEM”) with Fc modification: |
| | L235E, S228P, |
| | T366W (C_{H}3A chain “Knob”), |
| | T366S, L368A, Y407V (C_{H}3B chain “hole”) |
| PEM LE-RLI-DA x2 | RLI-15 mutations: D61A |
| | Pembrolizumab (“PEM”) with Fc modification: |
| | L235E, S228P |
| PEM LE-RLI-NA x1 | RLI-15 mutations: N65A |
| | Pembrolizumab (“PEM”) with Fc modification: |
| | L235E, S228P |
| | T366W (C_{H}3A chain “Knob”), |
| | T366S, L368A, Y407V (C_{H}3B chain “hole”) |
| PEM LE-RLI-NA x2 | RLI-15 mutations: N65A |
| | Pembrolizumab (“PEM”) with Fc modification: |
| | L235E, S228P |
| PEM LE-RLI-ND x1 | RLI-15 mutations: N65D |
| | Pembrolizumab (“PEM”) with Fc modification: |
| | L235E, S228P |
| | T366W (C_{H}3A chain “Knob”), |
| | T366S, L368A, Y407V (C_{H}3B chain “hole”) |
| PEM LE-RLI-ND x2 | RLI-15 mutations: N65D |
| | Pembrolizumab (“PEM”) with Fc modification: |
| | L235E, S228P |
| PEM LE-RLI-NQD x1 | RLI-15 mutations: D30N, E64Q, N65D |
| | Pembrolizumab (“PEM”) with Fc modification: |
| | L235E, S228P |
| | T366W (C_{H}3A chain “Knob”), |
| | T366S, L368A, Y407V (C_{H}3B chain “hole”) |
| PEM LE-RLI-NQD x2 | RLI-15 mutations: D30N, E64Q, N65D |
| | Pembrolizumab (“PEM”) with Fc modification: |
| | L235E, S228P |
| PEM YTE-RLI x1 | Pembrolizumab (“PEM”) with Fc modification: |
| | M252Y, S254T, T256E, S228P, |
| | T366W (C_{H}3A chain “Knob”), |
| | T366S, L368A, Y407V (C_{H}3B chain “hole”) |
| PEM LE/YTE-RLI x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM YTE-RLI NA x1 | RLI-15 mutations: N65A |
| | Pembrolizumab (“PEM”) with Fc modification: |
| | M252Y, S254T, T256E, S228P, |
| | T366W (C_{H}3A chain “Knob”), |
| | T366S, L368A, Y407V (C_{H}3B chain “hole”) |
| PEM LE/YTE-RLI NA | RLI-15 mutations: N65A |
| x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM LE/YTE-RLI | RLI-15 mutations: Q101D, Q108A |
| QDQA x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM LE/YTE-RLI | RLI-15 mutations: D61A, N65A |
| DANA x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| PEM LE/YTE-RLI | RLI-15 mutations: D61A, N65A, Q101D |
| DANAQD x1 | Pembrolizumab (“PEM”) with Fc modification: |
| | L235E, M252Y, S254T, T256E, S228P, |
| | T366W (C_{H}3A chain “Knob”), |
| | T366S, L368A, Y407V (C_{H}3B chain “hole”) |
| RTX-RLI2 x2 | Rituximab with RLI2 conjugated to the |
| | Cₜ of both antibody heavy chains |
| RTX-RLI2 x1 | Rituximab with RLI2 conjugated to the Cₜ |
| | of the “knob” antibody heavy chain |
| | Rituximab with Fc modifications: |
| | T366W (C_{H}3A chain “Knob”), |
| | T366S, L368A, Y407V (C_{H}3B chain “hole”) |
| RTX-RLI2 AQ x2 | RLI-15 mutations: G175A, N176Q |
| RTX-RLI2 AQ x1 | RLI-15 mutations: G175A, N176Q |
| | Rituximab with Fc modifications: |
| | T366W (C_{H}3A chain “Knob”), |
| | T366S, L368A, Y407V (C_{H}3B chain “hole”) |
| RTX-L40-RLI2 x2 | Rituximab with RLI2 conjugated to the |
| | Cₜ of both antibody heavy chains |
| | L40: linker of 40 amino acids between the Cₜ of the antibody |
| RTX-L40-RLI2 AQ x2 | and RLI2 RLI-15 mutations: G175A, N176Q |
| | Rituximab with RLI2 conjugated to the Ct of both |
| | antibody heavy chains |
| | L40: linker of 40 amino acids between |
| | the Cₜ of the antibody and RLI2 |
| hCl1a LALA | hCl1a anti-Claudin 18.2 antibody with Fc modifications: |
| | L234A, L235A |
| hCl1a LALAPG | hCl1a anti-Claudin18.2 antibody with Fc modifications: |
| | L234A, L235A, P329G |
| hCl1a-RLI DANA | hCl1a with RLI2 conjugated to the Cₜ of the |
| (SOT202-DANA) | “knob” antibody heavy chain |
| | RLI-15 mutations: D61A, N65A |
| | hCl1a with Fc modification: |
| | T366W (C_{H}3A chain “Knob”), |
| | T366S, L368A, Y407V (C_{H}3B chain “hole”) |
| hCl1a DLE-RLI DANA | hCl1a with RLI2 conjugated to the Cₜ of the “knob” |
| (SOT202-DLE-DANA) | antibody heavy chain |
| | RLI-15 mutations: D61A, N65A |
| | hCl1a with Fc modification: |
| | S239D, A330L, 1332E |
| | T366W (C_{H}3A chain “Knob”), |
| | T366S, L368A, Y407V (C_{H}3B chain “hole”) |
| hCl1a DE-RLI DANA | hCl1a with RLI2 conjugated to the Cₜ of the |
| (SOT202-DE-DANA) | “knob” antibody heavy chain |
| | RLI-15 mutations: D61A, N65A |
| | hCl1a with Fc modification: |
| | S239D, I332E |
| | T366W (C_{H}3A chain “Knob”), |
| | T366S, L368A, Y407V (C_{H}3B chain “hole”) |
| hCl1a AAA-RLI DANA | hCl1a with RLI2 conjugated to the Cₜ of |
| | the “knob” antibody heavy chain |
| (SOT202-AAA-DANA) | RLI-15 mutations: D61A, N65A |
| | hCl1a with Fc modification: |
| | S298A, E333A, K334A |
| | T366W (C_{H}3A chain “Knob”), |
| | T366S, L368A, Y407V (C_{H}3B chain “hole”) |
| hCl1a TL-RLI DANA | hCl1a with RLI2 conjugated to the Cₜ of the |
| | “knob” antibody heavy chain |
| | RLI-15 mutations: D61A, N65A |
| | hCl1a with Fc modification: |
| | K392T, P396L |
| | T366W (C_{H}3A chain “Knob”), |
| | T366S, L368A, Y407V (C_{H}3B chain “hole”) |
| hCl1a IE-RLI DANA | hCl1a with RLI2 conjugated to the Cₜ of |
| | the “knob” antibody heavy chain |
| | RLI-15 mutations: D61A, N65A |
| | hCl1a with Fc modification: |
| | V264I, I332E |
| | T366W (C_{H}3A chain “Knob”), |
| | T366S, L368A, Y407V (C_{H}3B chain “hole”) |
| hCl1a-RLI DANA afuc | Afucosylated hCl1a with RLI2 conjugated to the |
| | Cₜ of the “knob” antibody heavy chain |
| (SOT202-afuc-DANA) | RLI-15 mutations: D61A, N65A |
| | hCl1a with Fc modification: |
| | T366W (C_{H}3A chain “Knob”), |
| | T366S, L368A, Y407V (C_{H}3B chain “hole”) |
| hCl1a DLE-RLI DANA | Afucosylated hCl1a with RLI2 conjugated to |
| afuc | the Cₜ of the “knob” antibody heavy chain |
| | RLI-15 mutations: D61A, N65A |
| | hCl1a with Fc modification: |
| | S239D, A330L, 1332E |
| | T366W (C_{H}3A chain “Knob”), |
| | T366S, L368A, Y407V (C_{H}3B chain “hole”) |
| hCl1a DE-RLI DANA | Afucosylated hCl1a with RLI2 conjugated |
| afuc | to the Cₜ of the “knob” antibody heavy chain |
| | RLI-15 mutations: D61A, N65A |
| | hCl1a with Fc modification: |
| | S239D, I332E |
| | T366W (C_{H}3A chain “Knob”), |
| | T366S, L368A, Y407V (C_{H}3B chain “hole”) |
| hCl1a AAA-RLI DANA | Afucosylated hCl1a with RLI2 conjugated |
| afuc | to the C of the “knob” antibody heavy chain |
| | RLI-15 mutations: D61A, N65A |
| | hCl1a with Fc modification: |
| | S298A, E333A, K334A |
| | T366W (C_{H}3A chain “Knob”), |
| | T366S, L368A, Y407V (C_{H}3B chain “hole”) |
| SOT202 (hCl1a-RLI | hCl1a with RLI2 conjugated to the Cₜ |
| NA) | of the “knob” antibody heavy chain |
| | RLI-15 mutations: N65A |
| | hCl1a with Fc modification: |
| | T366W (C_{H}3A chain “Knob”), |
| | T366S, L368A, Y407V (C_{H}3B chain “hole”) |
| hCl1a-RLI NA afuc | Afucosylated hCl1a with RLI2 conjugated to the |
| | Cₜ of the “knob” antibody heavy chain |
| | RLI-15 mutations: N65A |
| | hCl1a with Fc modification: |
| | T366W (C_{H}3A chain “Knob”), |
| | T366S, L368A, Y407V (C_{H}3B chain “hole”) |
| hCl1a LALAPG-RLI | hCl1a with RLI2 conjugated to the Cₜ of the |
| NA | “knob” antibody heavy chain |
| (SOT202-LALAPG) | RLI-15 mutations: N65A |
| | hCl1a with Fc modification: |
| | L234A, L235A, P329G |
| | T366W (C_{H}3A chain “Knob”), |
| | T366S, L368A, Y407V (C_{H}3B chain “hole”) |
| hCl1a LALAPG-RLI | hCl1a with RLI2 conjugated to the Cₜ |
| DANA | of the “knob” antibody heavy chain |
| (SOT202-LALAPG- | RLI-15 mutations: D61A, N65A |
| DANA) | hCl1a with Fc modification: |
| | L234A, L235A, P329G |
| | T366W (C_{H}3A chain “Knob”), |
| | T366S, L368A, Y407V (C_{H}3B chain “hole”) |

### DESCRIPTION OF THE INVENTION

In a first aspect the invention relates to an immunocytokine comprising a cytokine conjugate and an antibody or a functional fragment thereof. The cytokine conjugate comprises a polypeptide comprising the amino acid sequence of an interleukin 15 (IL-15) or a derivative thereof and the sushi domain of an interleukin 15-receptor alpha (IL-15Rα) or a derivative thereof. The antibody or functional variant thereof comprised in the immunocytokine is characterized by a heterodimeric Fc domain, a modified effector function (compared to the same immunocytokine with a wildtype Fc domain of the same IgG class) and/or having an increased in vivo half-life (compared to the same immunocytokine with a wildtype Fc domain of the same IgG class). The conjugate may be fused directly or indirectly to the C-terminus of both antibody heavy chains or antibody light chains, or, in case of a heterodimeric Fc domain, to the C-terminus of one antibody heavy chain. The increase in in vivo half-life of the immunocytokine may be achieved through Fc mutations that increase FcRn binding.

The Fc domain of the antibody or functional variant thereof may also comprise further modifications such as truncation of the C-terminal lysine of heavy chains, or in case of a heterodimeric Fc domain, of one or both heavy chains. Additionally, for indirect fusion a flexible linker composed of residues like glycine and serine may be introduced at the C-terminus of the heavy or light chains of the antibody so that the adjacent conjugate is free to move relative to the antibody Fc domain.

In one embodiment, the antibody or functional variant thereof comprised in the immunocytokine is not the antibody hCl1a, hCl1b, hCl1c, hCl1d, hCl1e, hCl1f, hCl1g, hCl1 h, hCl1i and hCl1j as disclosed in Table 4.

In one embodiment, the antibody or functional variant thereof comprised in the immunocytokine is the antibody hCl1a, hCl1b, hCl1c, hCl1d, hCl1e, hCl1f, hCl1g, hCl1 h, hCl1i and hCl1j as disclosed in Table 4.

In one embodiment, the invention provides an antibody or functional variant thereof binding to its target, the antibody being an IgG1, IgG2, IgG4, synthetic IgG or a bispecific antibody, or Fc-engineered versions thereof. In a preferred embodiment, the antibody is an IgG1 or IgG4 class antibody. When the target is present on tumour cells, the preferred antibody format is IgG1. When the target is present on immune cells, the preferred antibody format is IgG4. If the antibody format is an IgG1, the Fc region of immunoglobulins preferentially interacts with multiple Fcγ receptors (FcγR) and complement proteins (e.g., C1q), and mediates immune effector functions, such as elimination of targeted cells via antibody-dependent cellular cytotoxicity (ADCC), antibody-dependent cellular phagocytosis (ADCP) or complement-dependent cytotoxicity (CDC). For therapeutic approaches, it may be beneficial to enhance or silence Fc-mediated effector functions. Fc-mediated effector functions such as ADCC may be enhanced when the antibody is targeting tumour cells and silenced when the antibody is targeting check point inhibitors present on immune cells such as PD-1 or CTLA-4. When the antibody is targeting check point inhibitors present on immune cells such as PD-1 or CTLA-4, the antibody may be in the IgG4 format which is a poor inducer of Fc-mediated effector functions. In another embodiment, the antibody targeting a check point inhibitor such as PD-1 or CTLA-4 may be in the IgG1 format engineered to have strongly reduce or silenced ADCC and/or CDC activity, e.g., having reduced FcγR and C1q binding. Ample guidance on how to select IgG subclasses in developing anti-tumour therapeutic antibodies may be found in Yu J. et al (Yu, Song et al. 2020).

Antibody Fc-mediated function may be modulated using Fc-engineered immunoglobulins. Table 2 shows example of such Fc engineering.

TABLE 2
Examples of modifications to modulate antibody effector function. Unless otherwise noted, the
mutations are on the IgGI subclass. Adapted from Wang et al. (Wang, Mathieu et al. 2018).

| Engineering and intended | | |
|---|---|---|
| function | Mutation | Reference |
| Enhance ADCC | | |
| Increased FcγRIIIa binding | F243L/R292P/Y300L/V305I/P396L | (Stavenhagen, Gorlatov et al. 2007) |
| | S239D/1332E | (Lazar, Dang et al. 2006) |
| | S298A/E333A/K334A | (Shields, Namenuk et al. |
| | K392T/P396L | 2001) |
| | V264I/1332E | WO2006/088494 |
| | P247L/D270E/N241K | WO2004/099249 |
| | P2471/A339Q | WO2008/140603 |
| | in one heavy chain: L234Y/ | (Forero-Torres, de Vos et |
| | L235Q/G236W/S239M/H2- | al. 2012) |
| | 68D/D270E/S298A, | (Mimoto, Igawa et al. |
| | in the opposing heavy chain: D270E/ | 2013) |
| | K326D/A330M/K334E | (Nordstrom, Gorlatov et al. |
| | in the light chain: N65S. | 2011) |
| | in the heavy chain: L235V/ | |
| | F243L/R292P/Y300L/P39-6L | |
| Increased FcγRIIIa binding, | S239D/A330L/1332E | (Lazar, Dang et al. 2006) |
| decreased FcγRIIb binding | | |
| Enhance ADCP | | |
| Increased FcγRIIa binding, | G236A/S239D/1332E | (Richards, Karki et al. |
| | | 2008) |
| Increased FcγRIIIa binding | G236A/S239D/A330L/1332E | (Ahmed, Keremane et al. |
| | | 2016) |
| Enhance CDC | | |
| Increased C1q binding | K326W/E333S | (Idusogie, Wong et al. |
| | S267E/H268F/S324T | 2001) |
| | IgG1/IgG3 cross subclass | (Moore, Chen et al. 2010) |
| | | (Natsume, In et al. 2008) |
| Hexamerization | E345R/E430G/S440Y | (Diebolder, Beurskens et |
| | | al. 2014) |
| Reduce effector function | | |
| Aglycosylated | N297A or N297Q or N297G | (Tao and Morrison 1989, |
| | | Walker, Lund et al. 1989, |
| | | Bolt, Routledge et al. 1993, |
| | | Leabman, Meng et al. |
| | | 2013) |
| Reduced FcγR and C1q | IgG1: L234A/L235A or | (Xu, Alegre et al. 2000, |
| binding | L234A/L235A/P329G | Lo, Kim et al. 2017) |
| | IgG4: L235E | (Alegre, Collins et al. |
| | IgG4:F234A/L235A | 1992) |
| | E233P/F234V/L235A/D265A/L309V/ | (Xu, Alegre et al. 2000) |
| | R409K | (Zhang, Song et al. 2018) |
| | IgG2/IgG4 cross isotype | (Rother, Rollins et al. |
| | IgG2: H268Q/V309L/A330S/P331S | 2007) |
| | IgG2: | (An, Forrest et al. 2009) |
| | V234A/G237A/P238S/H268A/V309L/ | (Vafa, Gilliland et al. |
| | A330S/P331S | 2014) |
| Increase half-life | | |
| Increased FcRn | M252Y/S254T/T256E | (Dall'Acqua, Woods et al. |
| binding at pH 6.0 | M428L/N434S | 2002) |
| | | (Zalevsky, Chamberlain et |
| | | al. 2010) |
| Increased co-engagement | | |
| Increased FcγRIIb binding | S267E/L328F | (Chu, Vostiar et al. 2008) |
| Increased FcγRIIa binding, | N325S/L328F | (Shang, Daubeuf et al. |
| decreased FcγRIIIa binding | | 2014) |

Several laboratory methods exist for determining the efficacy of antibodies or effector cells in eliciting ADCC. Usually, a target cell line expressing a certain surface-exposed antigen is incubated with antibody or immunocytokine specific for that antigen. Upon incubation, effector cells expressing Fc receptor CD16 (Fc receptors FcγRIIIa (CD16a) and FcγRIIIb (CD16b)) are co-incubated with the antibody or immunocytokine-labelled target cells. Effector cells are typically PBMCs (peripheral blood mononuclear cell), of which a small percentage are NK cells; alternatively purified NK cells may be used. A further alternative is the use of the human NK cell line NK92 (ATCC CRL-2407) exogenously expressing human CD16 (NK92-hCD16). Over the course of a few hours a complex forms between the antibody, target cell, and effector cell which leads to lysis of the cell membrane of the target. If the target cell was pre-loaded with a label of some sort, that label is released in proportion to the amount of cell lysis. Cytotoxicity can be quantified by measuring the amount of label in solution compared to the amount of label that remains within healthy, intact cells. The label may be the radiolabel ⁵¹Cr, as described Perussia and Loza (Perussia and Loza 2000). Instead of using pre-loaded target cell, ADCC activity may also be measured using an LDH cytotoxicity assay. An LDH cytotoxicity assay is a colorimetric assay that provides a simple and reliable method for determining cellular cytotoxicity. Lactate dehydrogenase (LDH) is a cytosolic enzyme present in many different cell types that is released into the cell culture medium upon damage to the plasma membrane, such as plasma membrane damage occurring during ADCC. The LDH assay protocol is based on an enzymatic coupling reaction: LDH released from the cell oxidizes lactate to generate NADH, which then can react with water soluble tetrazolium salt (WST) to generate a yellow colour. The intensity of the generated colour correlates directly with the number of lysed cells. ADCC activity may also be measured as disclosed in Example 9.

As an alternative to ADCC assays using target and effector cells, Fc receptor binding of immunocytokines can also be tested by surface plasmon resonance (SPR), as described in Example 24.

In one embodiment, the modified effector function of the antibody or functional variant thereof comprised in the immunocytokine is a reduced antibody-dependent cell toxicity as compared to the same immunocytokine with a wildtype Fc domain of the same IgG class. The antibody effector functions may be reduced by reducing FcγR and C1q binding via the mutations listed in Table 2 in the corresponding section.

In one embodiment, when the antibody of functional variant thereof is an IgG1, reduced ADCC may be achieved through mutations selected from L234A/L235A, P329G, L234A/L235A/P329G, G236R/L328R, D265A, N297A, N297Q, N297G or L234A/L235A/G237A/P238S/H268A/A330S/P331S, preferably L234A/L235A/P329G.

In another embodiment, when the antibody of functional variant thereof is an IgG4, reduced ADCC may be achieved through mutations selected from L235E, F234A/L235A, F234A/L235A/P329G, P329G, S228P/L235E, S228P/F234A/L235A or E233P/F234V/L235A/D265A/R409K, preferably L235E.

In yet another embodiment, when the antibody of functional variant thereof is an IgG2, reduced ADCC may be achieved through mutation selected from H268Q/V309L/A330S/P331S or V234A/G237A/P238S/H268A/V309L/A330S/P331S.

In one embodiment, reduced ADCC may be achieved when the antibody of functional variant thereof is an IgG2 (IgG2a or IgG2b) and IgG4 hybrid or a functional variant thereof and comprises a CH1+hinge region from IgG2, and CH2+CH3 regions from IgG4 (IgG2 amino acids 118 to 260 and IgG4 amino acids 261 to 447).

In a preferred embodiment, reduced ADCC of an IgG1 antibody is achieved via the L234A/L235A (“LALA”) mutations, and may comprise the IgG1 Fc region of SEQ ID NO: 26.

In another preferred embodiment, reduced ADCC of an IgG1 antibody is achieved via the L234A/L235A/P329G (“LALAPG”) mutations, and may comprise the IgG1 Fc region of SEQ ID NO: 27.

Example 23 and FIG. 13 show that the immunocytokine hCl1a LALAPG-RLI DANA has nearly abolished ADCC activity when tested on A549-CLDN18.2 cells or PA-TU-8988 in the presence of NK92 cells, when compared to the hCl1a-RLI DANA immunocytokine of hCl1a antibody alone. The hCl1a LALA antibody has also reduced ADCC activity when compared to the hCl1a antibody, however the ADCC activity is not fully abolished.

In another preferred embodiment, when choosing an IgG4 antibody, the already poor induction of Fc-mediated effector functions may be further reduced via the L235E mutation, the F234A/L235A or the E233P/F234V/L235A/D265A/L309V/R409K mutations.

In a preferred embodiment, reduced ADCC of an IgG4 antibody is achieved via the L235E mutation, and may comprise the IgG4 FC region of SEQ ID NO: 43.

In another embodiment of the invention, the modified effector function of the antibody or functional variant thereof comprised in the immunocytokine is enhanced ADCC.

In one embodiment, the antibody may be modified to enhance ADCC through increased FcγRIIIa binding via the mutation listed in Table 2 in the corresponding section and/or by afucosylation.

In one embodiment, ADCC is enhanced in IgG1 antibodies or variants thereof via mutation selected from F243L/R292P/Y300L/V305I/P396L, S239D/I332E, S239D/I332E/A330L, S298A/E333A/K334A, K392T/P396L, V264I/I332E or L234Y/L235Q/G236W/S239M/H268D/D270E/S298A.

In a preferred embodiment, ADCC is enhanced in IgG1 antibodies or variants thereof via preferably from mutations selected from S239D/1332E (“DE”), S239D/1332E/A330L (“DLE”), S298A/E333A/K334A (“AAA”), K392T/P396L (“TL”) or V264I/I332E (“IE”).

In one embodiment, ADCC is enhanced in IgG1 antibodies via the DE mutations and may comprise the IgG1 Fc region of SEQ ID NO: 30.

In another embodiment, ADCC is enhanced in IgG1 antibodies via the DLE mutations and may comprise the IgG1 Fc region of SEQ ID NO: 31.

In another embodiment, ADCC is enhanced in IgG1 antibodies via the AAA mutations and may comprise the IgG1 Fc region of SEQ ID NO: 34.

In yet another embodiment, ADCC is enhanced in IgG1 antibodies via the TL mutations and may comprise the IgG1 Fc region of SEQ ID NO: 36.

In another embodiment, ADCC is enhanced in IgG1 antibodies via the IE mutations and may comprise the IgG1 Fc region of SEQ ID NO: 37.

In yet another embodiment, ADCC may also be enhanced by reducing the fucose content of the antibody via afucosylation (Pereira, Chan et al. 2018). Fucose (6-deoxy-L-galactose) is a common component of many N- and O-linked glycans produced in mammalian cells. Absence of core fucose on the Fc N-glycan of IgG1 at the conserved N-glycosylation site Asn297 (N297) in each of the CH2 domains has been shown to increase IgG1 Fc binding affinity to the FcγRIIIa present on immune effector cells such as natural killer cells and lead to enhanced ADCC activity. Fucosyltransferases (FUT) transfer a fucose residue from GDP-fucose to an acceptor substrate. FUT8 is the only α1,6-fucosyltransferase that transfers fucose via an α1,6 linkage to the innermost N-acetylglucosamine on N-glycans for core fucosylation of IgG1. Afucosylated antibodies may be produced in CHO cells where the FUT8 gene has been knocked-out (POTELLIGENT® technology). Antibodies produced in such a cell line have shown enhanced ADCC compared to the same antibody produced in conventional CHO cells (Yamane-Ohnuki, Kinoshita et al. 2004). Alternatively, antibodies may be produced in glycoengineered cell lines in which the fucose synthesis pathway has been deflected, also resulting in afucosylated antibodies (GlymaX®, ProBioGen) (Rosenlocher, Bohrsch et al. 2015, Dekkers, Plomp et al. 2016).

In one embodiment, the antibody may be modified to enhance ADCC through increased FcγRIIIa binding via afucosylation of the antibody.

In another embodiment, the antibody may be modified to enhance ADCC through increased FcγRIIIa binding via one of the mutations listed in Table 2 in the corresponding section, combined with afucosylation of the antibody.

In a preferred embodiment, the IgG1 antibody may be modified to enhance ADCC through increased FcγRIIIa binding via the AAA mutations, combined with afucosylation of the antibody.

The ADCC activity of immunocytokines with different Fc mutation enhancing ADCC activity, or afucosylated, or mutations combined with afucosylation is shown in Example 23 and FIG. 14, when measured by a cell based ADCC assay. All the mutations increased the ADCC activity of the tested immunocytokine to a similar extend, compared to the heterodimeric immunocytokine without the Fc domain mutations or the antibody alone. Likewise, afucosylation also enhanced ADCC activity of the immunocytokine. The combination of afucosylation with the AAA mutations also enhanced ADCC, whereas combination of DE or DLE with afucosylation even lowered ADCC activity compared to DE, DLE or afucosylation alone.

Fc receptor binding of immunocytokines with mutations modulating effector functions was also tested by SPR, as described in

Example 24. SPR allowed to evaluation the antibody Fc binding to ADCC-activating receptors FcγRIIIa V158, and FcγRIIIa F158 and ADCC-inhibitory receptor FcγRIIb. The SPR testing confirmed that overall, the immunocytokines with mutations enhancing ADCC show a higher A/I ration than the immunocytokine without mutations enhancing ADCC, unless the antibody glycosylation was affected by the mutation.

Introducing mutation into Fc domain may also impact the stability and developability of immunocytokines and may depend on each particular antibody used for the immunocytokine. More specifically, the meting temperature and glycosylation of immunocytokines with Fc mutations have been tested (see example 25). Overall, for the hCl1a-based immunocytokine, while the TL and IE mutations introduced unfavourable glycosylation and the DE and DLE mutations decreased the C_{H}2 domain melting temperature impacting its stability, the AAA mutations, optionally combined with afucosylation, did not impact the stability and developability of the hCl1a-based immunocytokine.

In another embodiment, modifications made in the Fc domain of the antibody to improve its stability may be the S228P mutation in IgG4 antibodies to avoid Fab arm exchange (Silva, Vetterlein et al. 2015) (SEQ ID NO: 39).

In one embodiment, the antibody Fc domains may be heterodimeric in order the have only one heavy chain fused to the cytokine. Heterodimerization may be achieved by mutations in the CH3 chains of each of the two Fc domains of the antibody (C_{H}3A chain and C_{H}3B chain). Table 3 below summarizes designs for heterodimeric Fc variants (Ha, Kim et al. 2016).

TABLE 3
Examples of designs of heterodimeric Fc variants

| Heterodimeric | Paired mutations | | |
|---|---|---|---|
| Fc name | C_{H}3A chain | C_{H}3B chain | Reference |
| KIH | T366W | T366S/L368A/Y407V | (Elliott, Ultsch et al. |
| | | | 2014) |
| KiH_{S-S} | T366W/S354C | T366S/L368A/Y407V/ | (Merchant, Zhu et al. |
| | | Y349C | 1998, Leaver-Fay, |
| | | | Froning et al. 2016) |
| HA-TF | S364H/F405A | Y349T/T394F | (Moore, Bautista et al. |
| | | | 2011) |
| ZW1 | T350V/L351Y/F405A/ | T350V/T366L/K392L/ | (Von Kreudenstein, |
| | Y407V | T394W | Escobar-Carbrera et al. |
| | | | 2013) |
| 7.8.60 | K360D/D399M/Y407A | E345R/Q347R/T366V/ | (Leaver-Fay, Froning et |
| | | K409V | al. 2016) |
| DD-KK | K409D/K392D | D399K/E356K | (Gunasekaran, Pentony |
| | | | et al. 2010) |
| EW-RVT | K360E/K409W | Q347R/D399V/F405T | (Choi, Kim et al. 2013, |
| | | | Choi, Seok et al. 2015) |
| EW-RVT_{S-S} | K360E/K409W/Y349C | Q347R/D399V/F405T/ | (Choi, Seok et al. 2015) |
| | | S354C | |
| SEED | IgA-derived 45 residues | IgG1-derived 57 residues | (Davis, Aperlo et al. |
| | on IgG1 CH3 | on IgA CH3 | 2010) |
| A107 | K370E/K409W | E357N/D399V/F405T | (Choi, Kim et al. 2015) |

In one embodiment, heterodimerization of the antibody may be achieved by using any one of the following heterodimeric Fc variants: KiH, KiHS-S, HA-TF, ZW1, 7.8.60, DD-KK, EW-RVT, EW-RVTS-S, SEED or A107.

In a preferred embodiment, heterodimerization of the antibody is achieved via the T366W mutation in the CH3 domain of one heavy chain (SEQ ID NO: 28) and the T366S/L368A/Y407V mutations in the CH3 domain of the opposing heavy chain (SEQ ID NO: 29), resulting in a “Knobs-into-Holes (KiH)” Fc variant. In Example 2, the potency of a homodimerized with two RLI2 conjugates is compared to the potency of an heterodimerized immunocytokine. Table 11 show that, although only one RLI2 conjugate is present in the heterodimeric immunocytokine (RTX-RLI x1), surprisingly its potency is nevertheless still above 50% of the potency of the homodimeric immunocytokine (RTX-RLI 2×).

Methods to produce heterodimeric immunocytokines can be found in Example 3. Measurements of the potency of such heterodimeric immunocytokines, compared to homodimeric immunocytokine can be found in Example 5 and Table 15. As one aim of the present invention is to reduce the potency of the conjugate, the inventors now show that, whereas the homodimeric immunocytokine having two RLL2 conjugates fused the C-termini had a minor reduction of potency, the heterodimeric immunocytokine having only one RLI2 conjugate showed an about 10 fold reduction in potency on kit225 cells.

Preferably, the RLI2 conjugate is fused to the knob heavy chain.

An exemplary sequence of an heterodimeric immunocytokine of the present invention can be found in SEQ ID NO: 21 (“HC knob-RLI2_{AQ}NA”), SEQ ID NO: 22 or SEQ ID NO: 101 (“HC hole” with or without the terminal lysine deletion) and SEQ ID NO: 23 (LC), preferably SEQ ID NO: 21, SEQ ID NO: 101 and SEQ ID NO: 23 for SOT201 (pembrolizumab-variant based heterodimeric immunocytokine); SEQ ID NO: 85 (“HC knob-RLI2_{AQ}DANA”), SEQ ID NO: 87 (“HC hole”) and SEQ ID NO: 88 (LC) for a hCl1a-variant based heterodimeric immunocytokine; SEQ ID NO: 111 (“HC knob-RLI2_{AQ}NA”), SEQ ID NO: 110 (“HC hole”) and SEQ ID NO: 88 ((LC) for another hCl1a-variant based heterodimeric immunocytokine; SEQ ID NO: 97 (“HC knob AAA-RLI2_{AQ}NA”), SEQ ID NO: 98 (“HC hole AAA”) and SEQ ID NO: 99 (LC) for an heterodimeric immunocytokine based on rituximab or SEQ ID NO: 94 (“HC knob-RLI2_{AQ}DANA”), SEQ ID NO: 95 (“HC hole”) and SEQ ID NO: 92 (LC) for an heterodimeric immunocytokine based on cetuximab.

In one embodiment, the heterodimeric Fc domain leads to higher yield of the immunocytokine upon expression in cell culture, compared to an immunocytokine with homodimeric Fc domain. Although it is generally expected that the heterodimeric antibody formats have lower expression due to mispairing of the heavy and light chains, surprisingly, it was observed for the heterodimeric immunocytokines, that heterodimeric constructs using the KiH technology had a higher expression compared to respective homodimeric constructs (see Example 3).

When the immunocytokine is not heterodimeric, the conjugate may also be fused to the C-termini of the light chains of the antibody (e.g., SEQ ID NO: 45). A linker consisting of glycines or serines and glycines may be between the C-terminus of the light chain and the N-terminus of the conjugate to allow for flexibility of the fused conjugate relative to the antibody.

Alternatively, a linker may be used for fusing RLI2_{AQ} to the C-terminus of one or both heavy chains. Such linker is preferably composed of glycines or glycines and serines, more preferably composed of GGGGS units with a length of 30 to 50 amino acids, especially the L40 linker of SEQ ID NO: 100.

In another embodiment, the invention relates to an immunocytokine wherein the in vivo half-life of the immunocytokine is increased and wherein the antibody or functional variant thereof is an IgG1 or an IgG4 antibody or a functional variant thereof and comprises a mutation selected from M252Y/S254T/T256E, M428L/N434S or T250Q/M428L.

The Fc domain plays a central role in the stability and serum half-life of antibodies. Antibody in vivo half-life may be increased via the M252Y/S254T/T256E or M428L/N434S mutation in the Fc domain increasing FcRn binding (Dall'Acqua, Woods et al. 2002, Zalevsky, Chamberlain et al. 2010).

In one embodiment, the half-life of antibodies of the IgG1 or IgG4 type is increased via the M252Y/S254T/T256E (“YTE”) mutations, respectively the Fc domain of SEQ ID NO: 35 and SEQ ID NO: 44.

In another embodiment, the invention relates to an immunocytokine wherein the antibody or functional variant thereof has reduced ADCC and wherein the antibody or functional variant thereof is an IgG4 antibody or a functional variant thereof and comprises a L235E mutation and a KiH-heterodimeric Fc domain. Reducing ADCC may be beneficial when the antibody target is a check-point inhibitor present on an immune cell, such as PD-1 or CTLA-4 on the surface of T cells, to avoid NK cell-induced cytotoxicity toward these immune cells. The IgG4 antibody may also optionally contain the S228P mutation to stabilize the antibody. The IgG4 Fc domain with the L235E mutation may be of the sequence SEQ ID NO: 43. The IgG4 CH1-hinge domain with the S228P mutation may be of the sequence SEQ ID NO: 39. KiH-heterodimeric Fc domain of the IgG4 antibody may be of the sequence SEQ ID NO: 41 (“knob”) and SEQ ID NO: 42 (“hole”). Optionally, one or preferably both heavy chains may have the terminal lysine deletion (dK), i.e. the sequence SEQ ID NO: 41 (“knob”) and SEQ ID NO: 42 (“hole”). In another embodiment, both heavy chains have the terminal lysine.

Example 10 to Example 26 relate to such an immunocytokine.

In one embodiment the conjugate of the immunocytokine is a fusion protein comprising, in N- to C-terminal order, the IL-15Rα sushi domain or a derivative thereof, a linker and the IL-15 or a derivative thereof, preferably wherein the IL-15Rα sushi domain comprises the sequence of SEQ ID NO: 5, more preferably the IL-15Rα sushi+fragment of SEQ ID NO: 6, and wherein the linker has a length of 18 to 22 amino acids and is composed preferably of glycines or serines and glycines, more preferably has the sequence of SEQ ID NO: 7, and wherein the IL-15 has the sequence of SEQ ID NO: 2.

The fusion protein of having the IL-15Rα sushi+fragment of SEQ ID NO: 6 fused via the flexible linker of SEQ ID NO: 7 to the N-terminus of the mature human IL-15 of SEQ ID NO: 2 is referred to as RLI2 for Receptor-Linker-Interleukin 2 or SO-C101 having the sequence of SEQ ID NO: 8, and is a clinical stage IL-2/IL-15Rβγ superagonist with low immunogenicity. This make such fusion protein a preferred conjugate to be used in an immunocytokine format.

In a preferred embodiment, the immunocytokine comprises an IL-15 variant which comprises at least one mutation increasing the homogeneity of the IL-15 variant with respect to post-translational modifications, preferably wherein the mutation reduces deamidation at N77 and/or glycosylation at N79 of IL-15 mature human IL-15 (SEQ ID NO: 2), more preferably wherein the mutation is selected from mutations G78A, G78V, G78L or G78I, and N79Q, N79S or N79T, most preferably wherein the mutation is G78A/N79Q, (the “AQ mutation”).

The IL-15 mutations increasing the homogeneity of the immunocytokine and the IL-15 mutations reducing the binding to the IL-2/IL-15Rβ and/or to the γ_{c} receptor may be used independently in immunocytokines of the invention or may be combined in immunocytokines of the invention.

In another preferred embodiment, the immunocytokine comprises an IL-15 variant which comprises at least one mutation that reduces the binding to the IL-2/IL-15Rβ and/or to the γ_{c} receptor, preferably wherein the mutated amino acid is selected from N1, N4, S7, D8, K10, K11, D30, D61, E64, N65, L69, N72, E92, Q101, Q108, I111 of IL-15 mature human IL-15 having the sequence of SEQ ID NO: 2, more preferably wherein the mutated amino acid is selected from D61, N65 and Q101, most preferably wherein the mutated amino acid is N65.

The mutation that reduces the binding to the IL-2/IL-15Rβ and/or to the γc receptor is preferably a substitution selected from N1D, N1A, N1G, N4D, S7Y, S7A, D8A, D8N, K10A, K11A, D30N, D61A, D61N, E64Q, N65D, N65A, N65E, N65R, N65K, L69R, N72R, Q101D, Q101E, Q108D, Q108A, Q108E and Q108R, preferably D8A, D8N, D61A, D61N, N65A, N65D, N72R, Q101D, Q101E and Q108A, more preferably D61A, N65A and Q101, most preferably N65A or a combined substitution selected from D8N/N65A, D61A/N65A or D61A/N65A/Q101D.

In another embodiment, the immunocytokine comprises an antibody or functional variant thereof, which binds to a tumour antigen, preferably selected from EGFR, HER2, FGFR2, FOLR1, CLDN18.2, CEA, GD2, O-Acetyl-GD-2, GM1, CAIX, EPCAM, MUC1, PSMA, c-Met, ROR1, GPC3, CD19, CD20, CD38; to a tumour extracellular matrix antigen, preferably selected from FAP, the EDA domain of fibronectin, the EDB domain of fibronectin and LRRC15, preferably FAP and the EDB domain of fibronectin; to a neovascularization antigen, preferably VEGF, or Endoglin; (CD105); or is an immunomodulatory antibody or a functional variant thereof, wherein the immunomodulatory antibody stimulates a co-stimulatory receptor, preferably selected from CD40 agonists, CD137/4-1BB agonists, CD134/OX40 agonists and TNFRSF18/GITR agonists, or wherein the immunomodulatory antibody inhibits an immunosuppressive receptor, preferably selected from PD-1 antagonists, CTLA-4 antagonists, LAG3 antagonists, TIGIT antagonists, inhibitory KIRs antagonists, BTLA/CD272 antagonists, HAVCR2/TIM-3/CD366 antagonists and ADORA2A antagonists, more preferably PD-1 antagonists.

Antibodies against the listed targets above are well known in the art or can be generated by standard immunization or phage display protocols. Non-human antibodies can be humanized. Examples of anti-EGFR antibodies are cetuximab, panitumumab, zalutumumab, nimotuzumab, and matuzumab. Examples of anti-HER2 antibodies are trastuzumab, permtuzumab or margetuximab. Examples of anti-CLDN18.2 antibodies are zolbetuximab and antibodies of the invention below. An example of an anti-CEA antibody is arcitumomab. An example of an anti-GD2 is hu14.18K322A. An example of an anti O-Acetyl-GD-2 is c.8B6. FGFR2, FOLR1, GM1, CAIX, EPCAM, MUC1, PSMA, c-Met, ROR1, GPC3, CD19, Examples of anti-CD20 antibodies are rituximab, ocrelizumab, obinutuzumab, ofatumumab, ibritumomab, tositumomab and ublituximab. Examples of anti-CD38 antibodies are daratumumab, MOR202 and isatuximab.

Examples anti-FAP antibodies are Sibrotuzumab and B12 (US 2020-0246383A1). An example of an anti-EDA domain antibody of fibronectin is the F8 antibody ((Villa, Trachsel et al. 2008), WO 2010/078945, WO 2014/174105), an example of an anti-EDB domain of fibronectin is the L19 antibody ((Pini, Viti et al. 1998), WO 1999/058570), and an example of an anti-LRRC15 antibody is Samrotamab/huM25 (WO 2017/095805).

Examples of anti-VEGF antibodies are bevacizumab and ranibizumab. An example of an anti-Endoglin antibody is TRC 105 (WO 2010039873A2).

Examples of anti-CD40 agonistic antibodies are selicrelumab, APX005M, ChiLob7/4, ADC-1013, SEA-CD40 and CDX-1140 (Vonderheide 2020). Examples of anti-CD137/4-1BB agonistic antibodies are urelumab and utomilumab (Chester, Sanmamed et al. 2018). Examples of anti-CD134/OX40 agonistic antibodies PF-04518600, MEDI6469, MOXR0916, MEDI0562, INCAGN01949 (Fu, Lin et al. 2020). An example of an anti-TNFRSF18/GITR agonistic antibody is DTA-1.

Examples of PD-1 antagonists are anti-PD-1 antibodies, anti-PD-L1 antibodies or anti-PD-L2 antibodies Examples of anti-PD-1 antagonistic antibodies are pembrolizumab, nivolumab, pidilizumab, toripalimab and tislelizumab (Dolgin 2020). Examples of anti-PD-L1 antagonistic antibodies are atezolizumab and avelumab. An example of an anti-CTLA-4 antagonistic antibody is ipilimumab. An example of an anti-LAG3 antagonistic antibody is relatlimab. Examples of anti-TIGIT antagonistic antibodies are Tiragolumab, Vibostolimab, Domvanalimab, Etigilimab, BMS-986207, EOS-448, COM902, ASP8374, SEA-TGT, BGB-A1217, IBI-939 and M6223.

An example of an anti-BTLA antagonistic antibody is TAB004. Examples of anti-HAVCR2/TIM-3 antagonistic antibodies are LY3321367, MBG453 and TSR-022.

A preferred embodiment is an immunocytokine, wherein the conjugate comprises the sequence of SEQ ID NO: 10 and the antibody comprises the pembrolizumab-derived heavy chain knob sequence of SEQ ID NO: 20, the pembrolizumab-derived heavy chain hole sequence of SEQ ID NO: 22, and the light chain sequence of SEQ ID NO: 16, wherein the conjugate is fused to the C-terminus heavy chain knob sequence without a linker, preferably SEQ ID NO: 21.

In one embodiment, the immunocytokine of the present invention comprises the conjugate of the sequence SEQ ID NO: 10) or SEQ ID NO: 11 and the antibody is an anti-CLDN18.2 heterodimeric IgG1 antibody variant having a VH and VL domain sequence of Table 4, the IgG1 variant being heterodimeric through the KiH mutation of Table 3, having enhanced ADCC activity through the DE, DLE, AAA, TL or IE mutations of Table 2 or through afucosylation, or through the combination of a mutation listed above and afucosylation.

In another embodiment, the immunocytokine of the present invention comprises the conjugate of the sequence SEQ ID NO: 10) or SEQ ID NO: 11 and the antibody is an anti-CLDN18.2 heterodimeric IgG1 antibody variant having a VH and VL domain sequence of Table 4, the IgG1 variant being heterodimeric through the KiH mutation of Table 3.

TABLE 4
VH and VL domain sequences of anti-CLDN18.2 antibodies

| | VH domain | VL domain |
|---|---|---|
| hCl1a | SEQ ID NO: 46 | SEQ ID NO: 47 |
| hCl1b | SEQ ID NO: 48 | SEQ ID NO: 49 |
| hCl1c | SEQ ID NO: 50 | SEQ ID NO: 51 |
| hCl1d | SEQ ID NO: 52 | SEQ ID NO: 53 |
| hCl1e | SEQ ID NO: 54 | SEQ ID NO: 55 |
| hCl1f | SEQ ID NO: 56 | SEQ ID NO: 57 |
| hCl1g | SEQ ID NO: 58 | SEQ ID NO: 59 |
| hCl1h | SEQ ID NO: 60 | SEQ ID NO: 61 |
| hCl1i | SEQ ID NO: 62 | SEQ ID NO: 63 |
| hCl1j | SEQ ID NO: 64 | SEQ ID NO: 65 |
| hGBA-1 | SEQ ID NO: 66 | SEQ ID NO: 67 |
| hGBA-2 | SEQ ID NO: 68 | SEQ ID NO: 69 |
| hGBA-3 | SEQ ID NO: 70 | SEQ ID NO: 71 |
| hGBA-4 | SEQ ID NO: 72 | SEQ ID NO: 73 |
| hGBA-5 | SEQ ID NO: 74 | SEQ ID NO: 75 |
| hGBA-6 | SEQ ID NO: 76 | SEQ ID NO: 77 |
| hGBA-7 | SEQ ID NO: 78 | SEQ ID NO: 80 |
| hGBA-8 | SEQ ID NO: 80 | SEQ ID NO: 81 |
| hGBA-9 | SEQ ID NO: 82 | SEQ ID NO: 83 |
| Zolbetixumab | SEQ ID NO: 89 | SEQ ID NO: 90 |

In a preferred embodiment, the immunocytokine of the present invention comprises the conjugate of the sequence SEQ ID NO: 10 and the anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3.

In a preferred embodiment, the immunocytokine of the present invention comprises the conjugate of the sequence SEQ ID NO: 10 and the anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having enhanced ADCC activity through the DE, DLE, AAA, TL or IE mutations of Table 2 or through afucosylation, or through the combination of a mutation listed above and afucosylation.

In another preferred embodiment, the immunocytokine of the present invention comprises the conjugate of the sequence SEQ ID NO: 10 and the anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having enhanced ADCC activity through afucosylation.

In a preferred embodiment, the immunocytokine comprises a conjugate of the sequence of SEQ ID NO: 11, and the antibody variant is a heterodimeric IgG1 anti-CLDN18.2 antibody having heavy chain knob sequence of SEQ ID NO: 84, heavy chain hole sequence of SEQ ID NO: 87 and the light chain sequence of SEQ ID NO: 88.

An exemplary sequence for a preferred immunocytokine may be SEQ ID NO: 85 (“HC knob”), SEQ ID NO: 87 (“HC hole”) and SEQ ID NO: 88 (LC).

Another exemplary sequence for a preferred immunocytokine may be SEQ ID NO: 86 (“HC knob”), SEQ ID NO: 87 (“HC hole”) and SEQ ID NO: 88 (LC).

Yet another exemplary sequence for a preferred immunocytokine may be SEQ ID NO: 111 (“HC knob”), SEQ ID NO: 110 (“HC hole”) and SEQ ID NO: 88 (LC).

In another preferred embodiment the immunocytokine of the present invention comprises the conjugate of the sequence SEQ ID NO: 10 and where the antibody variant is a heterodimeric IgG1 anti-CLDN18.2 antibody having heavy chain knob sequence of SEQ ID NO: 84, heavy chain hole sequence of SEQ ID NO: 87 and the light chain sequence of SEQ ID NO: 88.

Surprisingly, the RLI2AQ DANA mutant fused to the anti-Claudin18.2 antibody hCl1a showed higher ADCC compared to the RLI2AQ NA mutant.

In yet another embodiment, the immunocytokine of the present invention comprises the conjugate of the sequence SEQ ID NO: 10 and an anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having the S239D/I332E (DE) ADCC-enhancing mutation in the IgG1 Fc domain.

In yet another embodiment, the immunocytokine of the present invention comprises the conjugate of the sequence SEQ ID NO: 1 land an anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having the S239D/I332E (DE) ADCC-enhancing mutation in the IgG1 Fc domain.

In another embodiment, the immunocytokine of the present invention comprises a the fusion protein having the sequence of SEQ ID NO: 10 and the antibody variant is a heterodimeric IgG1 anti-EGFR antibody having the VH sequence of SEQ ID NO: 91 and the VL sequence of SEQ ID NO: 92, the IgG1 variant being heterodimeric through the KiH mutation of Table 3, having enhanced ADCC activity through FC mutations listed in Table 2 in the corresponding section or through afucosylation, or through the combination of mutations and afucosylation.

In another embodiment, the invention relates to a nucleic acid encoding the immunocytokines herein disclosed.

In yet another embodiment, the invention relates to a vector comprising the nucleic acid coding for the immunocytokines.

In a further embodiment, the invention relates to a host cell comprising the vector or nucleic acid coding for the immunocytokines.

Another embodiment of the invention relates to the immunocytokine, the nucleic acid or the vector for use in treatment.

Yet another embodiment of the invention relates to a pharmaceutical composition comprising the immunocytokine, the nucleic acid or the vector and a pharmaceutically acceptable carrier.

In another embodiment, the immunocytokine, the nucleic acid or the vector may be for use in the treatment of a subject suffering from, at risk of developing and/or being diagnosed for a neoplastic disease or an infectious disease.

In another embodiment, the invention relates to a method for treating a patient suffering from, at risk of developing and/or being diagnosed for a neoplastic disease or an infectious disease comprising administering the immunocytokine, the nucleic acid or the vector.

As the typical clinical development path is the combination with standard of care, the immunocytokines of the invention may be administration in combination with other agents, typically the standard of care of the specific indication is it approved in. The immunocytokine of the invention may be combined with a checkpoint inhibitor, which may be an anti-PD-1 antibody, an anti-PD-L1 antibody, an anti-PD-L2 antibody, an anti-LAG3, an anti-TIM-3, an anti-CTLA4 antibody or an anti-TIGIT antibody, preferably an anti-PD-L1 antibody or an anti-PD-1 antibody. These antibodies have in common that they block/antagonize cellular interactions that block or downregulate immune cells, especially T cells from killing cancer cells, accordingly these antibodies are all antagonistic antibodies. Examples of anti-PD-1 antibodies are pembrolizumab, nivolumab, cemiplimab (REGN2810), BMS-936558, SHR1210, IB1I308, PDR001, BGB-A317, BCD-100 and JS001; examples of anti-PD-L1 antibodies are avelumab, atezolizumab, durvalumab, KN035 and MGD013 (bispecific for PD-1 and LAG-3); an example for PD-L2 antibodies is sHIgM12; examples of anti-LAG-3 antibodies are relatlimab (BMS 986016), Sym022, REGN3767, TSR-033, GSK2831781, MGD013 (bispecific for PD-1 and LAG-3) and LAG525 (IMP701); examples of anti-TIM-3 antibodies are TSR-022 and Sym023; examples of anti-CTLA-4 antibodies are ipilimumab and tremelimumab (ticilimumab); examples of anti-TIGIT antibodies are tiragolumab (MTIG7192A, RG6058) and etigilimab.

SEQUENCES

SEQ ID NO: 1: human IL-15

| | | |
|---|---|---|
| 1 | MRISKPHLRS ISIQCYLCLL LNSHELTEAG IHVFILGCFS AGLPKTEANW VNVISDLKKI | |
| 061 | EDLIQSMHID ATLYTESDVH PSCKVTAMKC FLLELQVISL ESGDASIHDT VENLIILANN | |
| 121 | SLSSNGNVTE SGCKECEELE EKNIKEFLQS FVHIVQMFIN TS | 162 |

Signal peptide underlined
SEQ ID NO: 2: mature human IL-15

| | | |
|---|---|---|
| 1 | NWVNVISDLK KIEDLIQSMH IDATLYTESD VHPSCKVTAM KCFLLELQVI SLESGDASIH | |
| 061 | DTVENLIILA NNSLSSNGNV TESGCKECEE LEEKNIKEFL QSFVHIVQMF INTS | 114 |

with G78 and N79 bold/underlined
SEQ ID NO: 3: mature human IL-15_{AQ}

| | | |
|---|---|---|
| 1 | NWVNVISDLK KIEDLIQSMH IDATLYTESD VHPSCKVTAM KCFLLELQVI SLESGDASIH | |
| 061 | DTVENLIILA NNSLSSNAQV TESGCKECEE LEEKNIKEFL QSFVHIVQMF INTS | 114 |

with A78 and Q79 bold/underlined
SEQ ID NO: 4: human IL-15Rα

| | | |
|---|---|---|
| 1 | MAPRRARGCR TLGLPALLLL LLLRPPATRG ITCPPPMSVE HADIWVKSYS LYSRERYICN | |
| 61 | SGFKRKAGTS SLTECVLNKA TNVAHWTTPS LKCIRDPALV HQRPAPPSTV TTAGVTPQPE | |
| 121 | SLSPSGKEPA ASSPSSNNTA ATTAAIVPGS QLMPSKSPST GTTEISSHES SHGTPSQTTA | |
| 181 | KNWELTASAS HQPPGVYPQG HSDTTVAIST STVLLCGLSA VSLLACYLKS RQTPPLASVE | |
| 241 | MEAMEALPVT WGTSSRDEDL ENCSHHL | 267 |

SEQ ID NO: 5: sushi domain of IL-15Rα

| | | |
|---|---|---|
| 001 | CPPPMSVEHA DIWVKSYSLY SRERYICNSG FKRKAGTSSL TECVLNKATN VAHWTTPSLK | 60 |
| 061 | C | |

SEQ ID NO: 6: sushi+ fragment of IL-15Rα

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPP | 77 |

SEQ ID NO: 7: linker

| | | |
|---|---|---|
| 001 | SGGSGGGGSG GGSGGGGSGG | 20 |

SEQ ID NO: 8: RLI2 (SOT101)

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV ENLIILANNS LSSNGNVTES | |
| 181 | GCKECEELEE KNIKEFLQSF VHIVQMFINT S | 211 |

SEQ ID NO: 9: RLI2_{AQ}

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV ENLIILANNS LSSNAQVTES | |
| 181 | GCKECEELEE KNIKEFLQSF VHIVQMFINT S | 211 |

SEQ ID NO: 10: RLI2_{AQ} N162A (N65A) or RLI-15_{AQA}

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHDTV EALIILANNS LSSNAQVTES | |
| 181 | GCKECEELEE KNIKEFLQSF VHIVQMFINT S | 211 |

SEQ ID NO: 11: RLI2_{AQ} D158A/N162A (D158A/N162A)

| | | |
|---|---|---|
| 001 | ITCPPPMSVE HADIWVKSYS LYSRERYICN SGFKRKAGTS SLTECVLNKA TNVAHWTTPS | |
| 061 | LKCIRDPALV HQRPAPPSGG SGGGGSGGGS GGGGSGGNWV NVISDLKKIE DLIQSMHIDA | |
| 121 | TLYTESDVHP SCKVTAMKCF LLELQVISLE SGDASIHATV EALIILANNS LSSNAQVTES | |
| 181 | GCKECEELEE KNIKEFLQSF VHIVQMFINT S | 211 |

SEQ ID NO: 12: pembrolizumab heavy chain (HC)-human IgG4 κ isotype

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFLGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLTCLVK GFYPSDIAVE WESNGQPENN YKTTPPVLDS DGSFFLYSRL TVDKSRWQEG | 420 |
| 421 | NVFSCSVMHE ALHNHYTQKS LSLSLG | |

The pembrolizumab HC has stabilizing S228P mutation; for immunocytokines herein,
terminal K has been deleted (dK) to reduce heterogeneity.
SEQ ID NO: 13: pembrolizumab HC CDR1

| | |
|---|---|
| 001 | NYYMY |

SEQ ID NO: 14: pembrolizumab HC CDR2

| | |
|---|---|
| 001 | GINPSNGGTNFNEKFKN |

SEQ ID NO: 15: pembrolizumab HC CDR3

| | |
|---|---|
| 001 | RDYRFDMGFDY |

SEQ ID NO: 16: pembrolizumab light chain

| | | |
|---|---|---|
| 001 | EIVLTQSPAT LSLSPGERAT LSCRASKGVS TSGYSYLHWY QQKPGQAPRL LIYLASYLES | 060 |
| 061 | GVPARFSGSG SGTDFTLTIS SLEPEDFAVY YCQHSRDLPL TFGGGTKVEI KRTVAAPSVE | 120 |
| 121 | IFPPSDEQLK SGTASVVCLL NNFYPREAKV QWKVDNALQS GNSQESVTEQ DSKDSTYSLS | 180 |
| 181 | STLTLSKADY EKHKVYACEV THQGLSSPVT KSFNRGEC | |

SEQ ID NO: 17: pembrolizumab LC CDR1

| | |
|---|---|
| 001 | RASKGVSTSGYSYLH |

SEQ ID NO: 18: pembrolizumab LC CDR2

| | |
|---|---|
| 001 | LASYLES |

SEQ ID NO: 19: pembrolizumab LC CDR3

| | |
|---|---|
| 001 | QHSRDLPLT |

SEQ ID NO: 20 pembrolizumab HC knob: IgG4 S228P.L235E.T366W.dk

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLWCLVK GFYPSDIAVE WESNGQPENN YKTTPPVLDS DGSFFLYSRL TVDKSRWQEG | 420 |
| 421 | NVFSCSVMHE ALHNHYTQKS LSLSLG | 446 |

SEQ ID NO: 21: SOT201 HC knob: IgG4 S228P/L235E/T366W/dK-RLI2.N162A/G175A/N176Q

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLWCLVK GFYPSDIAVE WESNGQPENN YKTTPPVLDS DGSFFLYSRL TVDKSRWQEG | 420 |
| 421 | NVFSCSVMHE ALHNHYTQKS LSLSLGITCP PPMSVEHADI WVKSYSLYSR ERYICNSGFK | 480 |
| 481 | RKAGTSSLTE CVLNKATNVA HWTTPSLKCI RDPALVHQRP APPSGGSGGG GSGGGSGGGG | 540 |
| 541 | SGGNWVNVIS DLKKIEDLIQ SMHIDATLYT ESDVHPSCKV TAMKCFLLEL QVISLESGDA | 600 |
| 601 | SIHDTVEALI ILANNSLSSN AQVTESGCKE CEELEEKNIK EFLQSFVHIV QMFINTS | 657 |

SEQ ID NO: 22: HC hole: S228P.L235E.T366S.L368A.Y407V

| | | |
|---|---|---|
| 001 | QVQLVQSGVE VKKPGASVKV SCKASGYTFT NYYMYWVRQA PGQGLEWMGG INPSNGGTNF | 060 |
| 061 | NEKFKNRVTL TTDSSTTTAY MELKSLQFDD TAVYYCARRD YRFDMGFDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLSCAVK GFYPSDIAVE WESNGQPENN YKTTPPVLDS DGSFFLVSRL TVDKSRWQEG | 420 |
| 421 | NVFSCSVMHE ALHNHYTQKS LSLSLGK | |

SEQ ID NO: 23: SOT201 LC

| | | |
|---|---|---|
| 001 | EIVLTQSPAT LSLSPGERAT LSCRASKGVS TSGYSYLHWY QQKPGQAPRL LIYLASYLES | 060 |
| 061 | GVPARFSGSG SGTDFTLTIS SLEPEDFAVY YCQHSRDLPL TFGGGTKVEI KRTVAAPSVE | 120 |
| 121 | IFPPSDEQLK SGTASVVCLL NNFYPREAKV QWKVDNALQS GNSQESVTEQ DSKDSTYSLS | 180 |
| 181 | STLTLSKADY EKHKVYACEV THQGLSSPVT KSFNRGEC | 218 |

SEQ ID NO: 24: IgG1 CHI with hinge
ASTKGPSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCP
SEQ ID NO: 25: IgG1 Fc
APELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 26: IgG1 Fc LALA
APEAAGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 27: IgG1 Fc LALA PG
APEAAGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALGAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 28: IgG1 Fc KiH-knob
APELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLWCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 29: IgG1 Fc KiH-hole
APELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLSCAVKGFYPSDIAVE
WESNGQPENNYKTTPPVLDSDGSFFLVSKLTVDKSRWQQGNVFSCSVMHEALHNHYTQKSLSLSPGK
SEQ ID NO: 30: IgG1 Fc DE (S239D/1332E)
APELLGGPDVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPEEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 31: IgG1 Fc DLE (S239D/A330L/1332E)
APELLGGPDVFLFPPKPKDTLMISRTPEVTCVVVDVSHEDPEVKFNWYVDGVEVHNAKTKPREEQYNSTYRVVSV
LTVLHQDWLNGKEYKCKVSNKALPLPEEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 32: IgG1 Fc DAE (S239D/1332E/G236A)
APELLAGPDVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPEEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 33: IgG1 Fc GASDALIE (G236A/S239D/A330L/1332E)
APELLAGPDVFLFPPKPKDTLMISRTPEVTCVVVDVSHEDPEVKFNWYVDGVEVHNAKTKPREEQYNSTYRVVSV
LTVLHQDWLNGKEYKCKVSNKALPLPEEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 34: IgG1 Fc AAA (S298A/E333A/K334A)
APELLGGPSVFLFPPKPKDTLMISRTPEVTCVVVDVSHEDPEVKFNWYVDGVEVHNAKTKPREEQYNATYRVVSV
LTVLHQDWLNGKEYKCKVSNKALPAPIAATISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 35: IgG1 Fc YTE (M252Y/S254T/T256E)
APELLGGPSVFLFPPKPKDTLYITREPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 36: IgG1 Fc TL (K392T/P396L)
APELLGGPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLVKGFYPSDIAVE
WESNGQPENNYTTTPLVLDSDGSFFLYSKLTVDKSRWQQGNVFSCSVMHEALHNHYTQKSLSLSPGK
SEQ ID NO: 37: IgG1 Fc IE (V264I/1332E)
APELLGGPSVFLFPPKPKDTLMISRTPEVTCVVIDVSHEDPEVKFNWYVDGVEVHNAKTKPREEQYNSTYRVVSV
LTVLHQDWLNGKEYKCKVSNKALPAPEEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 38: CL (kappa)
RTVAAPSVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGEC
SEQ ID NO: 39: IgG4 CHI hinge S228P
ASTKGPSVFPLAPCSRSTSESTAALGCLVKDYFPEPVTVSWNSGALTSGVHTFPAVLQSSGLYSLSSVVTVPSSS
LGTKTYTCNVDHKPSNTKVDKRVESKYGPPCPPCP
SEQ ID NO: 40: IgG4 Fc
APEFLGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 41: IgG4 Fc KiH-knob
APEFLGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLWCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 42: IgG4 Fc KiH-hole
APEFLGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLSCAVKGFYPSDIAVE
WESNGQPENNYKTTPPVLDSDGSFFLVSRLTVDKSRWQEGNVFSCSVMHEALHNHYTQKSLSLSLGK
SEQ ID NO: 43: IgG4 Fc LE (L235E)
APEFEGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 44: IgG4 Fc YTE (M252Y/S254T/T256E)
APEFLGGPSVFLFPPKPKDTLYITREPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
SEQ ID NO: 45: LC (kappa)-RLI AQ
RTVAAPSVFIFPPSDEQLKSGTASVVCLLNNFYPREAKVQWKVDNALQSGNSQESVTEQDSKDSTYSLSSTLTLS
KADYEKHKVYACEVTHQGLSSPVTKSFNRGECITCPPPMSVEHADIWVKSYSLYSRERYICNSGFKRKAGTSSLT
ECVLNKATNVAHWTTPSLKCIRDPALVHQRPAPPSGGSGGGGSGGGSGGGGSGGNWVNVISDLKKIEDLIQSMHI
DATLYTESDVHPSCKVTAMKCFLLELQVISLESGDASIHDTVENLIILANNSLSSNAQVTESGCKECEELEEKNI
KEFLQSFVHIVQMFINTS
SEQ ID NO: 46: hCl1a VH
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSS
SEQ ID NO: 47: hCl1a VL
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIK
SEQ ID NO: 48: hCl1b VH
QVQLVQSGAEVKKPGASVKVSCKASGYTFTDYAMHWVRQAPGQRLEWMGWINTYTGKPTYSQKFQGRVTITRDTS
ASTAYMELSSLRSEDTAVYYCARAVFYGYTMDAWGQGTLVTVSS
SEQ ID NO: 49: hCl1b VL
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIK
SEQ ID NO: 50: hCl1c VH
QVQLVQSGAEVKKPGASVKVSCKASGYTFTDYAMHWVRQAPGQRLEWMGWINTYTGKPTYSQKFQGRVTITRDTS
ASTAYMELSSLRSEDTAVYYCARAVFYGYTMDAWGQGTLVTVSS
SEQ ID NO: 51: hCl1c VL
DIQMTQSPSSLSASVGDRVTITCRTSEDIYSNLAWYQQKPGKAPKLLIFAIKRLQDGVPSRFSGSGSGTDFTLTI
SSLQPEDFATYYCLQGSKFPLTFGQGTKVEIK
SEQ ID NO: 52: hCl1d VH
QVQLVQSGAEVKKPGASVKVSCKASGYTFTDYAMHWVRQAPGQRLEWMGWINTYTGKPTYSQKFQGRVTITRDTS
ASTAYMELSSLRSEDTAVYYCARAVFYGYTMDAWGQGTLVTVSS
SEQ ID NO: 53: hCl1d VL
DIQMTQSPSSLSASVGDRVTITCRTSEDIYSNFAWYQQKPGKAPKLLIYSVNRLQDGVPSRFSGSGSGTDFTLTI
SSLQPEDFATYYCLQGSKFPLTFGQGTKVEIK
SEQ ID NO: 54: hCl1e VH
QVQLVQSGAEVKKPGASVKVSCKASGYTFTDYAMYWVRQAPGQRLEWMGWINTYTGKPTYAQKFQGRVTITRDTS
ASTAYMELSSLRSEDTAVYYCARAVFYGYTMDAWGQGTLVTVSS
SEQ ID NO: 55: hCl1e VL
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIK
SEQ ID NO: 56: hCl1f VH
QVQLVQSGAEVKKPGASVKVSCKASGYTFTDYAMHWVRQAPGQRLEWMGWINAYTGKPTYAQKFQGRVTITRDTS
ASTAYMELSSLRSEDTAVYYCARAVFYGYTMDAWGQGTLVTVSS
SEQ ID NO: 57: hCl1f VL
DIQMTQSPSSLSASVGDRVTITCRASEDIYSNLAWYQQKPGKAPKLLIYSVKRLQDGVPSRESGSGSGTDFTLTI
SSLQPEDFATYYCLQGSNFPLTFGQGTKVEIK
SEQ ID NO: 58: hCl1g VH
QVQLVQSGAEVKKPGASVKVSCKASGYTFTDYAMHWVRQAPGQRLEWMGWINAYTGKPTYAQKFQGRVTITRDTS
ASTAYMELSSLRSEDTAVYYCARAVFYGYTMDAWGQGTLVTVSS
SEQ ID NO: 59: hCl1g VL
DIQMTQSPSSLSASVGDRVTITCRTSEDIYSNFAWYQQKPGKAPKLLIYSVNRLQDGVPSRFSGSGSGTDFTLTI
SSLQPEDFATYYCLQGSKFPLTFGQGTKVEIK
SEQ ID NO: 60: hCl1h VH
QVQLVQSGAEVKKPGASVKVSCKASGYTFTDYAMYWVRQAPGQRLEWMGWINAYTGKPTYAQKFQGRVTITRDTS
ASTAYMELSSLRSEDTAVYYCARAVYYGYTMDAWGQGTLVTVSS
SEQ ID NO: 61: hCl1h VL
DIQMTQSPSSLSASVGDRVTITCRASEDIYSNLAWYQQKPGKAPKLLIYSVKRLQDGVPSRFSGSGSGTDFTLTI
SSLQPEDFATYYCLQGSNFPLTFGQGTKVEIK
SEQ ID NO: 62: hCl1i VH
QVQLVQSGAEVKKPGASVKVSCKASGYTFTDYAMYWVRQAPGQRLEWMGWINAYTGKPTYAQKFQGRVTITRDTS
ASTAYMELSSLRSEDTAVYYCARAVYYGYTMDAWGQGTLVTVSS
SEQ ID NO: 63: hCl1i VL
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIK
SEQ ID NO: 64: hCl1j VH
QVQLVQSGAEVKKPGASVKVSCKASGYTFTDYAMYWVRQAPGQRLEWMGWINAYTGKPTYAQKFQGRVTITRDTS
ASTAYMELSSLRSEDTAVYYCARAVYYGYTMDAWGQGTLVTVSS
SEQ ID NO: 65: hCl1j VL
DIQMTQSPSSLSASVGDRVTITCRTSEDIYSNLAWYQQKPGKAPKLLIFAIKRLQDGVPSRFSGSGSGTDFTLTI
SSLQPEDFATYYCLQGSKFPLTFGQGTKVEIK
SEQ ID NO: 66: hGBA-1 VH
EVQLVQSGAEVKKPGESLKISCKGSGYSFTSYWIGWVRQMPGKGLEWMGNIYPGASDTRYAPSFQGQVTISADKS
ISTAYLQWSSLKASDTAMYYCARLWRGNSFDYWGQGTLVTVSS
SEQ ID NO: 67: hGBA-1 VL
DIVMTQSPDSLAVSLGERATINCKSSQSLLNSGNQKNYLAWYQQKPGQPPKLLIYWASTRESGVPDRFSGSGSGT
DFTLTISSLQAEDVAVYYCQNDYSYPFTFGQGTKVEIK
SEQ ID NO: 68: hGBA-2 VH
EVQLVQSGAEVKKPGESLKISCKGSGYSFTSYWIGWVRQMPGKGLEWMGNIYPGDADTRYAPSFQGQVTISADKS
ISTAYLQWSSLKASDTAMYYCARMWRGNSFDYWGQGTLVTVSS
SEQ ID NO: 69: hGBA-2 VL
DIVMTQSPDSLAVSLGERATINCKSSQSLLNSGNQKNYLAWYQQKPGQPPKLLIYWASTRESGVPDRFSGSGSGT
DFTLTISSLQAEDVAVYYCQNDYSYPFTFGQGTKVEIK
SEQ ID NO: 70: hGBA-3 VH
EVQLVQSGAEVKKPGESLKISCKGSGYSFTSYWIGWVRQMPGKGLEWMGIIYPGASDTNYAPSFQGQVTISADKS
ISTAYLQWSSLKASDTAMYYCARIWRGNSFDYWGQGTLVTVSS
SEQ ID NO: 71: hGBA-3 VL
DIVMTQSPDSLAVSLGERATINCKSSQSLLNSGNQKNYLAWYQQKPGQPPKLLIYWASTRESGVPDRESGSGSGT
DFTLTISSLQAEDVAVYYCQNDYSYPLTFGQGTKVEIK
SEQ ID NO: 72: hGBA-4 VH
EVQLVQSGAEVKKPGESLKISCKGSGYSFTSYWIGWVRQMPGKGLEWMGIIYPGDAYTRYSPSFQGQVTISADKS
ISTAYLQWSSLKASDTAMYYCTRLWRGNSFDAWGQGTLVTVSS
SEQ ID NO: 73: hGBA-4 VL
DIVMTQSPDSLAVSLGERATINCKSSQSLLNSGNQKNYLTWYQQKPGQPPKLLIYWASTRESGVPDRESGSGSGT
DFTLTISSLQAEDVAVYYCQNDYSYPLTFGQGTKVEIK
SEQ ID NO: 74: hGBA-5 VH
EVQLVQSGAEVKKPGESLKISCKGSGYSFTSYWIGWVRQMPGKGLEWMGIIYPGAAYTRYAPSFQGQVTISADKS
ISTAYLQWSSLKASDTAMYYCARLWRGNSFDYWGQGTLVTVSS
SEQ ID NO: 75: hGBA-5 VL
DIVMTQSPDSLAVSLGERATINCKSSQSLLNSGNQKNYLAWYQQKPGQPPKLLIYWASTRESGVPDRESGSGSGT
DFTLTISSLQAEDVAVYYCQQDYSYPFTFGQGTKVEIK
SEQ ID NO: 76: hGBA-6 VH
EVQLVQSGAEVKKPGESLKISCKGSGYTFTSYWIGWVRQMPGKGLEWMGNIYPGASYTRYSPSFQGQVTISADKS
ISTAYLQWSSLKASDTAMYYCTRQWRGNSFDYWGQGTLVTVSS
SEQ ID NO: 77: hGBA-6 VL
DIVMTQSPDSLAVSLGERATINCKSSQSLLNSGNQKNYLAWYQQKPGQPPKLLIYWASTRESGVPDRESGSGSGT
DFTLTISSLQAEDVAVYYCQNDYSYPFTFGQGTKVEIK
SEQ ID NO: 78: hGBA-7 VH
EVQLVQSGAEVKKPGESLKISCKGSGYSFTSYWIGWVRQMPGKGLEWMGNIYPGEAYTRYSPSFQGQVTISADKS
ISTAYLQWSSLKASDTAMYYCTRLWRGNSFDYWGQGTLVTVSS
SEQ ID NO: 79: hGBA-7 VL
DIVMTQSPDSLAVSLGERATINCKSSQSVLNSGNQKNYLTWYQQKPGQPPKLLIYWASTRESGVPDRESGSGSGT
DFTLTISSLQAEDVAVYYCQNDYSYPFTFGQGTKVEIK
SEQ ID NO: 80: hGBA-8 VH
EVQLVQSGAEVKKPGESLKISCKGSGYSFTSYWIGWVRQMPGKGLEWMGNIYPSESYTNYAPSFQGQVTISADKS
ISTAYLQWSSLKASDTAMYYCTRLWRGNSFDYWGQGTLVTVSS
SEQ ID NO: 81: hGBA-8 VL
DIVMTQSPDSLAVSLGERATINCKSSQSLLNSGNQKNYLAWYQQKPGQPPKLLIYWASTRESGVPDRESGSGSGT
DFTLTISSLQAEDVAVYYCQNDYSYPFTFGQGTKVEIK
SEQ ID NO: 82: hGBA-9 VH
EVQLVQSGAEVKKPGESLKISCKGSGYTFTSYWIGWVRQMPGKGLEWMGIIYPSAAYTRYAPSFQGQVTISADKS
ISTAYLQWSSLKASDTAMYYCTRMWRGNSFDYWGQGTLVTVSS
SEQ ID NO: 83: hGBA-9 VL
DIVMTQSPDSLAVSLGERATINCKSSQSVLNSGNQKNYLAWYQQKPGQPPKLLIYWASTRESGVPDRFSGSGSGT
DETLTISSLQAEDVAVYYCQQDYSYPFTFGQGTKVEIK
SEQ ID NO: 84: hCl1a HC AAA Knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPG
SEQ ID NO: 85: hCl1a HC AAA Knob RLI2 DANA
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHATVEALIILANNSLSSNAQVTESGCKECEELEEKNIKEFLQSFVHIVQMFINTS
SEQ ID NO: 86: hCl1a HC AAA Knob RLI2 NA
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 87: hCl1a HC AAA Hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLSCAVKGFY
PSDIAVEWESNGQPENNYKTTPPVLDSDGSFFLVSKLTVDKSRWQQGNVFSCSVMHEALHNHYTQKSLSLSPG
SEQ ID NO: 88: hCl1a LC
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID NO: 89: Zolbetuximab (IMAB362) VH
QVQLQQPGAELVRPGASVKLSCKASGYTFTSYWINWVKQRPGQGLEWIGNIYPSDSYTNYNQKFKDKATLTVDKS
SSTAYMQLSSPTSEDSAVYYCTRSWRGNSFDYWGQGTTLTVSS
SEQ ID NO: 90: Zolbetuximab (IMAB362) VL
DIVMTQSPSSLTVTAGEKVTMSCKSSQSLLNSGNQKNYLTWYQQKPGQPPKLLIYWASTRESGVPDRFTGSGSGT
DFTLTISSVQAEDLAVYYCQNDYSYPFTFGSGTKLEIK
SEQ ID NO: 91: Cetuximab VH
QVQLKQSGPGLVQPSQSLSITCTVSGFSLTNYGVHWVRQSPGKGLEWLGVIWSGGNTDYNTPFTSRLSINKDNSK
SQVFFKMNSLQSNDTAIYYCARALTYYDYEFAYWGQGTLVTVSA
SEQ ID NO: 92: Cetuximab VL
DILLTQSPVILSVSPGERVSFSCRASQSIGTNIHWYQQRTNGSPRLLIKYASESISGIPSRFSGSGSGTDFTLSI
NSVESEDIADYYCQQNNNWPTTFGAGTKLELK
SEQ ID NO: 93: Cetuximab HC AAA Knob
QVQLKQSGPGLVQPSQSLSITCTVSGFSLTNYGVHWVRQSPGKGLEWLGVIWSGGNTDYNTPFTSRLSINKDNSK
SQVFFKMNSLQSNDTAIYYCARALTYYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPG
SEQ ID NO: 94: Cetuximab HC AAA Knob RLI2 DANA
QVQLKQSGPGLVQPSQSLSITCTVSGFSLTNYGVHWVRQSPGKGLEWLGVIWSGGNTDYNTPFTSRLSINKDNSK
SQVFFKMNSLQSNDTAIYYCARALTYYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHATVEALIILANNSLSSNAQVTESGCKECEELEEKNIKEFLQSFVHIVQMFINTS
SEQ ID NO: 95 Cetuximab HC AAA Hole
QVQLKQSGPGLVQPSQSLSITCTVSGFSLTNYGVHWVRQSPGKGLEWLGVIWSGGNTDYNTPFTSRLSINKDNSK
SQVFFKMNSLQSNDTAIYYCARALTYYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNA
TYRVVSVLTVLHQDWLNGKEYKCKVSNKALPAPIAATISKAKGQPREPQVYTLPPSRDELTKNQVSLSCAVKGFY
PSDIAVEWESNGQPENNYKTTPPVLDSDGSFFLVSKLTVDKSRWQQGNVFSCSVMHEALHNHYTQKSLSLSPG
SEQ ID NO: 96: Rituximab heavy chain
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVFSCSVMHEALHNHYTQKSLSLSPG
SEQ ID NO: 97 Rituximab HC AAA Knob RLI AQNA
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NATYRVVSVLTVLHQDWLNGKEYKCKVSNKALPAPIAATISKAKGQPREPQVYTLPPSRDELTKNQVSLWCLVKG
FYPSDIAVEWESNGQPENNYKTTPPVLDSDGSFFLYSKLTVDKSRWQQGNVFSCSVMHEALHNHYTQKSLSLSPG
ITCPPPMSVEHADIWVKSYSLYSRERYICNSGFKRKAGTSSLTECVLNKATNVAHWTTPSLKCIRDPALVHQRPA
PPSGGSGGGGSGGGSGGGGSGGNWVNVISDLKKIEDLIQSMHIDATLYTESDVHPSCKVTAMKCFLLELQVISLE
SGDASIHDTVEALIILANNSLSSNAQVTESGCKECEELEEKNIKEFLQSFVHIVQMFINTS
SEQ ID NO: 98 Rituximab HC AAA Hole
QVQLQQPGAELVKPGASVKMSCKASGYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NATYRVVSVLTVLHQDWLNGKEYKCKVSNKALPAPIAATISKAKGQPREPQVYTLPPSRDELTKNQVSLSCAVKG
FYPSDIAVEWESNGQPENNYKTTPPVLDSDGSFFLVSKLTVDKSRWQQGNVFSCSVMHEALHNHYTQKSLSLSPG
SEQ ID NO: 99 Rituximab LC
QIVLSQSPAILSASPGEKVTMTCRASSSVSYIHWFQQKPGSSPKPWIYATSNLASGVPVRFSGSGSGTSYSLTIS
RVEAEDAATYYCQQWTSNPPTFGGGTKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
SEQ ID NO: 100: L40 linker
GGGGSGGGGSGGGGSGGGGSGGGGSGGGGSGGGGSGGGGS
SEQ ID NO: 101: SOT201 HC hole: S228P.L235E.T366S.L368A.Y407V/dK

| | | |
|---|---|---|
| 001 | QVQLVQSGVE VKKPGASVKV SCKASGYTFT NYYMYWVRQA PGQGLEWMGG INPSNGGTNE | 060 |
| 061 | NEKFKNRVTL TTDSSTTTAY MELKSLQFDD TAVYYCARRD YRFDMGEDYW GQGTTVTVSS | 120 |
| 121 | ASTKGPSVFP LAPCSRSTSE STAALGCLVK DYFPEPVTVS WNSGALTSGV HTFPAVLQSS | 180 |
| 181 | GLYSLSSVVT VPSSSLGTKT YTCNVDHKPS NTKVDKRVES KYGPPCPPCP APEFEGGPSV | 240 |
| 241 | FLFPPKPKDT LMISRTPEVT CVVVDVSQED PEVQFNWYVD GVEVHNAKTK PREEQFNSTY | 300 |
| 301 | RVVSVLTVLH QDWLNGKEYK CKVSNKGLPS SIEKTISKAK GQPREPQVYT LPPSQEEMTK | 360 |
| 361 | NQVSLSCAVK GFYPSDIAVE WESNGQPENN YKTTPPVLDS DGSFFLVSRL TVDKSRWQEG | 420 |
| 421 | NVFSCSVMHE ALHNHYTQKS LSLSLG | |

SEQ ID NO: 102: mPD1.VH-h1.HC.D265A.E356K.N399K.dk-RLI.N162A.G175A.N176Q
murine antiPD-1 (mIgG1 D265A HC1-RLI-15_{AQA})
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKKQMAKDKVSLTCMITNFFPEDIT
VEWQWNGQPAENYKNTQPIMKTDGSYFVYSKLNVQKSNWEAGNTFTCSVLHEGLHNHHTEKSLSHSPITCPPPMS
VEHADIWVKSYSLYSRERYICNSGFKRKAGTSSLTECVLNKATNVAHWTTPSLKCIRDPALVHQRPAPPSGGSGG
GGSGGGSGGGGSGGNWVNVISDLKKIEDLIQSMHIDATLYTESDVHPSCKVTAMKCFLLELQVISLESGDASIHD
TVEALIILANNSLSSNAQVTESGCKECEELEEKNIKEFLQSFVHIVQMFINTS
SEQ ID NO: 103: mPD1.VH-h1.HC.D265A.K409E.K439D.dk
murine antiPD-1 (mIgG1 D265A HC2)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTFRSV
SELPIMHQDWLNGKEFKCRVNSAAFGAPIEKTISKTKGRPKAPQVYTIPPPKEQMAKDKVSLTCMITNFFPEDIT
VEWQWNGQPAENYKNTQPIMNTDGSYFVYSELNVQKSNWEAGNTFTCSVLHEGLHNHHTEDSLSHSP
SEQ ID NO: 104: mPD1.VL-hk.LC
murine antiPD-1 (mIgG1 Light Chain)
DIVMTQGTLPNPVPSGESVSITCRSSKSLLYSDGKTYLNWYLQRPGQSPQLLIYWMSTRASGVSDRFSGSGSGTD
FTLKISGVEAEDVGIYYCQQGLEFPTFGGGTKLELKRTDAAPTVSIFPPSSEQLTSGGASVVCFLNNFYPKDINV
KWKIDGSERQNGVLNSWTDQDSKDSTYSMSSTLTLTKDEYERHNSYTCEATHKTSTSPIVKSFNRNEC
SEQ ID NO: 105: human IL-2

| | | |
|---|---|---|
| 001 | APTSSSTKKT QLQLEHLLLD LQMILNGINN YKNPKLTRML TFKFYMPKKA TELKHLQCLE | 060 |
| 061 | EELKPLEEVL NLAQSKNFHL RPRDLISNIN VIVLELKGSE TTFMCEYADE TATIVEFLNR | 120 |
| 121 | WITFCQSIIS TLT | 133 |

SEQ ID NO: 106: IL-2v

| | | |
|---|---|---|
| 001 | APASSSTKKT QLQLEHLLLD LQMILNGINN YKNPKLTRML TAKFAMPKKA TELKHLQCLE | 060 |
| 061 | EELKPLEEVL NGAQSKNFHL RPRDLISNIN VIVLELKGSE TTFMCEYADE TATIVEFLNR | 120 |
| 121 | WITFAQSIIS | |

SEQ ID NO: 107: IL-15 M1

| | | |
|---|---|---|
| 001 | AWVNVISDLK KIEDLIQSMH IDATLYTESN VHPSCKVTAM KCFLLGLQRI SLESGDASIH | 060 |
| 061 | DTVENLIILA NNSLSSNGNV TESGCKECEE LEEKNIKEFL QSFVHIVQMF INTS | 114 |

SEQ ID NO: 108: IL-15 M2

| | | |
|---|---|---|
| 001 | GWVNVISDLK KIEDLIQSMH IDATLYTESN VHPSCKVTAM KCFLLGLQRI SLESGDASIH | 060 |
| 061 | DTVQNLIILA NNSLSSNGNV TESGCKECEE LEEKNIKEFL QSFVHIVQMF INTS | 114 |

SEQ ID NO: 109: hCl1a HC Knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPG
SEQ ID NO: 110: hCl1a HC Hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLSCAVKGFY
PSDIAVEWESNGQPENNYKTTPPVLDSDGSFFLVSKLTVDKSRWQQGNVFSCSVMHEALHNHYTQKSLSLSPG
SEQ ID NO: 111: hCl1a HC Knob RLI2 NA
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLWCLVKGFY
PSDIAVEWESNGQPENNYKTTPPVLDSDGSFFLYSKLTVDKSRWQQGNVFSCSVMHEALHNHYTQKSLSLSPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 112: PD1-IL2v HC1: HC with IL2v (Fc knob, LALAPG),
IL2v.T3A.F42A.Y45A.L72G.C125A
EVQLLESGGGLVQPGGSLRLSCAASGFSFSSYTMSWVRQAPGKGLEWVATISGGGRDIYYPDSVKGRFTISRDNS
KNTLYLQMNSLRAEDTAVYYCVLLTGRVYFALDSWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVYTLPPCRDELTKNQVSLWCLVKGF
YPSDIAVEWESNGQPENNYKTTPPVLDSDGSFFLYSKLTVDKSRWQQGNVFSCSVMHEALHNHYTQKSLSLSPGG
GGGSGGGGSGGGGSAPASSSTKKTQLQLEHLLLDLQMILNGINNYKNPKLTRMLTAKFAMPKKATELKHLQCLEE
ELKPLEEVLNGAQSKNFHLRPRDLISNINVIVLELKGSETTFMCEYADETATIVEFLNRWITFAQSIISTLT
SEQ ID NO: 113: PD1-IL2v HC2: HC (Fc hole LALAPG)
EVQLLESGGGLVQPGGSLRLSCAASGFSFSSYTMSWVRQAPGKGLEWVATISGGGRDIYYPDSVKGRFTISRDNS
KNTLYLQMNSLRAEDTAVYYCVLLTGRVYFALDSWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALGAPIEKTISKAKGQPREPQVCTLPPSRDELTKNQVSLSCAVKGF
YPSDIAVEWESNGQPENNYKTTPPVLDSDGSFFLVSKLTVDKSRWQQGNVFSCSVMHEALHNHYTQKSLSLSP
SEQ ID NO: 114: PD1-IL2v LC
DIVMTQSPDSLAVSLGERATINCKASESVDTSDNSFIHWYQQKPGQSPKLLIYRSSTLESGVPDRFSGSGSGTDF
TLTISSLQAEDVAVYYCQQNYDVPWTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SEQ ID NO: 115: mPD1-IL2v HC1: mPD-1.VH-h1.HC.D265A.E356K.N399K.dk-
IL2v.T3A.F42A.Y45A.L72G.C125A murine antiPD-1 (mIgG1 D265A HC1-IL-2v)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTERSV
SELPIMHQDWLNGKEFKCRVNSAAFGAPIEKTISKTKGRPKAPQVYTIPPPKKQMAKDKVSLTCMITNFFPEDIT
VEWQWNGQPAENYKNTQPIMKTDGSYFVYSKLNVQKSNWEAGNTFTCSVLHEGLHNHHTEKSLSHSPAPASSSTK
KTQLQLEHLLLDLQMILNGINNYKNPKLTRMLTAKFAMPKKATELKHLQCLEEELKPLEEVLNGAQSKNFHLRPR
DLISNINVIVLELKGSETTFMCEYADETATIVEFLNRWITFAQSIISTLT
SEQ ID NO: 116: mPD1-IL2v HC2: mPD-1.VH-h1.HC.D265A.K409E.K439D.dk murine
antiPD-1 (mIgG1 D265A HC2)
EVQLQESGPGLVKPSQSLSLTCSVTGYSITSSYRWNWIRKFPGNRLEWMGYINSAGISNYNPSLKRRISITRDTS
KNQFFLQVNSVTTEDAATYYCARSDNMGTTPFTYWGQGTLVTVSSAKTTPPSVYPLAPGSAAQTNSMVTLGCLVK
GYFPEPVTVTWNSGSLSSGVHTFPAVLQSDLYTLSSSVTVPSSTWPSQTVTCNVAHPASSTKVDKKIVPRDCGCK
PCICTVPEVSSVFIFPPKPKDVLTITLTPKVTCVVVAISKDDPEVQFSWFVDDVEVHTAQTKPREEQINSTERSV
SELPIMHQDWLNGKEFKCRVNSAAFGAPIEKTISKTKGRPKAPQVYTIPPPKEQMAKDKVSLTCMITNFFPEDIT
VEWQWNGQPAENYKNTQPIMNTDGSYFVYSELNVQKSNWEAGNTFTCSVLHEGLHNHHTEDSLSHSP
SEQ ID NO: 117: mPD1-IL2v LC: mPD-1.VL-hk.LC murine antiPD-1 (mIgG1 Light Chain)
DIVMTQGTLPNPVPSGESVSITCRSSKSLLYSDGKTYLNWYLQRPGQSPQLLIYWMSTRASGVSDRFSGSGSGTD
FTLKISGVEAEDVGIYYCQQGLEFPTFGGGTKLELKRTDAAPTVSIFPPSSEQLTSGGASVVCFLNNFYPKDINV
KWKIDGSERQNGVLNSWTDQDSKDSTYSMSSTLTLTKDEYERHNSYTCEATHKTSTSPIVKSFNRNEC
SEQ ID NO: 118: hPD-1-IL15 (M1) HC1: xhPD-1. VH-h1.HC.L234A.L235A.G237A.T366S.
L368A.Y407V.dk-IL15m1.N1A.D30N.E46G.V49R anti-human PD-1
(Fc LALA KiH hole-IL-15m1)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPCREEMTKNQVSLSCAVKGFYPS
DIAVEWESNGQPENNYKTTPPVLDSDGSFFLVSKLTVDKSRWQQGNVFSCSVMHEALHNHYTQKSLSLSPGSGGG
GSGGGGSGGGGAWVNVISDLKKIEDLIQSMHIDATLYTESNVHPSCKVTAMKCFLLGLQRISLESGDASIHDTVE
NLIILANNSLSSNGNVTESGCKECEELEEKNIKEFLQSFVHIVQMFINTS
SEQ ID NO: 119: hPD-1-IL15 (M1) HC2: xhPD-1.VH-h1.HC.L234A.L235A.G237A.T366W.dk
anti-human PD-1 (Fc LALA KiH knob)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCTLPPSREEMTKNQVSLWCLVKGFYPS
DIAVEWESNGQPENNYKTTPPVLDSDGSFFLYSKLTVDKSRWQQGNVFSCSVMHEALHNHYTQKSLSLSPGK
SEQ ID NO: 120: hPD-1-IL15 (M1) LC: xhPD-1.VL-hk.LC anti-human PD-1 (Light Chain)
DIQMTQSPSSLSASVGDRVTITCKSSQSLWDSGNQKNFLTWYQQKPGKAPKLLIYWTSYRESGVPSRFSGSGSGT
DFTLTISSLQPEDFATYYCQNDYFYPLTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
SEQ ID NO: 121: hPD-1-IL15 (M2) HC1: xhPD-1.VH-h1.HC.L234A.L235A.G237A.T366S.
L368A.Y407V.dk-IL15m1.N1G.D30N.E46G.V49R.E64Q anti-human PD-1
(Fc LALA KiH hole-IL-15m2)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPCREEMTKNQVSLSCAVKGFYPS
DIAVEWESNGQPENNYKTTPPVLDSDGSFFLVSKLTVDKSRWQQGNVFSCSVMHEALHNHYTQKSLSLSPGSGGG
GSGGGGSGGGGGWVNVISDLKKIEDLIQSMHIDATLYTESNVHPSCKVTAMKCFLLGLQRISLESGDASIHDTVQ
NLIILANNSLSSNGNVTESGCKECEELEEKNIKEFLQSFVHIVQMFINTS
SEQ ID NO: 122: hPD-1-IL15 (M2) HC2: xhPD-1.VH-h1.HC.L234A.L235A.G237A.T366W.dk
anti-human PD-1 (Fc LALA KiH knob)
QVQLVQSGAEVKKPGSSVKVSCKASGYTFTSYWINWVRQAPGQGLEWMGNIYPGSSITNYAQKFQGRVTITADES
TSTAYMELSSLRSEDTAVYYCARLTTGTFAYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTHT
CPPCPAPEAAGAPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVCTLPPSREEMTKNQVSLWCLVKGFYPS
DIAVEWESNGQPENNYKTTPPVLDSDGSFFLYSKLTVDKSRWQQGNVFSCSVMHEALHNHYTQKSLSLSPGK
SEQ ID NO: 123: hPD-1-IL15 (M2) LC: xhPD-1.VL-hk.LC anti-human PD-1 (Light Chain)
DIQMTQSPSSLSASVGDRVTITCKSSQSLWDSGNQKNFLTWYQQKPGKAPKLLIYWTSYRESGVPSRFSGSGSGT
DFTLTISSLQPEDFATYYCQNDYFYPLTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSFNRGEC
SEQ ID NO: 124: Kadmon HC1: 2-8 S354C/T366W LALAPG improved linker
ITCPPPMSVEHADIWVKSYSLYSRERYICNSGFKRKAGTSSLTECVLNKATNVAHWTTPS
LKCIRDPALVHQRPAPPSGGGGSGGGGSGGGSGGGGSNWVNVISDLKKIEDLIQSMHIDA
TLYTESDVHPSCKVTAMKCFLLELQVISLESGDASIHDTVESLIILANNSLSSNGNVTES
GCKECEELEEKNIKEFLQSFVHIVQMFINTSGGGGSGGGGSGGGGSGGGGSGGGGGGGG
SEVQLLESGGGLVQPGGSLRLSCAASGFTFSSYWMSWVRQAPGKGLEWVSAISGSGGSTY
YADSVKGRFTISRDNSKNTLYLQMNSLRAEDTAVYYCASSPLQWVDVWGQGTTVTVSSAS
TKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPEAAGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALGAPIEKTISKAKGQPREPQVYTLPPCRDELT
KNQVSLWCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 125: Kadmon HC2: SEQ ID 223: 1-4 Y349C/T366S/L368A/Y407V LALAPG
EVQLLESGGGLVQPGGSLRLSCAASGFTFSSYWMSWVRQAPGKGLEWVSAISGSGGSTYY
ADSVKGRFTISRDNSKNTLYLQMNSLRAEDTAVYYCASSPLQWVDVWGQGTTVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPEAAGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALGAPIEKTISKAKGQPREPQVCTLPPSRDELTK
NQVSLSCAVKGFYPSDIAVEWESNGQPENNYKTTPPVLDSDGSFFLVSKLTVDKSRWQQG
NVFSCSVMHEALHNHYTQKSLSLSPGK
SEQ ID NO: 126: Kadmon LC: SEQ ID NO: 219-38B2:
DIQMTQSPSSLSASVGDRVTITCRASESISSWLAWYQQKPGKAPKLLIYDASSLESGVPS
RFSGSGSGTDFTLTISSLQPEDFATYYCQQGDSFPFTFGQGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 127: mSOT202 HC knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNLLGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLPAPIERTISKPKGSVRAPQVYVLPPPEEEMTKKQVTLWCMVTDEM
PEDIYVEWTNNGKTELNYKNTEPVLDSDGSYFMYSKLRVEKKNWVERNSYSCSVVHEGLHNHHTTKSFSRTPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 128: mSOT202 HC hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNLLGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLPAPIERTISKPKGSVRAPQVYVLPPPEEEMTKKQVTLSCAVTDEM
PEDIYVEWTNNGKTELNYKNTEPVLDSDGSYFMVSKLRVEKKNWVERNSYSCSVVHEGLHNHHTTKSFSRTPG
SEQ ID NO: 129: mSOT202 LC
DIQMTQSPSSLSASVGDRVTITCRASEDIYSNLAWYQQKPGKAPKLLIFSVKRLQDGVPSRFSGSGSGTDFTLTI
SSLQPEDFATYYCLQGSNFPLTFGQGTKVEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKTSTSPIVKSENRNEC
SEQ ID NO: 130: mSOT202 LALAPG HC knob
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNAAGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLGAPIERTISKPKGSVRAPQVYVLPPPEEEMTKKQVTLWCMVTDEM
PEDIYVEWTNNGKTELNYKNTEPVLDSDGSYFMYSKLRVEKKNWVERNSYSCSVVHEGLHNHHTTKSFSRTPGIT
CPPPMSVEHADIWVKSYSLYSRERYICNSGFKRKAGTSSLTECVLNKATNVAHWTTPSLKCIRDPALVHQRPAPP
SGGSGGGGSGGGSGGGGSGGNWVNVISDLKKIEDLIQSMHIDATLYTESDVHPSCKVTAMKCFLLELQVISLESG
DASIHDTVEALIILANNSLSSNAQVTESGCKECEELEEKNIKEFLQSFVHIVQMFINTS
SEQ ID NO: 131: mSOT202 LALAPG HC hole
QVQLVQSGAEVKKPGASVKVSCKASGYTFTDYAMHWVRQAPGQRLEWMGWINTYTGKPTYAQKFQGRVTITRDTS
ASTAYMELSSLRSEDTAVYYCARAVFYGYTMDAWGQGTLVTVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNAAGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLGAPIERTISKPKGSVRAPQVYVLPPPEEEMTKKQVTLSCAVTDEM
PEDIYVEWTNNGKTELNYKNTEPVLDSDGSYFMVSKLRVEKKNWVERNSYSCSVVHEGLHNHHTTKSFSRTPG
SEQ ID NO: 132: mSOT202 isotype HC knob
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLPAPIERTISKPKGSVRAPQVYVLPPPEKEMTKKQVTLTC
MVTDFMPEDIYVEWTNNGKTELNYKNTEPVLKSDGSYFMYSKLRVEKKNWVERNSYSCSVVHEGLHNHHTTKSFS
RTPGITCPPPMSVEHADIWVKSYSLYSRERYICNSGFKRKAGTSSLTECVLNKATNVAHWTTPSLKCIRDPALVH
QRPAPPSGGSGGGGSGGGSGGGGSGGNWVNVISDLKKIEDLIQSMHIDATLYTESDVHPSCKVTAMKCFLLELQV
ISLESGDASIHDTVEALIILANNSLSSNAQVTESGCKECEELEEKNIKEFLQSFVHIVQMFINTS
SEQ ID NO: 133: mSOT202 isotype HC hole
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNLLGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLPAPIERTISKPKGSVRAPQVYVLPPPEEEMTKKQVTLTC
MVTDEMPEDIYVEWTNNGKTELNYKNTEPVLDSDGSYFMYSELRVEKKNWVERNSYSCSVVHEGLHNHHTTDSFS
RTPG
SEQ ID NO: 134: mSOT202 isotype LC
DVQMTQSTSSLSASLGDRVTISCRASQDIKNYLNWYQQKPGGTVKLLIYYSSTLLSGVPSRFSGRGSGTDESLTI
TNLEREDIATYFCQQSITLPPTFGGGTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKTSTSPIVKSFNRNEC
SEQ ID NO: 135: mSOT202 isotype LALAPG HC knob
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLGAPIERTISKPKGSVRAPQVYVLPPPEKEMTKKQVTLTC
MVTDFMPEDIYVEWTNNGKTELNYKNTEPVLKSDGSYFMYSKLRVEKKNWVERNSYSCSVVHEGLHNHHTTKSES
RTPGITCPPPMSVEHADIWVKSYSLYSRERYICNSGFKRKAGTSSLTECVLNKATNVAHWTTPSLKCIRDPALVH
QRPAPPSGGSGGGGSGGGSGGGGSGGNWVNVISDLKKIEDLIQSMHIDATLYTESDVHPSCKVTAMKCFLLELQV
ISLESGDASIHDTVEALIILANNSLSSNAQVTESGCKECEELEEKNIKEFLQSFVHIVQMFINTS
SEQ ID NO: 136: mSOT202 isotype LALAPG HC hole
EVQLVESGGGLVKPGGSLKLSCAVSGFTFSDYAMSWIRQTPENRLEWVASINIGATYAYYPDSVKGRFTISRDNA
KNTLFLQMSSLGSEDTAMYYCARPGSPYEYDKAYYSMAYWGPGTSVTVSSAKTTAPSVYPLAPVCGDTTGSSVTL
GCLVKGYFPEPVTLTWNSGSLSSGVHTFPAVLQSDLYTLSSSVTVTSSTWPSQSITCNVAHPASSTKVDKKIEPR
GPTIKPCPPCKCPAPNAAGGPSVFIFPPKIKDVLMISLSPIVTCVVVDVSEDDPDVQISWFVNNVEVHTAQTQTH
REDYNSTLRVVSALPIQHQDWMSGKEFKCKVNNKDLGAPIERTISKPKGSVRAPQVYVLPPPEEEMTKKQVTLTC
MVTDFMPEDIYVEWTNNGKTELNYKNTEPVLDSDGSYFMYSELRVEKKNWVERNSYSCSVVHEGLHNHHTTDSES
RTPG
SEQ ID NO: 137: RLI-15_{AQ} peptide
ELQVISLESGDASIHDTVENLIILANNSLSSNAQV
SEQ ID NO: 138: RLI-15_{AQA} peptide
ELQVISLESGDASIHDTVEALIILANNSLSSNAQV
SEQ ID NO: 139: RLI-15 NA peptide
VEALIILANNSLSSNGNVTESGCKECEELEEK
SEQ ID NO: 140: RLI-15_{AQA} peptide
VEALIILANNSLSSNAQVTESGCKECEELEEK

### EXAMPLES

### Example 1: General Materials and Methods

### Potency Assay on Kit225

The activity of both IL-2 and IL-15 can be determined by induction of proliferation of kit225 cells as described by Hori et al. (1987). Kit225 cells (Hori, Uchiyama et al. 1987) were passaged in kit225 base medium and used for the potency assay at passage 4-7. Before the potency assay, kit225 cell were cultivated in kit225 base medium without IL-2 for 24 h (starvation period). 1×10⁴ kit225 cells were plated in 96-well plate and a serial dilution of RLI-15 and respective molecules PEM-RLI-15 was added to cells. Cells were incubated at 37° C., 5% CO₂ for 72±3 h. Following the incubation, 10 μl (10% of the volume in the well) of Alamar Blue was added to each well and, after 6 h, absorbance was measured at 560 nm with a 620 nm reference using a Tecan Spark absorbance microplate reader (set mixing before detection for 15 s). In some cases, when lower potency RLI2 mutants were tested, the incubation with kit225 cells was prolonged from 3 days (72 h±3 h) to 5 days.

Preferably, methods such as colorimetry or fluorescence are used to determine proliferation activation due to IL-2 or IL-15 stimulation, as for example described by Soman et al. using CTLL-2 cells (Soman, Yang et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

Concentration of Analyzed RLI Variants were:
RLI2 supernatant. 0.133 mg/ml (ELISA, average from 2 exps)
RLI2_{AQ} supernatant. 0.0297 mg/ml (ELISA, average from 2 exps)

### Properties of RLI2

Purity (RP-UPLC) 99.8%
Formulation 20 mM histidine, 6% (w/v) sorbitol, pH 6.5
Storage temperature −20° C.

### Kit225 Base Medium

RPMI (460 mL)+FBS (30 mL)+Glutamax (5 mL)+Penicillin-Streptomycin (5 mL)+cytokines added into the flasks (75 cm2); IL-2 (5 ng/mL). Cytokines were added to the medium just before cultivation.
   hPBMC Potency Assay

Buffy coats were obtained from healthy donors. PBMC were isolated by Ficoll Paque gradient, washed three times and resuspended in T cell complete medium in 96-well plate. Immunocytokines were added at the indicated concentrations and plates were incubated in 37° C. with 5% CO₂ for 7 days. The proliferation of immune cell population was detected by flow cytometry.

### T Cell Complete Medium

RPMI 1640 medium, CTS GlutaMAX-I 1×, 100 U/mL Penicillin-Streptomycin, 1 mM Sodium pyruvate, NEAA 1× (non-essential amino acid mix), 2-Mercaptoethanol 0.05 mM and 10% AB human serum (heat inactivated).

List of used antibodies

| Marker | Fluorochrome | Vendor | Cat. No. | Clone |
|---|---|---|---|---|
| CD16 | PE-Cy7 | BioLegend | 302016 | 3G8 |
| CD25 | BV605 | BD | 562660 | 2A3 |
| CD3 | APC ef780 | Thermo | 47-0037-42 | OKT3 |
| CD4 | BV421 | BioLegend | 317434 | OKT4 |
| CD45 | BV605 | BD | 564047 | HI30 |
| CD45 | PerCP-Cy5.5 | BD | 564105 | HI30 |
| CD45RA | PE-Cy7 | BioLegend | 304126 | HI100 |
| CD45RO | A700 | BioLegend | 304218 | UCHL1 |
| CD56 | A700 | BioLegend | 318316 | HCD56 |
| CD8 | PerCP-Cy5.5 | BioLegend | 344710 | SK1 |
| Foxp3 | A488 | Thermo | 53-4776-42 | PCH101 |
| Ki67 | PE | BioLegend | 350504 | ki-67 |
| NKG2D | APC | BioLegend | 320808 | 1D11 |
| PD-1 | FITC | BioLegend | 329904 | EH12 |
| Zombie Aqua | BV510 | BioLegend | 423102 | NA |

Isolation of human NK cells (hNK): Fresh blood from healthy donors was diluted in a 1:1 ration with cold PBS-EDTA, ph7.4 and PBMC were isolated by Ficoll-Paque gradient isolation. Isolated PBMCs were resuspended in complete culture medium. hNK cells were isolated from a the PBMC using the EasySep Human NK Cell Isolation kit (Stem Cell Technologies, USA) according to the manufacturer instructions. Isolate hNK cells of each donor were resuspended in NK medium with 10% serum at a concentration of 3×10⁶ cells/ml.

### PD-1/PD-L1 Blockade Bioassay

The assay was performed according to manufacturer's instructions (Promega PD-1/PD-L1 Blockade Bioassay J1250). In brief, PD-L1 aAPC/CHO-K1 cells were plated in 96 well plate and incubated 16-20 hours in a 37° C., 5% CO₂ incubator. After that PEM-RLI immunocytokines at the indicated concentrations and PD-1 Effector Cells were added to the cells and incubated for 6 hours in a 37° C., 5% CO₂ incubator. After the incubation period, Bio-Glo™ Reagent was added to the wells and incubated at room temperature for 15 min, luminescence measurement was performed.

### Cynomolgus Monkey Studies

Pharmacokinetics of indicated PEM-RLI molecules were tested in cynomolgus monkeys (n=2-3) after administration of indicated doses on day 1 or day 15. Blood for serum separation was collected at 1 h, 4 h, 8 h, 24 h, 48 h, 60 h, 72 h, 84 h, 96 h, 120 h and 168 h after administration (some timepoints may have been omitted in some cases). The concentration of immunocytokines in serum was determined by ELISA using the antibodies of Table 5. Blood for flow cytometry evaluation of selected immune cell populations (NK and CD8⁺ T cells) was collected at pre-dose, day 5, 8, 12, 15, 19, 22 and 26.

TABLE 5
List of used antibodies for the cynomolgus monkey studies

| Marker | Fluorochrome | Vendor | Cat. No. | Clone |
|---|---|---|---|---|
| CD45 | PE-Cy7 | BD biosciences | 561294 | D058-1283 |
| CD3 | APC-Cy7 or | BD biosciences | 557757 | SP34-2 |
| | PerCP | BD biosciences | 552851 | |
| CD4 | V450 | BD biosciences | 560811 | L200 |
| CD8 | HV605 or | BD biosciences | 564116 | SK1 |
| | V500 | | 561618 | |
| CD28 | APC | BD biosciences | 560683 | CD28.2 |
| CD95 | FITC | BD biosciences | 555673 | DX2 |
| CD122 | PE | BD biosciences | 557323 | Mik-ß2 |
| Fix. Viab. | eFluor ^{TM}506 or | eBioscience | 65-0866-14 | — |
| Dye | eFluor ^{TM} 780 | | 65-0865-14 | |
| Ki-67 | AF700 or | BD biosciences | 561277 | B56 |
| | A488 | | 558616 | |
| CD20 | PE or | BD biosciences | 555623 | 2H7 |
| | APC | BioLegend | 302310 | |
| CD25 | APC | eBioscience | 17-0257-42 | CD25-4E3 |
| Foxp3 | AF488 or | Biolegend | 320112 | 206D |
| | PE | | 320108 | |

List of antibodies used for Example 37 (Tscm cell panel)

| Marker | Fluorochrome | Vendor | Clone |
|---|---|---|---|
| CD3 | PE-Cy7 | eBioscience | 145-2C11 |
| CD8 | V500 | BD Biosciences | 53-6.7 |
| CD44 | APC | eBioscience | IM7 |
| CD122 | BV605 | BD Biosciences | 5H4 |
| CD62L | PE | eBioscience | W18021D |
| CD95 | FITC | eBioscience | 15A7 |
| CXCR3 | PE/Dazzle 594 | BioLegend | CXCR3-173 |
| CCR7 | PerCP/Cy5.5 | BioLegend | 4B12 |
| gdTCR | eF450 | eBioscience | eBioGL3 |
| Sca1 | A700 | BioLegend | D7 |
| FVD | eF780 | eBioscience | — |

### Mouse Efficacy Studies

The objective of these studies was to evaluate the in vivo therapeutic efficacy of PEM-RLI2 NA x1 and Pembrolizumab as a monotherapy in the treatment of HuCell MC38-hPD-L1 tumour cell line in female hPD1 single KI HuGEMM mice (C57BL/6-Pdcd1^{em1(hPDcD1)/Smoc}) (n=8 mice per group). Each mouse was inoculated subcutaneously in the right lower flank region with MC38-hPD-L1 tumour cells (1×10⁶) in 0.1 ml of PBS for tumour development. The randomization was started when the mean tumour size reached 108 mm³. 40 mice were enrolled in the study. All animals were randomly allocated to 5 study groups. Randomization was performed based on “Matched distribution” method (StudyDirector™ software, version 3.1.399.19). The date of randomization day was denoted as day 0 (DO). After tumour cells inoculation, the animals were checked daily (or more often as needed, at the discretion of the Study Director) for morbidity and mortality. Tumour volumes were measured three times per week in two dimensions using a caliper, and the volume were expressed in mm³ using the formula: “V=(L×W×W)/2, where V is tumour volume, L is tumour length (the longest tumour dimension) and W is tumour width (the longest tumour dimension perpendicular to L). PEM-RLI2 NA x1 was administered IV at 20 mg/kg at day 0 and pembrolizumab was administered IP at 5 mg/kg at days 0,3,6 and 9. Tumour observation was followed for 18 days. Concomitantly to this, PEM-RLI2 NA x1 (IL-15 with N65A and AQ mutation) was administered IV at 5, 10 at day 0. Tumour observation was followed for 6 days.

### Mixed Lymphocyte Reaction

Buffy coats were obtained from healthy donors. PBMC were isolated by Ficoll Paque gradient, washed three times. PBMC were isolated by Ficoll Paque gradient, washed three times. Pairs of hPBMCs donors were cultivated with equimolar concentration of pembrolizumab and PEM L-RLI NA x1 at 1 nM for six days. IFNγ production in cell supernatants was determined using human IFN-γ DuoSet ELISA (R&D systems, No. DY258B). Data are expressed as relative response of IFNγ production [%] and represent mean±SEM from—12 pairs of hPBMC healthy donors.

### SDS-PAGE and Anti-RLI Western-Blot Analysis

The purified proteins were analyzed by SDS-PAGE and anti-RLI Western blot.

Coomassie staining: protein bands are visualized according to their molecular weight in denatured conditions.

Briefly, 1 volume of loading buffer (containing or not beta mercaptoethanol) was added to 3 volumes of the sample to analyze (then more or less diluted into 1× loading buffer), homogenized and denatured 5 min at 95° C. Denatured sample is loaded on Criterion TGX gel and run in running buffer at constant voltage (300 V) and limited current (75 mA or 135 mA per gel depending on the gel type) in 1×TGS buffer for 18 min or 21 min depending on the gel type. Gel is removed from the cassette and washed 3 times 5 min in water, stained 20 min with Biosafe staining solution (Biorad) and washed 3 times 20 min in water before final de-stain wash 3 hours in water. Stained gel is then scanned with gel scanner.

Western-blot analysis: the gel is then transferred to a nitrocellulose membrane and used for Western-blot analysis with different antibodies. At the end of migration, the gel is used for protein transfer to nitrocellulose membrane. For the example of reference (Biorad #170-4155, Trans-BlotR Turbo™ Transfer Starter System), the transfer parameters are 2.5 A, 25 V, 7 minutes (for Criterion gels) or 2.5 A, 25 V, 3 minutes (for Mini-PROTEAN gels). After membrane saturation in iBind™ Flex solution, antibody incubation and wash steps are then done in iBind system. After revelation and when completely dry, the membrane is scanned for analysis. Primary antibody used was anti RLI2-PR01 antibody (Cytune, dilution 1:25000), secondary antibody used was donkey anti-Rabbit IgG-AP antibody (Santa Cruz Biotechnology, dilution 1:5000).

### Capillary Electrophoresis

Protein analysis by capillary electrophoresis relies on separation of LDS-labeled protein variants by a sieving matrix in a constant electric field. The Labchip GXII instrument uses a single sipper icrofluidic chip to characterize protein samples loaded on a 96-well plate. The microfluidic chip technology allows the separation and analysis of the protein samples. After laser-induced signal detection and analysis, the provided data are: relative protein concentration, molecular size and percent purity using ladder and marker calibration standards.

Samples are denatured by mixing 5 μL-sample and 35 μL of HT Protein Sample Buffer in presence or not of DTT at final concentration of 35 mM. If required, samples are prediluted at 1 mg/mL in HT Protein Sample Buffer. Denaturation is performed by heating mix at 100° C. for 5 min. Then, 70 μL of water are added and samples are centrifuged 10 minutes at 2,000 g. Samples (in a 96-well plate) are then loaded on LabChip GXII instrument for chip transfer and analysis.

TABLE 6
Summary of characteristics

| | RLI2 (wt) | RLI2 A | RLI2 AQ |
|---|---|---|---|
| Capillary | 6 bands between | 5 bands between | 4 bands between |
| Electrophoresis | 23 and 34 kDa | 23 and 34 kDa | 23 and 34 kDa |
| (non-reducing): | 86.5% | 82.2% | 95.0% |
| MW, Purity | | | |
| SDS-PAGE/anti-RLI | 92.4% | 90.5% | 93.7% |
| Western | | | |
| blot (non-reducing): | | | |
| Purity | | | |

### Glycosylation/Deamidation Mutants

TABLE 7
Overview of relevant amino acids for glycosylation and deamidation

| | Minor | | | Major | |
|---|---|---|---|---|---|
| | glycos- | | | glycos- | 3^{rd} potential |
| | ylation | Deamidation | | ylation | glycosylation |
| | site | site | | site | site |
| mature | N71 | N77 | G78A | N79Q | N160 |
| IL-15 | | | | | |
| RLI | N168 | N174 | G175A | N176Q | N209 |

The RL12 molecule has the major glycosylation site is N176 (RLI numbering) and a minor site at N168. No glycosylation is seen at N209. The glycans are complex, majorly biantennary, fucosylated, G0 to G2 with little sialylation. In cell culture about 40 to 50% of the protein are glycosylated with about 5% at N168. After purification as described above, about 14-25% of RLI2 are glycosylated. Whereas the different levels of glycosylation have not shown any impact on potency, stability and only a minor impact on pharmacokinetics with glycosylated RLI2 having a shorter half-life, heterogeneity of an active pharmacological ingredient is still problematic from a regulatory perspective.

A potential hot spot for deamidation identified in IL-15 expressed in E. coli (Nellis, Michiel et al. 2012) is N77 (IL-15 numbering)/N174 (RLI numbering). Although it has been described that N-glycosylation of N79 partially prevents N77 deamidation (Thaysen-Andersen, Chertova et al. 2016), the inventors indeed saw in mass spectrometry that N77 was deamidated in CHO-expressed RLI2 and identified deamidation as a real problem for potential heterogeneity of RLI2 and RLI-based products and therefore deamidation should be avoided.

FIG. 1A shows that RLI2 wt (without a mutation) indeed is a heterogenous product with two major bands at about 20 and 25 kDa and a few minor bands, all being immune reactive to the anti-RLI2 antibody and thereby being different modifications of the RLI2 protein.

The inventors wanted to avoid mutating N77 as an obvious way to abolish deamidation of it and thereby removing the polar amide, as the conservative substitution to glutamine would not have resolved the deamidation risk. The single substitution G78A (IL-15 numbering)/G175A (RLI numbering) in RLI2 (RLI2 A) was introduced instead to abolish potential deamidation at position N77. Whereas loss of deamidation would not be visible on the Coomassie staining or the Western blot, the major acidic peak (pI 6.0) in RP-UPLC was significantly reduced in cIEF as it would be expected for loss of deamidation, which confirms that deamidation hot spot N174 indeed was deamidated (data not shown). Also, mass spectrometry analysis of the PEM-RLI AQ constructs showed zero deamidation (data not shown).

Surprisingly the G78A mutation led to a slight increase in glycosylation (see FIG. 1A and better visible in FIG. 1B) with larger/more glycosylated species compared to RLI2 wt. An additional band appears indicating such new glycosylation pattern (see dashed box 3 in FIG. 1B). Also RP-UPLC peaks were slightly shifted (data not shown). This changed glycosylation pattern was unexpected as the influence of the deamination mutation G78A on glycosylation could not have been predicted.

By the additionally substituting N79 (IL-15 numbering)/N176 (RLI numbering) by Q (RLI2 AQ, RLI2_{AQ}), which was introduced to disrupt the main glycosylation site of IL-15, a marked reduction of larger species of RLI2 was observed (see dashed box 1 in FIG. 1B). The residual larger band (see solid box 2 in FIG. 1B) likely represents glycosylation at N71 (IL-15 numbering)/N168 (RLI numbering) of about 20% of the RLI molecule, which appears to be slightly increased compared to RLI2 wt and RLI2. The band of box 1 may represent RLI2 glycosylated at N176, whereas the band of box 3 may represent RLI2 glycosylated at N176 and N168. The band of box 3 may however also be RLI2 glycosylated with unfavorable Sialic acid glycan structures at N176. Without being bound by any theory, this surprising increase of glycosylation at N71 may be explained that the glycosylation at the major site N79 sterically hindered glycosylation at N71 in RLI2 wt, such hinderance being relieved once N79 is mutated.

Together, RLI2_{AQ}, and accordingly also IL-15_{AQ}, with the AQ substitutions represent an RLI2, or IL-15, variant with a highly improved homogeneity and a reduced risk for deamidation.

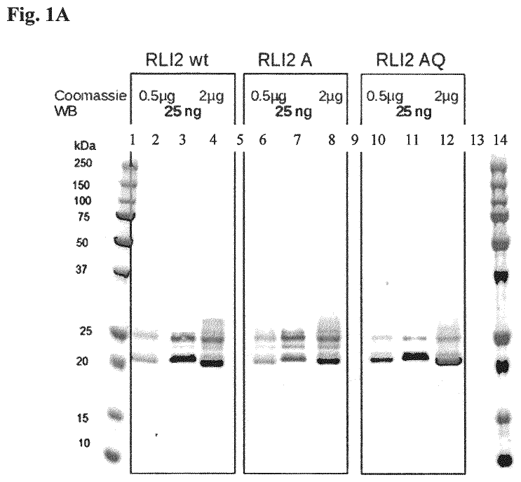

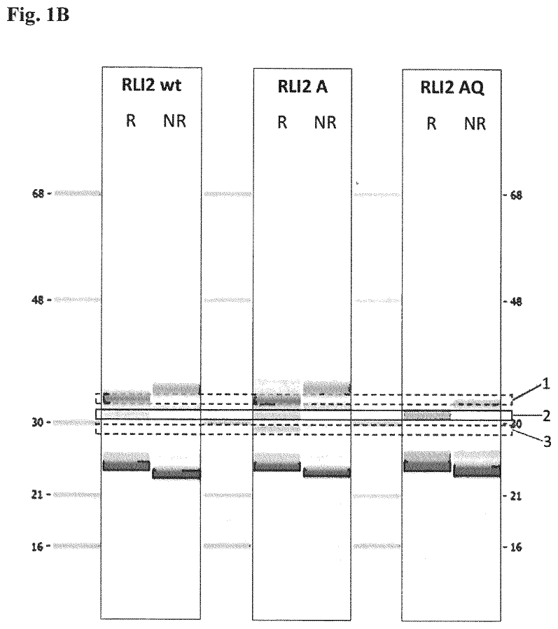

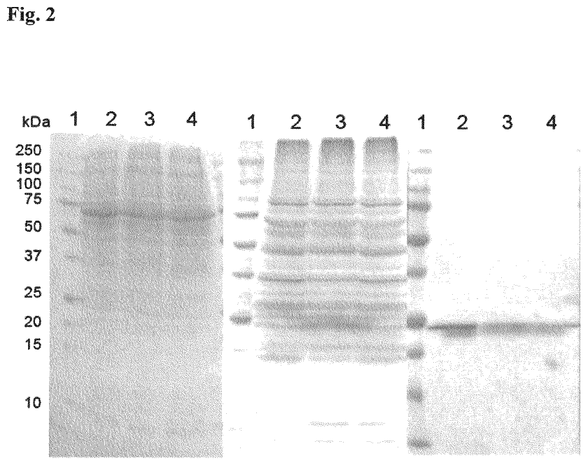

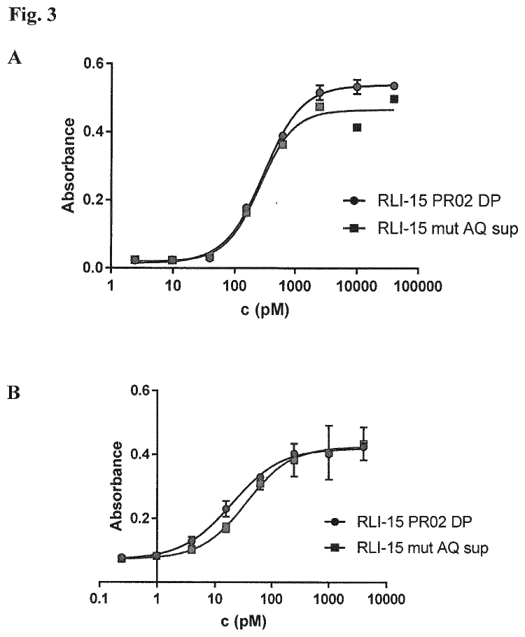

In order to compare the effect/impact of glycosylation on the biological activities of RLI variants, we have specifically inactivated the 3 potential glycosylation sites N71/N79/N160 of IL-15 (N168/N176/N209 for RLI) by site-directed mutagenesis (Stratagene Site Directed Mutagenesis XL Kit). N71 was substituted by S, N79 was substituted by Q and N160 was substituted by S, thereby generating RLI2_{N168S/N176Q/N209S} and RLI1_{N168S/N176Q/N209S}. In order to confirm the main N-glycosylation occupancy on N79 (=N176 of RLI) the RLI2_{N176Q} mutant was made. Transient expression in CHO cells lead to a unique 25 kDa band (see FIG. 2, right pane).

The RLI protein mutated only on its major glycosylation site (RLI2_{N176Q}) exhibited also a unique 25 kDa band, therefore confirming the main glycosylation occupancy on the N176 residue of RLI expressed in CHO (transient expression). Secretion yields of the deglycosylated mutants expressed in in transient CHO cells were similar to their glycosylation/original counterpart. Accordingly, there was no significant influence of the deglycosylation on the expression levels. Same was observed in the Pichia pastoris expression system (data not shown).

Furthermore, these mutations on the N-glycosylation sites appear to induce no significant influence on the in vitro proliferative activity of RLI on kit225 or 32Dβ cells. As usually, all RLI versions (RLI1 or RLI2, glycosylated or non-glycosylated, CHO or baculo or Pichia) were similarly stimulating the proliferation of the kit225 cell line.

### Potency of RLI2_{AQ} Variant

The activity of both IL-2 and IL-15 can be determined by induction of proliferation of kit225 cells as described by Hori et al. (1987). Kit225 cells (Hori, Uchiyama et al. 1987) were passaged in kit225 base medium and used for the potency assay at passage 4-7. Before the potency assay, kit225 cell were cultivated in kit225 base medium without IL-2 for 24 h (starvation period). 1×10⁴ kit225 cells were plated in 96-well plate and a serial dilution of RLI-15 and respective molecules PEM-RLI-15 was added to cells. Cells were incubated at 37° C., 5% CO₂ for 72±3 h. Following the incubation, 10 μl (10% of the volume in the well) of Alamar Blue was added to each well and, after 6 h, absorbance was measured at 560 nm with a 620 nm reference using a Tecan Spark absorbance microplate reader (set mixing before detection for 15 s). In some cases, when lower potency RLI2 mutants were tested, the incubation with kit225 cells was prolonged from 3 days (72 h±3 h) to 5 days.

Preferably, methods such as colorimetry or fluorescence are used to determine proliferation activation due to IL-2 or IL-15 stimulation, as for example described by Soman et al. using CTLL-2 cells (Soman, Yang et al. 2009). As an alternative to cell lines such as the kit225 cells, human peripheral blood mononuclear cells (PBMCs) or buffy coats can be used. A preferred bioassay to determine the activity of IL-2 or IL-15 is the IL-2/IL-15 Bioassay Kit using STAT5-RE CTLL-2 cells (Promega Catalog number CS2018B03/B07/B05).

Concentration of analyzed RLI variants were:
RLI2 supernatant. 0.133 mg/ml (ELISA, average from 2 exps)
RLI2_{AQ} supernatant. 0.0297 mg/ml (ELISA, average from 2 exps)

TABLE 8
EC50 values (nM) of RLI2 compared to RLI2_{AQ} from
supernatants determined by activation of 32Db cells or kit225 cells

| EC50 (nM) | RLI2 | RLI2_{AQ} |
|---|---|---|
| 32Db-plate_21 h (FIG. 3A) | 301.1 | 263.1 |
| kit225-plate_4 h (FIG. 3B) | 19.52 | 35.15 |

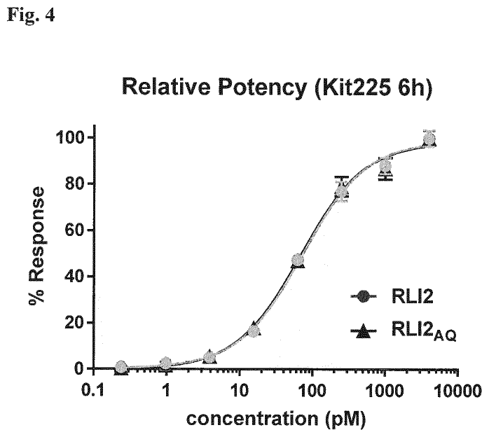

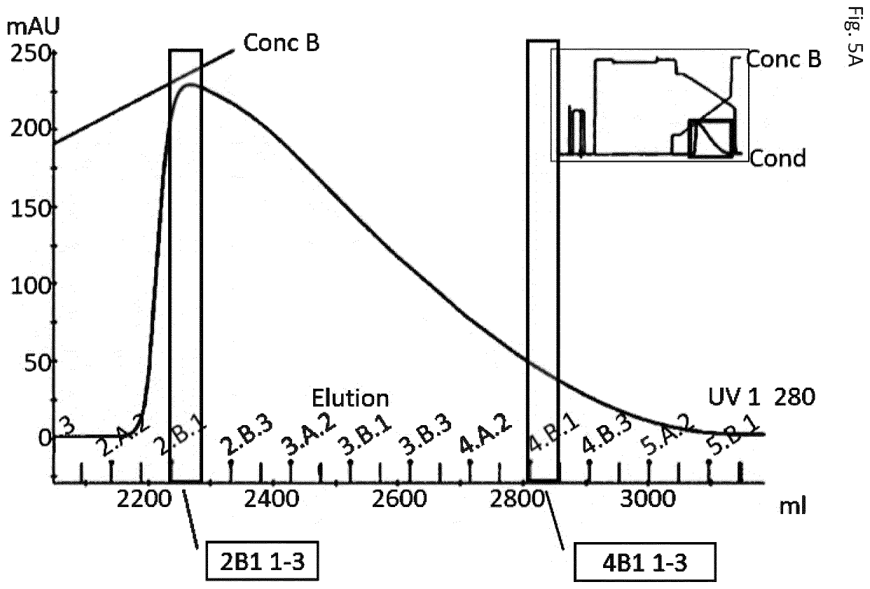

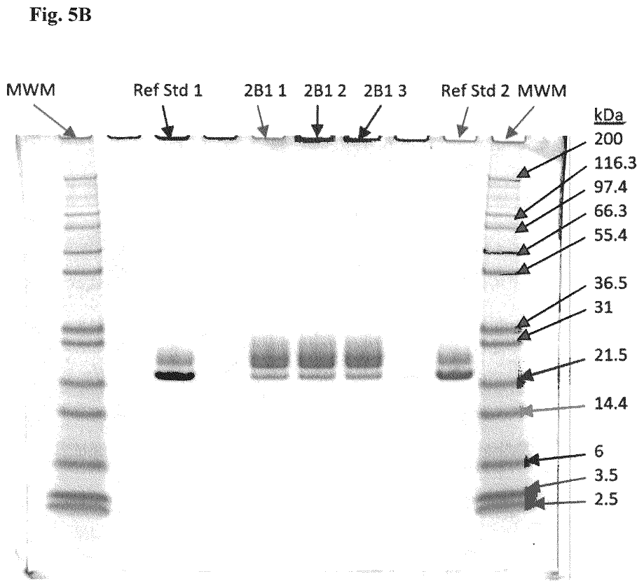

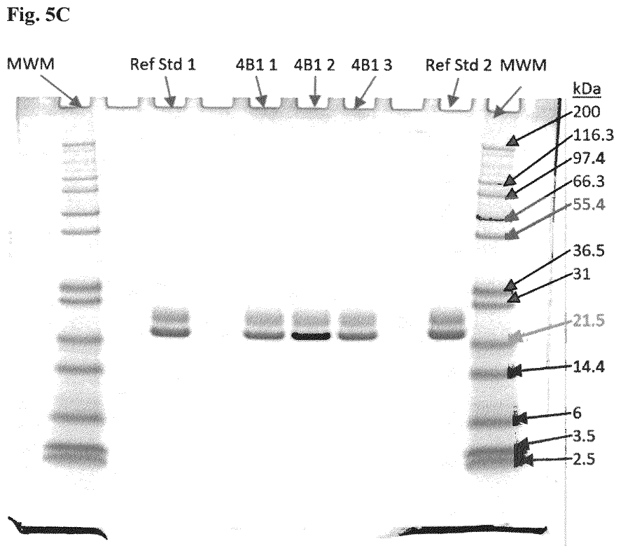

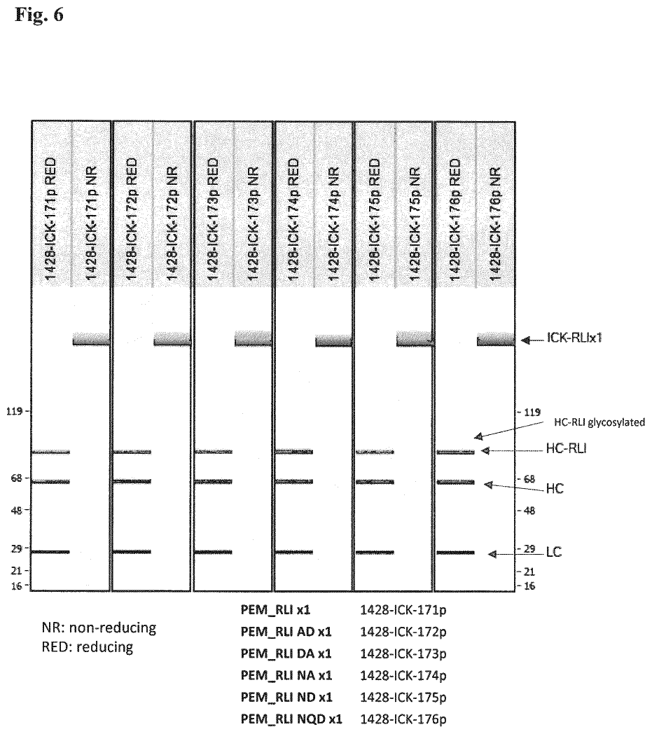

TABLE 9
Relative potency of RLI2 compared to RLI2_{AQ} from supernatants
determined by activation of Kit225 cell proliferation.

| | RLI2 | RLI2_{AQ} |
|---|---|---|
| kit225-6 h/EC50 (nM) (FIG. 4) | 72.47 | 69.5 |
| Relative potency | 100% | 96% |

Accordingly, the glycosylation mutant RLI2_{AQ} as supernatant showed a very similar potency to stimulate kit225 and/or 32 Db cells if compared to RLI2 from supernatant. This was surprising as for many glycoproteins loss of glycosylation leads to a lower activity.

Also in SPR (Biacore) binding experiments to the IL-2/IL-15 βγ receptor, no relevant difference in the kₒₙ rate, k_{off} rate and equilibrium constant K_{d} between RLI2 and RLI AQ was observed (data not shown).

In summary, RLI2_{AQ}, and accordingly also IL-15_{AQ}, with the AQ substitutions represents an RLI2, or IL-15, variant with a highly improved homogeneity, a reduced risk for deamidation with a comparable potency to activate immune cells.

### Cynomolgus PK/PD Study of Highly Glycosylated and Low Glycosylated RLI2

In order to compare highly glycosylated and low glycosylated RLI2 with respect to their PK and PD properties, a 200 l scale production campaign was run, harvested with SOSP and XOSP depth filters and protein was captured on a PPA column. Virus was inactivated by solvent detergent treatment and purification continued via a Capto Adhere column and a Hydroxyapatite type II column (flow through mode), followed by a second virus removal step by Nanofiltration. The RLI preparation was polished on an Capto Impres Phenyl column (CPI Phenyl HIC) and selected fractions for highly glycosylated RLI2 were pooled (RLI-15-HG), and selected fractions for low glycosylated RLI2 were pooled (RLI-15-LG), see FIG. 5A-C. Finally, UFDF filtration was performed on a 10 kDa cut-off UF membrane into final formulation buffer (20 mM Histidine, 6% Sorbitol pH6.5). RLI-15-HG shows most of RLI in the upper band for the glycosylated RLI isomer, whereas RLI-15-LG contains only a smaller fraction of glycosylated RLI isomer (FIGS. 5B and C).

A total of three male and three female cynomolgus monkeys were included in PK/PD study. Animals were allocated into two groups receiving RLI2 as RLI-15-HG and RL1-15-LG at 15 μg/′kg (nominal dose) by subcutaneous daily administration according to a cross-over dosing design. Administration was performed for 2 periods of 4 days (2×4), separated by a washout interval of 10 days (Day 1 to Day 4: RLI-15-LG for males and RLI-15-HG for females. Day 15 to Day 18: RLI-15-HG for males and RLI-15-LG for females). Pharmacodynamic parameters (including Ki67 expression in NK, CD4⁺ and CD8⁺ cells) were analyzed from the blood samples collected on pretreatment period. Day 5. Day 12. and Day 19. Blood samples for pharmacokinetic investigations were collected from all animals on Day 1 and Day 15, following the first administration in each treatment interval, at the following time-points: pre-dose. and 0.5, 1, 2, 6, 12 and 24 hours after administration. Bioanalysis was performed. Additionally, backup serum samples (D1 (predose). D15 (predose) and D16 (24 h)) were partially used for immunogenicity assessment (ADA determination).

Pharmacokinetic (PK) analysis was performed using non-compartmental analysis on Phoenix™ WinNonlin® software (version 6.4. Certara L.P.).

Pharmacokinetic profile: All treated animals were exposed to the test item as quantifiable amount of RLI2 were measured over a major part of the sampling period after administration on Day 1 and Day 15. The main pharmacokinetic parameters are summarized in Table 10.

TABLE 10
Main pharmacokinetic parameters

| | | RLI2 LG | | RLI2 HG | |
|---|---|---|---|---|---|
| | | male | female | male | female |
| AUC₀₋ₜ ± | ng · h/ml | 43.9 ± 6.7 | 84.3 ± 28.4 | 19.6 ± 3.2 | 37.9 ± 4.4 |
| SD | | | | | |
| AUC₀₋ₜ/ | ng · h/ml/ | 2.8 ± 0.3 | 5.6 ± 1.9 | 1.3 ± 0.3 | 2.5 ± 0.3 |
| dose ± SD | (mg/kg) | | | | |
| Cₘₐₓ ± SD | ng/ml | 6.1 ± 1.4 | 9.0 ± 2.5 | 3.0 ± 0.7 | 7.6 ± 4.8 |
| Cₘₐₓ/dose ± | ng/ml/ | 0.4 ± 0.1 | 0.6 ± 0.2 | 0.2 ± 0.1 | 0.5 ± 0.3 |
| SD | (mg/kg) | | | | |
| Tₘₐₓ (min- | h | 2 (2-2) | 2 (1-6) | 2 (2-2) | 2 (1-2) |
| max) | | | | | |

Exposure by means of Cₘₐₓ and AUC₀₋ₜ was different between male and female animals. Cₘₐₓ and AUC₀ₜ was about 2-fold higher in females than in males. Independent of this gender difference. a difference in the pharmacokinetics of RLI-15-HG and RLI-15-LG was also observed. Surprisingly, exposure by RLI-15-HG was lower than exposure by RLI-15-LG. The ratio between RLI-15-HG and RLI-15-LG were 0.606 and 0.453 for Cₘₐₓ and AUC₀₋ₜ respectively, independently on animal sex.

### DC-T Cell Based Assay for Determining Immunogenicity

Buffy coats were obtained from healthy donors. The blood was diluted with PBS-EDTA (to get 175 mL of diluted blood) and PBMCs were isolated by Ficoll Paque gradient (15 mL Ficoll+35 mL diluted blood). CD14⁺ monocytes were isolated using EasySep™ Human CD14 Positive Selection Kit II (17858, StemCell) according to manufacturer's instructions. CD14⁻ fraction was pipetted into a new falcon tube, the rest was centrifuged at 1200 rpm, 10 min, then resuspended in CryoStore media, frozen and temporarily stored at −80° C. Isolated CD14⁺ monocytes were resuspended in DC media (CellGro supplemented with IL-4 and GM-CSF). Cells were incubated at 37° C. with 5% CO₂ for 5 days, harvested and seeded into 48-well plates. iDCs were loaded with proteins for 4 h and maturated with a cytokine cocktail (TNF-α, IL-1β plus IL-4 and GM-CSF) overnight. Washing followed for 4 times with PBS and T cell medium. Cells were co-cultured with autologous, CFSE stained CD4⁺ T cells at a 1:10 ratio (negative magnetic separation) and cultivated for 7 days. CFSE dilution was detected by flow cytometry.

Mix of antibodies and viability dye used for evaluation of CD4⁺
T cell proliferation via flow cytometry (T cells stained
with CFSE (FITC)):

| Marker | Fluorochrome | μl/sample | Vendor | Cat. No. | Clone |
|---|---|---|---|---|---|
| CD3 | APC-eFluor 780 | 2 | TFS | 47-0037-42 | OKT3 |
| CD4 | PE-Cy7 | 2 | TFS | 25-0049-42 | RPA-T4 |
| CD8 | PerCP-Cy5.5 | 2 | Biolegend | 344710 | SK1 |
| Zombie | BV510 | 1 | BioLegend | 423102 | |
| Aqua | | | | | |
| PBS | | 43 | | | |

### Example 2: Anti-CD20 Immunocytokines Based on Rituximab (RTX)

The immunocytokine was based on rituximab (VH: SEQ ID NO: 96, VL: SEQ ID NO: 99). The immunocytokines listed in Table 11 were generated and tested for their provided assays.

TABLE 11
Potency of immunocytokines based on RTX

| | | | | | | | | Solid |
|---|---|---|---|---|---|---|---|---|
| | | Potency | | PD in vivo | | | | tumour |
| | CD20 | in vitro | Cytotoxicity | CD8⁺ T | | Metastatic | | A20-CD20/ |
| | binding | (hPBMC) | NK cells | NK cells | | Renca CT26 | | Balb/c |
| RLI2 | | ++++ | +++ | +++ | | +++ | | ++ |
| | | | | (D 1-4, 20 | μg) | (D 1-4,20 | μg) 90% | (1 mg/kg, 20 μg) |
| RTX-RLI2 x2 | + | +++ | +++ | +++ | | ++ | | D 1-D4 |
| | | | | (D 1, 3 | μg) | (D 1, 3 | μg) 50% | |
| RTX- RLI2_{AQ} | + | +++ | +++ | +++ | | ++ | | +++ |
| x2 | | Cyno +++ | | | | | | 0.15 mg/kg (3 μg) |
| | | | | | | | | 0.6 mg/kg |
| | | | | | | | | (toxic) |
| | | | | | | | | D 1, D 8 |
| RTX-RLI2_{AQ} | + | ++ | ++ | ++ | | ++ | | |
| x1 | | Cyno ++ | | | | | | |
| RTX-L40- | + | +++ | +++ | +++ | | ++ | | |
| RLI2_{AQ} x2 | | | | | | | | |

RTX-RLI immunocytokines showed an increases potency in vivo at a reduced dosing compared to RLI2 and different RTX-RLI immunocytokines were comparable amongst each other whether comprising RLI2 or RLI2_{AQ}, whether being x2 or x1 or whether with or without a linker.

### Example 3: Reduced Production Yields of Homodimeric RLI2 Immunocytokines

Immunocytokines were expressed transiently in CHO cells and purified using standard antibody purification protocols using Protein A. Briefly, Mab select sure (GE) was used to capture immunocytokine products due to the presence of the Fc. Nuvia HR-S(CEX) was used in a binding/elution mode to separate oligomerized immunocytokine material and partly the uncoupled antibody RTX or PEM, as well as endotoxin and DNA contaminants. Preparative gel filtration (Superdex 200) was used for removing residual oligomerized ICK uncoupled antibody. The immunocytokines were concentrated to 2 mg/ml using Vivaspin 30 kDa.

Upstream production of RTX immunocytokines (RTX-ICKs) resulted in the presence of contaminants in supernatants representing 25-kDa and 50-kDa proteins, naked RTX or RTX-RLI x2 (RTX KiH-RLI x1), oligomerized RTX-RLI with a difference of productions between homodimeric having 2 RLI2 molecules and heterodimeric constructs having one RLI2 molecule using the KiH technology. Although it is generally expected that the heterodimeric antibody formats have lower expression due to mispairing of the heavy chains, surprisingly, it was observed for the heterodimeric immunocytokines, that heterodimeric constructs using the KiH technology had a higher expression compared to respective homodimeric constructs. Accordingly, unoptimized production yields were about 3 fold higher for RTX-RLI2 x1 (220-300 mg/l) compared to RTX-RLI2 x2 (70-100 mg/l), and ever 6 fold higher for PEM-RLI2 x1 (90-120 mg/l) compared to PEM-RLI2 x2 (10-20 mg/l), whereas in these not optimized expression the IgG4 PEM constructs generally had a worse expression compared to the IgG1 RTX constructs. Without being bound by any theory, the inventors speculate that the significant loss in expression of correctly folded homodimeric immunocytokines is linked to the interference of two RLI molecules linked to each heavy chain of the to be folded antibody with the proper antibody folding, as the RLI molecules have the tendency to interact with each other and thereby limit the freedom of the heavy chain C-termini to form the proper homodimer.

Whereas both KiH mutations and the L235E mutation did not significantly impact the production yields of the PEM-RLI2 constructs, the YTE mutation—alone or in combination with L235E—reduced expression levels by a factor of 2.

### Example 4: IL-15 Muteins for Reduced In Vitro Potency

Mutations were introduced within the IL-15 part of the RLI2 conjugate in order to reduce the binding and thereby the in vitro potency of the RLI conjugate to the IL-2Rβ and/or γ receptor, and to reduce heterogenicity of the RLI2-containing products. Indicated amino acid substitutions were made in the mature human IL-15 sequence (see Table 12).

TABLE 12
Amino acid substitutions in IL-15 and respective position in RLI2

| mature human IL-15 position | RLI2 position |
|---|---|
| D8A | D105A |
| K10A | K107A |
| D30N | D127N |
| D61A | D158A |
| E64Q | E161Q |
| N65D | N162D |
| N65A | N162A |
| G78A | G175A |
| N79Q | N176Q |
| Q101D | Q198D |
| Q108A | Q205A |

TABLE 13
Potency of RLI2 muteins having IL-15 variants (fused to
pembrolizumab-derived antibody) on kit225 cells

| | Affected | | | | PEM- |
|---|---|---|---|---|---|
| | binding | | | Potency | (x1 and |
| | to | Mutation* | | in vitro | x2 RLI2) |
| | subunit | (IL-15) | abbrev. | (kit225) | 12 ICKs |
| RLI2 wt | — | — | — | 1x | √ |
| RLI2 | β | D8A | | — | |
| muteins | | D61A | DA | −182x | √ |
| | | N65D | ND | −461x | √ |
| | | N65A | NA | −1128x | √ |
| | | D61A/N65A | DANA | | |
| | | K10A | KA | −2.3x | √ |
| | | | | | K10A, |
| | | | | | Q101D |
| | γ | Q101D | QD | −2.7x | |
| | | Q101A | QA | −2.1x | |
| | γ | Q101D/Q108A | QDQA | | |
| | β/γ | D30N/E64Q/N65D | NQD | −15 670x | √ |
| | | D61A/N65A/ | DANAQD | | |
| | | Q101D | | | |

Tested IL-15 substitutions affecting the binding to the IL-2Rβ3 and/or γ markedly reduced the potency of the RLI molecule on kit225 cells. The single mutant N65A lead to a similar reduction of potency as the NQD triple mutant (see Table 13). Other substitutions only had a minor influence on the potency.

TABLE 14
Potency of RLI-15 muteins (as such, without being fused to an
antibody) on kit225 cells

| | EC50 | Relative potency to |
|---|---|---|
| Sample | kit225 cells [pM] | SOT101 |
| SOT101 | 43.58 | — |
| RLI KAQD (AD) | 84.09 | 51.83% |
| RLI DA | 451.2 | 9.66% |
| RLI ND | 473.4 | 9.21% |
| RLI NA | 3847 | 1.13% |
| | | Relative potency to |
| | | PEM-RLI NQD x1 |
| PEM-RLI NQD x1 | 21617 | — |
| RLI DANA | 38619 | 55.98% |
| RLI DANAQD | 105930 | 20.41% |

Also for RLI-15 muteins tested without being bound to an antibody, the NA mutation lead to an about 2 log reduction of activity, here measured as EC50 on kit 225 cells.

### Example 5: Comparison of Homodimeric and Heterodimeric CD20-Targeted Immunocytokine

Immunocytokines based on the anti-CD20 antibody rituximab were generated by fusion of the RLI2 wt conjugate to the C-terminus of the antibody heavy chains (“x2”) or by using a KiH variant of rituximab by fusion of one RLI2 mutein to one C-terminus of one heavy chain (“x1”). Immunocytokines based on ntuximab (“RTX”) were tested for their in vitro potency on kit225 cells (see Example 1 and Table 15).

TABLE 15
Potency of homodimeric or heterodimeric RTX-RLI2
immunocytokines on kit225 cells

| ICK | Average relative potency on kit225 cells |
|---|---|
| RLI2 | 100% |
| RTX-RLI2 x2 | 47% |
| RTX-RLI2 AQ x2 | 55% |
| RTX-RLI2 x1 | 9% |
| RTX-L40-RLI2 x2 | 79% |

Whereas the homodimeric RTX-RLI 2× immunocytokine having two RLI2 conjugates fused the C-termini had a minor reduction of potency, the heterodimeric immunocytokine having only one RLI2 conjugate showed an about 10 fold reduction in potency on kit225 cells.

Similar activity was observed on activation (Ki67⁺ cells) of hNK cells CD8⁺ T cells after 7 days stimulation in vitro.

TABLE 16
EC50 (pM) of selected immune cell population from human PBMC

| | RTX- | RTX- | RTX- | RTX-L40- |
|---|---|---|---|---|
| | RLI2 | RLI2_{AQ} | RLI2_{AQ} x1 | RLI2_{AQ} |
| NK cells | 25 | 24 | 48 | 31 |
| Ki67⁺ | | | | |
| CD8⁺ Ki67⁺ | 349 | 376 | 1128 | 299 |
| T cells | | | | |
| CD8⁺ Ki67⁺ | 254 | 306 | 772 | 246 |
| memory T | | | | |
| cells | | | | |
| Kit225 | 50 | 47 | 272 | 31 |

Potency of immunocytokines on kit225 can be correlated with potency of immunocytokines on activated human NK cells, CD8⁺ T cells and CD8⁺ memory T cells. Only the RTX-RLI2 x1 had a about 3 fold reduction.

### Example 6: PD Activity of Anti-CD20 Immunocytokines Based on Rituximab

Immunocytokines were tested for their PD activity on immune cells from spleen after administration of the equimolar doses of RTX-ICKs administered IV at day 1 in healthy Balb/c mice (2 mice/group). RLI2 was injected SC at 20 μg/mouse daily at four consecutive days (day 1-day 4). The activation of immune cell population was detected at day 5 by flow cytometry. Following antibodies (Table 17) were used for the PD study (mouse).

TABLE 17
antibody panel for flow cytometry

| | | | |
|---|---|---|---|
| Marker | Fluorochrome | Vendor | Clone |

T cell panel

| | | | |
|---|---|---|---|
| CD3 | PE-Cy7 | eBioscience | 145-2C11 |
| CD4 | PerCP | eBioscience | RM4-5 |
| CD8 | V500 | eBioscience | 53-6.7 |
| CD44 | APC | eBioscience | IM7 |
| CD122 | eF450 | eBioscience | TM-b1 |
| NKG2D | PE | eBioscience | CX5 |
| CD62L | FITC | BD Biosciences | MEL-14 |
| FVD | eF780 | Invitrogen | — |
| Ki67- | A700 | Invitrogen | SolA 15 |

NK/T regulatory cell panel

| | | | |
|---|---|---|---|
| CD3 | PE-Cy7 | eBioscience | 145-2C11 |
| CD4 | PerCP | eBioscience | RM4-5 |
| CD8 | V500 | eBioscience | 53-6.7 |
| CD49b | eF450 | BD Biosciences | DX5 |
| CD25 | APC | eBioscience | PC61.5 |
| FVD | e780 | Invitrogen | — |
| Ki67 | A700 | Invitrogen | SolA15 |
| Foxp3 | PE | eBioscience | FJK-16S |

There was no difference in PD activity between RTX-RL12 x2, RTX-RLI AQ x2 and RTX-L40-RL12 x2 in vivo (FIG. 15). The PD activity of RTX-RL12 x1 in equimolar amount was lower due to only one R1A2 molecule/antibody, but only of about around 20-30% less in relative number of immune cells in comparison to double RTX-RLI x2 molecule.

### Example 7: Anti-Metastatic Activity of Anti-CD20 Immunocytokines Based on Rituximab in Renca Mouse Metastatic Model In Vivo

The anti-metastatic activity of anti-CD20 immunocytokines at the equimolar doses was tested in Renca renal cell carcinoma metastatic model in Balb/c mice. 3 μg/dose ICKs was injected IV at D1, lungs were harvested at Day 16 and the lung wet weight was determined.

No significant difference was observed in anti-metastatic activity of RLI2_{AQ} moiety between RTX-RLI2_{AQ} x2, RTX-RLI2_{AQ} x2, RTX-L40-RLI2_{AQ} x2 and RTX-RLI2_{AQ} x1 in vivo (FIG. 16). Single injection of 3 μg/dose resulted in 30-40% decreased of metastasis in comparison to the control.

### Example 8: Anti-Tumour Efficacy of Anti-CD20 RTX-RLI2_{AQ} Immunocytokines in A20-hCD20/Balb/c Mice

Anti-tumour efficacy of RTX-RLI2_{AQ} x2 immunocytokines was tested in Balb/c mice s.c. implanted with the A20-hCD20 tumour cell line (CrownBiosciences, USA). Mice were randomised intotreatment groups based on tumour volumes using a matched distribution function provided by the StudyDirector animal management software package (v3.0, StudyLog Systems, US) to achieve a minimum amount of variation between and within groups at day 1. RTX-RLI2_{AQ} x2 was administered at 0.15 mg/kg at day 1 and 8 and RLI2 was administered at 1 mg/kg for four consecutive days at day 1-4. Tumour volume was measured twice weekly for the duration of the study, the measurement was performed in two dimensions using a caliper and the volume was expressed in mm³ using the formula “V=(L×W×W)/2”, where V is tumour volume, L is tumour length (the longest tumour dimension) and W is tumour width (the longest tumour dimension perpendicular to L).

Two i.v. injections of RTX-RLI2_{AQ} x2 showed a significant anti-tumour efficacy in the A20-hCD20/balbc mouse tumour model when compared to control. Similar efficacy was shown for RLI2 when administered 4 times at a nearly 10 fold higher dose (or even higher comparing equimolar doses due to the larger molecular weight of the immunocytokine) compared to the RTX-RLI immunocytokine (FIG. 17).

### Example 9: ADCC Activity Anti-CD20 Immunocytokines Based on Rituximab Compared to Rituximab Alone

Daudi cell line was incubated with indicated concentrations of RTX and RTX-RLI2 molecules with or without NK92-CD16 cells. The Daudi cell death was assessed as percentage of DAPI positive cells and detected by flow cytometry.

4×10⁴ Daudi tumour cells (B cell lymphoma expressing CD20) per/well were seeded in 96-well plate. NK92-CD16 cells were added at ratio 1:5 together with a serial dilution of RTX-RLI2_{AQ} molecules (concentration 0.001, 0.01, 0.1, 1, 10 and 100 nM). Cells were incubated for 4 h at 37° C. in humidified 5% CO₂. After incubation, cells were stained with CD56-Alexa Fluor700, CD19-PE antibodies for NK cells and tumour cell distinctions, and with DAPI to identify dead tumour cells (CD19+DAPI+ cell) and analysed by flow cytometry.

ADCC activity of RTX-RLI2_{AQ} molecules was slightly lower than Rituximab control. However, only 60% of cells were killed by ADCC activity of Rituximab alone, compared to 70% or 80% for the RTX-RLI AQ and RTX-RLI 1×, respectively (FIG. 18).

### Example 10: IL-15 N65A Mutation in a PD-1-Targeted Immunocytokine Shows Diminished Potency on Kit225 Cells

Immunocytokines based on the anti-PD-1 antibody pembrolizumab were generated in various formats. Pembrolizumab is a humanized IgG4-K antibody having the stabilizing S228P mutation in the Fc part of the antibody. Variations of pembrolizumab (“PEM”) were tested in order to improve the construct for the use in an immunocytokine. Although the IgG4 antibody class is known to have relatively low ADCC activity, the L235E mutation (Alegre, Collins et al. 1992) (“LE”) was introduced in order to further reduce ADCC (SEQ ID NO: 43). More complex ADCC inactivating mutations were avoided in order to limit the potential of immunogenicity/anti-drug antibodies. Either one or two RLI2 molecules were genetically fused to the C-terminus of the PEM antibody. In case of homodimeric PEM variants (“x2”) one RLI2 molecule was fused to each heavy chain, whereas heterodimeric PEM variants (“x1”) were made using the knob-in-hole (KiH) technology (Elliott, Ultsch et al. 2014), whereas one RLI2 molecule was fused to the knob heavy chain having the T336W substitution (SEQ ID NO: 41), whereas the hole heavy chain (with no RLI2 fusion) comprised the T366S/L368A/Y407V substitutions (SEQ ID NO: 42). When RLI2 was fused to a heavy chain, the terminal lysine (K) was deleted (“dK”) in order to reduce heterogeneity of the product. Further, different RLI2 muteins were used to fuse to the heavy chain of the antibody. All RLI2 molecules had the AQ (G78A/N79Q) substitution for reducing the heterogeneity of the product, and the following substitutions reducing the binding of RLI2 to the IL-2/IL-15Rβγ were tested in the PEM-RLI immunocytokines: DA, NA, ND, AD (K10A Q101D), and NQD. Made PEM-RLI immunocytokines are listed in Table 18, left column.

The potency of several homodimeric or heterodimeric PEM-RLI2_{AQ} immunocytokines with the provided IL-15 substitutions was compared by measuring the in vitro EC50 on kit225 cells (Table 18) with RLI2 being used as a standard and set to 100% for relative potency. The aim was to identify the least potent mutein of RLI2 on kit225 cells. Shown results are mean of 2-5 experiments.

TABLE 18
Potency of PEM-RLI2 mutants on kit225

| | No. of RLI | EC50 kit225 cells | Relative potency/ |
|---|---|---|---|
| Sample | molecules | pM | kit225 cells |
| RLI | 1x | 51 | 100% |
| PEM LE-RLI x1 | 1x | 335 | 15% |
| PEM LE-RLI x2 | 2x | 109 | 47% |
| PEM LE-RLI-AD x1 | 1x | 603 | 8.5% |
| PEM LE-RLI-AD x2 | 2x | 122 | 42% |
| PEM LE-RLI-DA x1 | 1x | 4484 | 1.1% |
| PEM LE-RLI-DA x2 | 2x | 1620 | 3.1% |
| PEM LE-RLI-NA x1 | 1x | 13983 | 0.36% |
| PEM LE-RLI-NA x2 | 2x | 10663 | 0.47% |
| PEM LE-RLI-ND x1 | 1x | 9238 | 0.55% |
| PEM LE-RLI-ND x2 | 2x | 5270 | 0.97% |
| PEM LE-RLI-NQD x1 | 1x | ND | — |
| PEM LE-RLI-NQD x2 | 2x | 116988 | 0.044% |

RLI: RLI2_{AQ}; ND . . . not detected (limited sensitivity of the assay)

The RLI2_{AQ}NA within PEM-RLI-NA x1 was identified as the least potent RLI mutein with a single mutation lowering the IL-2/IL-15Rβγ, which still is about 10 fold more active than the NQD mutation, which has three amino acid substitutions, thereby having a relatively higher risk of immunogenicity.

Heterodimeric PEM-RLI immunocytokines were analysed by capillary electrophoresis under reducing and non-reducing conditions (FIG. 1). All immunocytokines showed high purity with clear separation of the antibody heavy chain, heavy chain+RLI (HC-RLI) and light chains. The faint band just above the HC-RLI band represent glycosylated RLI on the heavy chain. Surprisingly, glycosylation seemed to be reduced for the NA mutant.

### Example 11: PEM-RLI x1 or PEM-RLI NA x1 Molecules with Fc Variants (LE, YTE or LE-YTE) Show No Differences in their Potency on Kit225 Cells In Vitro

The potency of several heterodimeric PEM-RLI x1 and PEM-RLI NA x1 with Fc variants designed to reduce ADCC (LE substitutions), or increase in vivo half-life through increased FcRn binding (YTE substitutions) or the combination of both (LE-YTE) were compared in vitro by determining the EC50 using kit225 cells (Table 19) with RLI2 being used as a standard and set to 100% for relative potency. The data represent mean of 2 experiments.

TABLE 19
Potency of PEM-RLI NA molecules with
altered Fc part of the antibody

| | | EC50 | Relative |
|---|---|---|---|
| | No. of | kit225 | potency/ |
| | RLI-15 | cells | kit225 |
| Sample | molecules | [pM] | cells |
| RLI2 | 1x | 31 | 100% |
| PEM-RLI x1 | 1x | 302 | 10.2% |
| PEM LE-RLI x1 | 1x | 201 | 15.4% |
| PEM YTE-RLI x1 | 1x | 251 | 12.3% |
| PEM LE/YTE-RLI x1 | 1x | 212 | 14.6% |
| PEM-RLI NA x1 | 1x | 11580 | 0.24% |
| PEM LE-RLI NA x1 | 1x | 11024 | 0.28% |
| PEM YTE-RLI NA x1 | 1x | 10384 | 0.29% |
| PEM LE/YTE-RLI NA x1 | 1x | 9235 | 0.33% |

RLI: RLI2_{AQ};

Fc variants of PEM in the heterodimeric fusion with RLI2_{AQ} having no IL-15 inactivating NA mutation in the RIM conjugate (PEM-LE, YTE, or LE/YTE-RLI x1) demonstrated similar potency as PEM-RLI x1 on kit225. Similarly, all compared constructs having the inactivating IL-15 NA mutation in the RLI conjugate showed similar (reduced) potency on kit225 cells independent of the tested Fc variant. Thus, tested mutations in the antibody Fc region did not influence the potency of PEM-RLI constructs.

### Example 12: Evaluation of PEM LE-RLI NA x1, PEM LE/YTE-RLI NA x1 and PEM-RLI NQD x1 Molecules in Potency on Kit225 In Vitro

Selected PEM-RLI immunocytokine constructs from two batches were compared with respect to potency in comparison to RL12 in vitro using the kit225 cells. The potency of molecules was assessed as EC50 and also calculated as a relative potency related to the RL12 molecule. The data represent one experiment. As shown in Table 20 a high batch-to-batch consistency was observed with regard to the potency of tested immunocytokines measured by determining the EC50 on kit225 cells.

TABLE 20
Relative potency of PEM-RLI constructs

| | EC50 | | EC50 | |
|---|---|---|---|---|
| | kit225 | Relative | kit225 | Relative |
| | cells | potency | cells | potency |
| | pM | batch 1 | pM | batch 2 |
| RLI2 | 18.9 | 100% | — | — |
| PEM LE/YTE-RLI NA x1 | 6097 | 0.32% | 5890 | 0.33% |
| PEM LE-RLI NA x1 | 11898 | 0.17% | 9981 | 0.20% |
| PEM-RLI NQD x1 | 186903 | 0.01% | 107489 | 0.02% |

### Example 13: Comparison of PEM LE-RLI NAx1, PEM LE/YTE-RLI NAx1 and PEM-RLI NQD x1 Molecules in Potency on Kit225 and hPBMC In Vitro

Selected PEM-RLI immunocytokine constructs bearing one or two RLI2 molecules (PEM-RLIs) with/without the LE/YTE Fc modification. Indicated PEM-RLI immunocytokines were used at various concentrations for stimulation of human PBMCs from 6 healthy donors for 7 days in vitro. The potency on human NK and CD8⁺ T cells was compared to the potency on kit225 cells (see Table 21). Surprisingly, for the N65A IL-15 mutant (“NA”) no difference in potency of molecules carrying one or two RLI2 molecules (compare x1 and x2 immunocytokines) was observed with respect to activation of human NK or CD8⁺ T cells or on kit225 in vitro. As known from other experiments, the LE/YTE in the Fc part of the antibody has no influence on the potency of the fused RLI molecule. The immunocytokine containing the IL-15 NQD mutein had a further reduces potency by a factor of about 10. Human PBMC potency data are mean of 6 donors. Kit225 data are mean of 2-3 experiments.

TABLE 21
Relative potency of PEM-RLIs on kit225 and hPBMC

| | EC50 | EC50 | EC50 |
|---|---|---|---|
| | kit225 | hNK | CD8⁺ |
| | cells | cells | T cells |
| | pM | pM | pM |
| RLI2 | 33.6 | 10 | 100 |
| PEM-RLI x1 | 333 | 342 | 512 |
| PEM LE/YTE-RLI NA x1 | 9235 | 3334 | 18020 |
| PEM-RLI NA x2 | 10663 | 3350 | 20430 |
| PEM-RLI NQD x1 | 147196 | 37620 | 154500 |

### Example 14: Evaluation of Low Potency PEM-RLI Mutants Attached to HC or LC on Kit225 Cells In Vitro

Several low potency IL-15 muteins in the PEM-RLI immunocytokines with or without mutated Fc antibody part (LE-YTE) were compared with respect to their potency in comparison to PEM LE/YTE-RLI2 NA x1 as a reference. In “Lc” immunocytokines, the RLI conjugate was fused to the C-terminus of the light chains of the antibody (whereas all other constructs have the RLI conjugate fused to the C-terminus of one of both heavy chains). The in vitro potency testing was accomplished using kit225 cell line with an altered protocol (prolonged cell incubation). The potency of molecules was assessed as EC50 and also calculates as a relative potency related to the PEM LE/YTE-RLI NA x1 molecule. The data in Table 22 represent mean of 2-4 experiments.

TABLE 22
Potency of PEM-RLI2_{AQ} molecules on kit225

| | | EC50 | Relative |
|---|---|---|---|
| | No. of | (kit225 | potency/ |
| | RLI2 | cells) | kit225 |
| | molecules | pM | cells |
| PEM LE/YTE -RLI2 NA x1 | 1x | 4 756 | 100% |
| PEM -RLI2 NQD x1 | 1x | 64 758 | 7.3% |
| PEM LE/YTE -RLI2 QDQA x1 | 1x | 9 449 | 50.3% |
| PEM LE/YTE -RLI2 DANA x1 | 1x | 480 157 | 1.0% |
| PEM LE/YTE -RLI2 DANAQD x1 | 1x | NA | NA |
| PEM LE/YTE-Lc-RLI2 DA x2 | 2x | 657 | 724% |
| PEM LE/YTE-Lc-RLI2 NA x2 | 2x | 3 493 | 136% |
| PEM LE/YTE-Lc-RLI2 DANA x2 | 2x | 272 966 | 1.7% |

The combinations of substitutions QDQA (Q101D/Q108A), NQD (D30N/E64Q/N65D), DANA (D61A/N65A) and DANAQD (D61A/N65A/Q101D) further reduced potency of the PEM-RLI immunocytokine constructs until not measurable for the DANAQD construct. Immunocytokines with the RLI conjugate fused to the light chains of the antibody showed similar potency compared to the constructs with only one RLI conjugate having the same IL-15 mutations on one heavy chain of the antibody.

### Example 15: Comparison of Low Potency Mutants with or without Mutated Fc Part

The aim was to evaluate and compare potency of several lower potency muteins than PEM-RLI NA x1 with or without mutated Fc antibody part (LE-YTE). These molecules are fusion proteins of pembrolizumab (IgG4) and RLI-15 (PEM-RLI-15). RL2 was used as a standard. The in vitro potency testing was accomplished using the kit225 cell line. The potency of molecules was assessed as EC50 and also calculates as a relative potency related to the naked RLI-15 molecule. The data represent mean of several experiments after 3.5 or 7 day of kit225 proliferation.

TABLE 23
Potency of PEM-RLI immunocytokines on kit225
(WP9) (3 final candidate molecules)

| | EC50 | x-fold lower |
|---|---|---|
| | (kit225 | potency cells |
| | cells) | than RLI2 on |
| | pM | kit225 cells) |
| RLI2 | 63 | 1x |
| PEM LE-RLI2 NA x1 | 23 835 | 385x |
| PEM-RLI2 NQD x1 | 29 394 | ~3 245x |
| PEM LE-RLI2 DANA x1 | 1 154 948 | ~107 085x |
| PEM LE/YTE-RLI2 DANAQD x1 | 2 529 824 | ~334 235x |

### Example 16: Pembrolizumab Functionality is Neither Affected by Fusion of RLI2 or RLI2 Muteins Nor by Fc Modifications of the Antibody

The functionality of the anti-PD-1 antibody derivative of pembrolizumab was determined by measuring the blockade of the PD-1/PD-L1 interaction using the bioluminescent cell-based assay “PD-1/PD-L1 Blockade Bioassay” (J1250, Promega) according to the instructions for use. Indicated PEM RLI immunocytokines were tested to evaluate their potency with respect to their activity to block the PD-1/PD-L1 interaction (see Table 24). No significant difference was observed between the tested immunocytokines. Therefore, the functionality (PD-1 blocking) of the PEM part of the immunocytokines has been preserved independently of the number of RLI2 molecules attached, of mutations in the RLI2 conjugate or the mutations in the Fc part of the antibody.

TABLE 24
Potency of PEM-RLI molecules in the PD-
1/PD-L1 Blockade Bioassay (Promega).

| | EC50 [pM] |
|---|---|
| PEM-RLI x1 | 1397 |
| PEM-RLI NA x1 | 1383 |
| PEM-RLI NA x2 | 1401 |
| PEM LE/YTE-RLI NA x1 | 1440 |

### Example 17: PEM-RLI NA x1 Immunocytokine Displays Anti-Tumour Efficacy in Mouse Tumour Model

The in vivo therapeutic efficacy of the PEM-RLI NA x1 immunocytokine was compared to Pembrolizumab as monotherapy in the treatment of HuCell MC38-hPD-L1 tumours in female human PD-1 single KI HuGEMM mice (n=8 mice per group). The treatment started when the mean tumour size reached 108 mm³ at randomization day 0. PEM-RLI NA x1 was administered IV at 20 mg/kg at day 0 and pembrolizumab was administered IP at 5 mg/kg at days 0,3,6 and 9.

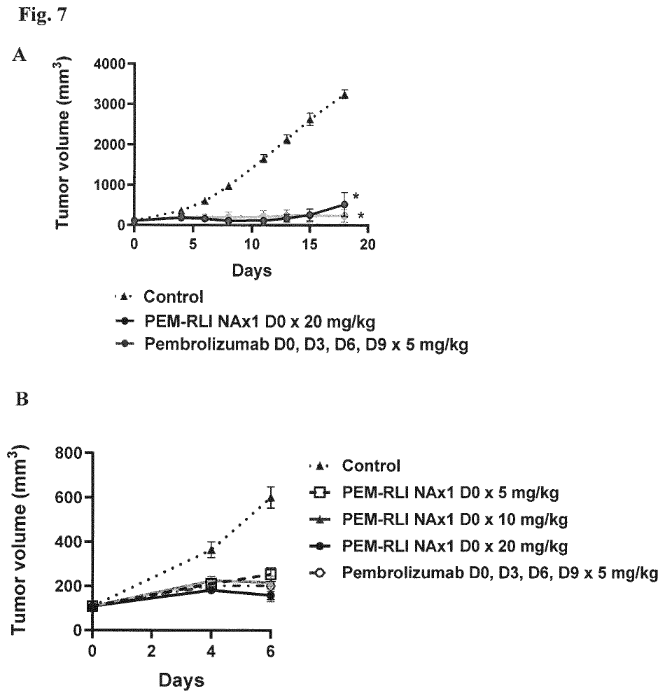

PEM-RLI NA x1 strongly decreased tumour volume in this model in comparison to the control untreated group (p-value was <0.05) and similarly to the pembrolizumab treatment group (see FIG. 7). While no marked difference to pembrolizumab was seen for the immunocytokine, it should be noted that a single injection of the immunocytokine achieved a similar result as four administrations of pembrolizumab. Further, as mouse is known to be about 10 fold less sensitive to RLI, the full functionality of the PEM-RLI NA x1 cannot be tested in this mouse model and accordingly treatment effect in humans is expected to be better.

### Example 18: PEM LE/YTE-RLI NA x1 Molecule Enhances IFN-γ Production in Mixed Lymphocytes Reaction Over Pembrolizumab and RLI-15 Monotherapies

For evaluation of the potential of PEM-RLI constructs to enhance T-cell activation and IFNγ production, a mixed lymphocyte reaction (MLR) was employed. MLR is an in vitro assay in which leukocytes, from two genetically distinct individuals of the same species, are cocultured resulting in cell blast transformation, DNA synthesis and proliferation. Generation of the MLR occurs as a consequence of the incompatibility of the allogeneic determinants, which are expressed on the surface of cell populations and which are encoded by the major histocompatibility complex (MHC).

T-cell activation via IFNγ production was evaluated for PEM RLI-NA x2, PEM RLI-NQD x1, PEM LE/YTE-RLI NA x1 and PEM YTE-RLI x1 molecules in a MLR assay in vitro. PEM-RLI constructs were compared to RLI2 and Pembrolizumab. The respective RLI-15 muteins without being fused to an antibody, which would be much more suitable as corresponding controls, were not available at the time of the study.

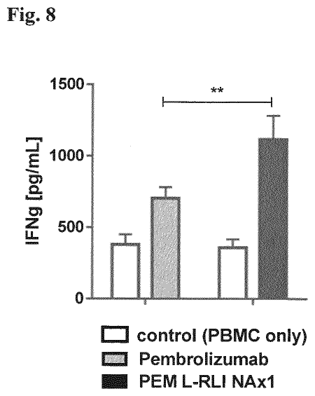

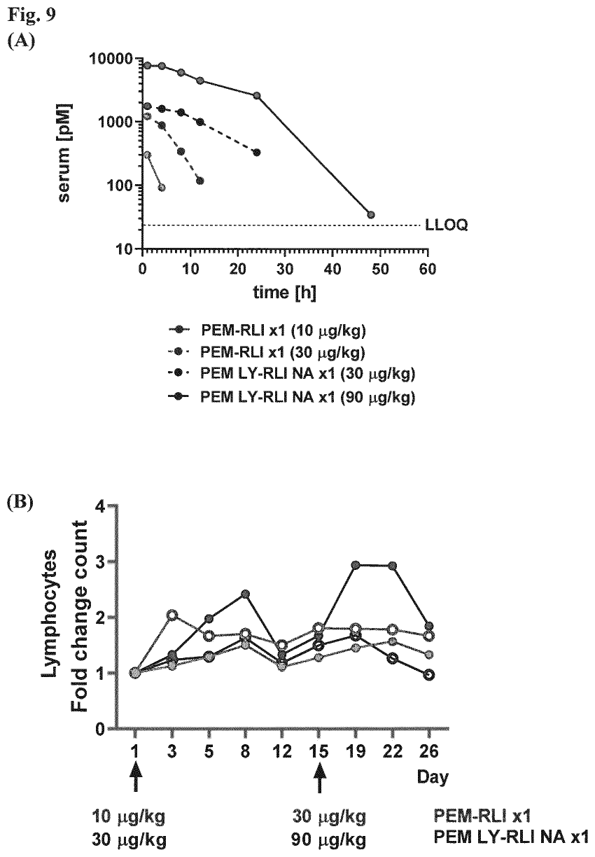

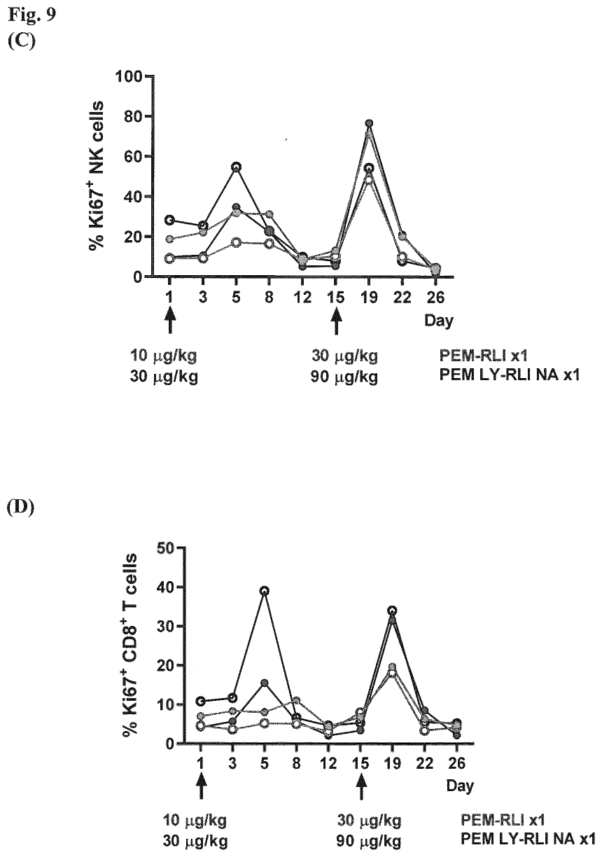

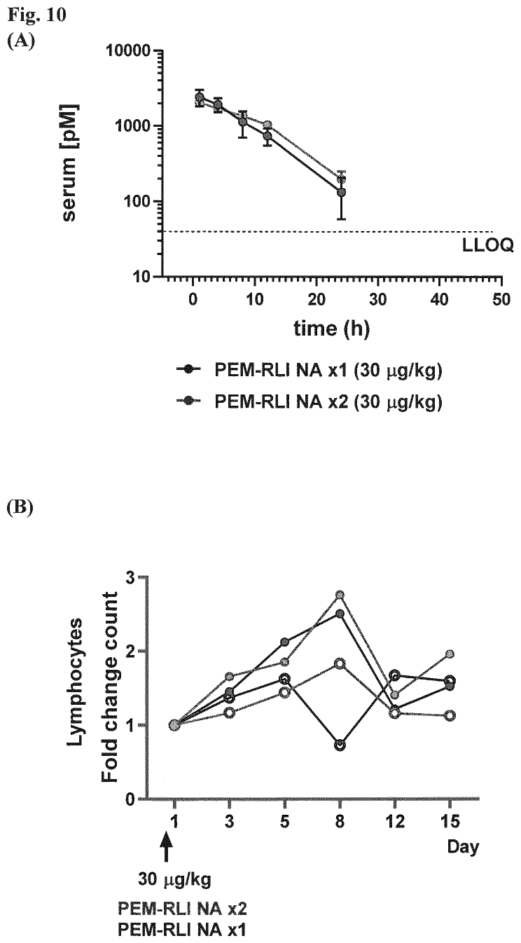

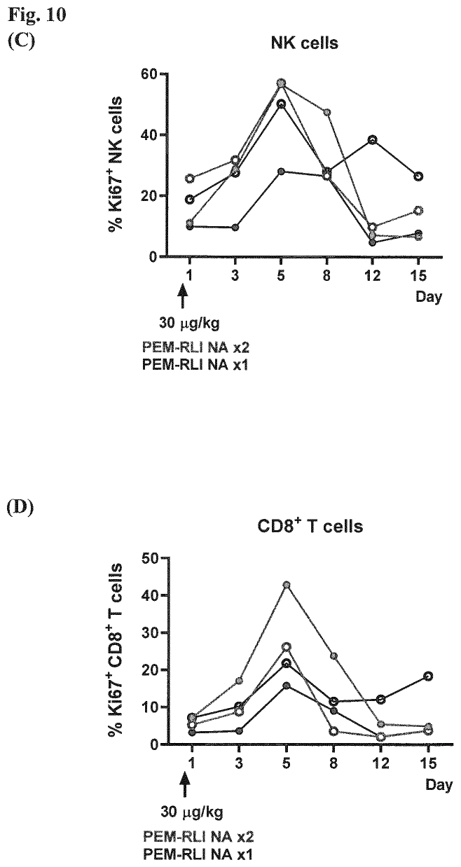

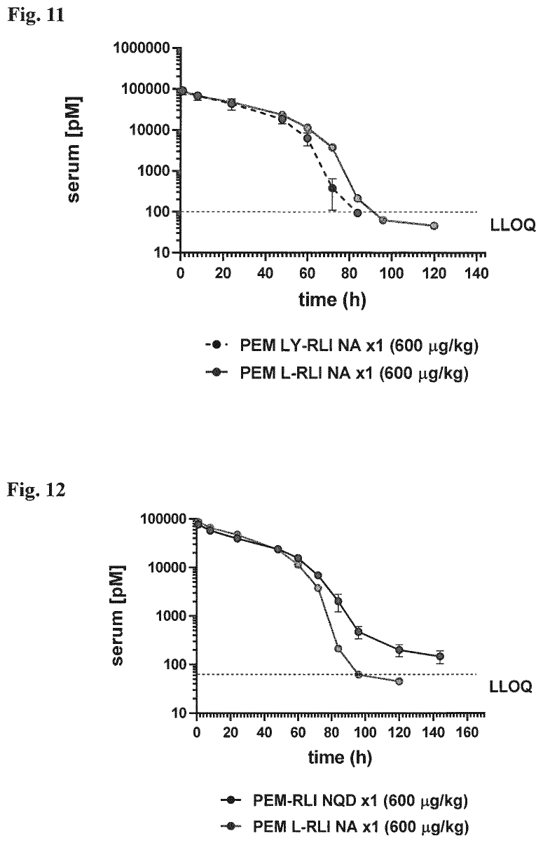

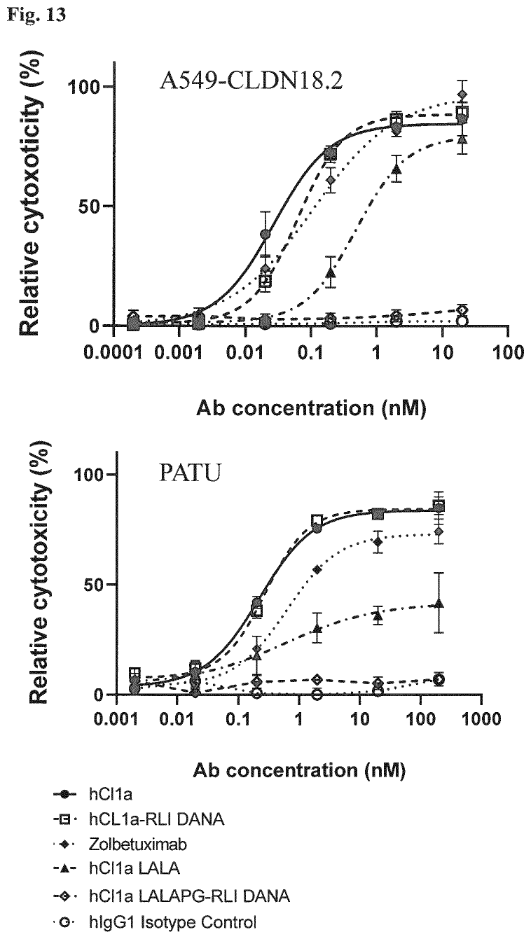

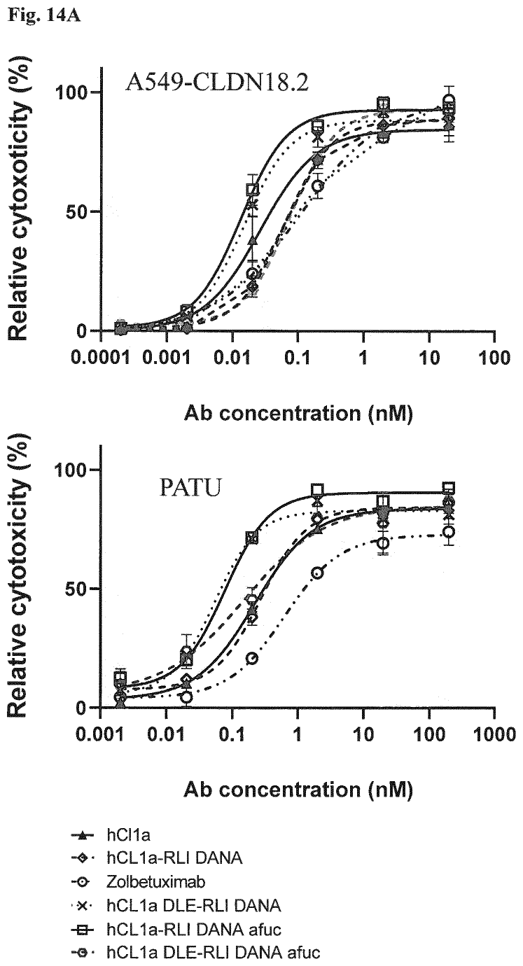

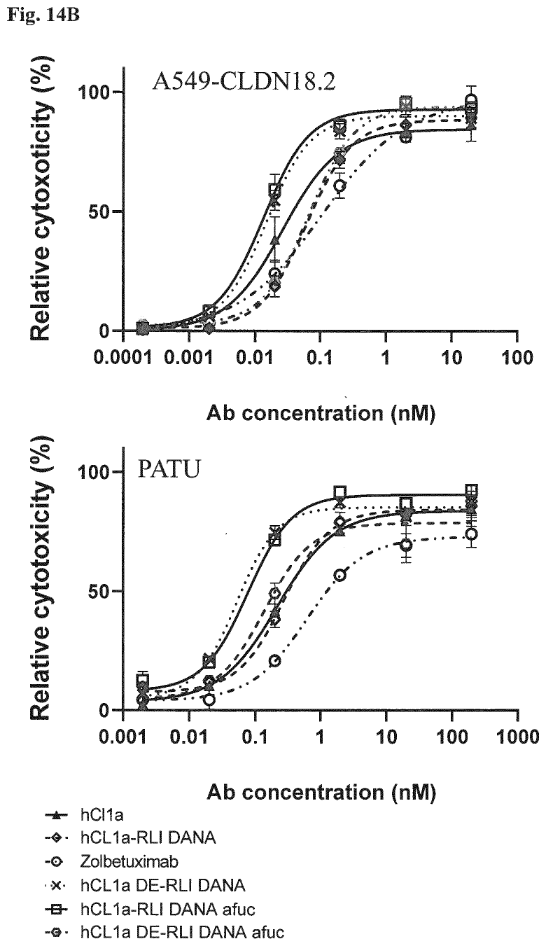

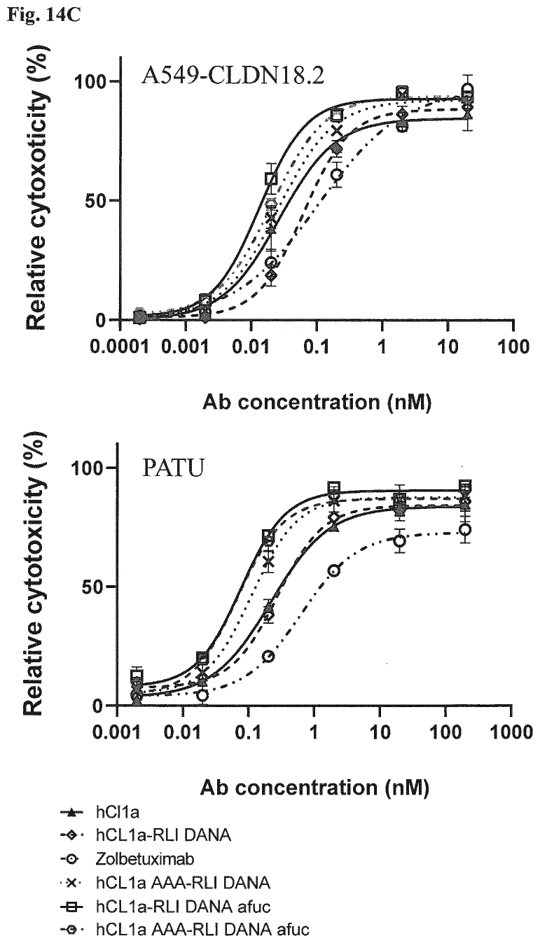

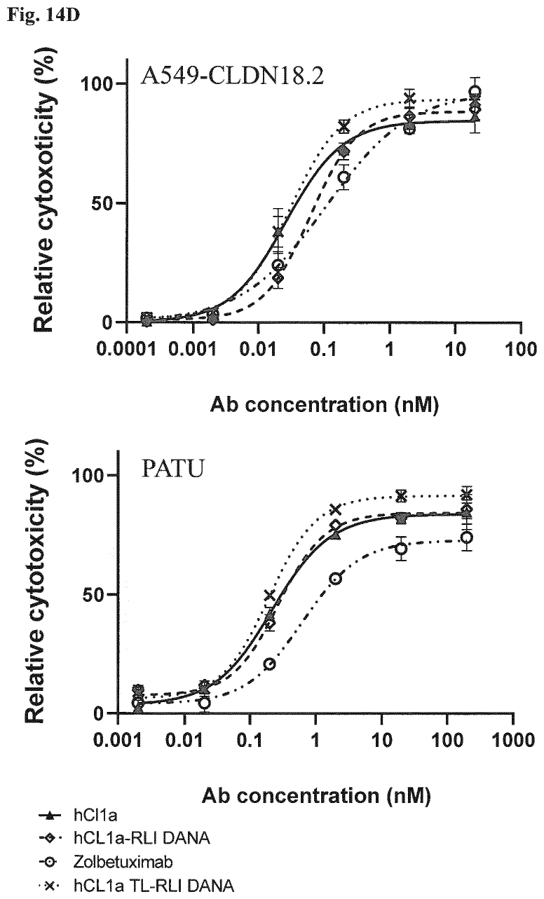

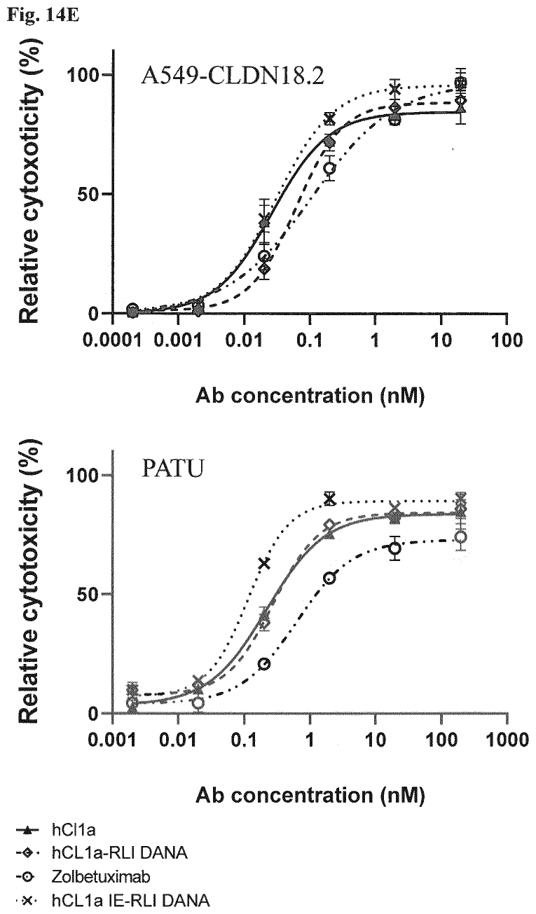

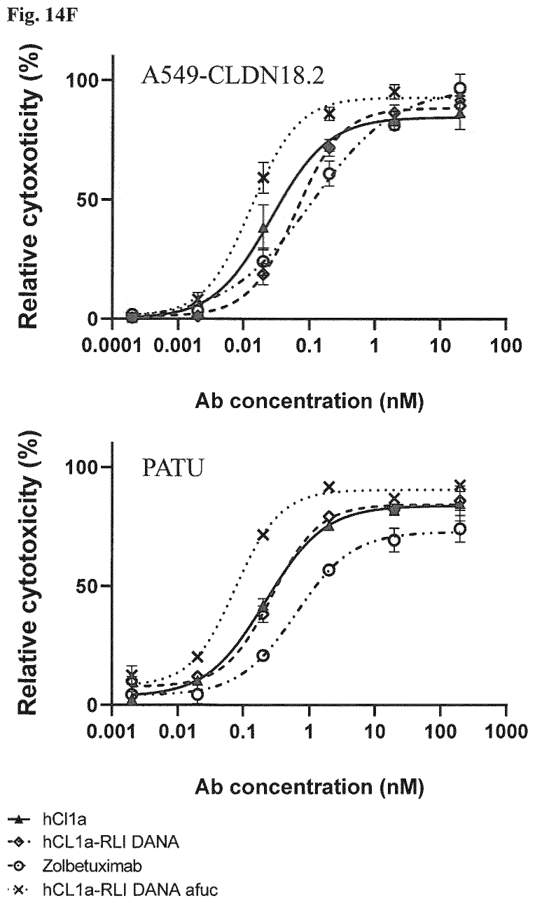

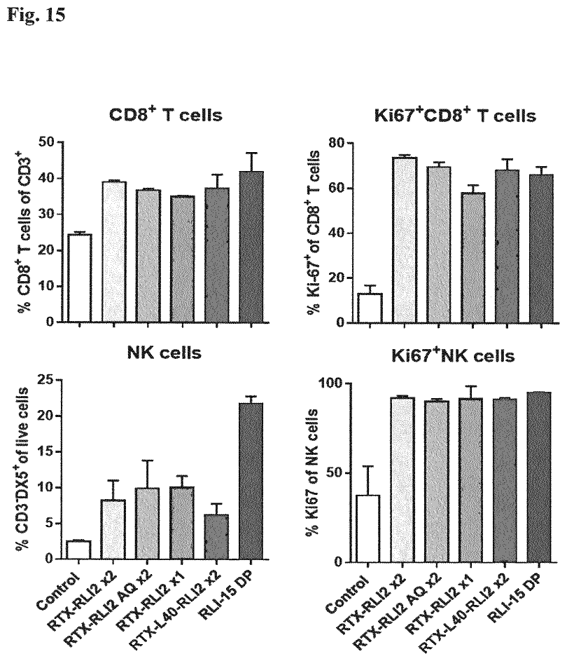

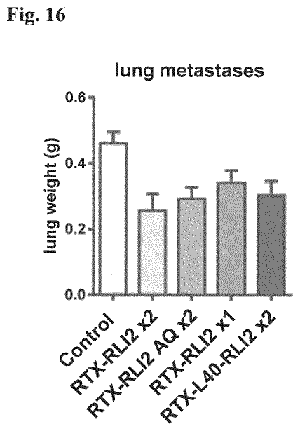

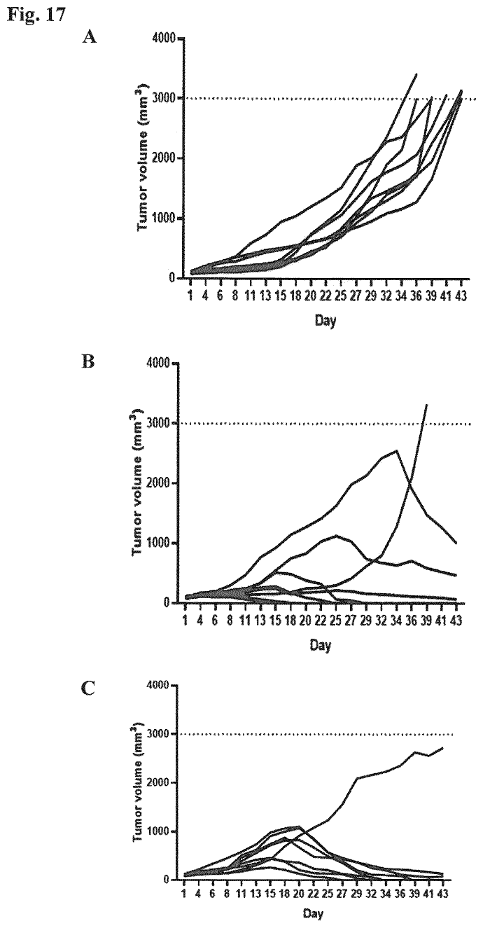

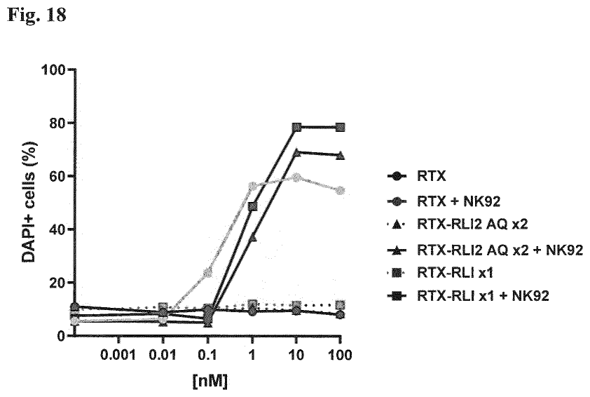

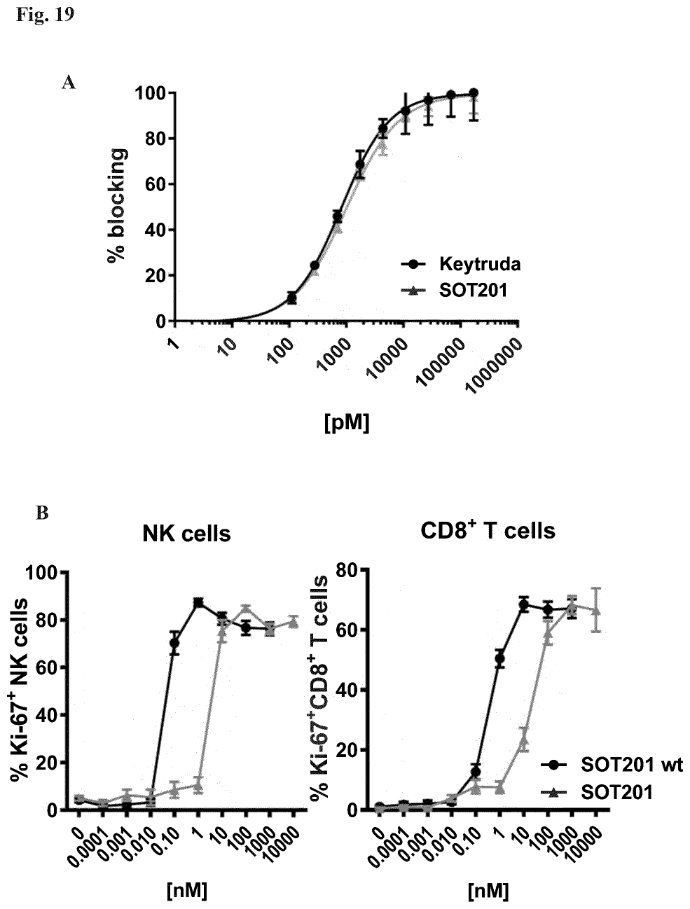

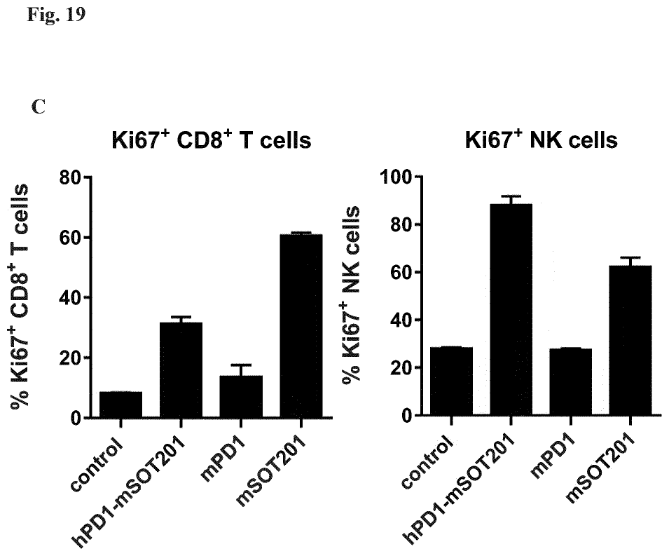

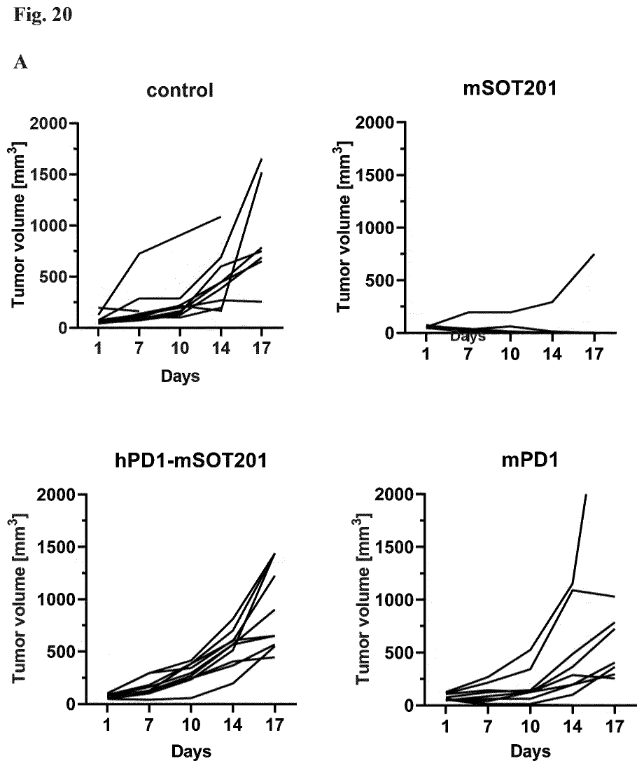

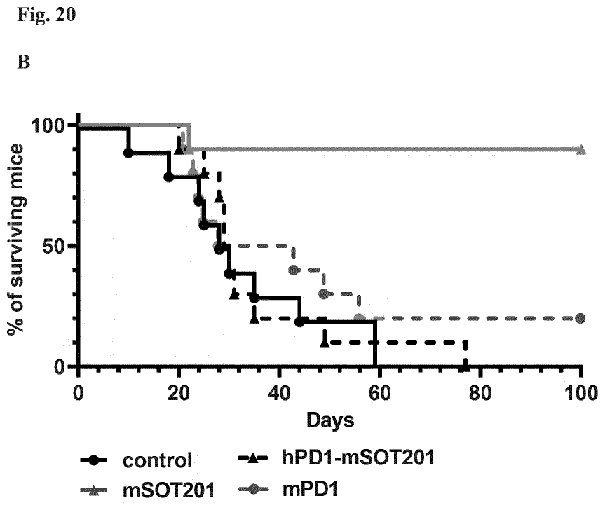

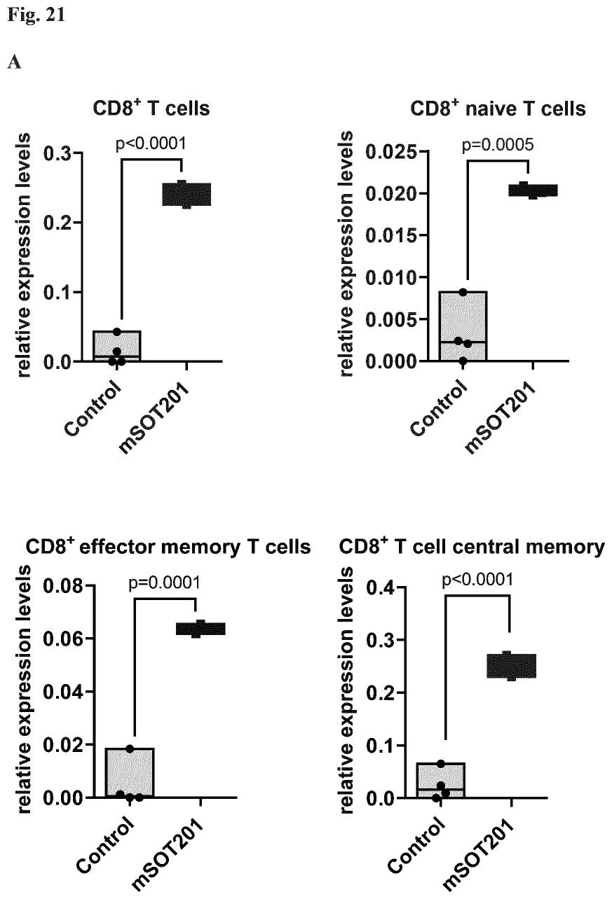

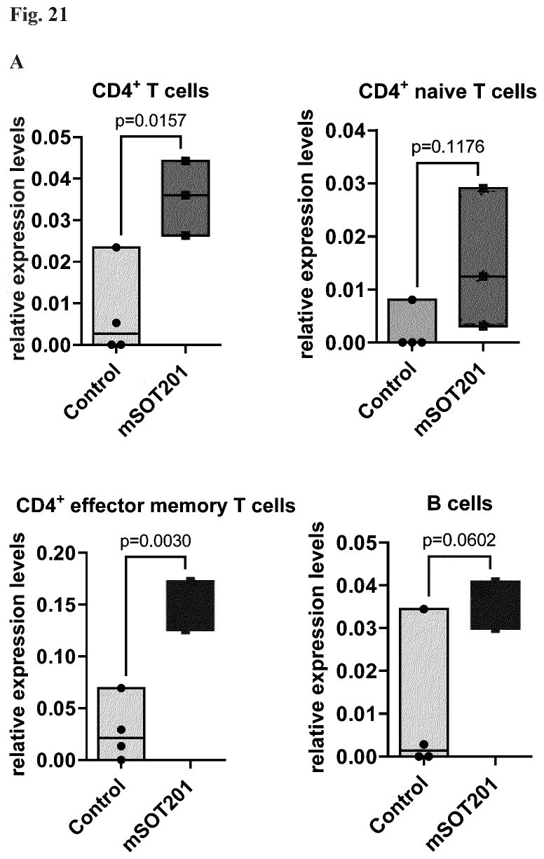

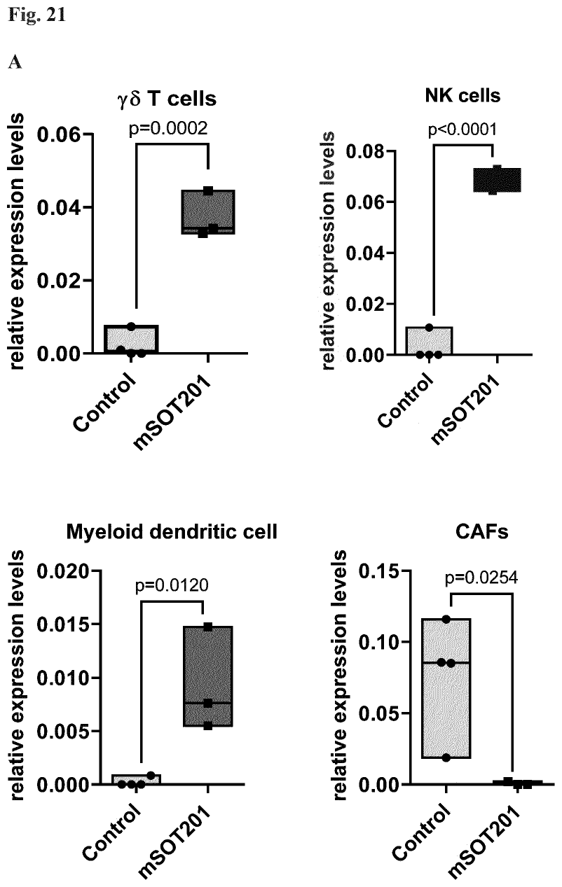

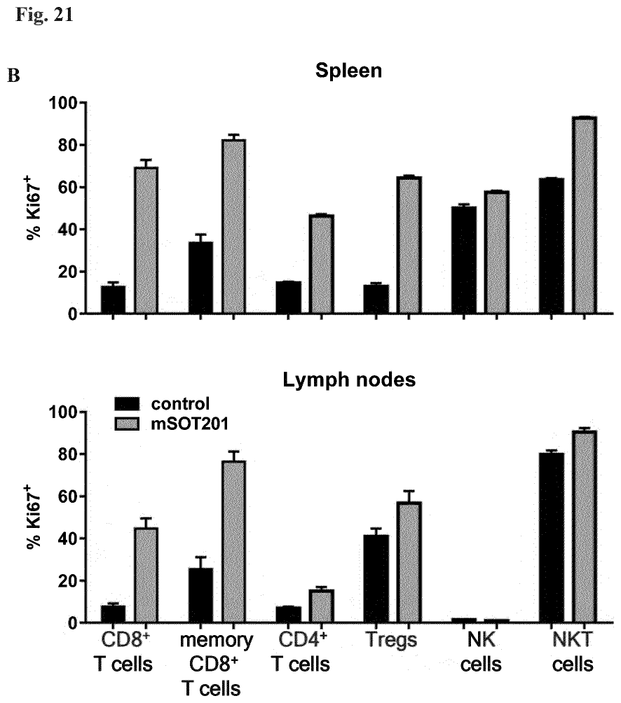

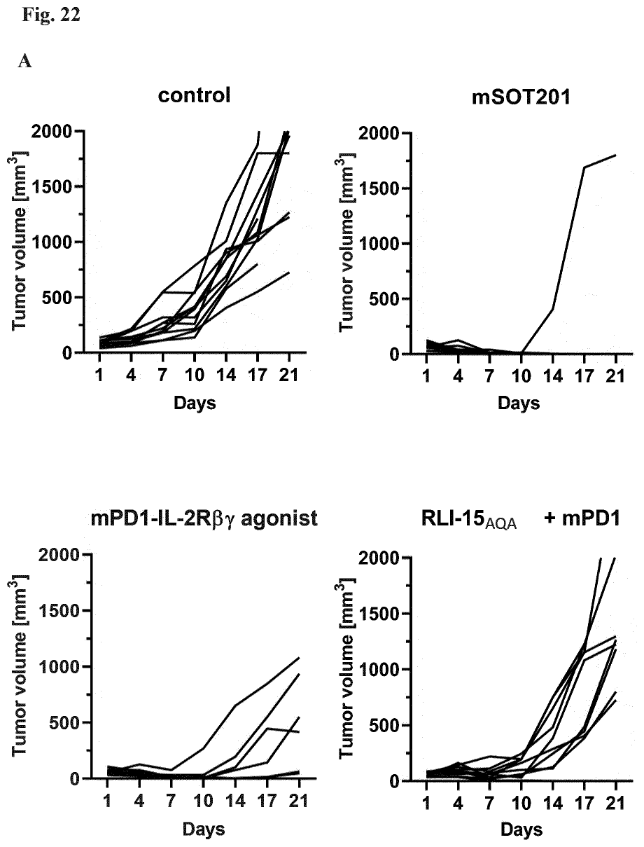

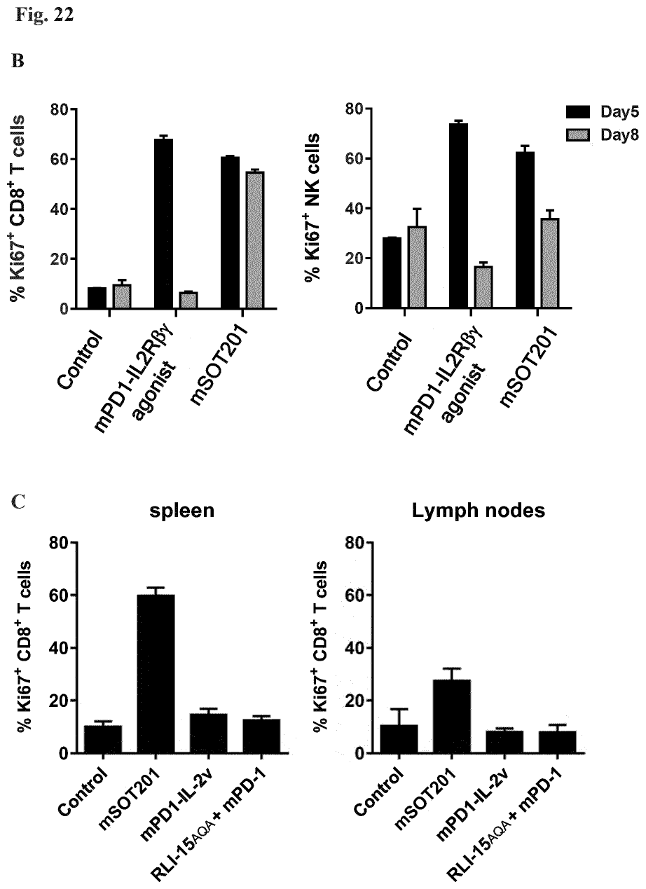

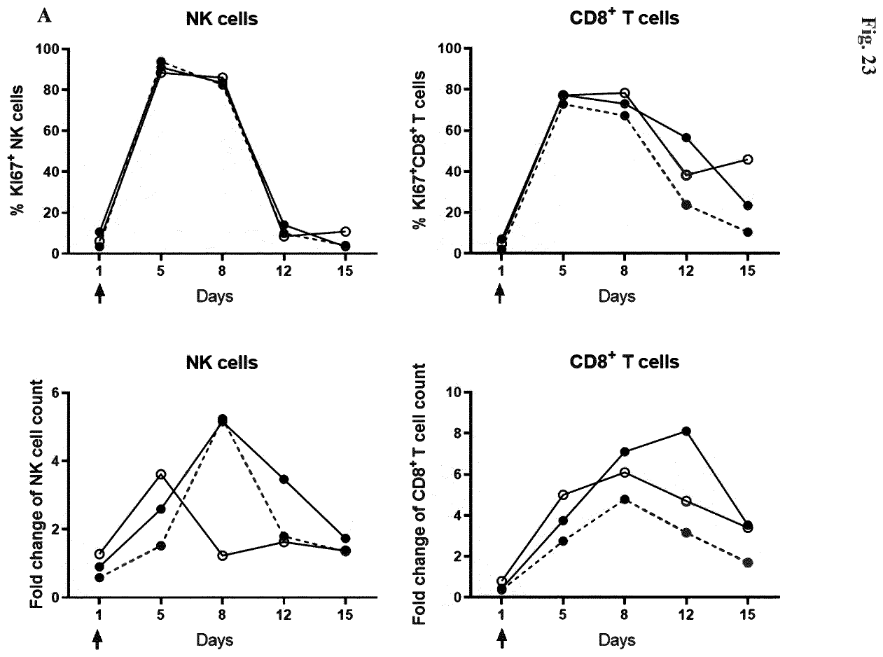

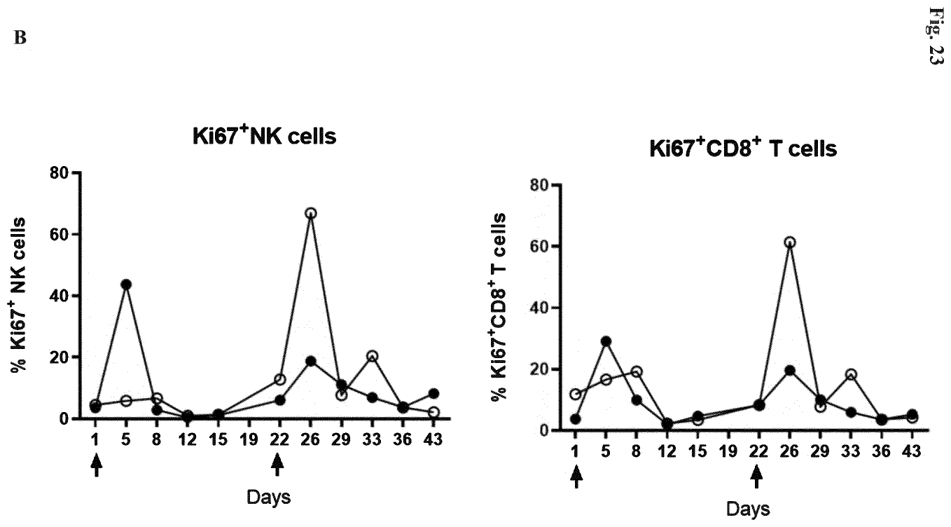

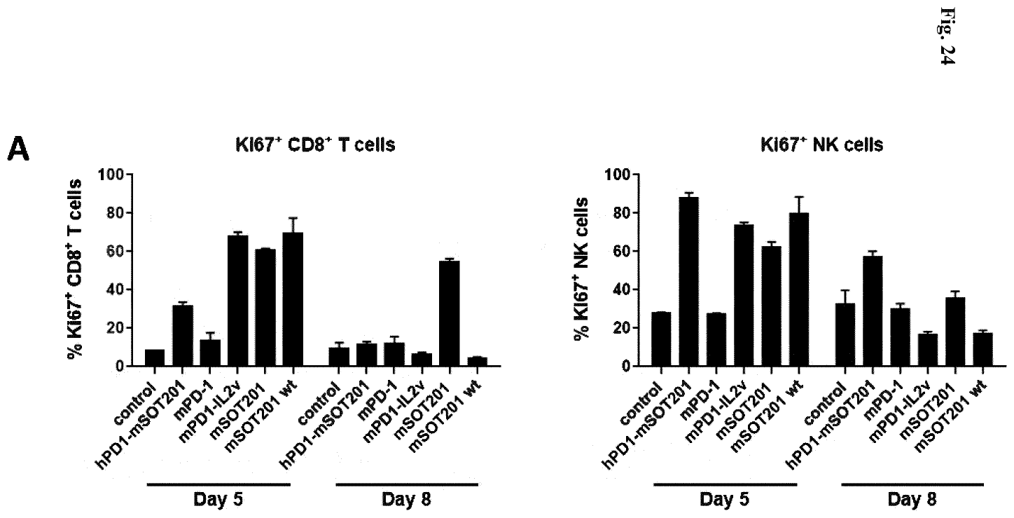

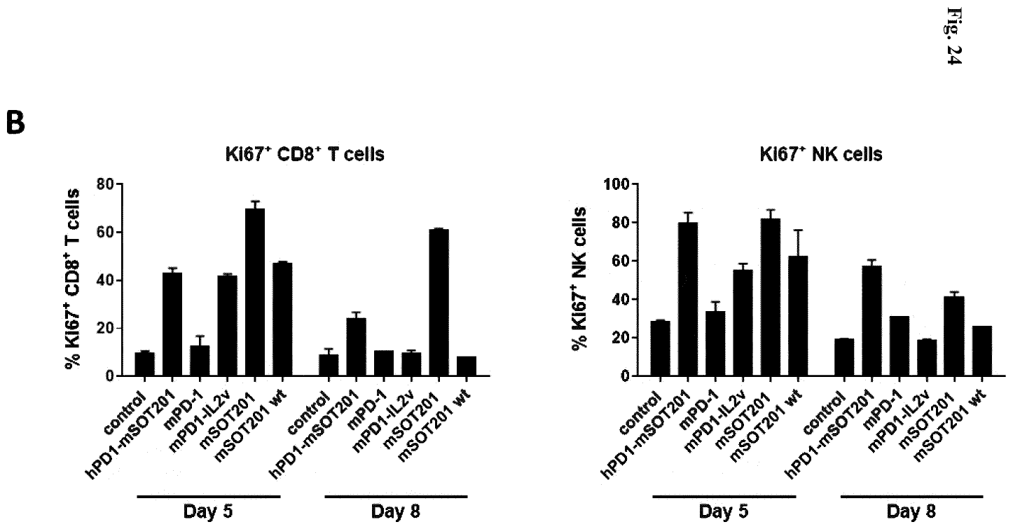

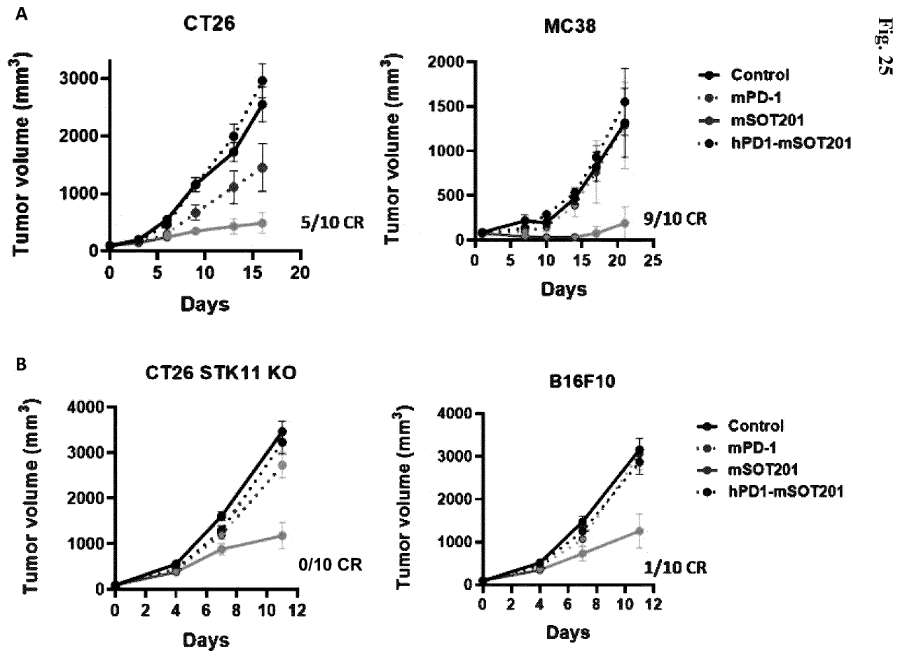

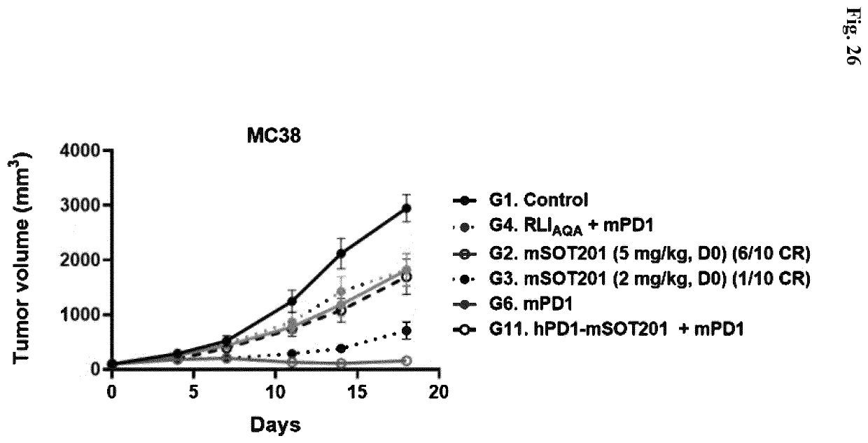

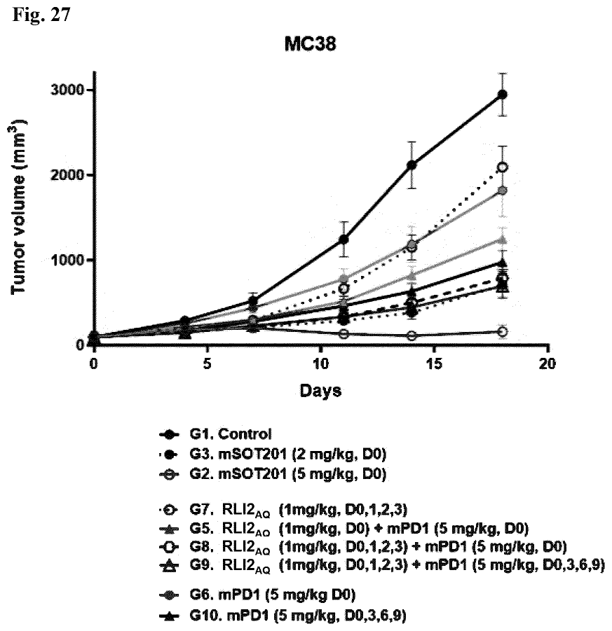

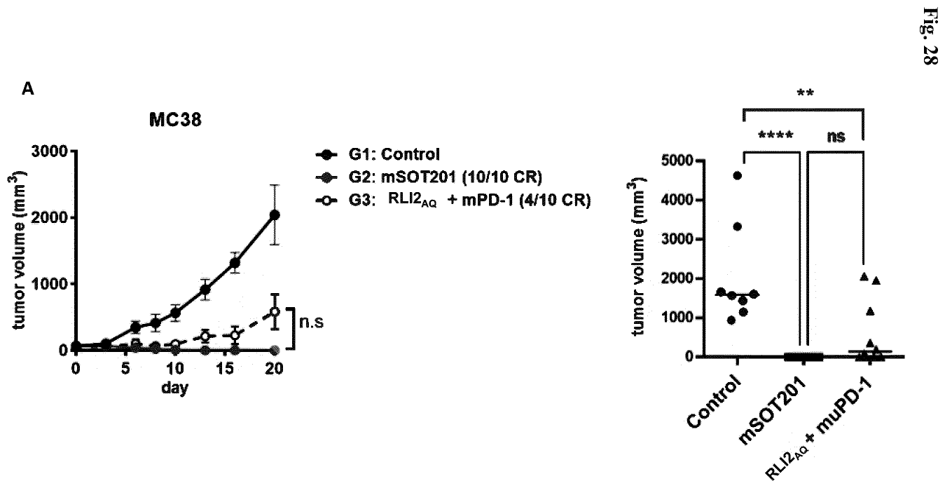

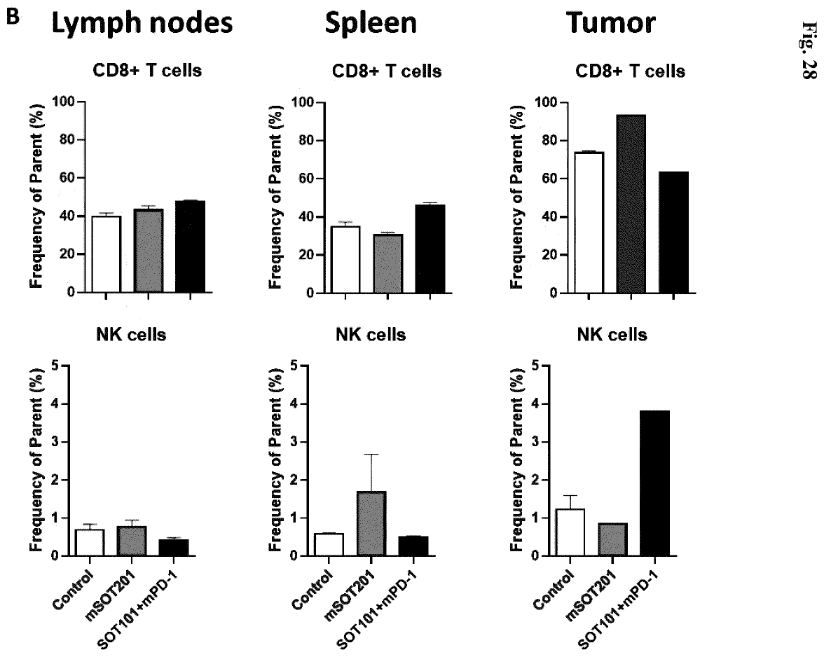

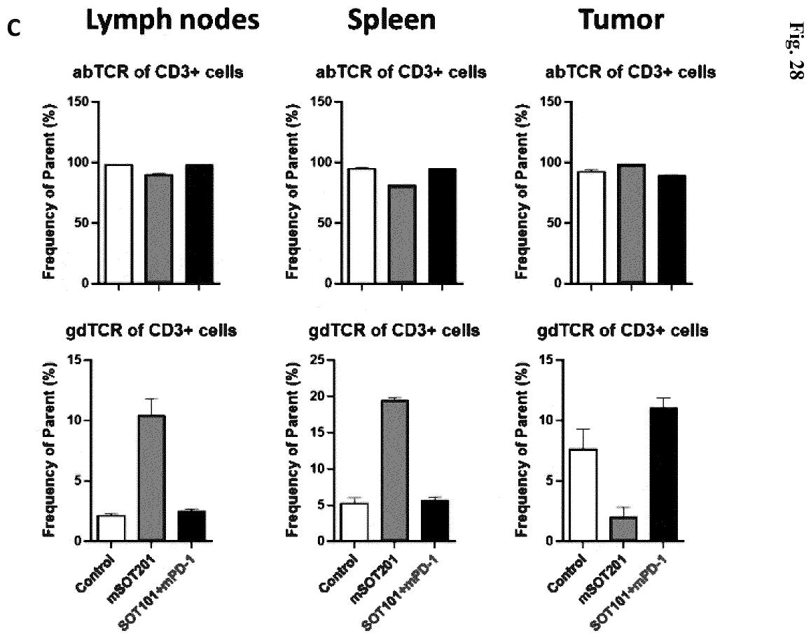

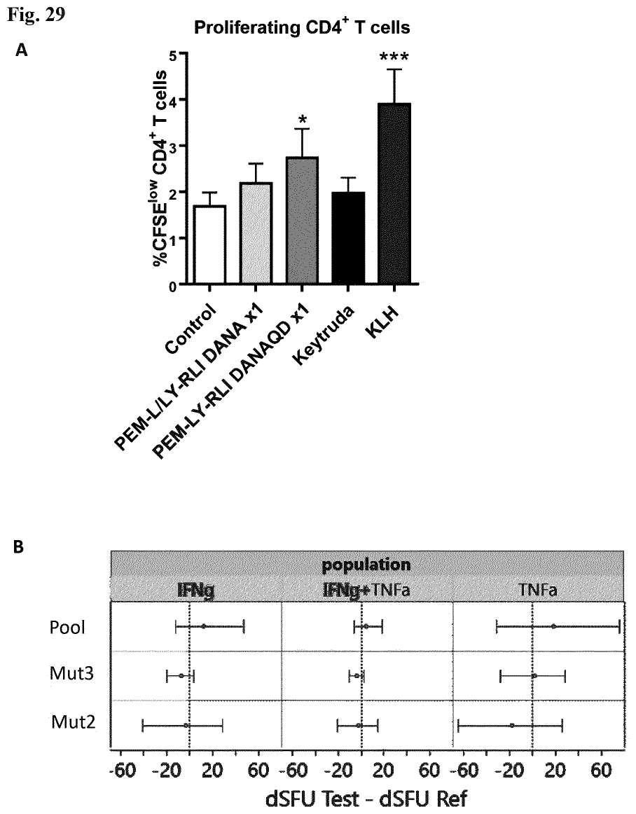

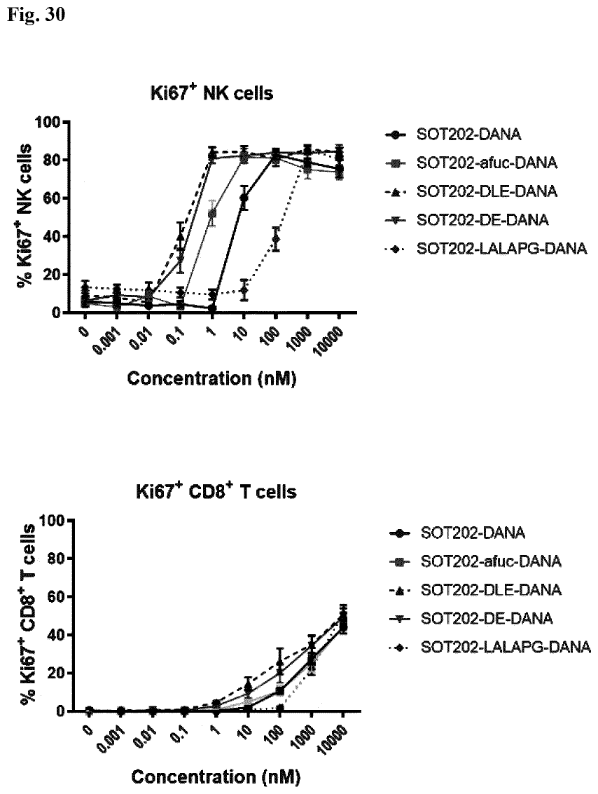

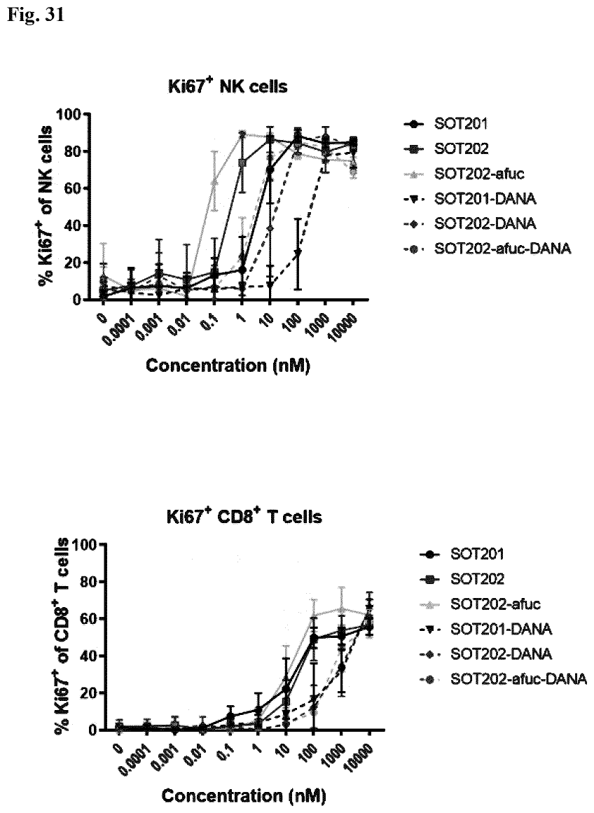

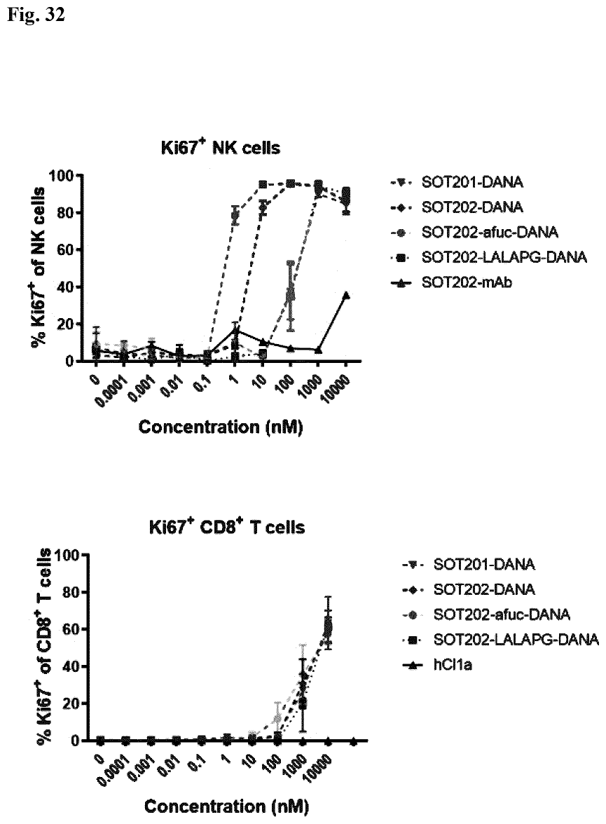

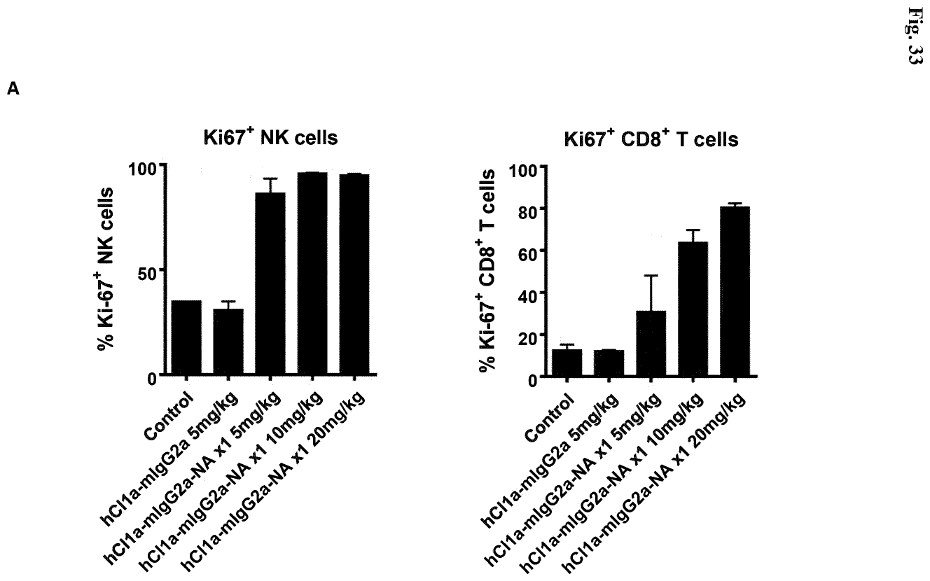

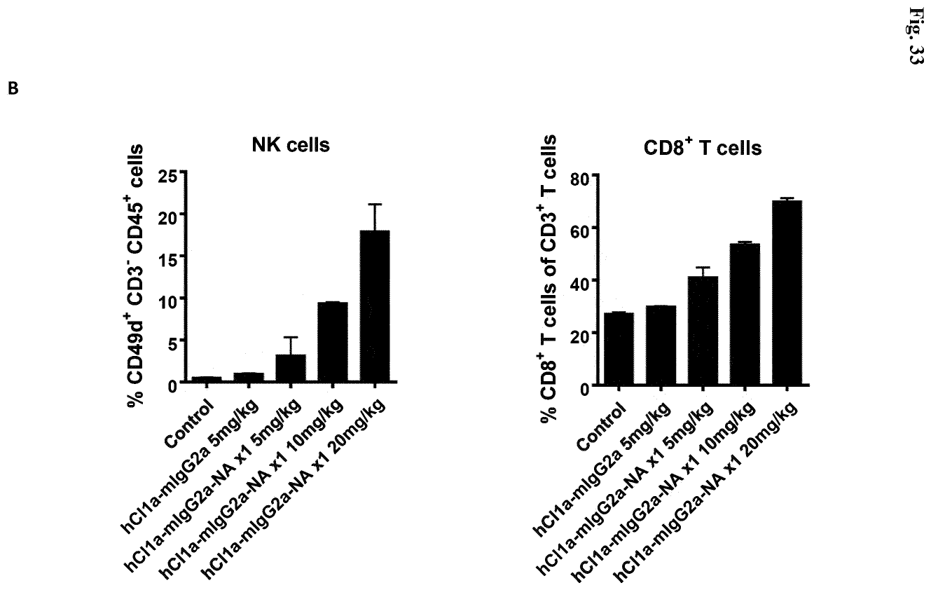

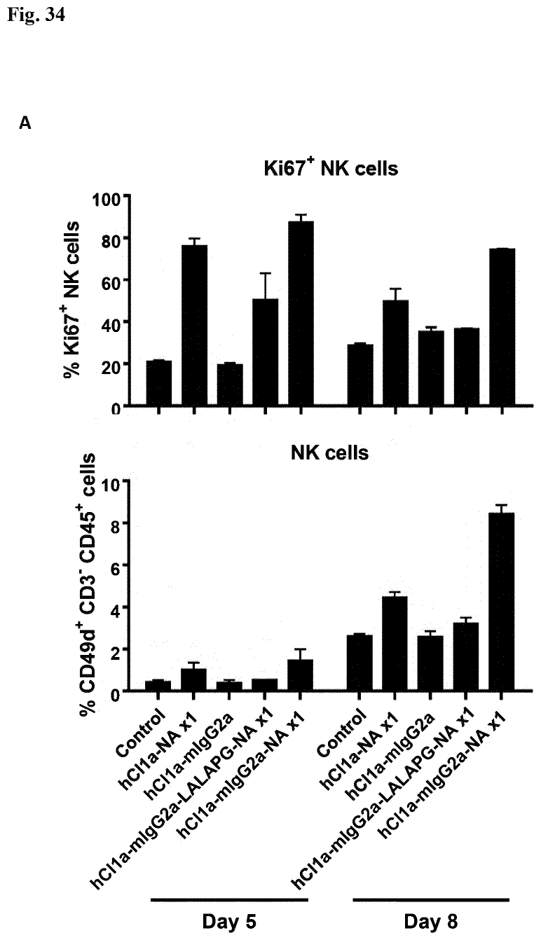

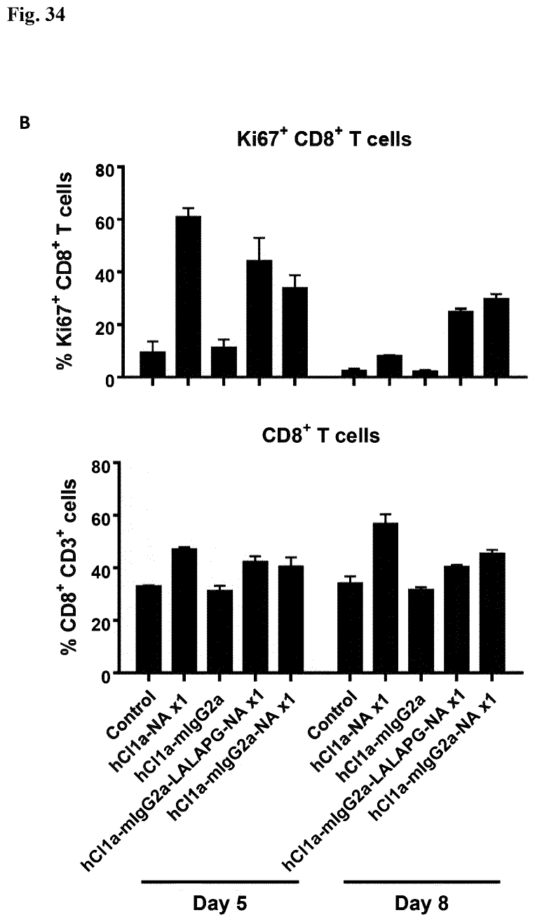

IFNγ production increased when mismatched human PBMC donor pairs were incubated with PEM LE/YTE-RLI NA x1 (1000 nM) in comparison to an equimolar amount of pembrolizumab and adjusted RLI2 concentration lowered 300× to equal the potency of RLI2 NA mutein. The data represent mean±SE of 6 donor pairs for pembrolizumab and PEM LE/YTE-RLI NA x1 and 3 donor pairs for RLI2 (see FIG. 8).

### Example 19: PEM LE/YTE-RLI NA x1 Molecule Shows Prolonged Half-Life In Vivo Over PEM-RLI Wt x1 Molecule which Correlates with a High PD Activity

PEM-RLI x1 and PEM LE/YTE-RLI NA x1 pharmacokinetics were tested in cynomolgus monkeys (n=2) after the administration of 10 or 30 μg/kg (PEM-RLI x1) and 30 or 90 μg/kg (PEM LE/YTE-RLI NA x1) on day 1 and day 15, respectively. Blood for serum separation was collected at 1 h, 4 h, 8 h, 12 h, 24 h, 48 h, 60 h, 72 h, 96 h, 120 h and 168 h. The concentration of PEM-RLI x1 and PEM LE/YTE-RLI NA x1 in serum was determined by ELISA. Blood for flow cytometry evaluation of selected immune cell populations (NK and CD8⁺ T cell proliferation—Ki67⁺, lymphocyte count) was collected at pre-dose, day 5, 8, 12, 15, 19, 22 and 26.

PEM LE/YTE-RLI NA x1 molecule with a decreased RLI-15 affinity to IL-2/15Rβγ displayed a significantly prolonged half-life over PEM-RLI x1 after IV administration in cynomolgus monkeys (FIG. 9A). PEM LE/YTE-RLI NA x1 molecule with an RLI mutein that has a markedly decreased affinity to the IL-2Rβγ and markedly reduced potency retained in the fusion with the PEM antibody high PD activity as shown by an increased number of lymphocytes (FIG. 9B) and by an increase in NK (FIG. 9C) and CD8⁺ T cell (FIG. 9D) proliferation determined by Ki67 positivity (FIG. 9.

### Example 20: PEM-RLI NA x2 Molecule Shows No Benefit Over PEM-RLI NA x1 Molecule (PK and PD Profile)

PEM-RLI NA x1 and PEM-RLI NAx2 pharmacokinetics was tested in the cynomolgus monkeys (n=2) after the administration of 30 μg/kg on day 1 to evaluate the benefit of two RLI2 molecules over one RLI2 molecule attached to the antibody. Blood for serum separation was collected at 1 h, 4 h, 8 h, 12 h, 24 h, 48 h, 72 h, 96 h and 168 h. The concentration of PEM-RLI NA x1 and PEM-RLI NA x2 in serum was determined by ELISA. Blood for flow cytometry evaluation of selected immune cell populations (NK and CD8⁺ T cell proliferation—Ki67⁺, lymphocyte count) was collected at pre-dose, day 5, 8, 12, 15, 19, 22 and 26.

There was no benefit of the PEM-RLI immunocytokine carrying two RLI2 molecules with a decreased affinity to IL-2/15Rβγ attached to the antibody over one molecules of RLI2 in terms of pharmacokinetics determined by serum concentration (FIG. 10A) or pharmacodynamics determined by lymphocyte count (fold change, FIG. 10B), % Ki67⁺ NK cells (FIG. 10C) and % Ki67⁺ CD8⁺ T cells (FIG. 10D) after IV administration in cynomolgus monkeys.

### Example 21: PEM-RLI NA x1 Molecule with LE Mutation Display Longer Half-Life than Molecule with LE/YTE Mutation

PEM LE/YTE-RLI NA x1 and PEM LE-RLI NAx1 pharmacokinetics were tested in the cynomolgus monkeys (n=3) after the administration of 600 μg/kg IV on day 1. Blood for serum separation was collected at 1 h, 8 h, 24 h, 48 h, 60 h, 72 h, 84 h, 96 h and 120 h. The concentration of PEM LE/YTE-RLI NA x1 and PEM LE-RLI NAx1 in serum was determined by ELISA. Whereas the YTE mutation has been reported to increase FcRn binding and thereby increase plasma half-life, in the immunocytokine format with one RLI NA mutein half-life was surprisingly reduced compared to the construct with the LE mutation only (FIG. 11).

### Example 22: Lower Potency Mutant PEM-RLI NQD x1 Display Longer-Half Life than PEM-RLI NAx1 Molecule

PEM LE-RLI NA x1 and PEM-RLI NQD x1 (NQD for D30N/E64Q/N65D) pharmacokinetics were tested in the cynomolgus monkeys (n=3) after the administration of 600 μg/kg IV on day 1. Blood for serum separation was collected at 1 h, 8 h, 24 h, 48 h, 60 h, 72 h, 84 h, 96 h, 120 h and 144 h. The concentration of PEM L-RLI NA x1 and PEM-RLI NQD x1 in serum was determined by ELISA. The PEM-RLI construct with the triple mutant NQD, that had shown a further reduced potency compared to the NA mutant (see Table 13) exhibited a further increase half-life in vivo compared to the PEM-RLI construct having the N65A substitution (FIG. 12).

### Example 23: ADCC Activity of Immunocytokines Based on Anti-Claudin18.2 hCl1a Antibody with Modified Effector Functions

### Cell Lines:

Human cell lines PA-TU-8988S (Creative Bioarray, catalog number CSC-C0326) and A549 (ATCC CCL-185) overexpressing Claudin 18.2 (A549-Cldn18.2) were grown in DMEM medium (Gibco) supplemented with 10% fetal bovine serum, 2 mM glutamine (GlutaMAX, Gibco), 100 U/ml penicillin, 0.1 mg/ml streptomycin (Invitrogen) and 2 ug/ml puromycin (Gibco).

A549 cells were co-transfected by electroporation with a transposase expression construct (pcDNA3.1-hy-mPB), a construct bearing transposable full-length huCLDN18.2 (pPB-Puro-huCLDN18.2) along with a puromycin resistance cassette and a construct carrying EGFP as transfection control (pEGFP-N3) (Waldmeier, Hellmann et al. 2016). Upon electroporation, cells were allowed to recover for two days in growth media at 37° C. in a humidified incubator in 5% CO2 atmosphere. Transfection was verified by FC analysis of the EGFP expression. Cells expressing CLDN18.2 were then selected by the addition of puromycin into culture at 1 μg/ml, and further expanded to allow the generation of frozen stocks in FCS with 10% DMSO. The expression of CLDN18.2 in the transfected cells was analyzed by FC.

In order to have a more homogenous PA-TU-8988S cell population, the cells were sorted by FACS to select only cells with a the higher CLDN18.2 expression. In brief, PA-TU-8988S cells suspended in FACS buffer (PBS, 2% FCS) were incubated on ice for 30 min with Zolbetuximab at 2 g/ml. After wash in FACS buffer, the cells were incubated with the PE-labelled Fcγ specific IgG goat anti-human secondary antibody (eBioscience) on ice for 30 min. After wash, the stained cells were resuspended in FACS buffer, analyzed and sorted by a FACSAria™ instrument, separating medium expressing cells from high expressing cells. After sorting the collected PA-TU-8988S-High cells (PaTu) were resuspended in growth media, expanded and frozen aliquots were preserved in liquid N2.

The human NK cell line NK92 (ATCC CRL-2407) exogenously expressing human CD16 (NK92-hCD16, here referred to as NK92) was generated as described in Clemenceau et al 2013 (Clemenceau, Vivien et al. 2013). The cells were grown in RPMI 1640 medium (Gibco) supplemented with 10% AB human serum (One Lambda), 2 mM glutamine (GlutaMAX, Gibco) and 5 ng/ml IL-2 (Peprotech). All cells were maintained at 37° C. in a humidified atmosphere containing 5% CO₂.

### Cell Based ADCC Assay:

A549-Cldn18.2 or PaTu cells were seeded into 96-well plates at an appropriate concentration (A549-Cldn18.2—20.000 cells, PaTu—30.000 cells) and incubated for 24 h. NK92 cells or isolated human NK cells were collected by centrifugation, washed and resuspended in ADCC assay medium (RPMI 1640 (no phenol red) supplemented with 2 mM glutamine and 10% heat-inactivated (56° C. for 20 min) pooled complement human serum (Innovative Research)). The medium from 96-well plates containing adhered cells (target cells T) was removed and NK92 cells in suspension in the ADCC assay medium (effector cells E) were added to the adherent target cells at an E:T ratio of 10 for A549-Cldn18.2 and of 5 for PA-TU-8988S cells. Antibodies or immunocytokines (ICK) to be tested were added in a concentration range of 0.001-100 nM or 0.0001-10 μg/ml. A human IgG1 isotype antibody (Ultra-LEAF™ Purified Human IgG1 Isotype Control Recombinant Antibody, Biolegend, cat. no. 403502) was included as an unspecific control. The mixture was incubated over-night at 37° C. After 24 h, cytotoxicity was measured, expressed as the activity of lactate dehydrogenase enzyme released from dead cells, using the LDH Cytotoxicity Assay (Abcam, ab65393) according to manufacturer's instructions: 10 μl of supernatant was transferred into a new 96-well plate, mixed with the LDH substrate and the developed colour change was measured using spectrophotometer at an OD of 450 nm. Cytotoxicity was calculated according to this formula: Cytotoxicity (%)=((Test Sample−effector cell control−low control)/(High Control−low control))×100; “test sample”: effector/target mix; “effector cell control”: one well with NK92 cells only (determines LDH activity released from effector cells); “low control”: one well with target cells only (determines a spontaneous release of LDH activity form untreated target cells); “high control”: one well with target cells permeabilized with lysis buffer (determines the maximal releasable LDH activity).

FIG. 13 show the ADCC activity of immunocytokines based on the hCl1a antibody with modified effector function. All the tested immunocytokines had heterodimeric Fc domains, with one RLI2_{AQ} conjugate fused to the C-terminus of one of the heavy chains. When immunocytokines with mutations of effector domain reducing ADCC were tested, the immunocytokine hCl1a LALAPG-RLI DANA showed nearly abolished ADCC activity when tested on A549-CLDN18.2 cells (upper panel) or PA-TU-8988S (lower panel) in the presence of NK92 cells, when compared to the hCl1a-DANA immunocytokine of hCl1a antibody alone. The hCl1a-LALA antibody showed also reduced ADCC activity when compared to the hCl1a antibody, however the ADCC activity was not fully abolished. The addition of the conjugate did not affect the ADCC activity of the immunocytokines when ADCC activity was reduced, when compared to the ADCC activity of the antibody alone. Table 25 recapitulates the ADCC EC50 values measured for each tested immunocytokine or antibody. The EC₅₀ values were determined using the Graphpad Prism Software with the built-in “log(AGONIST) vs. response −variable slope (four parameters)” EC50 determination.

When immunocytokines with mutations of effector domain enhancing ADCC were tested, all the tested immunocytokines based on the hCl1a antibody with DLE, DE, AAA, TE or IE mutations in the Fc domain showed enhanced ADCC activity, when compared to the same immunocytokine without those mutations or the antibody alone (FIG. 14).

Afucosylation was also tested to enhance ADCC activity. FIG. 14F shows that, in A549-Cldn18.2 and PA-TU-8988S cells, the afucosylated immunocytokine hCl1a-DANA afuc has enhanced ADCC activity when compared to hCl1a-DANA, and comparable ADCC activity to the immunocytokines with the DE and DLE mutations described above. However, when afucosylation was combined with mutations of effector domain enhancing, afucosylation surprisingly negatively affected the ADCC enhancement induced by the DE or DLE mutations (see FIGS. 14B and A). Nevertheless, enhanced ADCC activity was maintained when afucosylation was combined with the AAA mutations (FIG. 14.C)

TABLE 25
ADCC EC50 values for tested immunocytokines and antibodies

| | EC50 | |
|---|---|---|
| Antibody or ICK | A549-CLDN18.2 | PA-TU-8988S |
| Zolbetuximab | 0.106 | 0.628 |
| hCl1a | 0.02653 | 0.2196 |
| hCl1a LALA | 0.4899 | Not measurable |
| hCl1a LALAPG | Not measurable | 0.5635 |
| hCl1a-RLI DANA | 0.062 | 0.274 |
| hCl1a DLE-RLI DANA | 0.01514 | 0.05339 |
| hCl1a DE-RLI DANA | 0.0149 | 0.05198 |
| hCl1a AAA-RLI DANA | 0.02535 | 0.1116 |
| hCl1a TL-RLI DANA | 0.03036 | 0.1961 |
| hCl1a IE-RLI DANA | 0.03055 | 0.1157 |
| hCl1a-RLI DANA afuc | 0.01332 | 0.07711 |
| hCl1a DLE-RLI DANA afuc | 0.07504 | 0.1519 |
| hCl1a DE-RLI DANA afuc | 0.06663 | 0.1378 |
| hCl1a AAA-RLI DANA afuc | 0.02034 | 0.06905 |

### Example 24: Evaluation of Antibody Fc Binding to ADCC-Activating Receptors FcγRIIIa V158, and FcγRIIIa F158 and ADCC-Inhibitory Receptor FcγRIIb by Surface Plasmon Resonance (SPR)

The human FcγRIIIa receptor (hFcγRIIIa; CD16a) exists as two polymorphic variants at position 158, hFcγRIIIaV158 and hFcγRIIIaF158. FcγRIIIa activates ADCC activities, while FcγRIIb inhibits ADCC. The ADCC activity of the immunocytokines, when their affinity to the receptor is measured by SPR, can be expressed as the ratio of the EC50 binding affinity to FcγRIIIa to the EC50 binding affinity to FcγRIIb.

SPR experiments were performed on a Biacore 8K (Cytiva, Chicago, IL, USA), using CM5 sensor chips (Cytiva) with an immobilization using THE His tag antibodies (Genscript). FcγRIIIa V158, FcγRIIIa F158 or FcγRIIb protein were used for capture with a contact time of 30 sec at a flow rate of 10 l/min in a 1×HBS-EP+ running buffer. Association/dissociation rates were measured for each tested immunocytokine at a flow rate of 30 μl/min with concentration serial dilution in a suitable range with an association time/dissociation time of 300 s/300 s except for constructs with DLE and DE with and without afucosylation, where association/dissociation time of 120 s/1200 s was applied. Table 26 below summarizes the results of the SPR measurements.

TABLE 26
SPR data

| | Kd FcγRIIIa | Kd FcγRIIIa | | | |
|---|---|---|---|---|---|
| Antibody or | V158 - | F158 - | Kd | A/I | A/I |
| immunocytokine | high affinity | low affinity | FcγRIIb | (V158) | (F158) |
| Zolbetuximab | 8.64 × 10⁻⁸ | 4.23 × 10⁻⁷ | 1.39 × 10⁻⁵ | 160.9 | 32.9 |
| hCl1a | 1.1 × 10⁻⁷ | 4.48 × 10⁻⁷ | 9.22 × 10⁻⁶ | 83.8 | 20.6 |
| hCl1a-RLI DANA | 9.91 × 10⁻⁸ | 3.81 × 10⁻⁷ | 6.58 × 10⁻⁶ | 66.4 | 17.3 |
| hCl1a DLE-RLI DANA | 5.71 × 10⁻⁹ | 1.16 × 10⁻⁸ | 2.65 × 10⁻⁶ | 464.1 | 228.4 |
| hCl1a DE-RLI DANA | 7.06 × 10⁻⁹ | 1.87 × 10⁻⁷ | 1.78 × 10⁻⁶ | 525.1 | 95.2 |
| hCl1a AAA-RLI DANA | 3.56 × 10⁻⁸ | 1.01 × 10⁻⁷ | 2.46 × 10⁻⁵ | 691.0 | 243.6 |
| hCl1a TL-RLI DANA | 4.76 × 10⁻⁸ | 1.39 × 10⁻⁷ | 2.9 × 10⁻⁶ | 60.9 | 20.9 |
| hCl1a IE-RLI DANA | 2.44 × 10⁻⁸ | 6.73 × 10⁻⁸ | 3.68 × 10⁻⁶ | 150.8 | 54.7 |
| hCl1a-RLI DANA afuc | 2.74 × 10⁻⁸ | 8.81 × 10⁻⁸ | 4.29 × 10⁻⁶ | 156.6 | 48.7 |
| hCl1a DLE-RLI DANA afuc | 1.62 × 10⁻¹⁰ | 2.37 × 10⁻¹⁰ | 2.22 × 10⁻⁶ | 13703.7 | 9367.1 |
| hCl1a DE-RLI DANA afuc | 1.85 × 10⁻¹⁰ | 3.74 × 10⁻¹⁰ | 1.7 × 10⁻⁶ | 9189.2 | 4545.5 |
| hCl1a AAA-RLI DANA afuc | 1.73 × 10⁻⁸ | 4.61 × 10⁻⁸ | 1.75 × 10⁻⁵ | 1011.6 | 379.6 |

A/I ratio = (affinity towards FcgRIIIa)/(affinity towards FcgRIIb) were Affinity = 1/Kd.
“afuc” for afucosylated

The A/I ratio allows to evaluate the binding strength towards the ADCC-activating receptors (“A”; FcgRIII) compared to the binding strength towards the ADCC-inhibiting receptors (“B”; FcγRIIb). The higher the ratio, the stronger is the binding to the activating receptors of the antibody or immunocytokine.

The SPR data confirm that overall, all the immunocytokines with mutations enhancing ADCC show a higher A/I ration than the immunocytokine without mutations enhancing ADCC, a part of the TL mutations. The comparatively low A/I ratio for the TL mutations may be due to the increased glycosylation of such mutations (see example 25)

### Example 25: Stability/Developability of Immunocytokines Based on hCl1a with Enhanced ADCC Activity

Immunocytokines based on hCl1a having the DLE, DE, AAA, TL or IE mutation enhancing ADCC, or being afucosylated, where evaluated for their stability and developability, by evaluating the melting temperature of the C_{H}2 domain, sequence liabilities and glycosylation (N-Glycan) profiles.

Melting temperature of the C_{H}2 domain was measured by Differential scanning calorimetry (DSC) using a MicroCal PEAQ-DSC Automated system (Malvern Panalytical). In brief, the immunocytokine sample was diluted in its storage buffer to 1 mg/ml. The heating was performed from 20° C. to 100° C. at a rate of 1° C./min. Protein solution was then cooled in situ and an identical thermal scan was run to obtain the baseline for subtraction from the first scan.

For N-glycan analysis, the protein was firstly reduced with DTT, and then transfer to an HPLC column with glass-insert vial for injection. The protein was separated by reversed-phase chromatography and detected by Waters/XEVOG2XS-QTOF on-line LC-MS combined with UV detector. The molecular weight of detected glycan chains was matched with known N-glycan types, and the N-glycan relative abundance was calculated and represented by the intensity of the detected peaks.

Amino acid sequences of immunocytokine constructs bearing ADCC enhancement mutations were analysed for the presence of following additional sequence liabilities (not present in constructs without ADCC enhancement mutations) as described in Table 27.

TABLE 27
Known sequence liabilities.

| Sequence liabilities | Searched hotspots |
|---|---|
| N-glycosylation | NX[S/T] where X is any common |
| | amino acid except proline |
| Asparagine deamidation (Robinson | NG, NS, NN, NT, NH |
| and Robinson 2004, Lu, Nobrega et | |
| al. 2019) | |
| Aspartate isomerisation (Robinson | DG, DS, DD, DT, DH |
| and Robinson 2004, Lu, Nobrega et | |
| al. 2019) | |
| Unpaired cysteines | C |
| Methionine oxydation | M |

The TL mutation introduced a N-glycosylation sequence liability (mutation K392T in close proximity to N390 in the IgG1 sequence). No sequence liability was introduced by the other mutations (see Table 28).

TABLE 28
Stability and developability summary.

| Modification | Sequence liabilities | N-glycans | Melting temperature |
|---|---|---|---|
| WT | 4 | 4 | 4 |
| afucosylation | 4 | 4 | 4 |
| DLE | 4 | 4 | 2 |
| DE | 4 | 4 | 2 |
| AAA | 4 | 3 | 4 |
| TL | 1 | 1 | 4 |
| IE | 4 | 2 | 4 |

Score 4: Parameter is in the range expected for a mAb-based drug product;
Score 3: Careful monitoring/evaluation of quality attribute required during development;
Score 2: Considerable impact on timeline and/or cost is likely;
Score 1: High risk which cannot be controlled adequately.

Overall, afucosylation had no impact on stability and developability, and thus may be used to enhance ADCC activity of the immunocytokine. DLE and DE mutations caused a considerable decrease in Tm1 (melting temperature of the C_{H}2 domain) (see Table 29), potentially impacting the stability in solution of the immunocytokine. However, these mutations did not impact the glycosylation of the immunocytokines. The sequence liabilities introduced by the TL mutations resulted in the introduction of undesired sialylated and high mannose glycan species (see Table 30). These species may negatively impact the pK of the immunocytokines. Likewise, immunocytokines with the IE mutations had a high proportion of mannose species, potentially impacting their properties. Immunocytokines with the AAA mutations resulted in the increase of mannose species (see Table 30). However, production of afucosylated immunocytokine partially reverted the glycosylation to acceptable levels with regards to developability. Therefore, when enhancement of the immunocytokine based on hCl1a is desired, the AAA mutations, optionally combined with afucosylation, may be the recommended mutations affecting the least its stability and developability. Afucosylation had no impact on evaluated properties. DLE and DE mutations caused a considerable decrease in Tm, potentially destabilising the molecule. TL mutation introduced an additional glycosylation site into Fc. Construct with IE mutation had a high proportion of mannose species.

TABLE 29
Melting temperatures (“afuc” for afucosylated).

| Immunocytokine or antibody | Tm1 (° C.) |
|---|---|
| hCl1a | 69.5 |
| hCl1a-RLI DANA | 71.8 |
| hCl1a-RLI DANA afuc | 72.6 |
| hCl1a DLE-RLI DANA | 51.1 |
| hCl1a DLE-RLI DANA afuc | 51.3 |
| hCl1a DE-RLI DANA | 51.3 |
| hCl1a DE-RLI DANA afuc | 52.4 |
| hCl1a AAA-RLI DANA | 63.0 |
| hCl1a AAA-RLI DANA afuc | 64.9 |
| hCl1a TL-RLI DANA | 68.0 |
| hCl1a IE-RLI DANA | 58.3 |

TABLE 30
N-glycan analysis

| Immunocytokine or | Man5 species | | Sialic acid species | |
|---|---|---|---|---|
| antibody | CHOK1 | FUT8KO | CHOK1 | FUT8KO |
| hCl1a | 7.8% | Not tested | 0 | Not tested |
| hCl1a-RLI DANA | 10.1% | 5.4% | 0 | 0 |
| hCl1a DLE-RLI DANA | 1.6% | 0 | 0 | 0 |
| hCl1a DE-RLI DANA | 2.4% | 0 | 0 | 0 |
| hCl1a AAA-RLI DANA | 19.2% | 7.5% | 0 | 0 |
| hCl1a TL-RLI DANA | 16.4% | Not tested | 23.0% | Not tested |
| hCl1a IE-RLI DANA | 21.6% + 4% | Not tested | 0 | Not tested |
| | Man6 | | | |

### Example 26: Mouse In-Vivo Efficacy Studies

The purpose of this study is to test the in vivo therapeutic efficacy of hCl1a-RLI immunocytokines in a mouse model. Female NMRI nude mice are implanted at 5-7 weeks of age with pancreatic human cell line derived xenograft BXPC3 (ATCC CRL-1687™) exogenously expressing Claudin18.2 (BXPC3-CLDN18.2). Tumours are implanted by unilateral subcutaneous injection. The animals ae randomized based on the tumour volume around 100 mm³. Mice are allocated to different groups (n=7 per group) and treated according to the Table 31 at day 1. The animals are checked twice weekly for weight loss and tumour volume. Tumour volume is measured by caliper and is expressed in mm³ using the formula: “V=(L×W×W)/2, where V is tumour volume, L is tumour length (the longest tumour dimension) and W is tumour width (the longest tumour dimension perpendicular to L). Mice are euthanized reaching a tumour burden of 2000 mm³ or experiencing significant body weight loss (overall more than 30%, or more than 20% in two consecutive days).

TABLE 31
mouse treatment regimen (“afuc” for afucosylated)

| | | Total | | | mice/ |
|---|---|---|---|---|---|
| Group | Test item | daily dose | Schedule | Route | group |
| 1 | Vehicle (0.9% NaCl) | 5 mg/kg | D1 | i.v. | 7 |
| 2 | hCl1a-RLI NAx1 | 5 mg/kg | D1 | i.v. | 7 |
| 3 | hCl1a-RLI NAx1 | 5 mg/kg | D1 | i.v. | 7 |
| | afuc | | | | |
| 4 | hCl1a | 5 mg/kg | D1 | i.v. | 7 |
| 5 | Zolbetuximab | 5 mg/kg | D1 | i.v. | 7 |

### Example 27: Anti-PD-1 Antibody and SOT201 Synergize in Activation of CD8⁺ T Cells

SOT201 is a heterodimeric immunocytokine with an antibody derived from the humanized IgG 4 pembrolizumab with T366W—knob/T366S, L368A, Y407V—hole substitutions, L235E substitution, and deleted terminal K of the heavy chains, fused to RLI-15_{AQA} at the C-terminus of the knob heavy chain, see SEQ ID NO: 21, SEQ ID NO: 101, SEQ ID NO: 23). SOT201 and Keytruda© (pembrolizumab) were compared in the PD-1/PD-L1 blockade assay according to Example 1. FIG. 19A shows that SOT201 effectively blocks PD-1/PD-L1 interactions similarly to the anti-PD-1 antibody Keytruda. Determined K_{D} values for SOT201 and pembrolizumab are shown in Table 32.

TABLE 32
K_{D} values for SOT201 and pembrolizumab at 4° C. and 37° C.

| | |
|---|---|
| 4° C. binding | 37° C. binding |
| K_{D} [nM] | K_{D} [nM] |

| | 1 h | 4 h | 1 h | 4 h |
|---|---|---|---|---|
| SOT201 | 0.28 | 0.26 | 0.23 | 0.21 |
| Keytruda ® | 0.23 | 0.23 | 0.22 | 0.21 |
| (pembrolizumab) | | | | |

Human PBMC from 11 healthy donors were stimulated for 7 days in vitro with SOT201 having the RLI2AQ N65A (RLI-15_{AQA}) variant or with a control molecule having identical antibody heavy and light chains as SOT201 but with the RLI2_{AQ} variant without a reduced binding of the IL-15 moiety to the IL-2/IL-15Rβγ (“SOT201 wt”). Cell proliferation was determined by measuring Ki-67⁺ NK cells and CD8⁺ T cells by flow cytometry analysis. SOT201 activates proliferation of NK and CD8⁺ T cells at higher EC50 concentration in comparison to the comparable immunocytokine molecule with an RLI-15 molecule without reduced receptor binding (SOT201 wt) (FIG. 19B).

A murine surrogate SOT201 (mSOT201, see SEQ ID NO: 102, SEQ ID NO: 103 and SEQ ID NO: 104) comprising the anti-murine PD-1 antibody RMP1-14 (BioXCell, Lebanon, NH, USA) with analogous substitutions for heterodimerization (E356K, N399K/K409E, K439D), ADCC silencing (D265A) and stabilization (dK) fused to RLI-15_{AQA} was compared to single activity controls represented by the monoclonal anti-murine PD-1 antibody RMP1-14 as such (mPD1) and the anti-human PD1 mouse IgG1-RLI-15_{AQA} (hPD1-mSOT201), which does not exert any PD-1 blocking activity in the C57BL/6 mouse, as an RLI-15_{AQA} control with a similar in vivo half-life as mSOT201. Cell proliferation (Ki67) was detected in spleen by flow cytometry 5 days after IV injection of compounds at equimolar amount to 5 mg/kg of mSOT201 in healthy C57BL/6 mice (n=2/group). The anti-PD-1 antibody and the RLI-15_{AQA} mutein moieties in the murine surrogate mSOT201 showed a synergistic effect on CD8⁺ T cell proliferation (FIG. 19C).

### Example 28: Tumor Regression in MC38 Mouse Model

C57BL/6 mice (hPD1-transgenic) were implanted with syngeneic MC38 cell line. Test agents mSOT201, hPD1-mSOT201 and mPD1 were injected IV on day 1 (randomization day, tumor volumes 80-100 mm³) (n=10/group) at equimolar amounts to 5 mg/kg mSOT201 and compared to control (NaCl). mSOT201 induced tumor regression in 9 out of 10 mice after a single IV administration, whereas in comparison the monoclonal anti-mouse PD-1 antibody (mPD1) and the anti-human PD-1 mouse IgG1-RLI-15 mutein immunocytokine (hPD1-mSOT201) exerting no anti-PD-1 effect in mice only showed minor effects on tumor growth compared to the control mice (FIG. 20A). Similarly, the 15 synergistic activity of the anti-murinePD-1 antibody and the RLI-15_{AQA} mutein in the fusion protein (mSOT201) compared to the anti-mousePD-1 antibody alone (mPD1) or the anti-humanPD1 mouse IgG1-RLI-15 mutein immunocytokine (hPD1-mSOT201) as a control for the RLI-15_{AQA} mutein alone is shown in the surviving mice in the time course up to 100 days post treatment (FIG. 20B).

### Example 29: Induction of Pathways and Genes Connected to Anti-Tumor Immunity in MC38 Tumors and Activation of Immune Cells in Spleen and Lymph Nodes

RNA isolation: RNA samples were isolated from tumors of syngeneic MC38 tumor bearing C57BL/6 mice 7 days after a single IV administration of mSOT201 (5 mg/kg). 3 mice were treated with mSOT201 (5 mg/kg) IV on day 1 (randomization day, tumor volumes 80-100 mm3), 4 control mice were left untreated. RNA was isolated from tumour tissue by using RNeasy MicroKit. The quality of RNA samples was checked using the Agilent Bioanalyzer RNA Nano Chip and the Qubit HS RNA assay.

RNA seg analysis: The sequencing libraries were prepared from RNA samples by the SMARTer® Stranded Total RNA-Seq Kit v3—Pico Input Mammalian Kit (Takara Bio USA, Inc.), library quality control was performed employing the capillary gel electrophoresis system (Agilent Bioanalyzer with the HS DNA chip) and the Qubit HS DNA Assay, and sequencing was done on NovaSeq 6000 using the NovaSeq 6000 300 cycles Reagent Kit in 2×151 bp run.

Data analysis: Raw data were processed according to the standard RNA-seq pipeline including the following steps: quality control (via FastQC and FastqScreen), adapter trimming (trimmed 8 bp in Read2 by using seqtk), mapping to the reference genome GRCm39 (using HISAT2) and transcript counting (with ht-seq). The obtained output, quantification files containing the number of transcripts for each sample, were further processed via R packages and ggplot2, tydiverse, dplyr. Raw counts were normalized via DESeq2 median of ration normalization. Differential gene expression analysis was performed using DESeq2 (version 1.24.0) in R (abs(log 2FC)=1, FDR<0.05). Heatmaps were created using ComplexHeatmap package in R. Functional and enrichment analysis of DEGs was performed using the ClusterProfiler and the web-based tool Gene ontology (GO). To calculate TPM values for cell population analysis, salmon tool was used on trimmed fastq files. Analysis of cell population was performed by TIMER 2.0 and xCell tools.

Results: Differential expression analysis (abs(log 2FC)=1, FDR<0.05) resulted in upregulation of 800 mouse genes and downregulation of 1910 mouse genes in mSOT201-treated tumors compared to control samples. Enrichment analysis of gene Ontology (GO) terms mainly identified upregulated DEGs linked to activation of as T cells, γδ T cells, B cells, NK cells, cytotoxicity, cell killing, cytokine production, cell chemotaxis and cell adhesion while downregulated genes were linked to tumor development and tumor signaling. These data indicate that mSOT201 activates both innate as well as adaptive immunity in the tumor microenvironment. Next, we employed “metagene” markers to estimate the relative abundance of different immune cell populations in the tumor microenvironment. In line with the whole-transcriptome findings, mSOT201-treated samples were enriched for gene sets associated with CD8⁺ T cells (p<0.001), CD8⁺ naïve T cells (p<0.0005), CD8⁺ effector memory T cells (p=0.001), CD8⁺ T cell central memory (p<0.001), γδ T cells (p=0.0002), NK cells (p<0.001), CD4⁺ T cells (p=0.0157), CD4⁺ naïve T cells (p=0.1176), CD4⁺ effector memory T cells (p=0.003), B cells (p=0.0602), myeloid dendritic cells (p=0.0120). On the other hand, the gene sets associated with cancer-associated fibroblast was markedly reduced (p=0.0254) (FIG. 21A).

mSOT201 induced proliferation of selected immune cell populations in spleen and lymph nodes in MC38 tumor bearing mice (FIG. 21B). Cell proliferation (Ki67) was detected by flow cytometry on day 7 after the mSOT201 treatment of the established tumors (80-100 mm³) (n=2).

### Example 30: EC50 Values of Different IL2/IL-15Rβγ Agonists on Kit225 Cells

EC50 values of RLI-15 (SOT101), SOT201 (PEM-RLI-15_{AQA}), hPD-1-IL-2v and αhPD1-IL-15m M1 were determined as described in Example 1. In hPD-1-IL-2v one IL-2 mutein IL-2v (SEQ ID NO: 106) is fused to the C-terminus of one heavy chain of an anti-humanPD-1 antibody as described in WO 2018/184964a1 (with sequences of Seq id no.: 22, 23 and 25 therein). In αhPD1-IL-15m M1 one IL-15 mutein with the mutations N1A-D30N-E46G-V49R (SEQ ID NO: 107) is fused to the C-terminus of one heavy chain of an anti-humanPD-1 antibody as described in WO 2019/166946a1 (see FIG. 1D therein, SEQ ID NO: 89, 74 and 65 therein). EC50 values are show in Table 33.

TABLE 33
EC50 of selected IL-2/IL-15Rβγ agonists on kit225 cells

| | EC50 [pM] |
|---|---|
| SOT101 (RLI-15) | 35 |
| SOT201 (PEM-RLI-15_{AQA}) | 15160 |
| PD1-IL-2v | 3018 |
| αhPD1-IL-15m M1 | 3307 |

A further interesting candidate to be tested is the αhPD1-IL-15m M2 with on IL-15 mutein with mutations N1G-D30N-E46G-V49R-E64Q (SEQ ID NO: 108) is fused to the C-terminus of one heavy chain of an anti-humanPD-1 antibody as described in WO 2019/166946a1 (see FIG. 1C therein, Seq id no: 90, 74 and 65 therein).

Accordingly, SOT201 has a substantially lower EC50 on kit225 cells than PD1-IL-2v and αhPD1-IL-15m M1, expected to allow for higher dosing and longer half-life in vivo to exert also a stronger and longer lasting effect with respect to the activity disrupting the anti-PD-1/PD-L1 interaction.

### Example 31: Comparison of mSOT201 with mPD1-IL-2Rβγ Agonist in the MC38 Tumor Model

mSOT201 (mouse SOT201 surrogate) was compared to control (NaCl), the anti-murinePD-1 antibody RMP1-14 fused to the IL-2v IL-2 mutein (mPD1-IL-2Rβγ agonist) and the combination of the RLI-15_{AQA} and the mPD1 antibody in the MC38 tumor model in a single IV administration as described in Example 28. The dosing of mPD1-IL-2Rβγ was selected to match the NK and CD8⁺ T cell proliferation on day 5 of 5 mg/kg of mSOT201 after IV administration in healthy C57/BL6 mice, resulting in an equivalent dose of 0.25 mg/kg mPD1-IL-2Rβγ. Cell proliferation (Ki67⁺) was detected by flow cytometry. mSOT201 induced activation of CD8⁺ T cells and NK cells which persisted up to day 8 in contrast to the mPD1-IL-2Rβγ agonist (FIG. 22B).

mPD1-IL-2Rβγ is an IL-2/IL-15Rβγ agonist where the IL-2 mutein IL-2v (SEQ ID NO: 106) comprises the substitutions F42A, Y45A and L72G relative to the IL-2 sequence reducing the affinity to the IL-2Rα (see WO 2018/184964A1, e.g., bridging para. of pages 27 and 28) and the further substitutions T3A to eliminate O-glycosylation at position 3 (bridging para. of pages 28 and 29) and C125A to increase expression or stability (page 30, 3^{rd} para.).

The murine surrogate of SOT201 (mSOT201) induced tumor regression in 9 out of 10 MC38 tumor-bearing mice after a single IV administration comparing to 5 out of 10 for the mPD1-IL-2Rβγ agonist, whereas the combination of the RLI-1^{5A}QA with the mPD1 antibody only led to a delay of tumor growth compared to the control mice (FIG. 22A). mSOT201 induced proliferation of NK and CD8⁺ T cells in MC38 tumor bearing mice which persisted 7 days after dosing in contrast to the mPD1-IL-2Rβγ agonist and the equimolar amount of RLI-15_{AQA} in combination with mPD1. The treatment of MC38 tumors was at randomization day 1, tumor volumes 100 mm³ (n=10/group).

Further, mSOT201_induced a strikingly longer activation of CD8⁺ T cells and NK cells still persisting at day 8 in contrast to mPD1-IL-2Rβγ agonist, which showed marked reductions of proliferating cells at day 8 (FIG. 22B).

SOT201 also induced proliferation of NK and CD8⁺ T cells in spleen and lymph nodes of MC38 tumor bearing mice which persisted 7 days after dosing in contrast to mPD1-IL-2v and the equimolar amount of the combination of RLI-15_{AQA} and the mPD1 antibody (FIG. 22C).

### Example 32: PK Profile of SOT201 in Cynomolgus Monkeys

SOT201 was administered IV at 0.6 mg/kg on day 1 to cynomolgus monkeys and proliferation (Ki67+) and absolute cell numbers of NK and CD8⁺ T cells were determined over time by flow cytometry and haematology. SOT201 induced high proliferation and expansion of NK (˜90% at day 5) and CD8⁺ T cells (about 80% at day 5) in blood of cynomolgus monkeys after an IV administration (FIG. 23A). Pharmacokinetic parameters are shown in Table 34.

TABLE 34
Pharmacokinetic parameters of SOT201 in cynomolgus monkeys

| | Dose | No. and sex | AUCₗₐₛₜ | Cₘₐₓ | Tₘₐₓ | T_{1/2} |
|---|---|---|---|---|---|---|
| Day | [mg/kg] | of animals | [ng · h/ml] | [ng/ml] | [h] | [h] |
| 1 | 0.6 | 3F | 456,751.96 | 14,633.52 | 1 | 17.63 |

SOT201 induced activation of NK and CD8⁺ T cells after a repetitive IV administration in cynomolgus monkeys (FIG. 23B)

### Example 33: PD Activity of Mouse SOT201 Surrogates

The first aim of the study was to evaluate whether the treatment with mouse surrogate molecule mSOT201 (see Example 27) has an additive/synergistic effect on the CD8⁺ T cell proliferation, when compared to the treatment with hPD1-mSOT201 or mPD-1 in C57BL/6 mice. The second aim of the study was to compare the pharmacodynamic activity of mSOT201 wt mouse surrogate molecule with a mouse surrogate molecule mPD1-IL2v in C57BL/6 mice. The description of tested mouse surrogate molecules is described in Table 35. PD activity was evaluated on day 5 and day 8. FACS analysis was performed as described above.

TABLE 35
Description of the mouse surrogate molecules

| Molecule | Molecule characteristics |
|---|---|
| mSOT201 | Mouse anti-PD-1 antibody (clone RMP1-14) mouse |
| | IgG1-D265A |
| | RLI-15_{AQA} genetically fused to anti-PD-1 antibody |
| hPD1-mSOT201 | Human anti-PD-1 antibody (pembrolizumab) mouse |
| | IgG1-D265A |
| | RLI-15_{AQA} genetically fused to anti-PD-1 antibody |
| mPD-1 | Mouse anti-PD-1 antibody (clone RMP1-14) mouse |
| | IgG1-D265A |
| mSOT201 wt | Mouse anti-PD-1 antibody (clone RMP1-14) mouse |
| | IgG1-D265A |
| | RLI2 genetically fused to anti-PD-1 antibody |
| mPD1-IL2v | Mouse anti-PD-1 antibody (clone RMP1-14) mouse |
| | IgG1-D265A |
| | IL2v: IL-2 mutein with T3A F42A Y45A L72G |
| | C125A substitutions genetically fused to anti-PD-1 |
| | antibody (see SEQ ID NO: 112, SEQ ID NO: 113, |
| | SEQ ID NO: 114) |

TABLE 36
Potency of mouse surrogates in comparison to human molecules
in kit225 assay

| | EC50 | Relative potency to |
|---|---|---|
| | kit225 cells [pM] | SOT101 |
| SOT101 | 35.9 | — |
| SOT201 | 9998 | 0.36% |
| hPD1-mSOT201 | 19983 | 0.18% |
| mSOT201 | 26786 | 0.13% |
| SOT201 wt (with RLI2_{AQ}) | 248.8 | 14.43% |
| mSOT201 wt (with RLI2_{AQ}) | 558.4 | 6.42% |
| IL-2 | 672.9 | 5.33% |
| mPD1-IL2v | 3082 | 1.16% |
| PD1-IL2v | 3018 | 1.19% |

As pembrolizumab does not recognize the murine PD-1, the hPD-1-mSOT201 represents a control for an RLI-15_{AQA} bound to a non-binding antibody with a similar PK profile and therefore reflects the PD activity of the RLI-15_{AQA} Molecule with such PK profile. The mPD-1 molecule reflects the PD activity of the anti-PD-1 antibody alone. With respect to the activation of CD8⁺ T cells, mSOT201 shows a more than additive effect (i.e. synergistic) compared to its single component surrogates hPD1-mSOT201 and mPD-1 at Day 5 and even more at Day 8 dosed at equimolar amounts. In comparison, both mPD1-IL2v and mSOT201 wt (both having a more active IL-2/IL-15Rβγ agonist), dosed lower given their expected high activity at Day 5, show a bit higher activation of CD8⁺ T cells on Day 5, but such effect is only short lasting, as at Day 8 activation of CD8⁺ T cells is much stronger for mSOT201. Looking at activated NK cells, differences are not so pronounced. As expected, mPD-1 does not activate NK cells, whereas hPD1-mSOT201, mPD1-IL2v, mSOT201 and mSOT201 wt strongly activate at Day 5, with mSOT201 somewhat weaker than the others. At Day 8, again mSOT201 exhibits a stronger activation of NK cells compared to mPD1-IL2v and mSOT201 wt. (FIG. 24 A)

A similar picture was observed, when to mSOT201, hPD1-mSOT201 and mPD-1 were dosed at double the amounts of A, whereas mSOT201 wt and mPD1-IL2v were dosed at lower amounts (see FIG. 24 B), as they likely already had reached maximal activation of cells in experiment A. As expected, mSOT201 wt and mPD1-IL2v showed a reduced activation of both CD8⁺ T cells and NK cells, which again was down at control level at Day 8 for CD8⁺ T cells.

These data show, that SOT201 having a marked reduced binding to IL-2/IL-15Rβγ together with its 10 anti-PD-1 moiety is both a strong and long-lasting activator of NK and CD8⁺ T cells, whereas molecules with higher IL-2/IL-15Rβγ agonistic activity show a much shorter activation especially of CD8⁺ T cells. It is hypothesized that the avidity effect of simultaneously binding PD-1 and the IL-2/IL-15Rβγ of PD-1 expressing CD8⁺ T cells in cis (i.e., on the same CD8⁺ T cell) or in trans (i.e., between different CD8⁺ T cells in close proximity) leads to such preferential activation of CD8⁺ T cells.

### Example 34: Anti-Tumor Efficacy Activity of mSOT201 in PD-1 Sensitive and PD-1 Treatment Resistant Mouse Models

The aim of the study was to evaluate the anti-tumor activity of mSOT201 in anti-PD-1 treatment sensitive (CT26, MC38) and in anti-PD-1 treatment resistant (B16F10, CT26 STK11 ko) mouse models. The description of tested mouse surrogate molecules is described in Table 37.

TABLE 37
Description of the mouse surrogate molecules

| Molecule | Molecule characteristics |
|---|---|
| mSOT201 | Mouse anti-PD-1 antibody (clone RMP1-14) mouse |
| | IgG1-D265A |
| | RLI-15_{AQA} genetically fused to anti-PD-1 antibody |
| hPD1-mSOT201 | Human anti-PD-1 antibody (pembrolizumab) mouse |
| | IgG1-D265A |
| | RLI-15_{AQA} genetically fused to anti-PD-1 antibody |
| mPD-1 | Mouse anti-PD-1 antibody (clone RMP1-14) mouse |
| | IgG1-D265A |

The murine surrogate molecule of SOT201—mSOT201—as compared to its single component surrogates mPD-1 and hPD1-mSOT201 shows a synergistic effect in the tested PD-1 sensitive tumor models CT26 and MC38 with 5 out of 10 and 9 out of 10 complete responses. (FIG. 25 A)

Even in tumor models known to be resistant to anti-PD-1 therapy, mSOT201 showed a synergistic effect compared to its single components, although the therapeutic effect was not as strong as for the sensitive models showing only 1 complete response out of 10 mice for the B16F10 model.

### Example 35: Anti-Tumor Efficacy Activity of mSOT201 vs RLI-15_{AQA} Mutein+Anti-PD-1 Antibody

The aim of the study was to evaluate the anti-tumor activity of mSOT201 vs. RLI-15 AQA mutein+anti-PD-1 treatment in MC38 mouse models. The description of tested mouse surrogate molecules is described in Table 38.

TABLE 38
Description of the mouse surrogate molecules

| Molecule | Molecule characteristics |
|---|---|
| mSOT201 | Mouse anti-PD-1 antibody (clone RMP1-14) mouse |
| | IgG1-D265A |
| | RLI-15_{AQA} genetically fused to anti-PD-1 antibody |
| hPD1-mSOT201 | Human anti-PD-1 antibody (pembrolizumab) mouse |
| | IgG1-D265A |
| | RLI-15_{AQA} genetically fused to anti-PD-1 antibody |
| RLI-15_{AQA} | RLI-15 mutein |
| mPD-1 | Mouse anti-PD-1 antibody (clone RMP1-14) mouse |
| | IgG1-D265A |

The fusion of the anti-PD-1 moiety with the IL-2/IL-15βγ agonist RLI-15_{AQA} (at two doses, G2 and G3) showed a strong synergistic effect compared to the combination of the individual equimolar components (G4: RLI-15_{AQA}+mPD1, or G11: hPD1-mSOT201+mPD1), see FIG. 26. It is hypothesized that the temporal and spatial linkage of activation of PD-1 positive immune cells is mechanistically stronger than the activation of immune cells by the individual components.

### Example 36: Anti-Tumor Efficacy Activity of mSOT201 vs SOT101+Anti-PD-1 Antibody

The aim of the study was to evaluate the anti-tumor activity of mSOT201 vs SOT101+anti-PD-1 treatment in the MC38 mouse model. The description of tested mouse surrogate molecules is described in the Table 39.

TABLE 39
Description of the mouse surrogate molecules

| Molecule | Molecule characteristics |
|---|---|
| mSOT201 | Mouse anti-PD-1 antibody (clone RMP1-14) mouse IgG1- |
| | D265A |
| | RLI-15_{AQA} genetically fused to anti-PD-1 antibody |
| RLI2_{AQ} | RLI-15 having G78A/N79Q stabilizing mutations but no |
| | reduced IL-2/IL-15Rβγ binding |
| mPD-1 | Mouse anti-PD-1 antibody (clone RMP1-14) mouse IgG1- |
| | D265A |

A single dose of mSOT201 of 2 mg/kg (G3) showed about the same therapeutic effect as combined therapies with 4 administrations of 1 mg/kg RLI2_{AQ}+a single dose of 5 mg/kg mPD1 (G8) or with 4 administrations of 1 mg/kg RLI2_{AQ}+a four doses of 5 mg/kg mPD1 (G9). However, a single dose of mSOT201 of 5 mg/kg (G2) outperforms the multiple administrations of the individual components (G8 and G9).

### Example 37: Mechanistical Studies on Differences in Immune Cell Activation Under of mSOT201 Vs SOT101+Anti-PD-1 Antibody Treatment

The aim of the study was to evaluate the anti-tumor activity of a similar efficacious dose of mSOT201 vs SOT101+anti-PD-1 treatment in the MC38 mouse model. The description of tested mouse surrogate molecules is described in the Table 39.

Differences in the relative number of various immune cell populations upon both treatments were detected in tumor, spleen and lymph nodes. The relative expansion of CD8⁺ T cells and αβTCR bearing CD3⁺ cells did not change between both treatments in spleen and lymph nodes. However, in tumor mSOT201 induced a higher relative increase in CD8⁺ T cells whereas the combined RLI2_{AQ}+anti-PD-1 treatment increased more NK cells. Interestingly, mSOT201 induced a higher percentage of γδTCR bearing CD3⁺ cells in spleen and lymph nodes, while the combined RLI2_{AQ}+anti-PD-1 treatment induced a higher percentage of γδTCR bearing CD3⁺ cells mainly in tumors. (see FIG. 28).

### Example 38: DC-T Cell-Based Assay and Fluorospot Assay for Determining Immunogenicity

The aim of the study was to assess the immunogenicity risk of pembrolizumab-based immunocytokines bearing one RLI-15 mutein (PEM-RLI-15 candidate molecules) in vitro. The DC-T cell assay method was used for this purpose, where the test products were first incubated with immature dendritic cells (iDCs) leading to later presentation to autologous T cells as processed peptides of the candidate molecules loaded on the MHC molecules of the matured DCs (mDCs). After a 7-day co-incubation period, T cell proliferation was measured as a surrogate marker for anti-drug antibody formation. The detection of T cell proliferation induced by DCs was used to mitigate the stimulatory activity of the RLI-15 component in the test system that can have a strong influence on the result, which shall not be attributed to immunogenicity. Keyhole limpet hemocyanin (KLH) was used as a positive control, as KLH is known to induce a strong immune response induction. Pembrolizumab was used as a negative control. Control DCs loaded with no protein were used as control for assessment of unspecific T cell proliferation.

TABLE 40
PEM-RLI-15 candidate molecules for DC-T cell-based assay

| Molecule | Molecule characteristics |
|---|---|
| pembrolizumab | humanized anti-PD-1 antibody, Manufacturer MSD |
| (KEYTRUDA ®) | |
| PEM L-RLI | RLI-15 mutations: D158A (D61A), N162A (N65A), |
| DANA ×1 | G175A, N176Q |
| | Fc modification: L235E, S228P, T366S, L368A, |
| | Y407V, T366W |
| PEM LY-RLI | RLI-15 mutations: D158A (D61A), N162A (N65A), |
| DANA ×1 | G175A, N176Q |
| | Fc modification: L235E, M252Y/S254T/T256E, |
| | S228P, T366S, L368A, Y407V, T366W |
| PEM LY-RLI | RLI-15 mutations: D158A (D61A), N162A (N65A), |
| DANAQD ×1 | Q198D (Q101D), G175A, N176Q |
| | Fc modification: L235E, M252Y/S254T/T256E, |
| | S228P, T366S, L368A, Y407V, T366W |

PEM-RLI-15 candidate molecules according to Table 40 were used at two concentrations each for the stimulation of iDCs. Maturation of DCs was induced by proinflammatory cytokines. After 24 h, mDCs were washed and incubated with autologous CD4⁺ T cells that were pre-stained with CFSE. Proliferation of T cells was evaluated based on CFSE detection by flow cytometry after 7 days.

The assay could not be conducted with SOT201 (PEM L-RLI N65A x1), due still too high activity of the RLI N65A mutein leading to the direct T cell activation and spill over the RLI-15 activity.

DCs generated from human CD14⁺ monocytes (11 healthy donors from 3 separate experiments) were incubated with 10 μg/ml (not shown) or 50 μg/ml PEM-RLI-15 candidate molecules, pembrolizumab or KLH for 24 h in the presence of maturation signal (proinflammatory cytokines TNFα and IL-1β). Washed mDCs loaded with proteins were subsequently cultured with autologous, CFSE stained CD4⁺ T cells. T cell proliferation was measured after 7 days by flow cytometry. Proportion of proliferating CD4⁺ T cells was evaluated based on CFSE signal, where CFSE^{low} cells were considered as cycling cells. KLH was used as a positive control, pembrolizumab as a negative control (see FIG. 29). The PEM-RLI-15 candidate molecule PEM L-RLI DANA x1/PEM LY-RLI DANA x1 did not induce significant proliferation of T cells compared to the negative control reflecting a low immunogenicity risk (positive response detected in 1 out of 11 donors). Candidate molecule PEM LY-RLI DANAQD x1 induced significant proliferation of T cells compared to negative control (p=0.0208, Paired t test), pointing to potential immunogenicity risk (positive response detected in 4 out of 11 donors) for this RLI-15 mutein having 3 mutations for reducing the binding to IL-2/IL-15Rβγ.

As a too active RLI-15 mutein is stimulating the immune response, the DC-T cell assay is not suitable to test the immunogenicity of the RLI-15_{AQA} as compared to RLI-15 (wildtype sequence). Accordingly, pairs of peptides having introduced substitutions were generated spanning the substitutions and tested in the Fluorospot assay.

TABLE 41
Tested peptides

| peptide | RLI-15 | Substitutions | sequence |
|---|---|---|---|
| WT2 | RLI-15_{AQ} | wt\|G₁₇₅A\|N₁₇₆Q | ELQVISLESGDASIHDTVENLIILANNSLSSNAQV |
| Mut2 | RLI-15_{AQA} | N₆₅A\|G₁₇₅A\|N₁₇₆Q | ELQVISLESGDASIHDTVEALIILANNSLSSNAQV |
| WT3 | RLI-15 NA | N₆₅A\|wt\|wt | VEALIILANNSLSSNGNVTESGCKECEELEEK |
| Mut3 | RLI-15_{AQA} | N₆₅A\|G₁₇₅A\|N₁₇₆Q | VEALIILANNSLSSNAQVTESGCKECEELEEK |

CD8-depleted PBMCs of 40 donors were seeded and incubated with test peptides in RPMI+10% huAb and IL-7. Medium was refreshed on day 1 with IL-7 and day 4 with IL-7 and IL-2. On day 7, CD8-depleted PBMC were harvested and rested overnight, seeded the next day on FluoroSpot plates and re-stimulated with the peptides. On day 9, INF-γ and TNF-α FluoroSpot plates were developed.

FIG. 29B shows that for all test conditions, the confidence intervals overlap with 0 meaning that there is no evidence of a shift in the mean dSFU comparing mutant peptides with the paired wildtype sequence. Therefore, for both the N65A substitution and the G75A/N176Q pair of substitutions, a relevant increase in the immunogenicity is not seen.

### Example 39: Potency of Different Anti-PD-1 IL-2/IL-15Rβγ Agonist Immunocytokines

The following anti-PD-1 IL-2/IL-15Rβγ agonist immunocytokines (Table 42) were made to compare their activities.

TABLE 42
anti-PD-1 IL-2/IL-15Rβγ agonist immunocytokines

| Molecule | Molecule characteristics |
|---|---|
| SOT201 | heterodimeric anti-PD-1 antibody (derived from IgG4 |
| | pembrolizumab with reduced FcγR binding) |
| | fused C-terminally to one RLI-15_{AQA} |
| | (SEQ ID NO: 21, SEQ ID NO: 101, SEQ ID NO: 23) |
| PD1-IL2v | heterodimeric anti-PD-1 antibody (abolished FcγR |
| | binding through LALAPG substitutions) |
| | fused C-terminally to one IL-2 mutein with T3A, F42A, |
| | Y45A, L72G, C125A substitutions (Klein, Codarri-Deak |
| | et al. 2019) |
| | (SEQ ID NO: 112, SEQ ID NO: 113, SEQ ID NO: 114) |
| αhPD1- | heterodimeric anti-PD-1 antibody (abolished FcγR |
| IL15m (M2) | binding through LALAGA substitutions) |
| | fused C-terminally to one IL-15 mutein with N1G, |
| | D30N, E46G, V49R, E64Q (Xu, Carrascosa et al. 2021) |
| | (SEQ ID NO: 121, SEQ ID NO: 122, SEQ ID NO: 123) |
| αhPD1- | heterodimeric anti-PD-1 antibody (abolished FcγR |
| IL15m1 (M1) | binding through LALAGA substitutions) |
| | fused C-terminally to one IL-15 mutein with N1A, |
| | D30N, E46G, V49R |
| | (SEQ ID NO: 118, SEQ ID NO: 119, SEQ ID NO: 120) |
| Kadmon | heterodimeric anti-PD1 antibody (abolished FcγR |
| | binding through LALAPG substitutions) |
| | N-terminally fused to one RLI-15_{N65S} mutein (Lu, |
| | Polonskaya et al. 2020) |
| | (SEQ ID NO: 124, SEQ ID NO: 125, SEQ ID NO: 126) |

The potency of the anti-PD-1 IL-2/IL-15Rβγ agonist immunocytokines was determined on kit225 cells (see Table 43) and hPBMC (see Table 44).

TABLE 43
Potency of anti-PD-1 IL-2/IL-15βγ agonist immunocytokines in
kit225 assay

| | EC50 | Relative potency to |
|---|---|---|
| | kit225 cells [pM] | SOT201 |
| SOT201 | 17917 | — |
| PD1-IL2v | 3938 | 455% |
| αhPD1-IL15m2 (M2) | 25372 | 70.62% |
| αhPD1-IL15m1 (M1) | 2682 | 668% |

TABLE 44
Potency of anti-PD-1 IL-2/IL-15Rβγ agonist immunocytokines
on hPBMC

| | | Relative | | Relative |
|---|---|---|---|---|
| | EC50 | potency | EC50 | potency |
| | Ki67⁺CD8⁺ | CD8⁺ | Ki67⁺NK | NK cells |
| | T cells | T cells to | cells | to |
| | [pM] | SOT201 | [pM] | SOT201 |
| SOT201 | 18828 | — | 5120 | — |
| PD1-IL2v | 4789 | 366 | 858 | 611 |
| αhPD1-IL15m2 (M2) | 6423 | 95 | 7004 | 69 |
| αhPD1-IL15m1 (M1) | 6925 | 430 | 728 | 686 |

### Example 40: PD-1/PD-L1 Blocking Activity of Anti-PD-1 IL-2/IL-15Rβγ Agonist Immunocytokines

To assess the blocking activity of the PD-1/PD-L1 axis, of anti-PD-1 IL-2/IL-15Rβγ agonist immunocytokines were tested using the PD-1/PD-L1 Blockade Bioassay (Promega, No. J1250) as described above. Results are shown in Table 45.

TABLE 45
PD-1/PD-L1 blocking anti-PD-1 IL-2/IL-15Rβγ agonist
immunocytokines in Promega blocking assay

| | EC50 | Relative potency vs |
|---|---|---|
| Sample | [pM] | Keytruda ® |
| Keytruda ® | 1765 | — |
| SOT201 | 2773 | 63.65% |
| PD1-IL2v | 4568 | 38.63% |
| αhPD1-IL15m (M2) | 6580 | 26.82% |

SOT201 shows the highest PD-1/PD-L1 blocking activity of the three tested anti-PD-1 IL-2/IL-15Rβγ agonist immunocytokines.

### Example 41: Potency of Human and Mouse Surrogate SOT202 Molecules with Modified Effector Functions on Kit225 Cells

SOT202 is a heterodimeric immunocytokine with an antibody derived from the humanized IgG 1 hCl1a with T366W—knob/T366S, L368A, Y407V—hole substitutions, and deleted terminal K of the heavy chains, fused to RLI-15_{AQA} at the C-terminus of the knob heavy chain (see SEQ ID NO: 111, SEQ ID NO: 110 and SEQ ID NO: 88). In the following examples, the term SOT202-XXX indicates molecules where further mutations of modification have been made to SOT202, such as the DANA mutation in RLI2 as shown in Table 13. For clarity, SOT202-DANA differs from SOT202 only by the additional DA (D61A) mutation, as SOT202 already contains the NA (N65A) mutation (numbers refer to IL-15 numbering). Mutation in the effector domain of the IgG1 molecule modifying ADCC properties of the antibody such as the AAA, DE and DLE mutations as shown in Table 2. The term “afuc” stands for an afucosylation IgG1 molecule. Afucosylated antibodies have also modified ADCC properties.

The activity of human and murine surrogate SOT202 ADCC-modified molecules on the induction of proliferation of kit225 cells was assessed as described in Example 1, and the EC50 and relative potency compared to SOT101 is shown in Table 46 and Table 47. The murine SOT202 was generated by replacing the human hIgG1 constant domain of SOT202 by its murine equivalent of mIgG2a (mSOT202: SEQ ID NO: 112, SEQ ID NO: 128 and SEQ ID NO: 129; mSOT2020 LALAPG: SEQ ID NO: 130, SEQ ID NO: 131 and SEQ ID NO: 129; mSOT202 isotype: mSOT202 isotype HC knob, SEQ ID NO: 133 and SEQ ID NO: 134; mSOT202 LALAPG isotype: SEQ ID NO: 135, SEQ ID NO: 136. SEQ ID NO: 134).

TABLE 46
Potency of human SOT202 ADCC-modified molecules on kit225 cells

| | EC50 | Relative potency |
|---|---|---|
| Sample | kit225 cells [pM] | to SOT101 |
| SOT101 | 44.42 | — |
| SOT202 (hCl1a-NA ×1) | 14269 | 0.31% |
| SOT202-afuc | 14316 | 0.31% |
| (hCl1a-afuc-NA ×1) | | |
| | | Relative potency |
| | EC50 | to PEM-RLI |
| Sample | kit225 cells [pM] | NQD ×1 |
| PEM-RLI NQD ×1 | 47198 | — |
| SOT202-DANA | 678300 | 6.96% |
| SOT202-afuc-DANA | 463652 | 10.18% |
| SOT202-AAA-DANA | 426399 | 11.07% |
| SOT202-DE-DANA | 534132 | 8.84% |
| SOT202-DLE-DANA | 646221 | 7.30% |

This potency assay shows that SOT202 displays the same potency on kit225 cells as SOT201 (see Table 36) and that ADCC modifications did not affect the potency of the immunocytokines. Therefore, the toolbox allows to tune ADCC activity of the antibodies without affecting the potency of the immunocytokines with respect to activation of kit225 cells.

TABLE 47
Potency of human SOT202 molecules and mouse SOT202 surrogates
on kit225 cells

| | EC50 | Relative potency |
|---|---|---|
| | kit225 cells [pM] | to SOT101 |
| SOT101 | 31.49 | — |
| SOT202 (hCl1a-NA ×1) | 9610 | 0.33% |
| mSOT202 | 24011 | 0.13% |
| SOT202 LALAPG | 7828 | 0.40% |
| mSOT202 LALAPG | 17969 | 0.18% |
| SOT202 LALAPG isotype | 13792 | 0.23% |
| mSOT202 LALAPG isotype | 10798 | 0.29% |
| SOT202 isotype | 9560 | 0.33% |
| mSOT202 isotype | 12475 | 0.25% |

As for human SOT202, the ADCC modification (LALAPG mutation) did not affect the potency of the mouse SOT202 surrogates with regards to activation of kit225 cells. However, mouse SOT202 surrogates are less potent than their human counterparts, likely is due to the kit225 cells expressing o CD16 required for co-signaling with IL-15Rβγ on human NK cells and mouse NK cells.

### Example 42: Potency of Human SOT202 ADCC-Modified Molecules on Human NK and CD8⁺ T Cells

The activity of human ST202 ADCC-modified molecules on the induction of proliferation of human NK and CD8⁺ T cells was assessed as described in Example 1 (hPBMC potency assay), and the EC50 and relative potency compared to S8T202 is shown in

### FIG. 30 and Table 48.

TABLE 48
Potency of human SOT202 ADCC-modified molecules on human
NK and CD8⁺ T cells

| | | Relative | | |
|---|---|---|---|---|
| | | potency | | Relative |
| | EC50 | CD8⁺ T | EC50 | potency |
| | Ki67⁺CD8⁺ | cells to | Ki67⁺ | NK cells to |
| | T cells | SOT202- | NK cells | SOT202- |
| | [pM] | DANA | pM | DANA |
| SOT202-DANA | 454350.00 | — | 6742.50 | — |
| SOT202-afuc-DANA | 547200.00 | 83% | 783.45 | 861% |
| SOT202-DLE-DANA | 100170.00 | 454% | 117.90 | 5719% |
| SOT202-DE-DANA | 198300.00 | 229% | 186.65 | 3612% |
| SOT202-LALAPG- | 1157000.00 | 39% | 126500.00 | 5% |
| DANA | | | | |

SOT202-DANA with DLE and DE mutation enhancing ADCC greatly increased the human NK cell activity when compared to SOT202-DANA without ADCC-modifications. Afucosylated SOT202 also increased ADCC activity, but to a lesser extent than the DE and DLE mutations. On the other hand, mutations reducing ADCC, such as the LALAPG mutations, almost abolished activation of NK cells. These mutations had only minor effects on CD8⁺ T cells activation. Without being bound by a theory, it is assumed that higher binding to CD16 receptors via enhancing mutations synergizes with the IL-15Rβγ signaling.

### Example 43: Potency of Human SOT202 Molecules on Human NK and CD8⁺ T Cells Compared to SOT201 Molecules

The activity of human SOT202 molecules on the induction of proliferation of human NK and CD8⁺ T cells was compared to the activity of SOT201. EC50 and relative potency compared to SOT202 and SOT201 is shown in

### FIG. 31 and Table 49.

TABLE 49
Potency of human SOT202 molecules on human NK and CD8+ T cells,
compared to SOT201

| | | Relative | | Relative |
|---|---|---|---|---|
| | | potency | EC50 | potency |
| | EC50 | CD8⁺ | Ki67⁺NK | NK cells |
| | Ki67⁺CD8⁺ | T cells to | cells | to |
| | T cells [pM] | SOT201 | [pM] | SOT201 |
| SOT201 (PEM L-RLI | 10850.00 | — | 3333.00 | — |
| NA ×1) | | | | |
| SOT202 (hCl1a-NA ×1) | 22360.00 | 49% | 345.70 | 964% |
| SOT202-afuc (hCl1a- | 12930.00 | 84% | 79.79 | 4177% |
| afuc-NA ×1) | | | | |
| SOT201-DANA (PEM | 628600.00 | 2% | 190300.00 | 2% |
| L-RLI DANA ×1) | | | | |
| SOT202-DANA | 703300.00 | 2% | 12300.00 | 27% |
| SOT202-afuc-DANA | 382100.00 | 3% | 2232.00 | 149% |

The activity of human SOT202 molecules on the induction of proliferation of human NK and CD8⁺ T cells was compared to the activity of SOT201-DANA. EC50 and relative potency compared to SOT202 and SOT201 is shown in

FIG. 32 and Table 49. Decreased stimulatory activity of molecules with DANA mutations, compared to molecules with NA mutations only, confirms the lower stimulatory activity of this mutation as already described in previous examples. SOT202 molecules (SOT202 having the NA mutation) with enhanced ADCC activity via afucosylation increase NK cells activity, but not CD8⁺ T cells activity, and confirms the results shown in Example 42. SOT201 is based on an IgG4 antibody, and as such, an IgG4 antibody, has intrinsic low ADCC activity.

TABLE 50
Potency of human SOT202 molecules on human NK and CD8+ T cells,
compared to SOT201-DANA

| | | Relative | | Relative |
|---|---|---|---|---|
| | | potency | | potency |
| | | CD8⁺ | EC50 | NK cells |
| | EC50 | T cells to | Ki67⁺NK | to |
| | Ki67⁺CD8⁺ | SOT201- | cells | SOT201- |
| | T cells [pM] | DANA | [pM] | DANA |
| SOT201-DANA | 1158000.00 | — | 117700.00 | — |
| (PEM L-RLI DANA ×1) | | | | |
| SOT202-DANA | 975800.00 | 119% | 3697.00 | 3184% |
| SOT202-afuc-DANA | 539000.00 | 215% | 524.40 | 22445% |
| SOT202-LALAPG- | 1237000.00 | 94% | 129000.00 | 91% |
| DANA | | | | |

SOT202 and SOT201 molecules have the same potency on human CD8⁺ T cells, but not on NK cells. Afucosylation increased human NK cell activity.

### Example 44: mSOT202 Activates Immune Cells in Spleen of Healthy C57BL/6 Mice

A murine SOT202 was generated by replacing the human hIgG1 constant domain of SOT202 by its murine equivalent of mIgG2a (SEQ ID NO: 127, SEQ ID NO: 128 and SEQ ID NO: 129. Cell proliferation (Ki67) was detected in spleen by flow cytometry 5 days after IV injection of compounds at 5, 10 or 20 mg/kg of mSOT202 in healthy C57BL/6 mice. mSOT202 showed dose-dependent stimulation of NK and CD8⁺ T cells

### (FIGS. 33(A) and (B)).

### Example 45: mSOT202 Induces Synergy Between ADCC Activity and the RLI2 Stimulation on NK Cell Proliferation

Cell proliferation (Ki67) was detected in spleen by flow cytometry 5 days and 10 days after IV injection of mSOT202 molecules at 5 mg/kg in healthy C57BL/6 mice. The proliferation activity on NK cells of mSOT202 (hCl1a-mIgG2a-NA 1×) was higher than the effect of hCl1a-mIgG2a (molecule without RLI2) added to the effect of mSOT202-LALAPG (hCl1a-mIgG2a-LALAPG-NA 1×, having no ADCC activity) showing a synergy between the ADCC activity of the antibody in mSOT202 and the proliferation activity of RLI2 (FIG. 34(A)) believed to be due to CD16 signalling. ADCC may therefore contribute to the increase activity of NK cells. Synergy could not be measured in this experimental model for CD8⁺ T cell stimulation (FIG. 34(B)).

The invention is described by the following embodiments:
1. An immunocytokine comprising:
   a. a conjugate comprising a polypeptide comprising an interleukin 15 (IL-15) or a derivative thereof and the sushi domain of an interleukin 15-receptor alpha (IL-15Rα) or a derivative thereof, and
   b. an antibody or a functional variant thereof,
wherein the antibody or functional variant thereof is characterized by:
   i. a heterodimeric Fc domain,
   ii. a modified effector function, and/or
   iii. an increased in vivo half-life;
wherein the conjugate is fused directly or indirectly to the C-terminus of both antibody heavy chains or antibody light chains, or, in case of i., to the C-terminus of one antibody heavy chain.
2. The immunocytokine of embodiment 1, wherein the modified effector function is reduced antibody-dependent cell toxicity and wherein the antibody or functional variant thereof
   a. is an IgG1 antibody or a functional variant thereof and comprises a mutation selected from L234A/L235A, P329G, L234A/L235A/P329G, G236R/L328R, D265A, N297A, N297Q, N297G or L234A/L235A/G237A/P238S/H268A/A330S/P331S,
   b. is an IgG4 antibody or a functional variant thereof and comprises a mutation selected from L235E, F234A/L235A, F234A/L235A/P329G, P329G, S228P/L235E, S228P/F234A/L235A or E233P/F234V/L235A/D265A/R409K,
   c. is a IgG2 (IgG2a or IgG2b) and IgG4 hybrid or a functional variant thereof and comprises a CH1 and hinge region from IgG2, and CH2 and CH3 regions are from IgG4 (IgG2 amino acids 118 to 260 and the IgG4 amino acids 261 to 447), or
   d. is an IgG2 antibody or a functional variant thereof and comprises a mutation selected from H268Q/V309L/A330S/P331S or V234A/G237A/P238S/H268A/V309L/A330S/P331S,
   wherein numbering is according to EU numbering.
3. The immunocytokine of embodiment 2, wherein the antibody or functional variant thereof
   (a) is an IgG4 antibody or a functional variant thereof and comprises a L235E mutation, or
   (b) is an IgG1 antibody or a functional variant thereof and comprises a L234A/L235A mutation.
4. The immunocytokine of embodiment 1, wherein the modified effector function is enhanced antibody-dependent cell toxicity and wherein the antibody or functional variant thereof:
   a. is an IgG1 antibody or a functional variant thereof and comprises a mutation selected from F243L/R292P/Y300L/V305I/P396L, S239D/I332E, S239D/I332E/A330L, S298A/E333A/K334A, K392T/P396L, V264I/I332E or L234Y/L235Q/G236W/S239M/H268D/D270E/S298A, preferably from S239D/I332E, S239D/I332E/A330L, S298A/E333A/K334A, K392T/P396L, V264I/I332E, in one heavy chain and further comprises a D270E/K326D/A330M/K334E mutation in the opposing heavy chain, and/or
   b. is an afucosylated IgG1, IgG2 or IgG4 antibody or a functional variant thereof,
wherein numbering is according to EU numbering.
5. The immunocytokine of any of the embodiments 1 to 4 wherein the heterodimeric Fc domain is selected from KiH, KiH_{S-S}, HA-TF, ZW1, 7.8.60, DD-KK, EW-RVT, EW-RVT_{S-S}, SEED and A107, preferably KiH.
6. The immunocytokine of any of the embodiments 1 to 5, wherein the heterodimeric Fc domain leads to higher yield of the immunocytokine upon expression in cell culture, compared to an immunocytokine with homodimeric Fc domain.
7. The immunocytokine of any of the embodiment 1 to 6, wherein the half-life of the immunocytokine is increased and wherein the antibody or functional variant thereof is an IgG1 or an IgG4 antibody or a functional variant thereof and comprises a mutation selected from M252Y/S254T/T256E, M428L/N434S or T250Q/M428L, wherein numbering is according to EU numbering.
8. The immunocytokine of any of the embodiments 1 to 3, wherein the antibody or functional variant thereof has reduced antibody-dependent cellular cytotoxicity and wherein the antibody or functional variant thereof is an IgG4 antibody or a functional variant thereof and comprises a L235E mutation and a KiH heterodimerized Fc domain.
9. The immunocytokine of any of embodiments 1 to 8, wherein the conjugate is a fusion protein comprising, in N- to C-terminal order, the IL-15Rα sushi domain or a derivative thereof, a linker and the IL-15 or a derivative thereof, preferably wherein the IL-15Rα sushi domain comprises the sequence of SEQ ID NO: 5, and
   wherein the linker has a length of 18 to 22 amino acids and is composed preferably of glycines or serines and glycines, more preferably has the sequence of SEQ ID NO: 7, and wherein the IL-15 has the sequence of SEQ ID NO: 2.
10. The immunocytokine of any of embodiments 1 to 9, wherein the IL-15 variant comprises:
   a. at least one mutation increasing the homogeneity of the IL-15 variant with respect to post-translational modifications,
      preferably wherein the mutation reduces deamidation at N77 and/or glycosylation at N79 of IL-15 (SEQ ID NO: 2),
      more preferably wherein the mutation is selected from mutations G78A, G78V, G78L or G78I, and N79Q, N79S or N79T,
      most preferably wherein the mutation is G78A/N79Q; and/or
   b. at least one mutation that reduces the binding to the IL-2/IL-15Rβ and/or to the γ_{c} receptor, preferably wherein the mutated amino acid is selected from N1, N4, S7, D8, K10, K11, D30, D61, E64, N65, L69, N72, E92, Q101, Q108, 1111 of IL-15 (SEQ ID NO: 2), more preferably wherein the mutated amino acid is selected from D61, N65 and Q101, most preferably wherein the mutated amino acid is N65.
11. The immunocytokine of any of embodiments 1 to 10, wherein the at least one mutation that reduces the binding to the IL-2/IL-15Rβ and/or to the γc receptor is a substitution selected from N1D, N1A, N1G, N4D, S7Y, S7A, D8A, D8N, K10A, K11A, D30N, D61A, D61N, E64Q, N65D, N65A, N65E, N65R, N65K, L69R, N72R, Q101D, Q101E, Q108D, Q108A, Q108E and Q108R, preferably D8A, D8N, D61A, D61N, N65A, N65D, N72R, Q101D, Q101E and Q108A, more preferably D61A, N65A and Q101D, most preferably N65A or a combined substitution selected from D8N/N65A, D61A/N65A or D61A/N65A/Q101D.
12. The immunocytokine of any of the embodiments 1 to 11, wherein the antibody or functional variant thereof:
   a. binds to a tumor antigen, preferably selected from EGFR, HER2, FGFR2, FOLR1, CLDN18.2, CEA, GD2, O-Acetyl-GD-2, GM1, CAIX, EPCAM, MUC1, PSMA, c-MET, ROR1, GPC3, CD19, CD20, CD38;
   b. binds to a tumor extracellular matrix antigen, preferably selected from FAP, the EDA domain of fibronectin, the EDB domain of fibronectin and LRRC15, preferably FAP and the EDB domain of fibronectin;
   c. binds to a neovascularization antigen, preferably VEGF, or Endoglin;
   d. is an immunomodulatory antibody or a functional variant thereof, wherein the immunomodulatory antibody stimulates a co-stimulatory receptor, preferably selected from CD40 agonists, CD137/4-1BB agonists, CD134/OX40 agonists and TNFRSF18/GITR agonists, or
      wherein the immunomodulatory antibody inhibits an immunosuppressive receptor, preferably selected from PD-1 antagonists, CTLA-4 antagonists, LAG3 antagonists, TIGIT antagonists, inhibitory KIRs antagonists, BTLA antagonists, HAVCR2 antagonists and ADORA2A antagonists, more preferably PD-1 antagonists.
13. The immunocytokine of any of the embodiment 1, wherein the cytokine domain comprises the sequence of SEQ ID NO: 10; and
   the antibody comprises:
   i. the heavy chain knob sequence of SEQ ID NO: 20,
   ii. the heavy chain hole sequence of SEQ ID NO: 22, and, and
   iii. the light chain sequence of SEQ ID NO: 16;
wherein the cytokine domain is fused to the C-terminus heavy chain knob sequence without a linker.
14. The immunocytokine of any of the embodiment 1, wherein the cytokine domain comprises the sequence of SEQ ID NO: 10; and the antibody comprises:
   i. the heavy chain knob sequence of SEQ ID NO: 84,
   ii. the heavy chain hole sequence of SEQ ID NO: 87, and
   iii. the light chain sequence of SEQ ID NO: 88;
wherein the cytokine domain is fused to the C-terminus heavy chain knob sequence without a linker.
15. The immunocytokine of any of the embodiment 1, wherein the cytokine domain comprises the sequence of SEQ ID NO: 10; and the antibody comprises:
   i. the heavy chain knob sequence of SEQ ID NO: 93,
   ii. the heavy chain hole sequence of SEQ ID NO: 95 and
   iii. the light chain sequence of SEQ ID NO: 92;
wherein the cytokine domain is fused to the C-terminus heavy chain knob sequence without a linker.
16. A nucleic acid encoding the immunocytokine of any of the embodiments 1 to 15.
17. A vector comprising the nucleic acid of embodiment 16.
18. A host cell comprising the nucleic acid of embodiment 16 or the vector of embodiment 17.
19. The immunocytokine of any of the embodiments 1 to 15, the nucleic acid of embodiment 16 or the vector of embodiment 17 for use in treatment.
20. A pharmaceutical composition comprising the immunocytokine of any of the embodiments 1 to 15, the nucleic acid of embodiment 16 or the vector of embodiment 17 and a pharmaceutically acceptable carrier.
21. The immunocytokine of any of the embodiments 1 to 15, the nucleic acid of embodiment 16 or the vector of embodiment 17 for use in the treatment of a subject suffering from, at risk of developing and/or being diagnosed for a neoplastic disease or a an infectious disease.
22. A method for treating a patient suffering from, at risk of developing and/or being diagnosed for a neoplastic disease or an infectious disease comprising administering the immunocytokine of any of the embodiments 1 to 15, the nucleic acid of embodiment 16 or the vector of embodiment 17.

The invention is also described by the following embodiments:
1. An immunocytokine comprising:
   a. a conjugate comprising a polypeptide comprising an interleukin 15 (IL-15) or a derivative thereof and the sushi domain of an interleukin 15-receptor alpha (IL-15Rα) or a derivative thereof, and
   b. a PD-1 antibody or a functional variant thereof,
   wherein the antibody or functional variant thereof is characterized by:
   i. a heterodimeric Fc domain,
   ii. a modified effector function, and/or
   iii. an increased in vivo half-life;
wherein the conjugate is fused directly or indirectly to the C-terminus of both antibody heavy chains or antibody light chains, or, in case of i., to the C-terminus of one antibody heavy chain.
2. The immunocytokine of embodiment 1, wherein the modified effector function is reduced antibody-dependent cell toxicity and wherein the antibody or functional variant thereof
   a. is an IgG1 antibody or a functional variant thereof and comprises a mutation selected from L234A/L235A, P329G, L234A/L235A/P329G, G236R/L328R, D265A, N297A, N297Q, N297G or L234A/L235A/G237A/P238S/H268A/A330S/P331S,
   b. is an IgG4 antibody or a functional variant thereof and comprises a mutation selected from L235E, F234A/L235A, F234A/L235A/P329G, P329G, S228P/L235E, S228P/F234A/L235A or E233P/F234V/L235A/D265A/R409K,
   c. is a IgG2 (IgG2a or IgG2b) and IgG4 hybrid or a functional variant thereof and comprises a CH1 and hinge region from IgG2, and CH2 and CH3 regions are from IgG4 (IgG2 amino acids 118 to 260 and the IgG4 amino acids 261 to 447), or
   d. is an IgG2 antibody or a functional variant thereof and comprises a mutation selected from H268Q/V309L/A330S/P331S or V234A/G237A/P238S/H268A/V309L/A330S/P331S,
   wherein numbering is according to EU numbering.
3. The immunocytokine of embodiment 2, wherein the antibody or functional variant thereof
   (a) is an IgG4 antibody or a functional variant thereof and comprises a L235E mutation, or
   (b) is an IgG1 antibody or a functional variant thereof and comprises a L234A/L235A mutation.
4. The immunocytokine of any of the embodiments 1 to 3 wherein the heterodimeric Fc domain is selected from KiH, KiH_{S-S}, HA-TF, ZW1, 7.8.60, DD-KK, EW-RVT, EW-RVT_{S-S}, SEED and A107, preferably KiH.
5. The immunocytokine of any of the embodiments 1 to 4 wherein the heterodimeric Fc domain leads to higher yield of the immunocytokine upon expression in cell culture, compared to an immunocytokine with homodimeric Fc domain.
6. The immunocytokine of any of the embodiments 1 to 5, wherein the half-life of the immunocytokine is increased and wherein the antibody or functional variant thereof is an IgG1 or an IgG4 antibody or a functional variant thereof and comprises a mutation selected from M252Y/S254T/T256E, M428L/N434S or T250Q/M428L, wherein numbering is according to EU numbering.
7. The immunocytokine of any of the embodiments 1 to 6, wherein the antibody or functional variant thereof has reduced antibody-dependent cellular cytotoxicity and wherein the antibody or functional variant thereof is an IgG4 antibody or a functional variant thereof and comprises a L235E mutation and a KiH heterodimerized Fc domain.
8. The immunocytokine of any of embodiments 1 to 7, wherein the conjugate is a fusion protein comprising, in N- to C-terminal order, the IL-15Rα sushi domain or a derivative thereof, a linker and the IL-15 or a derivative thereof, preferably wherein the IL-15Rα sushi domain comprises the sequence of SEQ ID NO: 5, and wherein the linker has a length of 18 to 22 amino acids and is composed preferably of glycines or serines and glycines, more preferably has the sequence of SEQ ID NO: 7, and wherein the IL-15 has the sequence of SEQ ID NO: 2.
9. The immunocytokine of any of the embodiments 1 to 8, wherein the cytokine domain comprises the sequence of SEQ ID NO: 10; and the antibody comprises:
   i. the heavy chain knob sequence of SEQ ID NO: 20
   ii. the heavy chain hole sequence of SEQ ID NO: 22, and
   iii. the light chain sequence of SEQ ID NO: 23
wherein the cytokine domain is fused to the C-terminus heavy chain knob sequence without a linker.

Another aspect of the invention is described in the following embodiments:
1. An immunocytokine comprising:
   a. a conjugate comprising a polypeptide comprising an interleukin 15 (IL-15) or a derivative thereof and the sushi domain of an interleukin 15-receptor alpha (IL-15Rα) or a derivative thereof, and
   b. an Claudin18.2 antibody or a functional variant thereof,
   wherein the antibody or functional variant thereof is characterized by:
   i. a heterodimeric Fc domain,
   ii. a modified effector function, and/or
   iii. an increased in vivo half-life;
wherein the conjugate is fused directly or indirectly to the C-terminus of both antibody heavy chains or antibody light chains, or, in case of i., to the C-terminus of one antibody heavy chain.
2. The immunocytokine of embodiment 1, wherein the modified effector function is enhanced antibody-dependent cell toxicity and wherein the antibody or functional variant thereof:
   a. is an IgG1 antibody or a functional variant thereof and comprises a mutation selected from F243L/R292P/Y300L/V305I/P396L, S239D/I332E, S239D/I332E/A330L, S298A/E333A/K334A, K392T/P396L, V264I/I332E or L234Y/L235Q/G236W/S239M/H268D/D270E/S298A, preferably from S239D/I332E, S239D/I332E/A330L, S298A/E333A/K334A, K392T/P396L, V264I/I332E, in one heavy chain and further comprises a D270E/K326D/A330M/K334E mutation in the opposing heavy chain, and/or
   b. is an afucosylated IgG1, IgG2 or IgG4 antibody or a functional variant thereof,
wherein numbering is according to EU numbering.
3. The immunocytokine of any of the embodiments 1 to 2 wherein the heterodimeric Fc domain is selected from KiH, KiH_{S-S}, HA-TF, ZW1, 7.8.60, DD-KK, EW-RVT, EW-RVT_{S-S}, SEED and A107, preferably KiH.
4. The immunocytokine of any of the embodiments 1 to 3, wherein the heterodimeric Fc domain leads to higher yield of the immunocytokine upon expression in cell culture, compared to an immunocytokine with homodimeric Fc domain.
5. The immunocytokine of any of the embodiments 1 to 4, wherein the half-life of the immunocytokine is increased and wherein the antibody or functional variant thereof is an IgG1 or an IgG4 antibody or a functional variant thereof and comprises a mutation selected from M252Y/S254T/T256E, M428L/N434S or T250Q/M428L, wherein numbering is according to EU numbering.
6. The immunocytokine of any of embodiments 1 to 5, wherein the conjugate is a fusion protein comprising, in N- to C-terminal order, the IL-15Rα sushi domain or a derivative thereof, a linker and the IL-15 or a derivative thereof, preferably wherein the IL-15Rα sushi domain comprises the sequence of SEQ ID NO: 5, and wherein the linker has a length of 18 to 22 amino acids and is composed preferably of glycines or serines and glycines, more preferably has the sequence of SEQ ID NO: 7, and wherein the IL-15 has the sequence of SEQ ID NO: 2.
7. The immunocytokine of any of embodiments 1 to 6, wherein the conjugate comprises the sequence SEQ ID NO: 10 or SEQ ID NO: 11 and the antibody is an anti-CLDN18.2 heterodimeric IgG1 antibody variant having a VH and VL domain sequence of Table 4, the IgG1 variant being heterodimeric through the KiH mutation of Table 3, having enhanced ADCC activity through the DE, DLE, AAA, TL or IE mutations of Table 2 or through afucosylation, or through the combination of a mutation listed above and afucosylation.
8. The immunocytokine of any of embodiments 1 to 7, wherein the conjugate comprises the sequence SEQ ID NO: 10 and the antibody is an anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having enhanced ADCC activity through the DE, DLE, AAA, TL or IE mutations of Table 2 or through afucosylation, or through the combination of a mutation listed above and afucosylation.
9. The immunocytokine of any of embodiments 1 to 7, wherein the conjugate comprises the sequence SEQ ID NO: 10 and the antibody is an anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having enhanced ADCC activity through afucosylation.
10. The immunocytokine of any of embodiments 1 to 7, wherein the conjugate comprises the sequence of SEQ ID NO: 11, and the antibody variant is a heterodimeric IgG1 anti-CLDN18.2 antibody having heavy chain knob sequence of SEQ ID NO: 84, heavy chain hole sequence of SEQ ID NO: 87 and the light chain sequence of SEQ ID NO: 88.
11. The immunocytokine of any of embodiments 1 to 7, wherein the conjugate comprises the sequence of SEQ ID NO: 10 and the antibody variant is a heterodimeric IgG1 anti-CLDN18.2 antibody having heavy chain knob sequence of SEQ ID NO: 84, heavy chain hole sequence of SEQ ID NO: 87 and the light chain sequence of SEQ ID NO: 88.
12. The immunocytokine of any of embodiment 1 to 7, wherein the conjugate comprises the sequence SEQ ID NO: 10 and the antibody is an anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having the S239D/I332E (DE) ADCC-enhancing mutation in the IgG1 Fc domain.
13. The immunocytokine of any of embodiment 1 to 7, wherein the conjugate comprises the sequence SEQ ID NO: 11 and the antibody is an anti-CLDN18.2 heterodimeric IgG1 antibody variant having a VH and VL domain sequence of SEQ ID NO: 46 and SEQ ID NO: 47, respectively, the IgG1 variant being heterodimeric through the KiH mutation of Table 3, having the S239D/I332E (DE) ADCC-enhancing mutation in the IgG1 Fc domain.
14. An immunocytokine of the sequence SEQ ID NO: 85 (“HC knob”), SEQ ID NO: 87 (“HC hole”) and SEQ ID NO: 88 (LC).
15. An immunocytokine of the sequence SEQ ID NO: 86 (“HC knob”), SEQ ID NO: 87 (“HC hole”) and SEQ ID NO: 88 (LC).

### REFERENCES

Ahmed, A. A., S. R. Keremane, J. Vielmetter and P. J. Bjorkman (2016). “Structural characterization of GASDALIE Fc bound to the activating Fc receptor FcgammaRIIIa.” J Struct Biol 194(1): 78-89.
Alegre, M. L., A. M. Collins, V. L. Pulito, R. A. Brosius, W. C. Olson, R. A. Zivin, R. Knowles, J. R. Thistlethwaite, L. K. Jolliffe and J. A. Bluestone (1992). “Effect of a single amino acid mutation on the activating and immunosuppressive properties of a “humanized” OKT3 monoclonal antibody.” J Immunol 148(11): 3461-3468.
An, Z., G. Forrest, R. Moore, M. Cukan, P. Haytko, L. Huang, S. Vitelli, J. Z. Zhao, P. Lu, J. Hua, C. R. Gibson, B. R. Harvey, D. Montgomery, D. Zaller, F. Wang and W. Strohl (2009). “IgG2m4, an engineered antibody isotype with reduced Fc function.” MAbs 1(6): 572-579.
Bolt, S., E. Routledge, I. Lloyd, L. Chatenoud, H. Pope, S. D. Gorman, M. Clark and H. Waldmann (1993). “The generation of a humanized, non-mitogenic CD3 monoclonal antibody which retains in vitro immunosuppressive properties.” Eur J Immunol 23(2): 403-411.
Chester, C., M. F. Sanmamed, J. Wang and I. Melero (2018). “Immunotherapy targeting 4-1BB: mechanistic rationale, clinical results, and future strategies.” Blood 131(1): 49-57.
Choi, H. J., Y. J. Kim, D. K. Choi and Y. S. Kim (2015). “Engineering of Immunoglobulin Fc Heterodimers Using Yeast Surface-Displayed Combinatorial Fc Library Screening.” PLoS One 10(12): e0145349.
Choi, H. J., Y. J. Kim, S. Lee and Y. S. Kim (2013). “A heterodimeric Fc-based bispecific antibody simultaneously targeting VEGFR-2 and Met exhibits potent antitumor activity.” Mol Cancer Ther 12(12): 2748-2759.
Choi, H. J., S. H. Seok, Y. J. Kim, M. D. Seo and Y. S. Kim (2015). “Crystal structures of immunoglobulin Fc heterodimers reveal the molecular basis for heterodimer formation.” Mol Immunol 65(2): 377-383.
Chu, S. Y., I. Vostiar, S. Karki, G. L. Moore, G. A. Lazar, E. Pong, P. F. Joyce, D. E. Szymkowski and J. R. Desjarlais (2008). “Inhibition of B cell receptor-mediated activation of primary human B cells by coengagement of CD19 and FcgammaRIIb with Fc-engineered antibodies.” Mol Immunol 45(15): 3926-3933.
Clemenceau, B., R. Vivien, C. Pellat, M. Foss, G. Thibault and H. Vie (2013). “The human natural killer cytotoxic cell line NK-92, once armed with a murine CD16 receptor, represents a convenient cellular tool for the screening of mouse mAbs according to their ADCC potential.” MAbs 5(4): 587-594.
Conlon, K. C., M. D. Miljkovic and T. A. Waldmann (2019). “Cytokines in the Treatment of Cancer.” J Interferon Cytokine Res 39(1): 6-21.
Dall'Acqua, W. F., R. M. Woods, E. S. Ward, S. R. Palaszynski, N. K. Patel, Y. A. Brewah, H. Wu, P. A. Kiener and S. Langermann (2002). “Increasing the affinity of a human IgG1 for the neonatal Fc receptor: biological consequences.” J Immunol 169(9): 5171-5180.
Davis, J. H., C. Aperlo, Y. Li, E. Kurosawa, Y. Lan, K. M. Lo and J. S. Huston (2010). “SEEDbodies: fusion proteins based on strand-exchange engineered domain (SEED) CH3 heterodimers in an Fc analogue platform for asymmetric binders or immunofusions and bispecific antibodies.” Protein Eng Des Sel 23(4): 195-202.
Dekkers, G., R. Plomp, C. A. Koeleman, R. Visser, H. H. von Horsten, V. Sandig, T. Rispens, M. Wuhrer and G. Vidarsson (2016). “Multi-level glyco-engineering techniques to generate IgG with defined Fc-glycans.” Sci Rep 6: 36964.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros and P. W. Parren (2014). “Complement is activated by IgG hexamers assembled at the cell surface.” Science 343(6176): 1260-1263.
Dolgin, E. (2020). “Antibody engineers seek optimal drug targeting TIGIT checkpoint.” Nat Biotechnol 38(9): 1007-1009.
Elliott, J. M., M. Ultsch, J. Lee, R. Tong, K. Takeda, C. Spiess, C. Eigenbrot and J. M. Scheer (2014). “Antiparallel conformation of knob and hole aglycosylated half-antibody homodimers is mediated by a CH2-CH3 hydrophobic interaction.” J Mol Biol 426(9): 1947-1957.
Forero-Torres, A., S. de Vos, B. L. Pohlman, M. Pashkevich, D. M. Cronier, N. H. Dang, S. P. Carpenter, B. W. Allan, J. G. Nelson, C. A. Slapak, M. R. Smith, B. K. Link, J. E. Wooldridge and K. N. Ganjoo (2012). “Results of a phase 1 study of AME-133v (LY2469298), an Fc-engineered humanized monoclonal anti-CD20 antibody, in FcgammaRIIIa-genotyped patients with previously treated follicular lymphoma.” Clin Cancer Res 18(5): 1395-1403.
Fu, Y., Q. Lin, Z. Zhang and L. Zhang (2020). “Therapeutic strategies for the costimulatory molecule OX40 in T-cell-mediated immunity.” Acta Pharmaceutica Sinica B 10(3): 414-433. Godar, M., H. de Haard, C. Blanchetot and J. Rasser (2018). “Therapeutic bispecific antibody formats: a patent applications review (1994-2017).” Expert Opinion on Therapeutic Patents 28(3): 251-276.
Gunasekaran, K., M. Pentony, M. Shen, L. Garrett, C. Forte, A. Woodward, S. B. Ng, T. Born, M. Retter, K. Manchulenko, H. Sweet, I. N. Foltz, M. Wittekind and W. Yan (2010). “Enhancing antibody Fc heterodimer formation through electrostatic steering effects: applications to bispecific molecules and monovalent IgG.” J Biol Chem 285(25): 19637-19646.
Ha, J.-H., J.-E. Kim and Y.-S. Kim (2016). “Immunoglobulin Fc Heterodimer Platform Technology: From Design to Applications in Therapeutic Antibodies and Proteins.” Frontiers in Immunology 7(394).
Hori, T., T. Uchiyama, M. Tsudo, H. Umadome, H. Ohno, S. Fukuhara, K. Kita and H. Uchino (1987). “Establishment of an interleukin 2-dependent human T cell line from a patient with T cell chronic lymphocytic leukemia who is not infected with human T cell leukemia/lymphoma virus.” Blood 70(4): 1069-1072.
Idusogie, E. E., P. Y. Wong, L. G. Presta, H. Gazzano-Santoro, K. Totpal, M. Ultsch and M. G. Mulkerrin (2001). “Engineered antibodies with increased activity to recruit complement.” J Immunol 166(4): 2571-2575.
Klein, C., L. Codarri-Deak, V. Nicolini, S. Seeber, L. Lauener, M. Richard, E. Bommer, M. Karagianni, J. Sam, R. Schlenker, M. Mariani, P. P. Schwalie, S. Herter, M. Bacac, I. Waldhauer, A. Freimoser-Grundschober, V. Teichgraeber and P. Umana (2019). “Abstract 1552: A novel PD1-IL2v immunocytokine for preferential <em>cis</em>-activation of IL-2R signaling on PD-1 expressing T cell subsets strongly potentiates anti-tumor T cell activity of PD-1 checkpoint inhibition and IL-2R-beta-gamma agonism.” Cancer Research 79(13 Supplement): 1552-1552.
Lazar, G. A., W. Dang, S. Karki, O. Vafa, J. S. Peng, L. Hyun, C. Chan, H. S. Chung, A. Eivazi, S. C. Yoder, J. Vielmetter, D. F. Carmichael, R. J. Hayes and B. I. Dahiyat (2006). “Engineered antibody Fc variants with enhanced effector function.” Proc Natl Acad Sci USA 103(11): 4005-4010.
Leabman, M. K., Y. G. Meng, R. F. Kelley, L. E. DeForge, K. J. Cowan and S. Iyer (2013). “Effects of altered FcgammaR binding on antibody pharmacokinetics in cynomolgus monkeys.” MAbs 5(6): 896-903.
Leaver-Fay, A., K. J. Froning, S. Atwell, H. Aldaz, A. Pustilnik, F. Lu, F. Huang, R. Yuan, S. Hassanali, A. K. Chamberlain, J. R. Fitchett, S. J. Demarest and B. Kuhlman (2016). “Computationally Designed Bispecific Antibodies using Negative State Repertoires.” Structure 24(4): 641-651.
Lo, M., H. S. Kim, R. K. Tong, T. W. Bainbridge, J. M. Vernes, Y. Zhang, Y. L. Lin, S. Chung, M. S. Dennis, Y. J. Zuchero, R. J. Watts, J. A. Couch, Y. G. Meng, J. K. Atwal, R. J. Brezski, C. Spiess and J. A. Ernst (2017). “Effector-attenuating Substitutions That Maintain Antibody Stability and Reduce Toxicity in Mice.” J Biol Chem 292(9): 3900-3908. Lu, D., Z. Polonskaya, T.-P. Chang, S. Martomo, X. Luna, Z. Zhang, F. Miyara and J. Patel (2020). “Abstract 525: A novel anti-PD1-IL15 immunocytokine potentiates anti-tumor T cell activity of PD-1 checkpoint inhibition and IL-2R-beta-gamma agonism.” Cancer Research 80(16_Supplement): 525-525.
Lu, X., R. P. Nobrega, H. Lynaugh, T. Jain, K. Barlow, T. Boland, A. Sivasubramanian, M. Vasquez and Y. Xu (2019). “Deamidation and isomerization liability analysis of 131 clinical-stage antibodies.” MAbs 11(1): 45-57.
Merchant, A. M., Z. Zhu, J. Q. Yuan, A. Goddard, C. W. Adams, L. G. Presta and P. Carter (1998). “An efficient route to human bispecific IgG.” Nat Biotechnol 16(7): 677-681.
Mimoto, F., T. Igawa, T. Kuramochi, H. Katada, S. Kadono, T. Kamikawa, M. Shida-Kawazoe and K. Hattori (2013). “Novel asymmetrically engineered antibody Fc variant with superior FcgammaR binding affinity and specificity compared with afucosylated Fc variant.” MAbs 5(2): 229-236.
Moore, G. L., C. Bautista, E. Pong, D. H. Nguyen, J. Jacinto, A. Eivazi, U. S. Muchhal, S. Karki, S. Y. Chu and G. A. Lazar (2011). “A novel bispecific antibody format enables simultaneous bivalent and monovalent co-engagement of distinct target antigens.” MAbs 3(6): 546-557.
Moore, G. L., H. Chen, S. Karki and G. A. Lazar (2010). “Engineered Fc variant antibodies with enhanced ability to recruit complement and mediate effector functions.” MAbs 2(2): 181-189.
Natsume, A., M. In, H. Takamura, T. Nakagawa, Y. Shimizu, K. Kitajima, M. Wakitani, S. Ohta, M. Satoh, K. Shitara and R. Niwa (2008). “Engineered antibodies of IgG1/IgG3 mixed isotype with enhanced cytotoxic activities.” Cancer Res 68(10): 3863-3872.
Nellis, D. F., D. F. Michiel, M. S. Jiang, D. Esposito, R. Davis, H. Jiang, A. Korrell, G. C. t. Knapp, L. E. Lucernoni, R. E. Nelson, E. M. Pritt, L. V. Procter, M. Rogers, T. L. Sumpter, V. V. Vyas, T. J. Waybright, X. Yang, A. M. Zheng, J. L. Yovandich, J. A. Gilly, G. Mitra and J. Zhu (2012). “Characterization of recombinant human IL-15 deamidation and its practical elimination through substitution of asparagine 77.” Pharm Res 29(3): 722-738.
Nordstrom, J. L., S. Gorlatov, W. Zhang, Y. Yang, L. Huang, S. Burke, H. Li, V. Ciccarone, T. Zhang, J. Stavenhagen, S. Koenig, S. J. Stewart, P. A. Moore, S. Johnson and E. Bonvini (2011). “Anti-tumor activity and toxicokinetics analysis of MGAH22, an anti-HER2 monoclonal antibody with enhanced Fcgamma receptor binding properties.” Breast Cancer Res 13(6): R123.
Pereira, N. A., K. F. Chan, P. C. Lin and Z. Song (2018). “The “less-is-more” in therapeutic antibodies: Afucosylated anti-cancer antibodies with enhanced antibody-dependent cellular cytotoxicity.” MAbs 10(5): 693-711.
Perussia, B. and M. J. Loza (2000). “Assays for antibody-dependent cell-mediated cytotoxicity (ADCC) and reverse ADCC (redirected cytotoxicity) in human natural killer cells.” Methods Mol Biol 121: 179-192.
Pini, A., F. Viti, A. Santucci, B. Carnemolla, L. Zardi, P. Neri and D. Neri (1998). “Design and use of a phage display library. Human antibodies with subnanomolar affinity against a marker of angiogenesis eluted from a two-dimensional gel.” J Biol Chem 273(34): 21769-21776.
Richards, J. O., S. Karki, G. A. Lazar, H. Chen, W. Dang and J. R. Desjarlais (2008). “Optimization of antibody binding to FcgammaRIIa enhances macrophage phagocytosis of tumor cells.” Mol Cancer Ther 7(8): 2517-2527.
Robinson, N. E. and A. B. Robinson (2004). “Prediction of primary structure deamidation rates of asparaginyl and glutaminyl peptides through steric and catalytic effects.” J Pept Res 63(5): 437-448.
Robinson, T. O. and K. S. Schluns (2017). “The potential and promise of IL-15 in immuno-oncogenic therapies.” Immunol Lett 190: 159-168.
Rosenlocher, J., V. Bohrsch, M. Sacharjat, V. Blanchard, C. Giese, V. Sandig, C. P. R. Hackenberger and S. Hinderlich (2015). “Applying Acylated Fucose Analogues to Metabolic Glycoengineering.” Bioengineering (Basel) 2(4): 213-234.
Rother, R. P., S. A. Rollins, C. F. Mojcik, R. A. Brodsky and L. Bell (2007). “Discovery and development of the complement inhibitor eculizumab for the treatment of paroxysmal nocturnal hemoglobinuria.” Nat Biotechnol 25(11): 1256-1264.
Shang, L., B. Daubeuf, M. Triantafilou, R. Olden, F. Depis, A. C. Raby, S. Herren, A. Dos Santos, P. Malinge, I. Dunn-Siegrist, S. Benmkaddem, A. Geinoz, G. Magistrelli, F. Rousseau, V. Buatois, S. Salgado-Pires, W. Reith, R. Monteiro, J. Pugin, O. Leger, W. Ferlin, M. Kosco-Vilbois, K. Triantafilou and G. Elson (2014). “Selective antibody intervention of Toll-like receptor 4 activation through Fc gamma receptor tethering.” J Biol Chem 289(22): 15309-15318.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Silva, J. P., O. Vetterlein, J. Jose, S. Peters and H. Kirby (2015). “The S228P mutation prevents in vivo and in vitro IgG4 Fab-arm exchange as demonstrated using a combination of novel quantitative immunoassays and physiological matrix preparation.” J Biol Chem 290(9): 5462-5469.
Soman, G., X. Yang, H. Jiang, S. Giardina, V. Vyas, G. Mitra, J. Yovandich, S. P. Creekmore, T. A. Waldmann, O. Quinones and W. G. Alvord (2009). “MTS dye based colorimetric CTLL-2 cell proliferation assay for product release and stability monitoring of interleukin-15: assay qualification, standardization and statistical analysis.” J Immunol Methods 348(1-2): 83-94.
Spiess, C., Q. Zhai and P. J. Carter (2015). “Alternative molecular formats and therapeutic applications for bispecific antibodies.” Mol Immunol 67(2 Pt A): 95-106.
Stavenhagen, J. B., S. Gorlatov, N. Tuaillon, C. T. Rankin, H. Li, S. Burke, L. Huang, S. Vijh, S. Johnson, E. Bonvini and S. Koenig (2007). “Fc optimization of therapeutic antibodies enhances their ability to kill tumor cells in vitro and controls tumor expansion in vivo via low-affinity activating Fcgamma receptors.” Cancer Res 67(18): 8882-8890.
Tao, M. H. and S. L. Morrison (1989). “Studies of aglycosylated chimeric mouse-human IgG. Role of carbohydrate in the structure and effector functions mediated by the human IgG constant region.” J Immunol 143(8): 2595-2601.
Thaysen-Andersen, M., E. Chertova, C. Bergamaschi, E. S. Moh, O. Chertov, J. Roser, R. Sowder, J. Bear, J. Lifson, N. H. Packer, B. K. Felber and G. N. Pavlakis (2016). “Recombinant human heterodimeric IL-15 complex displays extensive and reproducible N- and O-linked glycosylation.” Glycoconj J 33(3): 417-433.
Toutain, P. L. and A. Bousquet-Melou (2004). “Plasma terminal half-life.” J Vet Pharmacol Ther 27(6): 427-439.
Vafa, O., G. L. Gilliland, R. J. Brezski, B. Strake, T. Wilkinson, E. R. Lacy, B. Scallon, A. Teplyakov, T. J. Malia and W. R. Strohl (2014). “An engineered Fc variant of an IgG eliminates all immune effector functions via structural perturbations.” Methods 65(1): 114-126.
Villa, A., E. Trachsel, M. Kaspar, C. Schliemann, R. Sommavilla, J. N. Rybak, C. Rosli, L. Borsi and D. Neri (2008). “A high-affinity human monoclonal antibody specific to the alternatively spliced EDA domain of fibronectin efficiently targets tumor neo-vasculature in vivo.” Int J Cancer 122(11): 2405-2413.
Von Kreudenstein, T. S., E. Escobar-Carbrera, P. I. Lario, I. D'Angelo, K. Brault, J. Kelly, Y. Durocher, J. Baardsnes, R. J. Woods, M. H. Xie, P. A. Girod, M. D. Suits, M. J. Boulanger, D. K. Poon, G. Y. Ng and S. B. Dixit (2013). “Improving biophysical properties of a bispecific antibody scaffold to aid developability: quality by molecular design.” MAbs 5(5): 646-654.
Vonderheide, R. H. (2020). “CD40 Agonist Antibodies in Cancer Immunotherapy.” Annual Review of Medicine 71(1): 47-58.
Waldmann, T. A. (2015). “The shared and contrasting roles of IL2 and IL15 in the life and death of normal and neoplastic lymphocytes: implications for cancer therapy.” Cancer Immunol Res 3(3): 219-227.
Waldmann, T. A., S. Dubois, M. D. Miljkovic and K. C. Conlon (2020). “IL-15 in the Combination Immunotherapy of Cancer.” Frontiers in Immunology 11(868).
Waldmeier, L., I. Hellmann, C. K. Gutknecht, F. I. Wolter, S. C. Cook, S. T. Reddy, U. Grawunder and R. R. Beerli (2016). “Transpo-mAb display: Transposition-mediated B cell display and functional screening of full-length IgG antibody libraries.” MAbs 8(4): 726-740.
Walker, M. R., J. Lund, K. M. Thompson and R. Jefferis (1989). “Aglycosylation of human IgG1 and IgG3 monoclonal antibodies can eliminate recognition by human cells expressing Fc gamma RI and/or Fc gamma RII receptors.” Biochem J 259(2): 347-353.
Wang, X., M. Mathieu and R. J. Brezski (2018). “IgG Fc engineering to modulate antibody effector functions.” Protein Cell 9(1): 63-73.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200(1): 16-26.
Xu, Y., L. C. Carrascosa, Y. A. Yeung, M. L. Chu, W. Yang, I. Djuretic, D. C. Pappas, J. Zeytounian, Z. Ge, V. de Ruiter, G. R. Starbeck-Miller, J. Patterson, D. Rigas, S. H. Chen, E. Kraynov, P. P. Boor, L. Noordam, M. Doukas, D. Tsao, J. N. Ijzermans, J. Guo, D. J. Grunhagen, J. Erdmann, J. Verheij, M. E. van Royen, P. G. Doornebosch, R. Feldman, T. Park, S. Mahmoudi, M. Dorywalska, I. Ni, S. M. Chin, T. Mistry, L. Mosyak, L. Lin, K. A. Ching, K. C. Lindquist, C. Ji, L. M. Londono, B. Kuang, R. Rickert, J. Kwekkeboom, D. Sprengers, T. H. Huang and J. Chaparro-Riggers (2021). “An Engineered IL15 Cytokine Mutein Fused to an Anti-PD1 Improves Intratumoral T-cell Function and Antitumor Immunity.” Cancer Immunol Res 9(10): 1141-1157.
Yamane-Ohnuki, N., S. Kinoshita, M. Inoue-Urakubo, M. Kusunoki, S. Iida, R. Nakano, M. Wakitani, R. Niwa, M. Sakurada, K. Uchida, K. Shitara and M. Satoh (2004). “Establishment of FUT8 knockout Chinese hamster ovary cells: an ideal host cell line for producing completely defucosylated antibodies with enhanced antibody-dependent cellular cytotoxicity.” Biotechnol Bioeng 87(5): 614-622.
Yu, J., Y. Song and W. Tian (2020). “How to select IgG subclasses in developing anti-tumor therapeutic antibodies.” J Hematol Oncol 13(1): 45.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28(2): 157-159.
Zhang, T., X. Song, L. Xu, J. Ma, Y. Zhang, W. Gong, Y. Zhang, X. Zhou, Z. Wang, Y. Wang, Y. Shi, H. Bai, N. Liu, X. Yang, X. Cui, Y. Cao, Q. Liu, J. Song, Y. Li, Z. Tang, M. Guo, L. Wang and K. Li (2018). “The binding of an anti-PD-1 antibody to FcγTRI has a profound impact on its biological functions.” Cancer Immunology, Immunotherapy 67(7): 1079-1090.
WO 2018/184964A1
WO2019/166946A1

1. An immunocytokine comprising:
a. a conjugate comprising a polypeptide comprising an interleukin 15 (IL-15) or a derivative thereof and the sushi domain of an interleukin 15-receptor alpha (IL-15Rα) or a derivative thereof,
wherein the IL-15 derivative comprises at least one mutation that reduces the binding to the IL-2/IL-15Rβ and/or to the γ_{c} receptor, and
b. an antibody or a functional variant thereof,
wherein the functional variant of the antibody is characterized by:
i. a heterodimeric Fc domain,
ii. a modified effector function, and/or
iii. an increased in vivo half-life;
wherein the conjugate is fused directly or indirectly to the C-terminus of both antibody heavy chains or antibody light chains, or, in case of i., to the C-terminus of one antibody heavy chain.

2. The immunocytokine of claim 1, wherein the immunocytokine comprises a functional variant of an antibody.

3. The immunocytokine of claim 1 or 2, wherein the modified effector function is reduced antibody-dependent cell toxicity and wherein the antibody or functional variant thereof
a. is an IgG1 antibody or a functional variant thereof and comprises a mutation selected from L234A/L235A, P329G, L234A/L235A/P329G, G236R/L328R, D265A, N297A, N297Q, N297G or L234A/L235A/G237A/P238S/H268A/A330S/P331S,
b. is an IgG4 antibody or a functional variant thereof and comprises a mutation selected from L235E, F234A/L235A, F234A/L235A/P329G, P329G, S228P/L235E, S228P/F234A/L235A or E233P/F234V/L235A/D265A/R409K,
c. is a IgG2 (IgG2a or IgG2b) and IgG4 hybrid or a functional variant thereof and comprises a CH1 and hinge region from IgG2, and CH2 and CH3 regions are from IgG4 (IgG2 amino acids 118 to 260 and the IgG4 amino acids 261 to 447), or
d. is an IgG2 antibody or a functional variant thereof and comprises a mutation selected from H268Q/V309L/A330S/P331S or V234A/G237A/P238S/H268A/V309L/A330S/P331S,
wherein numbering is according to EU numbering.

4. The immunocytokine of claim 3, wherein the antibody or functional variant thereof
(a) is an IgG4 antibody or a functional variant thereof and comprises a L235E mutation, or
(b) is an IgG1 antibody or a functional variant thereof and comprises a L234A/L235A mutation.

5. The immunocytokine of claim 1, wherein the modified effector function is enhanced antibody-dependent cell toxicity and wherein the antibody or functional variant thereof:
a. is an IgG1 antibody or a functional variant thereof and comprises a mutation selected from F243L/R292P/Y300L/V305I/P396L, S239D/I332E, S239D/I332E/A330L, S298A/E333A/K334A, K392T/P396L, V264I/I332E or L234Y/L235Q/G236W/S239M/H268D/D270E/S298A,
preferably from S239D/I332E, S239D/I332E/A330L, S298A/E333A/K334A, K392T/P396L, V264I/I332E, in one heavy chain and further comprises a D270E/K326D/A330M/K334E mutation in the opposing heavy chain, and/or
b. is an afucosylated IgG1, IgG2 or IgG4 antibody or a functional variant thereof,
wherein numbering is according to EU numbering.

6. The immunocytokine of any of the claims 1 to 5 wherein the heterodimeric Fc domain is selected from KiH, KiH_{S-S}, HA-TF, ZW1, 7.8.60, DD-KK, EW-RVT, EW-RVT_{S-S}, SEED and A107, preferably KiH.

7. The immunocytokine of any of the claims 1 to 6, wherein the heterodimeric Fc domain leads to higher yield of the immunocytokine upon expression in cell culture, compared to an immunocytokine with homodimeric Fc domain.

8. The immunocytokine of any of the claims 1 to 7, wherein the half-life of the immunocytokine is increased and wherein the antibody or functional variant thereof is an IgG1 or an IgG4 antibody or a functional variant thereof and comprises a mutation selected from M252Y/S254T/T256E, M428L/N434S or T250Q/M428L,
wherein numbering is according to EU numbering.

9. The immunocytokine of any of the claims 1 to 4, wherein the antibody or functional variant thereof has reduced antibody-dependent cellular cytotoxicity and wherein the antibody or functional variant thereof is an IgG4 antibody or a functional variant thereof and comprises a L235E mutation and a KiH heterodimerized Fc domain.

10. The immunocytokine of any of claims 1 to 9, wherein the conjugate is a fusion protein comprising, in N- to C-terminal order, the IL-15Rα sushi domain or a derivative thereof, a linker and the IL-15 or a derivative thereof, preferably wherein the IL-15Rα sushi domain comprises the sequence of SEQ ID NO: 5, and
wherein the linker has a length of 18 to 22 amino acids and is composed preferably of glycines or serines and glycines, more preferably has the sequence of SEQ ID NO: 7, and preferably wherein the IL-15 has the sequence of SEQ ID NO: 2.

11. The immunocytokine of any of claims 1 to 10, wherein the IL-15 variant comprises:
a. at least one mutation increasing the homogeneity of the IL-15 variant with respect to post-translational modifications,
preferably wherein the mutation reduces deamidation at N77 and/or glycosylation at N79 of IL-15 (SEQ ID NO: 2),
more preferably wherein the mutation is selected from mutations G78A, G78V, G78L or G78I, and N79Q, N79S or N79T,
most preferably wherein the mutation is G78A/N79Q; and/or
b. at least one mutation that reduces the binding to the IL-2/IL-15Rβ and/or to the γ_{c} receptor,
wherein the mutated amino acid is selected from N1, N4, S7, D8, K10, K11, D30, D61, E64, N65, L69, N72, E92, Q101, Q108, I111 of IL-15 (SEQ ID NO: 2), preferably wherein the mutated amino acid is selected from D61, N65 and Q101, more preferably wherein the mutated amino acid is N65.

12. The immunocytokine of claim 11, wherein the at least one mutation that reduces the binding to the IL-2/IL-15Rβ and/or to the γ_{c} receptor is a substitution selected from N1D, N1A, N1G, N4D, S7Y, S7A, D8A, D8N, K10A, K11A, D30N, D61A, D61N, E64Q, N65D, N65A, N65E, N65R, N65K, L69R, N72R, Q101D, Q101E, Q108D, Q108A, Q108E and Q108R, preferably D8A, D8N, D61A, D61N, N65A, N65D, N72R, Q101D, Q101E and Q108A, more preferably D61A, N65A and Q101D, most preferably N65A or a combined substitution selected from D8N/N65A, D61A/N65A or D61A/N65A/Q101D.

13. The immunocytokine of any of the claims 1 to 12, wherein the antibody or functional variant thereof:
a. binds to a tumor antigen, preferably selected from EGFR, HER2, FGFR2, FOLR1, CLDN18.2, CEA, GD2, O-Acetyl-GD-2, GM1, CAIX, EPCAM, MUC1, PSMA, c-MET, ROR1, GPC3, CD19, CD20, CD38;
b. binds to a tumor extracellular matrix antigen, preferably selected from FAP, the EDA domain of fibronectin, the EDB domain of fibronectin and LRRC15, preferably FAP and the EDB domain of fibronectin;
c. binds to a neovascularization antigen, preferably VEGF, or Endoglin;
d. is an immunomodulatory antibody or a functional variant thereof,
wherein the immunomodulatory antibody stimulates a co-stimulatory receptor, preferably selected from CD40 agonists, CD137/4-1BB agonists, CD134/OX40 agonists and TNFRSF18/GITR agonists, or
wherein the immunomodulatory antibody inhibits an immunosuppressive receptor, preferably selected from PD-1 antagonists, CTLA-4 antagonists, LAG3 antagonists, TIGIT antagonists, inhibitory KIRs antagonists, BTLA antagonists, HAVCR2 antagonists and ADORA2A antagonists, more preferably PD-1 antagonists.

14. The immunocytokine of claim 1, wherein
the cytokine domain comprises the sequence of SEQ ID NO: 10; and
the antibody comprises:
i. the heavy chain knob sequence of SEQ ID NO:20
ii. the heavy chain hole sequence of SEQ ID NO: 22 or SEQ ID NO: 101, preferably SEQ ID NO 101, and
iii. the light chain sequence of SEQ ID NO: 16;
wherein the cytokine domain is fused to the C-terminus heavy chain knob sequence without a linker.

15. The immunocytokine of claim 1, wherein
the cytokine domain comprises the sequence of SEQ ID NO: 10; and
the antibody comprises:
i. the heavy chain knob sequence of SEQ ID NO:84
ii. the heavy chain hole sequence of SEQ ID NO: 87, and
iii. the light chain sequence of SEQ ID NO: 88;
wherein the cytokine domain is fused to the C-terminus heavy chain knob sequence without a linker.

16. The immunocytokine of claim 1, wherein
the cytokine domain comprises the sequence of SEQ ID NO: 10; and
the antibody comprises:
i. the heavy chain knob sequence of SEQ ID NO:93,
ii. the heavy chain hole sequence of SEQ ID NO: 95, and
iii. the light chain sequence of SEQ ID NO: 92;
wherein the cytokine domain is fused to the C-terminus heavy chain knob sequence without a linker.

17. The immunocytokine of claim 1, wherein
the cytokine domain comprises the sequence of SEQ ID NO: 10; and
the antibody comprises:
i. the heavy chain knob sequence of SEQ ID NO: 109,
ii. the heavy chain hole sequence of SEQ ID NO: 110, and
iii. the light chain sequence of SEQ ID NO: 88;
wherein the cytokine domain is fused to the C-terminus heavy chain knob sequence without a linker.
